SEQUENCE LISTING (20 sequences)

SEQ ID NO: 1 - protein, 6 aa, Artificial Sequence
  Description of Artificial Sequence Synthetic 6xHis tag
HHHHHH

SEQ ID NO: 2 - protein, 7 aa, Tobacco etch virus
ENLYFQG

SEQ ID NO: 3 - protein, 190 aa, Artificial Sequence
  Description of Artificial Sequence Synthetic polypeptide
MGSSHHHHHHSSGENLYFQGSMTEYKLVVVGAGGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 4 - protein, 190 aa, Artificial Sequence
  Description of Artificial Sequence Synthetic polypeptide
MGSSHHHHHHSSGENLYFQGSMTEYKLVVVGACGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 5 - protein, 190 aa, Artificial Sequence
  Description of Artificial Sequence Synthetic polypeptide
MGSSHHHHHHSSGENLYFQGSMTEYKLVVVGACGVGKVALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 6 - protein, 190 aa, Artificial Sequence
  Description of Artificial Sequence Synthetic polypeptide
MGSSHHHHHHSSGENLYFQGSMTEYKLVVVGACGVGKSALIIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 7 - protein, 190 aa, Artificial Sequence
  Description of Artificial Sequence Synthetic polypeptide
MGSSHHHHHHSSGENLYFQGSMTEYKLVVVGACGVGKSALFIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 8 - protein, 190 aa, Artificial Sequence
  Description of Artificial Sequence Synthetic polypeptide
MGSSHHHHHHSSGENLYFQGSMTEYKLVVVGACGVGKSALTIQLIQNHFVDEYDPTIEDSYRKQVVIDGETCLLDFLDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 9 - protein, 190 aa, Artificial Sequence
  Description of Artificial Sequence Synthetic polypeptide
MGSSHHHHHHSSGENLYFQGSMTEYKLVVVGACGVGKSALTIQLIQNHFVDEYDPTIEDSYRKQVVIDGETCLLDILETAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 10 - protein, 190 aa, Artificial Sequence
  Description of Artificial Sequence Synthetic polypeptide
MGSSHHHHHHSSGENLYFQGSMTEYKLVVVGACGVGKSALTIQLIQNHFVDEYDPTIEDSYRKQVVIDGETCLLDILFTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 11 - protein, 190 aa, Artificial Sequence
  Description of Artificial Sequence Synthetic polypeptide
MGSSHHHHHHSSGENLYFQGSMTEYKLVVVGACGVGKSALTIQLIQNHFVDEYDPTIEDSYRKQVVIDGETCLLDILRTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 12 - protein, 190 aa, Artificial Sequence
  Description of Artificial Sequence Synthetic polypeptide
MGSSHHHHHHSSGENLYFQGSMTEYKLVVVGACGVGKSALTIQLIQNHFVDEYDPTIEDSYRKQVVIDGETCLLDILDAAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 13 - protein, 190 aa, Artificial Sequence
  Description of Artificial Sequence Synthetic polypeptide
MGSSHHHHHHSSGENLYFQGSMTEYKLVVVGACGVGKSALTIQLIQNHFVDEYDPTIEDSYRKQVVIDGETCLLDILDVAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 14 - protein, 190 aa, Artificial Sequence
  Description of Artificial Sequence Synthetic polypeptide
MGSSHHHHHHSSGENLYFQGSMTEYKLVVVGACGVGKSALTIQLIQNHFVDEYDPTIEDSYRKQVVIDGETCLLDILDFAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 15 - protein, 190 aa, Artificial Sequence
  Description of Artificial Sequence Synthetic polypeptide
MGSSHHHHHHSSGENLYFQGSMTEYKLVVVGACGVGKSALTIQLIQNHFVDEYDPTIEDSYRKQVVIDGETCLLDILDTAAQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 16 - protein, 190 aa, Artificial Sequence
  Description of Artificial Sequence Synthetic polypeptide
MGSSHHHHHHSSGENLYFQGSMTEYKLVVVGACGVGKSALTIQLIQNHFVDEYDPTIEDSYRKQVVIDGETCLLDILDTAWQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 17 - protein, 190 aa, Artificial Sequence
  Description of Artificial Sequence Synthetic polypeptide
MGSSHHHHHHSSGENLYFQGSMTEYKLVVVGACGVGKSALTIQLIQNHFVDEYDPTIEDSYRKQVVIDGETCLLDILDTAGQEEYSAMRDQWMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 18 - protein, 190 aa, Artificial Sequence
  Description of Artificial Sequence Synthetic polypeptide
MGSSHHHHHHSSGENLYFQGSMTEYKLVVVGAVGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEK

SEQ ID NO: 19 - protein, 8 aa, Tobacco etch virus
ENLYFQGG

SEQ ID NO: 20 - protein, 327 aa, Artificial Sequence
  Description of Artificial Sequence Synthetic polypeptide
MSPILGYWKIKGLVQPTRLLLEYLEEKYEEHLYERDEGDKWRNKKFELGLEFPNLPYYIDGDVKLTQSMAIIRYIADKHNMLGGSPKERAEISMLEGAVLDIRYGVSRIAYSKDFETLKVDFLSKLPEMLKMFEDRLSHKTYLNGDHVTHPDFMLYDALDVVLYMDPMSLDAFPKLVSFKKRIEAIPQIDKYLKSSKYIAWPLQGWQATFGGGDHPPKSDLVPRGSGSENLYFQGGLNDIFEAQKIEWRSNTIRVFLPNKQRTVVNVRNGMSLHDCLMKALKVRGLQPECCAVFRLLHEHKGKKARLDWNTDAASLIGEELQVDFLD

(19) United States
(12) Patent Application Publication
(10) Pub. No.: US 2019/0134056 A1
(43) Pub. Date: May 9, 2019

(54) K-RAS MUTATIONS AND ANTAGONISTS

(71) Applicant: The Trustees of the Stevens Institute of Technolog, Hoboken, NJ (US)

(72) Inventors: Peter TOLIAS, Westfield, NJ (US); Alvin S. STERN, Fair Lawn, NJ (US); Kuo-Sen HUANG, Livingston, NJ (US); Michael Lloyd SABIO, Ridgewood, NJ (US); Sidney Wolf TOPIOL, Fair Lawn, NJ (US)

(73) Assignee: The Trustees of the Stevens Institute of Technolog, Hoboken, NJ (US)

(21) Appl. No.: 15/917,527

(22) Filed: Mar. 9, 2018

Related U.S. Application Data
(60) Provisional application No. 62/469,848, filed on Mar. 10, 2017.

(51) Int. Cl. A61K 31/553; A61K 31/519; A61K 31/4709; A61K 31/4704; A61K 31/498; A61K 31/5415; A61K 31/536; A61K 31/4155; A61K 31/415; A61K 31/4196; A61K 31/4025; A61K 31/40; A61K 31/4453; A61K 31/4184; A61K 31/454; A61K 31/404; A61K 31/4439; A61K 31/527; A61K 31/517; A61K 31/522; A61K 31/4188; A61K 31/428; A61K 31/4985; A61K 31/435; A61K 31/422

(52) U.S. Cl. CPC A61K 31/553; A61K 31/422; A61K 31/4709; A61K 31/4704; A61K 31/498; A61K 31/5415; A61K 31/536; A61K 31/4155; A61K 31/415; A61K 31/4196; A61K 31/4025; A61K 31/40; A61K 31/4453; A61K 31/4184; A61K 31/454; A61K 31/404; A61K 31/4439; A61K 31/527; A61K 31/517; A61K 31/522; A61K 31/4188; A61K 31/428; A61K 31/4985; A61K 31/435; A61K 31/519

(57) ABSTRACT

The present application relates to K-Ras mutations, to polynucleotides encoding mutant K-Ras polypeptides, and to methods of identifying small molecule antagonists using K-Ras mutations. The present application also relates to K-Ras small molecule antagonists and use thereof in the treatment of tumors.

### CROSS-REFERENCE TO RELATED APPLICATION

The present application claims priority to U.S. Provisional Application No. 62/469,848, filed Mar. 10, 2017 and entitled K-RAS MUTATIONS AND ANTAGONISTS, the disclosures of which provisional application, including its specification, claims, abstract, appendix, and drawings, are incorporated herein by reference in their entireties.

### FIELD OF INVENTION

The present disclosure relates generally to cancer therapeutics, methods of identifying cancer therapeutics, and methods of treating cancer. More specifically, the disclosure relates to mutations of K-Ras polypeptides, polynucleotides encoding mutant K-Ras polypeptides, and to methods of identifying small-molecule antagonists of K-Ras using the K-Ras mutations. The disclosure relates to the use of K-Ras polypeptides or mutants thereof, polynucleotides encoding K-Ras polypeptides or mutants thereof, and small molecules that act on K-Ras polypeptides or mutations thereof to facilitate the discovery of small-molecule K-Ras antagonists. The present disclosure also relates to K-Ras small-molecule antagonists, pharmaceutical compositions comprising such antagonists, and their use in the treatment of cancer and cancer tumors.

### BACKGROUND

RAS proteins represent prototypical members of a large family of small GTP-binding proteins. The human RAS superfamily consists of more than 100 members, which can be divided into six subfamilies. Three prototypical RAS proteins include H-Ras, N-Ras, and K-Ras. While they are highly homologous in amino acid sequence and are ubiquitously expressed, K-Ras is the only one that is essential for normal development as shown by mouse genetic studies. K-Ras can be expressed as two different splice variants, referred to as 4A and 4B, through alternative splicing within exon 4. The 4B variant is the dominant form commonly known as K-Ras.

K-Ras is a membrane-bound GTPase that cycles between an active GTP-bound form and an inactive GDP-bound form due to the hydrolysis of the bound GTP. The switches between these two states are controlled by two classes of proteins: guanosine nucleotide exchange factors (known as GEFs) and GTPase-activating proteins (known as GAPs). As their names suggest, GEFs assist with the exchange of bound GDP with GTP, whereas GAPs stimulate the hydrolytic ability of RAS to convert bound GTP to GDP.

K-Ras mediates a myriad of intracellular signaling events through its numerous effector pathways. Signaling by receptor tyrosine kinases (RTKs), in particular the epidermal growth factor receptor (EGFR), is a widely utilized and well-understood model for studying K-Ras activation. The activation of EGFR upon ligand binding and its subsequent auto-phosphorylation create a docking site for the adaptor protein growth-factor-receptor-bound protein 2 (GRB2), which binds to the GEF Son of Sevenless (SOS) in the cytosol. The recruitment of this protein complex to the phosphorylated receptor enables SOS to function as the exchange factor for K-Ras, resulting in nucleotide exchange and the GTP-bound form of K-Ras.

Among numerous downstream effectors of K-Ras, the best characterized include RAF and phosphoinositide-3 kinase (PI3K), as well as the GEFs for the RAS-like (Ral) small GTPases (RalGEFs). The major axes of RAS signaling through the RAF/MEK/ERK and PI3K/AKT cascades ultimately control processes such as cell growth and survival. This is accomplished in part by ERK-regulated activation of transcription factors that promote cell cycle progression, and by AKT-mediated inactivation of pro-apoptotic proteins for apoptosis suppression. In addition, several alternate effectors of K-Ras have been described in an extensive body of literature, which regulate processes such as cell migration, endocytosis, changes in cytoskeleton, and calcium signaling.

Nearly 30% of human cancers possess activating RAS mutations, 85% of which are K-Ras mutations. The vast majority of K-Ras mutations are located in codons 12 and 13 (in the P-loop), and the remainder in codons 61, 146 (in the base-binding loops), and other residues. While the hotspot codon 12 and 13 mutations of K-Ras do not interfere with its ability to associate with GAPs, they alter the position of a catalytic glutamate residue at codon 61. This results in the reduced GTPase activity of K-Ras and decreased rate of GTP hydrolysis by 3-9 fold compared to that of wild-type (WT) K-Ras. Recent studies have illustrated that these mutations play a significant role in tumor cell survival and tumor progression. The functional consequences of K-Ras mutations include increased cellular proliferation, suppression of apoptosis, altered cell metabolism, and changes in the tumor microenvironment. For example, GTP-bound K-Ras enables the upregulation of growth factors and transcription factors known to promote cell cycle progression, such as c-JUN and c-FOS. Recent studies identified the Yes-associated protein 1 (YAP 1) transcriptional co-activator as a key mediator of the oncogenic effect of K-Ras. From a therapeutic standpoint, suppression of apoptosis is probably one of the most important consequences of K-Ras mutations. Activated or hyperactivated K-Ras can inhibit the apoptotic signaling cascade through its effector PI3K, which in turn activates AKT, a potent pro-survival kinase that inhibits apoptosis via several mechanisms, such as the phosphorylation and subsequent inactivation of the pro-apoptotic Bcl-2 family protein BAD, and the inhibitory phosphorylation of the initiator caspase-9.

The structural basis for the Ras cycle and Ras hyperactivation are well understood. Over 40 crystal structures of H-Ras have been solved, including both wild-type and mutants bound to GDP or analogs of GTP. Likewise, the structures of H-Ras in complex with many of its binding partners are known. The nucleotide-binding pocket is bordered by four main regions: the phosphate-binding loop (P-loop, residues 10-17), Switch 1 (residues 30-40), Switch 2 (residues 60-76), and the base-binding loops (residues 116-120 and 145-147), (Hall et al. PNAS, 2002, 19, 12138-12142 and Vetter 2001 Science). The Switch regions govern interactions between Ras and its binding partners by adopting different conformations when bound to GTP or GDP. Threonine-35 and glycine-60 make key hydrogen bonds with the γ-phosphate of GTP, holding the Switch 1 and Switch 2 regions in the active conformation, respectively. Upon hydrolysis of GTP and release of phosphate, these two regions are free to relax into the inactive GDP conformation.

The regions bordering the nucleotide pocket also contain the most common sites of Ras mutation in cancer. The vast majority of oncogenic mutations occur at residues 12 or 13 in the P-loop, or residue 61 in Switch 2. Structural data suggest that mutation of glycine-12 or glycine-13 would sterically occlude the critical arginine residue of the GAP and thus prohibit inactivation of Ras signaling. Mutation of glutamine-61 similarly impairs GAP-mediated Ras inactivation.

Despite numerous attempts, developing small-molecule inhibitors of K-Ras has proven to be extremely challenging. Several recent reports have described novel small molecules that interfere with GEF binding to lock K-Ras in an inactive state. For example, in silico and NMR-based screens were used to identify small molecules that bind to a distinct pocket on K-Ras and inhibit SOS-mediated nucleotide exchange to prevent the activation of WT or mutant K-Ras. More specific approaches have been utilized to identify small-molecule inhibitors that covalently bind to mutant K-Ras. A disulphide-fragment-based screening approach was used to identify electrophilic compounds that can form a disulfide bond with the cysteine residue in the G12C mutant of K-Ras (Ostrem et al., Nature 2013 Nov. 28; 503(7477):548-51). These compounds are found to covalently bind within a pocket referred to as the “irreversible site.” While these compounds do not affect WT K-Ras, they can preferentially bind the G12C mutant, disrupt SOS binding, and increase its affinity for GDP to prevent the activation of mutant K-Ras. A similar approach identified a GDP analog as a covalent inhibitor of the G12C mutant (Lim et al., Angew Chem. Int. Ed. Engl. 2014 Jan. 3; 53(1):199-204). Additionally, NMR spectroscopy and molecular modeling techniques have been utilized to identify “small organic inhibitors” of the Ras exchange process. These molecules are said to bind to the Ras protein in a previously unidentified binding pocket without displacing GDP. These inhibitors contain a potentially reactive hydroxylamine group, which may covalently bind to the Ras proteins (Taveras et al., Bioorganic & Medicinal Chem 1997; 5(1): 125-133).

However, the covalent inhibitors of K-Ras G12C have certain disadvantages, including off-target effects due to their high reactivity, irreversibility due to covalent modifications, as well as adverse drug reactions caused by immunogenic drug-protein adducts. Thus, there is a need in the art for effective K-Ras inhibitors, methods for identifying K-Ras inhibitors, and K-Ras targeting anticancer compounds, as well as pharmaceutical compositions comprising K-Ras targeting anticancer compounds.

The foregoing discussion is solely to provide a better understanding of the context of the present disclosure and should not be construed in any way as an admission as to prior art.

### SUMMARY OF INVENTION

The present invention provides compounds for inhibiting K-Ras which are believed to be useful for treating various cancers. Also provided are pharmaceutical compositions comprising one or more compounds according to the invention in combination with one or more carriers or excipients. Related methods of treated cancer using the compounds of the invention are alss disclosed. In some embodiments, the cancer is associated with wild type or mutant K-Ras, such as K-Ras (G12C). These and other aspects of the invention will be apparent to those skilled in the art from the following detailed description, which is simply, by way of illustration, various modes contemplated for carrying out the invention. As will be realized, the invention is capable of additional, different obvious aspects, all without departing from the invention. Accordingly, the Figures and specification are illustrative in nature and not restrictive.

### BRIEF DESCRIPTION OF THE DRAWINGS

FIG. 1 depicts the 3-dimensional (“3D”) structure of mouse K-Ras protein, with binding sites A, B, C, and D illustrated as modeled using Protein Data Bank entry 3KKP.

FIGS. 2A and 2B show the 3D structure of human K-Ras(G12C), site A (FIG. 2A), along with the design of additional mutations (FIG. 2D) as modeled using Protein Data Bank entry 4LV6.

FIG. 3 illustrates the Ras/Raf binding assay and the results for Ras(G12C) and Ras(WT).

### DETAILED DESCRIPTION

In one aspect, the present disclosure provides mutant K-Ras proteins that can be used to enable the discovery of new molecules that modulate an activity of K-Ras. The new K-Ras mutants disclosed herein are designed to increase the availability of protein conformations that open up, or improve accessibility to, certain pockets in K-Ras (e.g., site A and/or site D), to allow the screening of new compounds that can bind site A more effectively, reversibly, and/or non-covalently. Reversible inhibitors can be subsequently optimized in accordance with the general principles of drug development and lead optimization known in the art, to select compounds with more favorable ADME (absorption, distribution, metabolism, and excretion) and toxicological profiles. In some embodiments, a small primer molecule can be used to enhance sensitivity in testing potential inhibitors of mutant K-Ras proteins. In some embodiments, in silico screening and computational chemistry studies can be used to identify such compounds, followed by biochemical in vitro and in vivo testing. In certain embodiments, one or more combinatorial chemical libraries can be used for the screening.

Also provided herein are pharmaceutical agents, such as “small molecule” (e.g., molecules having a molecule are weight less than 2K Daltons) pharmaceutical agents, that can be used as therapeutics on cancer patients that have alterations in their K-Ras gene and/or protein. The pharmaceutical agents according to the invention are contemplated to be highly selective and effective against cancer cells that contain mutated forms of the K-Ras gene (which are implicated in approximately 30% of all cancers). The compounds disclosed herein can be administered as a pharmaceutical drug(s) to patients with cancers that contain K-Ras mutations (e.g., G12C), for example, by incorporating them into dosage forms (e.g., solid dosage forms) for administration (e.g., oral administration) to a patient in need thereof. In some embodiments, a patient in need thereof is a patient suffering from cancer, in particular, a cancer in which the cancer cells express a mutant K-Ras protein, and include patients for which a clinical diagnosis has been made that cancer cells are characterized by a mutation in the K-Ras gene and/or the cancer cells contain or express a mutant K-Ras protein.

In another aspect, provided herein is a new process for computational and biochemical discovery of small molecule drugs against the K-Ras protein (e.g., in cancer patients) that uses: 1) the wild type K-Ras protein, 2) a genetically modified version of K-Ras protein (G12C) that is associated with a variety of different cancers, 3) genetically modified versions of the K-Ras protein that have synthetic mutations engineered to facilitate the entry and binding of drugs, and/or 4) genetically modified versions of the K-Ras protein (G12C) that also contain synthetic mutations engineered to facilitate the entry and binding of drugs. Also provided are pharmaceutical agents, such as small molecules, identified by the above mentioned process for identifying or screening drug candiates that can be bound and can inhibit the activity of K-Ras. Pharmaceutical compositions comprising therapeutically effective amounts of the pharmaceutical agents in combination with one or more excipients, such as a physiologically compatiable carrier, are also provided.

In another aspect, of the invention, a new protocol is provided for identifying molecules (e.g., small molecules) that modulate activity of K-Ras. These modulators of K-Ras may bind to or otherwise interact with sites that are allosteric to site A of K-Ras and/or any K-Ras mutants described herein. In some embodiments, the interaction of the modulators induces protein conformational changes which enhance the affinity of compounds at site A. Any such allosteric modulator may serve as a primer for the protein. The primer effect increases the sensitivity of the protein to site A ligands to facilitate the identification of active compounds.

### Definitions

For convenience, certain terms employed in the specification, including the examples and appended claims, are collected here. Unless defined otherwise, all technical and scientific terms used herein have the same meaning as commonly understood by one of ordinary skill in the art to which this disclosure pertains.

Unless otherwise explicitly defined, the following terms and phrases are intended to have the following meanings throughout this disclosure:

All percentages given herein refer to the weight percentages of a particular component relative to the entire composition, including the carrier, unless otherwise indicated. It will be understood that the sum of all weight % of individual components within a composition will not exceed 100%.

The terms “a” or “an,” as used in herein means one or more. As used herein, the term “consisting essentially of” is intended to limit the invention to the specified materials or steps and those that do not materially affect the basic and novel characteristics of the claimed invention, as understood from a reading of this specification. The term “physiologically compatible” means that the component is generally regarded as safe and non-toxic for contact with human tissues at the levels employed.

The phrase “individual in need thereof” denotes an individual having cancer. In some implementations, the indivual in need thereof is a patient that has been diagnosed with a cancer characterized by expression of a mutant K-Ras protein and/or the presence of a mutation in a gene encoding a K-Ras polypeptide. The term “prevent,” as used herein, includes delaying the onset of or progression of a disease or physiological manifestation of disease. The term “treat” includes reducing, diminishing, eliminating, ameliorating, forestalling, slowing the progression of, and/or delaying the onset of a given disease or physiological manifestation thereof.

The following definitions of various groups or substituents are used, unless otherwise described. Specific and general values listed below for radicals, substituents, and ranges, are for illustration only; they do not exclude other defined values or other values within defined ranges for the radicals and substituents. Unless otherwise indicated, alkyl, alkenyl, alkynyl, alkoxy, and the like denote straight, branched, and cyclic groups, as well as any combination thereof.

The term “hydrocarbon” refers to a radical or group containing carbon and hydrogen atoms. Examples of hydrocarbon radicals include, without limitation, alkyl, alkenyl, alkynl, aryl, aryl-alkyl, alkyl-aryl, and any combination thereof (e.g., alkyl-aryl-alkyl, etc.). As used herein, unless otherwise indicated, hydrocarbons may be monovalent or multivalent (e.g., divalent, trivalent, etc) hydrocarbon radicals. A radical of the form —(CH₂)ₙ—, including a methylene radical, i.e., —CH₂—, is regarded as an alkyl radical if it does not have unsaturated bonds between carbon atoms. Unless otherwise specified, all hydrocarbon radicals (including substituted and unsubstituted alkyl, alkenyl, alkynyl, aryl, aryl-alkyl, alkyl-aryl, etc.) will have from 1-20 carbon atoms. In other embodiments, hydrocarbons will have from 1-12 or from 1-8 or from 1-6 or from 1-3 carbon atoms, including for example, embodiments having one, two, three, four, five, six, seven, eight, nine, or ten carbon atoms.

A “substituted” hydrocarbon may have as a substituent one or more hydrocarbon radicals, substituted hydrocarbon radicals, or may comprise one or more heteroatoms. Examples of substituted hydrocarbon radicals include, without limitation, heterocycles, such as heteroaryls. Unless otherwise specified, a hydrocarbon substituted with one or more heteroatoms will comprise from 1-20 heteroatoms. In other embodiments, a hydrocarbon substituted with one or more heteroatoms will comprise from 1-12 or from 1-8 or from 1-6 or from 1-4 or from 1-3 or from 1-2 heteroatoms. Examples of heteroatoms include, but are not limited to, oxygen, nitrogen, sulfur, phosphorous, halogen (F, Cl, Br, I, etc.), boron, silicon, etc. In some embodiments, heteroatoms will be selected from the group consisting of oxygen, nitrogen, sulfur, phosphorous, and halogen (F, Cl, Br, I, etc.). In some embodiments, a heteroatom or group may substitute a carbon. In some embodiments, a heteratom or group may substitute a hydrogen. In some embodiments, a substituted hydrocarbon may comprise one or more heteroatoms in the backbone or chain of the molecule (e.g., interposed between two carbon atoms, as in “oxa”). In some embodiments, a substituted hydrocarbon may comprise one or more heteroatoms pendant from the backbone or chain of the molecule (e.g., covalented bound to a carbon atom in the chain or backbone, as in “oxo”).

In some embodiments, any hydrocarbon or substituted hydrocarbon disclosed herein may be substituted with one or more (e.g., from 1-6 or from 1-4 or from 1-3 or one or two or three) substituents X, where X is independently selected at each occurrence from one or more (e.g., 1-20) heteroatoms or one or more (e.g., 1-10) heteroatom-containing groups, or X is independently selected at each occurrence from —F, —Cl, —Br, —I, —OH, —OR*, —NH₂, —NHR*, —N(R*)₂, —N(R*)₃⁺, —N(R*)—OH, —N(→O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂⁻, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(═O)₁₋₂—R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂; where, independently at each occurrence, R* may be H or a C₁₋₁₀ or C₁₋₈ or C₁₋₆ or C₁₋₄ hydrocarbon, including without limitation alkyl, alkenyl, alkynyl, aryl (e.g., phenyl), alkyl-aryl (e.g., benzyl), aryl-alkyl (e.g., toluyl), etc. In some embodiments, X may comprise a C₁-C₈ or C₁-C₆ or C₂-C₄ perfluoroalkyl. In some embodiments, X may be a C₁-C₈ or C₂-C₆ or C₃-C₅ heterocycle (e.g., heteroaryl radical). The term “halo” or “halogen” refers to any radical of fluorine, chlorine, bromine or iodine. In some embodiments, X is independently selected at each occurrence from —OH, —SH, —NH₂, —N(R*)₂, —C(O)OR*, —C(O)NR*R*, —C(O)NR*R*, —C(O)OH, —C(O)NH₂, F, or —Cl. In some embodiments, X is F. In some embodiments, R* is hydrogen, methyl, ethyl, propyl, or isopropyl. In some embodiments, R* is hydrogen, methoxy, ethoxy, propoxy, or isopropoxy. In some embodiments, X is —CF₃ or —O—CF₃.

In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls. Moreover, where a moiety is substituted with an R substituent, the group may be referred to as “R-substituted.” Where a moiety is R-substituted, the moiety is substituted with at least one R substituent and each R substituent is optionally different.

Unless otherwise specified, any compound disclosed herein which has one or more chiral centers may be in the form of a racemic mixture with respect to each chiral center, or may exist as pure or substantially pure (e.g., great than about 98% ee) R or S enantiomers with respect to each chiral center, or may exist as as mixtures of R and S enantiomers with respect to each chiral center, wherein the mixture comprises an enantiomeric excess of one or the other configurations, for example an enantiomeric excess (of R or S) of more than 60% or more than 70% or more than 80% or more than 90%, or more than 95%, or more than 98%, or more than 99% enantiomeric excess. In some embodiments, any chiral center may be in the “S” or “R” configurations. Stereocenters in structures as used herein may be labeled with a “*.” However, “*” labeled atoms are not necessarily stereocenters (e.g., dependent on substituent R groups at * labeled stereocenters may be the same). Additionally, those stereocenters not labeled with a “*” are still meant to indicate chiral centers.

Any of the compounds of the present disclosure may be in the form of pharmaceutically acceptable salts. “Pharmaceutically acceptable salts,” as used herein, denotes salts that are physiologically compatable, as defined herein, and that possess the desired pharmacological activity of the parent compound. Such salts include: acid addition salts formed with inorganic acids such as hydrochloric acid, hydrobromic acid, sulfuric acid, nitric acid, phosphoric acid, and the like; or formed with organic acids such as acetic acid, benzenesulfonic acid, benzoic acid, camphorsulfonic acid, citric acid, ethanesulfonic acid, fumaric acid, glucoheptonic acid, gluconic acid, glutamic acid, glycolic acid, hydroxynaphtoic acid, 2-hydroxyethanesulfonic acid, lactic acid, maleic acid, malic acid, malonic acid, mandelic acid, methanesulfonic acid, muconic acid, 2-naphthalenesulfonic acid, propionic acid, salicylic acid, succinic acid, tartaric acid, p-toluenesulfonic acid, trimethylacetic acid, and the like; or salts formed when an acidic proton present in the parent compound either is replaced by a metal ion, e.g., an alkali metal ion, an alkaline earth ion, or an aluminum ion; or coordinates with an organic or inorganic base. Acceptable organic bases include diethanolamine, ethanolamine, N-methylglucamine, triethanolamine, tromethamine, and the like. Acceptable inorganic bases include aluminum hydroxide, calcium hydroxide, potassium hydroxide, sodium carbonate, and sodium hydroxide.

It will be understood that the description of compounds herein is limited by principles of chemical bonding known to those skilled in the art. Accordingly, where a group may be substituted by one or more of a number of substituents, such substitutions are selected so as to comply with principles of chemical bonding with regard to valencies, etc., and to give compounds which are not inherently unstable. For example, any carbon atom will be bonded to two, three, or four other atoms, consistent with the four valence electrons of carbon.

In general, and unless otherwise indicated, substituent (radical) prefix names are derived from the parent hydride by either (i) replacing the “ane” in the parent hydride with the suffixes “yl,” “diyl,” “triyl,” “tetrayl,” etc.; or (ii) replacing the “e” in the parent hydride with the suffixes “yl,” “diyl,” “triyl,” “tetrayl,” etc. (here the atom(s) with the free valence, when specified, is (are) given numbers as low as is consistent with any established numbering of the parent hydride). Accepted contracted names, e.g., adamantyl, naphthyl, anthryl, phenanthryl, furyl, pyridyl, isoquinolyl, quinolyl, and piperidyl, and trivial names, e.g., vinyl, allyl, phenyl, and thienyl are also used herein throughout. Conventional numbering/lettering systems are also adhered to for substituent numbering and the nomenclature of fused, spiro, bicyclic, tricyclic, polycyclic rings.

The term “alkyl” refers to a saturated hydrocarbon chain that may be a straight chain or branched chain, containing the indicated number of carbon atoms. For example, C₁-C₆ alkyl indicates that the group may have from 1 to 6 (inclusive) carbon atoms in it. Any atom can be optionally substituted, e.g., by one or more substituents. Examples of alkyl groups include without limitation methyl, ethyl, n-propyl, isopropyl, and tert-butyl.

As used herein, the term “straight chain Cₙ₋ₘ alkylene,” employed alone or in combination with other terms, refers to a non-branched divalent alkyl linking group having n to m carbon atoms (for example 0-10 or 1-8 or 1-6 or 1-4 or 1-3 or 1-2). In some embodiments, a divalent radical according to the disclosure (e.g., R^{L}, L₁, etc.) can be a straight chain Cₙ₋ₘ alkylene group. Any atom can be optionally substituted, e.g., by one or more substituents (e.g., heteroatoms or groups X). Examples of straight chain alkylene include methylene (i.e., —CH₂—).

The term “haloalkyl” refers to an alkyl group, in which at least one hydrogen atom is replaced by halo. In some embodiments, more than one hydrogen atom (e.g., 2, 3, 4, 5, 6, 7, 8, 9, 10, 11, 12, 13, or 14, etc.) are replaced by halo. In these embodiments, the hydrogen atoms can each be replaced by the same halogen (e.g., fluoro) or the hydrogen atoms can be replaced by a combination of different halogens (e.g., fluoro and chloro). “Haloalkyl” also includes alkyl moieties in which all hydrogens have been replaced by halo (sometimes referred to herein as perhaloalkyl, e.g., perfluoroalkyl, such as trifluoromethyl). Any atom can be optionally substituted, e.g., by one or more substituents.

As referred to herein, the term “alkoxy” refers to a group of formula —O(alkyl). Alkoxy can be, for example, methoxy (—OCH₃), ethoxy, propoxy, isopropoxy, butoxy, iso-butoxy, sec-butoxy, pentoxy, 2-pentoxy, 3-pentoxy, or hexyloxy. Likewise, the term “thioalkoxy” refers to a group of formula —S(alkyl). Finally, the terms “haloalkoxy” and “halothioalkoxy” refer to —O(haloalkyl) and —S(haloalkyl), respectively. The term “sulfhydryl” refers to —SH. As used herein, the term “hydroxyl,” employed alone or in combination with other terms, refers to a group of formula —OH.

The term “aralkyl” refers to an alkyl moiety in which an alkyl hydrogen atom is replaced by an aryl group. One of the carbons of the alkyl moiety serves as the point of attachment of the aralkyl group to another moiety. Any ring or chain atom can be optionally substituted, e.g., by one or more substituents. Non-limiting examples of “aralkyl” include benzyl, 2-phenylethyl, and 3-phenylpropyl groups.

The term “alkenyl” refers to a straight or branched hydrocarbon chain containing the indicated number of carbon atoms and having one or more carbon-carbon double bonds. Any atom can be optionally substituted, e.g., by one or more substituents. Alkenyl groups can include, e.g., vinyl, allyl, 1-butenyl, and 2-hexenyl. One of the double bond carbons can optionally be the point of attachment of the alkenyl substituent.

The term “alkynyl” refers to a straight or branched hydrocarbon chain containing the indicated number of carbon atoms and having one or more carbon-carbon triple bonds. Alkynyl groups can be optionally substituted, e.g., by one or more substituents. Alkynyl groups can include, e.g., ethynyl, propargyl, and 3-hexynyl. One of the triple bond carbons can optionally be the point of attachment of the alkynyl substituent.

The term “heterocyclyl” refers to a fully saturated, partially saturated, or aromatic monocyclic, bicyclic, tricyclic, or other polycyclic ring system having one or more constituent heteroatom ring atoms independently selected from O, N (it is understood that one or two additional groups (e.g., R^{N}) may be present to complete the nitrogen valence and/or form a salt), or S. The heteroatom or ring carbon can be the point of attachment of the heterocyclyl substituent to another moiety. Any atom can be optionally substituted, e.g., with one or more substituents (e.g. heteroatoms or groups X). Heterocyclyl groups can include, e.g., tetrahydrofuryl, tetrahydropyranyl, piperidyl (piperidino), piperazinyl, morpholinyl (morpholino), pyrrolinyl, and pyrrolidinyl. By way of example, the phrase “heterocyclic ring containing from 5-6 ring atoms, wherein from 1-2 of the ring atoms is independently selected from N, NH, N(C₁-C₆ alkyl), NC(O)(C₁-C₆ alkyl), O, and S; and wherein said heterocyclic ring is optionally substituted with from 1-3 independently selected R^{a}” would include (but not be limited to) tetrahydrofuryl, tetrahydropyranyl, piperidyl (piperidino), piperazinyl, morpholinyl (morpholino), pyrrolinyl, and pyrrolidinyl.

The term “heterocycloalkenyl” refers to partially unsaturated monocyclic, bicyclic, tricyclic, or other polycyclic hydrocarbon groups having one or more (e.g., 1-4) heteroatom ring atoms independently selected from O, N (it is understood that one or two additional groups may be present to complete the nitrogen valence and/or form a salt), or S. A ring carbon (e.g., saturated or unsaturated) or heteroatom can be the point of attachment of the heterocycloalkenyl substituent. Any atom can be optionally substituted, e.g., by one or more substituents. Heterocycloalkenyl groups can include, e.g., dihydropyridyl, tetrahydropyridyl, dihydropyranyl, 4,5-dihydrooxazolyl, 4,5-dihydro-1H-imidazolyl, 1,2,5,6-tetrahydro-pyrimidinyl, and 5,6-dihydro-2H-[1,3]oxazinyl.

The term “cycloalkyl” refers to a fully saturated monocyclic, bicyclic, tricyclic, or other polycyclic hydrocarbon groups. Any atom can be optionally substituted, e.g., by one or more substituents. A ring carbon serves as the point of attachment of a cycloalkyl group to another moiety. Cycloalkyl moieties can include, e.g., cyclopropyl, cyclobutyl, cyclopentyl, cyclohexyl, cycloheptyl, adamantyl, and norbornyl (bicycle[2.2.1]heptyl).

The term “cycloalkenyl” refers to partially unsaturated monocyclic, bicyclic, tricyclic, or other polycyclic hydrocarbon groups. A ring carbon (e.g., saturated or unsaturated) is the point of attachment of the cycloalkenyl substituent. Any atom can be optionally substituted, e.g., by one or more substituents. Cycloalkenyl moieties can include, e.g., cyclohexenyl, cyclohexadienyl, or norbornenyl.

As used herein, the term “cycloalkylene” refers to a divalent monocyclic cycloalkyl group having the indicated number of ring atoms.

As used herein, the term “heterocycloalkylene” refers to a divalent monocyclic heterocyclyl group having the indicated number of ring atoms.

The term “aryl” refers to an aromatic monocyclic, bicyclic (2 fused rings), or tricyclic (3 fused rings), or polycyclic (>3 fused rings) hydrocarbon ring system. One or more ring atoms can be optionally substituted, e.g., by one or more substituents. Aryl moieties include, e.g., phenyl and naphthyl.

The term “heteroaryl” refers to an aromatic monocyclic, bicyclic (2 fused rings), tricyclic (3 fused rings), or polycyclic (>3 fused rings) hydrocarbon groups having one or more heteroatom ring atoms independently selected from O, N (it is understood that one or two additional groups may be present to complete the nitrogen valence and/or form a salt), or S in the ring. One or more ring atoms can be optionally substituted, e.g., by one or more substituents. Examples of heteroaryl groups include, but are not limited to, 2H-pyrrolyl, 3H-indolyl, 4H-quinolizinyl, acridinyl, benzo[b]thienyl, benzothiazolyl, β-carbolinyl, carbazolyl, coumarinyl, chromenyl, cinnolinyl, dibenzo[b,d]furanyl, furazanyl, furyl, imidazolyl, imidizolyl, indazolyl, indolyl, isobenzofuranyl, isoindolyl, isoquinolyl, isothiazolyl, isoxazolyl, naphthyridinyl, oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrrolyl, quinazolinyl, quinolyl, quinoxalinyl, thiadiazolyl, thianthrenyl, thiazolyl, thienyl, triazolyl, and xanthenyl.

Examples of heterocyclic rings as used in this disclosure include the following:

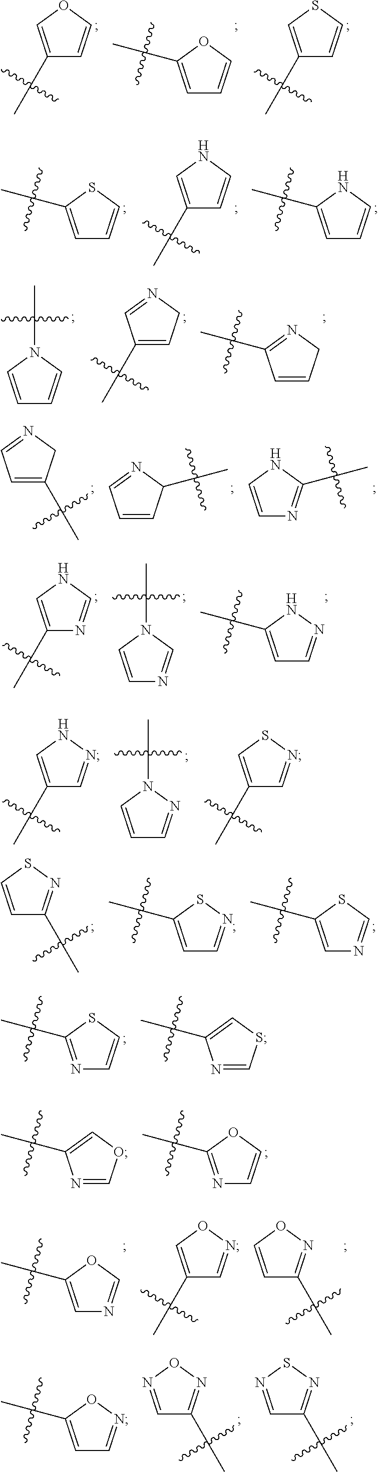

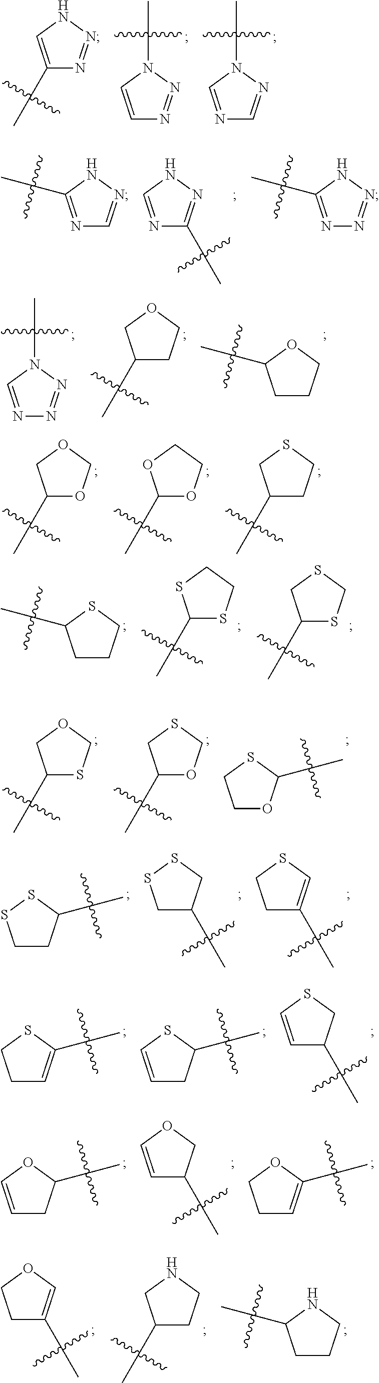

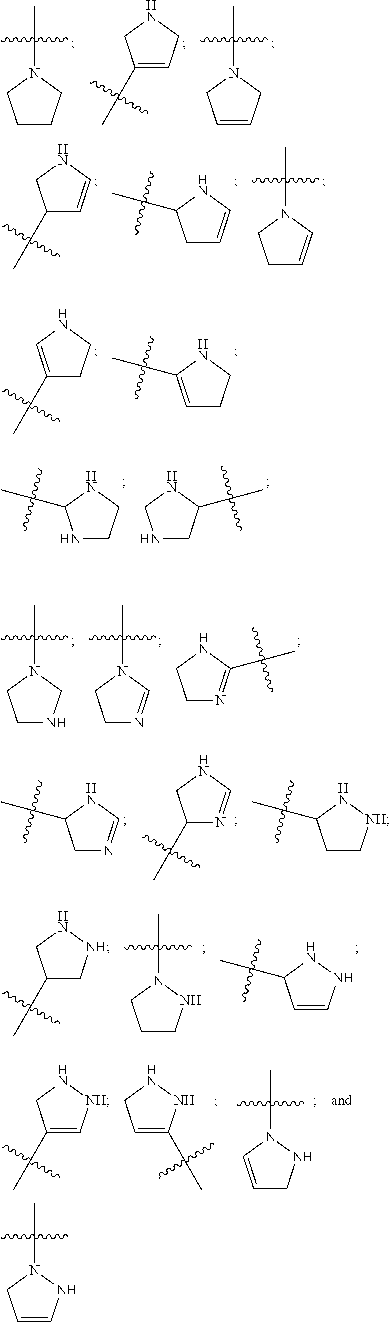

Any of the foregoing may be used where the present disclosure calls for a monovalent ring such as a heterocyclic radical, including those represented as R^{B} herein. Similarly, where a heterocycle according to the disclosure has two points of attachment (such as in the divalent radicals R^{Q}) the second point of attachment of any of the foregoing may be any suitable position containing a hydrogen atom.

The terms “arylcycloalkyl” and “arylheterocyclyl” refer to bicyclic, tricyclic, or other polycyclic ring systems that include an aryl ring fused to a cycloalkyl and heterocyclyl, respectively. Similarly, the terms “heteroarylheterocyclyl,” and “heteroarylcycloalkyl” refer to bicyclic, tricyclic, or other polycyclic ring systems that include a heteroaryl ring fused to a heterocyclyl and cycloalkyl, respectively. Any atom can be substituted, e.g., by one or more substituents. For example, arylcycloalkyl can include indanyl; arylheterocyclyl can include 2,3-dihydrobenzofuryl, 1,2,3,4-tetrahydroisoquinolyl, and 2,2-dimethylchromanyl.

The term “vicinal” refers to the configuration in which any two atoms or groups are, respectively, bonded to two adjacent atoms (i.e., the two atoms are directly bonded to one another). The term “geminal” describes a configuration in which any atoms or two functional groups are bonded to the same atom. As used herein, when any two groups are said to together form a ring, unless otherwise indicated, it is meant that a bond is formed between each of said two groups, with the valences of the atoms appropriately adjusted to accomadate at least a bond (e.g., a hydrogen atom may be removed from each group).

The descriptors “C═O” or “C(O)” or “carbonyl” refers to a carbon atom that is doubly bonded to an oxygen atom. “Alkyl carbonyl” has a common formula of R—C(O)— wherein R may be C₁₋₁₂ alkyl, C₂₋₁₂ alkenyl, C₂₋₁₂ alkynyl, C₃₋₁₂ cycloalkyl, C₆₋₁₂ aryl, C3-12 heteroaryl, or C₃₋₁₂ heterocyclyl.

The term “oxo” refers to double bonded oxygen which can be a substituent on carbon or other atoms. When oxo is a substituent on nitrogen or sulfur, it is understood that the resultant groups have the structures N→O⁻ and S(O) and SO₂, respectively.

As used herein, the term “cyano,” employed alone or in combination with other terms, refers to a group of formula —CN, wherein the carbon and nitrogen atoms are bound together by a triple bond. The term “azide” refers to a group of formula —N₃. The term “nitro” refers to a group of formula —NO₂. The term “amine” includes primary (—NH₂), secondary (—NHR), tertiary (—NRR′), and quaternary (—N⁺RR′R″) amine having one, two or three independently selected substituents such as straight chain or branched chain alkyl, alkenyl, alkynyl, cycloalkyl, aryl, heteroaryl, heterocycle, and the like.

In general, when a definition for a particular variable includes both hydrogen and non-hydrogen (halo, alkyl, aryl, etc.) possibilities, the term “substituent(s) other than hydrogen” refers collectively to the non-hydrogen possibilities for that particular variable.

In general, the limits (end points) of any range recited herein are within the scope of the invention and should be understood to be disclosed embodiments. Additionally, any half integral value within that range is also contemplated. For example, a range of about 0 to 4 expressly discloses 0, 0.5, 1, 1.5, 2, 2.5, 3, 3.5, 4, and any subset within that range (e.g., from about 1 to 2.5).

The term “substituent” refers to a group “substituted” on, e.g., an alkyl, haloalkyl, cycloalkyl, heterocyclyl, heterocycloalkenyl, cycloalkenyl, aryl, or heteroaryl group at any atom of that group, replacing one or more hydrogen atoms therein. In one aspect, the substituent(s) on a group are independently any one single, or any combination of two or more of the permissible atoms or groups of atoms delineated for that substituent. In another aspect, a substituent may itself be substituted with any one of the above substituents. Further, as used herein, the phrase “optionally substituted” means unsubstituted (e.g., substituted with an H) or substituted. It is understood that substitution at a given atom is limited by valency. Common substituents include halo (e.g. F), C₁₋₁₂ straight chain or branched chain alkyl, C₂₋₁₂ alkenyl, C₂₋₁₂ alkynyl, C₃₋₁₂ cycloalkyl, C₆₋₁₂ aryl, C3-12 heteroaryl, C₃₋₁₂ heterocyclyl, C₁₋₁₂ alkylsulfonyl, nitro, cyano, —COOR, —C(O)NRR′, —OR, —SR, —NRR′, and oxo, such as mono- or di- or tri-substitutions with moieties such as trifluoromethoxy, chlorine, bromine, fluorine, methyl, methoxy, pyridyl, furyl, triazyl, piperazinyl, pyrazoyl, imidazoyl, and the like, each optionally containing one or more heteroatoms such as halo, N, O, S, and P. R and R′ are independently hydrogen, C₁₋₁₂ alkyl, C₁₋₁₂ haloalkyl, C₂₋₁₂ alkenyl, C₂₋₁₂ alkynyl, C₃₋₁₂ cycloalkyl, C₄₋₂₄ cycloalkylalkyl, C₆₋₁₂ aryl, C₇₋₂₄ aralkyl, C₃₋₁₂ heterocyclyl, C₃₋₂₄ heterocyclylalkyl, C3-12 heteroaryl, or C₄₋₂₄ heteroarylalkyl. Unless otherwise noted, all groups described herein optionally contain one or more common substituents, to the extent permitted by valency. Further, as used herein, the phrase “optionally substituted” means unsubstituted (e.g., substituted with an H) or substituted. As used herein, the term “substituted” means that a hydrogen atom is removed and replaced by a substituent (e.g., a common substituent). It is understood by one of ordinary skill in the chemistry art that substitution at a given atom is limited by valency. The use of a substituent (radical) prefix names such as alkyl without the modifier “optionally substituted” or “substituted” is understood to mean that the particular substituent is unsubstituted. However, the use of “haloalkyl” without the modifier “optionally substituted” or “substituted” is still understood to mean an alkyl group, in which at least one hydrogen atom is replaced by halo.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines, and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger, and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853). The methods above may be used to synthesize single molecular species.

The term “Ras” refers to one or more of the family of human Ras GTPase proteins (e.g., K-Ras, H-Ras, N-Ras). The term “K-Ras” refers to the nucleotide sequences or proteins of human K-Ras (e.g., human K-Ras4A (NP_203524.1), human K-Ras4B (NP_004976.2), or both K-Ras4A and K-Ras4B).

The term “K-Ras” includes both the wild-type form of the nucleotide sequences or proteins as well as any mutants thereof. In some embodiments, “K-Ras” is wild-type K-Ras. In some embodiments, “K-Ras” is one or more mutant forms. The term “K-Ras” XYZ refers to a nucleotide sequence or protein of a mutant K-Ras wherein the Y numbered amino acid of K-Ras that has an X amino acid in the wildtype instead has a Z amino acid in the mutant (e.g., K-Ras G12C has a G in wildtype protein but a C at the number 12 position in the K-Ras G12C mutant protein). In some embodiments K-Ras refers to K-Ras4A and K-Ras4B. In some embodiments, K-Ras refers to K-Ras4A. In some embodiments, K-Ras refers to K-Ras4B.

The term “K-Ras inhibitor test compound” as used herein refers to a compound that is being characterized in an assay for the ability to inhibit an activity, function, or level (e.g., amount) of K-Ras protein.

The term “Raf” refers to one or more of the members of the family of human Raf proteins (e.g., c-Raf, A-Raf, and B-Raf).

The term “signaling pathway” as used herein refers to a series of interactions between cellular and optionally extra-cellular components (e.g., proteins, nucleic acids, small molecules, ions, and lipids) that conveys a change in one component to one or more other components, which in turn may convey a change to additional components, which is optionally propagated to other signaling pathway components. For example, binding of a K-Ras with a compound as described herein may result in a change in one or more protein-protein interactions of the K-Ras, resulting in changes in cell growth, proliferation, or survival.

As defined herein, the terms “inhibition,” “inhibit,” “inhibiting,” and the like in reference to a protein-inhibitor interaction means negatively affecting (e.g., decreasing or diminishing) the activity or function of the protein (e.g., decreasing the signaling pathway stimulated by GTP bound Ras (e.g., K-Ras, K-Ras G12C, K-Ras double mutants), nucleotide exchange, effector protein binding, effector protein activation, guanine exchange factor (GEF) binding, SOS binding, GEF-facilitated nucleotide exchange, phosphate release, nucleotide release, nucleotide binding) relative to the activity or function of the protein in the absence of the inhibitor (e.g., mutant K-Ras inhibitor, activitated K-Ras inhibitor). Inhibition includes, at least in part, partially or totally blocking stimulation, decreasing, preventing, or delaying activation, or inactivating, desensitizing, or down-regulating the signaling pathway or enzymatic activity or the amount of a protein (e.g., K-Ras, K-Ras G12C, K-Ras double mutants). In some embodiments, inhibition refers to inhibition of interactions of Ras (K-Ras, K-Ras G12C, K-Ras double mutants) with signaling pathway binding partners (e.g., PI3K, SOS, Raf).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity (e.g., GTPase activity, protein-protein interaction, signaling pathway) of a protein (e.g., Ras, K-Ras, mutant K-Ras, K-Ras G12C, K-Ras double mutants) in the absence of a compound as described herein.

“Contacting” is used in accordance with its plain ordinary meaning and refers to the process of allowing at least two distinct species (e.g., chemical compounds including biomolecules, or cells) to become sufficiently proximal to react, interact, or physically touch. It should be appreciated, however, that the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a compound as described herein and a protein or enzyme (e.g., Ras, K-Ras, H-Ras, N-Ras, K-Ras4A, K-Ras4B, mutant Ras, mutant K-Ras, K-Ras G12C, K-Ras G13C, K-Ras G12D, K-Ras G13D). In some embodiments, the protein may be K-Ras. In some embodiments, the protein may be a mutant K-Ras (e.g., K-Ras G12C, K-Ras G13C, K-Ras G12D, K-Ras G13D). In some embodiments, the protein may be K-Ras4A. In some embodiments, the protein may be K-Ras4B. In some embodiments contacting includes allowing a compound described herein to interact with a protein or enzyme that is involved in a signaling pathway.

The terms “treating” or “treatment” refers to any indicia of success in the treatment or amelioration of an injury, disease, pathology, or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. For example, the certain methods presented herein successfully treat cancer by decreasing the incidence of cancer and or causing remission of cancer. In some embodiments of the compositions or methods described herein, treating cancer includes slowing the rate of growth or spread of cancer cells, reducing metastasis, or reducing the growth of metastatic tumors. The term “treating” and conjugations thereof, include prevention of an injury, pathology, condition, or disease.

An “effective amount” is an amount sufficient for a compound to accomplish a stated purpose relative to the absence of the compound (e.g., achieve the effect for which it is administered, treat a disease, reduce enzyme activity, increase enzyme activity, reduce signaling pathway, reduce one or more symptoms of a disease or condition (e.g., reduce GTPase activity in a cell, increase GTPase activity, reduce signaling pathway stimulated by GTP bound Ras (e.g., K-Ras), reduce the signaling pathway activity of Ras, reduce the signaling pathway activity of K-Ras, reduce the signaling pathway activity of K-Ras(G12C), reduce the signaling pathway activity of a mutant K-Ras, increase the activity of Ras, increase the activity of K-Ras, increase the activity of K-Ras(G12C), increase the activity of a mutant K-Ras, inhibit the binding or interaction of K-Ras to Raf, inhibit the binding of K-Ras to SOS, inhibit the binding of K-Ras to a GEF, inhibit nucleotide exchange). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

“Disease” or “condition” refers to a state of being or health status of a patient or subject capable of being treated with the compounds or methods provided herein. In some embodiments, the disease is a disease related to (e.g., caused by) a mutant Ras. In some embodiments, the disease is a disease related to (e.g., caused by) a mutant K-Ras (e.g., K-Ras G12C, G13C, G12D, or G13D) or aberrant K-Ras signaling pathway activity (e.g., lung cancer, breast cancer, colon cancer, colorectal cancer, pancreatic cancer, leukemia). Examples of diseases, disorders, or conditions include, but are not limited to cancer. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma, leukemia (including AML, ALL, and CML), or multiple myeloma.

As used herein, the term “cancer” refers to all types of cancer, neoplasm or malignant tumors found in mammals (e.g., humans), including leukemia, carcinomas and sarcomas. Exemplary cancers that may be treated with a compound or method provided herein include cancer of the thyroid, endocrine system, brain, breast, cervix, colon, head, neck, liver, kidney, lung, non-small cell lung, melanoma, mesothelioma, ovary, sarcoma, stomach, uterus, Medulloblastoma, colorectal cancer, or pancreatic cancer. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, aleukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

“K-Ras associated cancer” (also referred to herein as “K-Ras related cancer” or “cancer associated with K-Ras”) refers to a cancer caused by aberrant K-Ras activity or signaling. K-Ras related cancers may include lung cancer, non-small cell lung cancer, breast cancer, leukemia, pancreatic cancer, colon cancer, or colorectal cancer. Other cancers that are associated with aberrant activity of one or more of Ras, K-Ras, H-Ras, N-Ras, mutant K-Ras (including K-Ras G12C, K-Ras G13C, K-Ras G12D, K-Ras G13D mutants), mutant N-Ras, and mutant H-Ras are well known in the art and determining such cancers are within the skill of a person of skill in the art.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The term “administer (or administering) a Ras inhibitor” means administering a compound that inhibits the activity or level (e.g., amount) or level of a signaling pathway of one or more Ras proteins (e.g., a Ras inhibitor, K-Ras inhibitor, N-Ras inhibitor, H-Ras inhibitor, mutant K-Ras inhibitor, K-Ras G12C inhibitor, K-Ras G13C inhibitor, K-Ras G12D inhibitor, K-Ras G13D inhibitor) to a subject.

Administration may include, without being limited by mechanism, allowing sufficient time for the Ras inhibitor to reduce the activity of one or more Ras proteins or for the Ras inhibitor to reduce one or more symptoms of a disease (e.g., cancer, wherein the Ras inhibitor may arrest the cell cycle, slow the cell cycle, reduce DNA replication, reduce cell replication, reduce cell growth, reduce metastasis, or cause cell death). The term “administer (or administering) a K-Ras inhibitor” means administering a compound that inhibits the activity or level (e.g., amount) or level of a signaling pathway of one or more K-Ras proteins (K-Ras, mutant K-Ras, K-Ras G12C, K-Ras G12D, K-Ras G13C, K-Ras G13D).

The term “associated” or “associated with” in the context of a substance or substance activity or function associated with a disease (e.g., a protein associated disease, a cancer associated with aberrant Ras activity, K-Ras associated cancer, mutant K-Ras associated cancer, activated K-Ras associated cancer, K-Ras G12C associated cancer, K-Ras G13C associated cancer, K-Ras G12D associated cancer, K-Ras G13D associated cancer) means that the disease (e.g., cancer) is caused by (in whole or in part), or a symptom of the disease is caused by (in whole or inpart) the substance or substance activity or function. For example, a cancer associated with aberrant Ras activity or function may be a cancer that results (entirely or partially) from aberrant Ras activity or function (e.g., enzyme activity, protein-protein interaction, signaling pathway) or a cancer wherein a particular symptom of the disease is caused (entirely or partially) by aberrant Ras activity or function. As used herein, what is described as being associated with a disease, if a causative agent, could be a target for treatment of the disease. For example, a cancer associated with aberrant Ras activity or function or a Ras associated cancer, may be treated with a Ras modulator or Ras inhibitor, in the instance where increased Ras activity or function (e.g., signaling pathway activity) causes the cancer. For example, a cancer associated with K-Ras G12C may be a cancer that a subject with K-Ras G12C is at higher risk of developing as compared to a subject without K-Ras G12C.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

### K-Ras Mutants

In one aspect, the present disclosure provides “tool” K-Ras proteins, e.g., K-Ras mutants that can be used to enable the discovery of new molecules that modulates an activity of K-Ras. The new K-Ras mutants disclosed herein are designed to increase the availability of protein conformations that open up, or improve accessibility to, site A, to allow for screening of new compounds that can bind site A more effectively, reversibly and/or non-covalently.

In another aspect, the present disclosure provides a protocol for using small molecules as allosteric primers binding at site D and that enhance the affinity and thereby the sensitivity of K-Ras proteins for small molecules binding at site A.

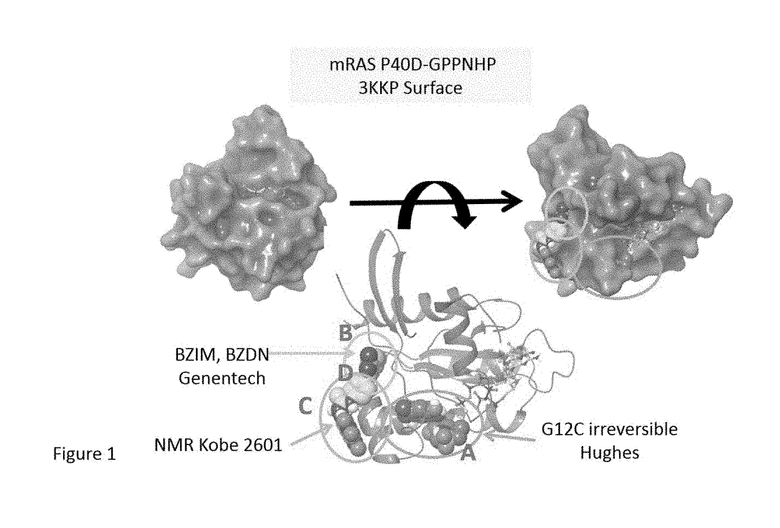

Ostrem et al., Nature 2013 Nov. 28; 503(7477):548-51 (incorporated by reference in its entirety) previously discovered compounds that covalently bind within a pocket of K-Ras G12C referred to as “irreversible site” or “site A” (see FIG. 1). However, these compounds irreversibly bind site A and have certain disadvantages, including off-target effect due to their high reactivity, irreversibility due to covalent modifications, as well as adverse drug reactions caused by immunogenic drug-protein adducts. Taveras et al., Bioorganic & Medicinal Chem 1997; 5(1): 125-133 (incorporated by reference in its entirety) identified “small organic inhibitors” of the Ras exchange process. These molecules are said to bind to the Ras protein in a previously unidentified binding pocket in the Switch 2 region without displacing GDP. These inhibitors contain a potentially reactive hydroxylamine group, which may covalently bind to the Ras proteins.

Maurer et al., Proc Natl Acad Sci USA. 2012 Apr. 3; 109(14): 5299-5304 (incorporated by reference in its entirety) identified another site that is adjacent to the Switch 1/2 regions (“site B” in FIG. 1). Shima et al., Proc Natl Acad Sci USA. 2013 May 14; 110(20): 8182-8187 (incorporated by reference in its entirety) identified another pocket (“site C” in FIG. 1). However, site B is deep but narrow, and site C is shallow, both of which are intrinsically limited in their cabability to bind drugable molecules. The overlapping region of site B and site C suggests a unified “site D” that is more tractable as a drug-binding pocket.

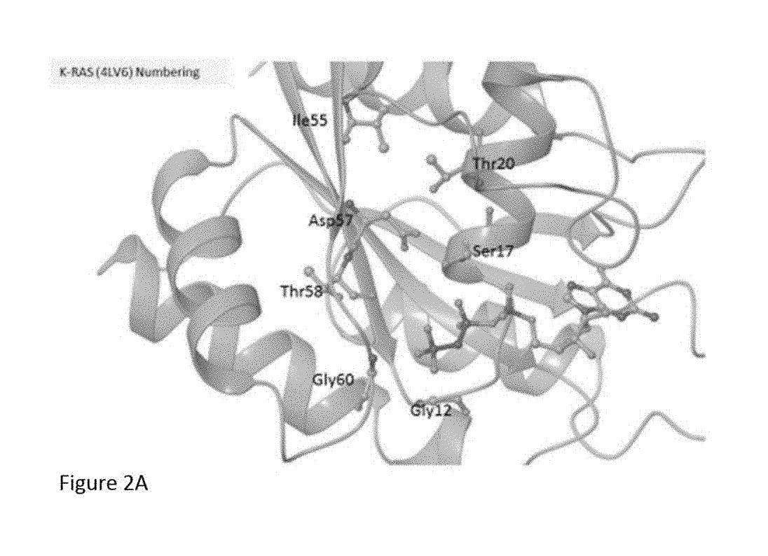

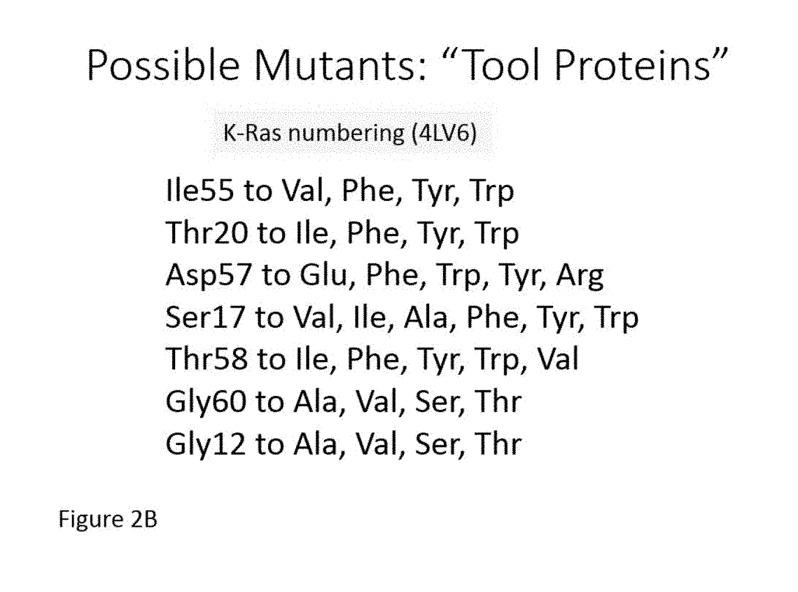

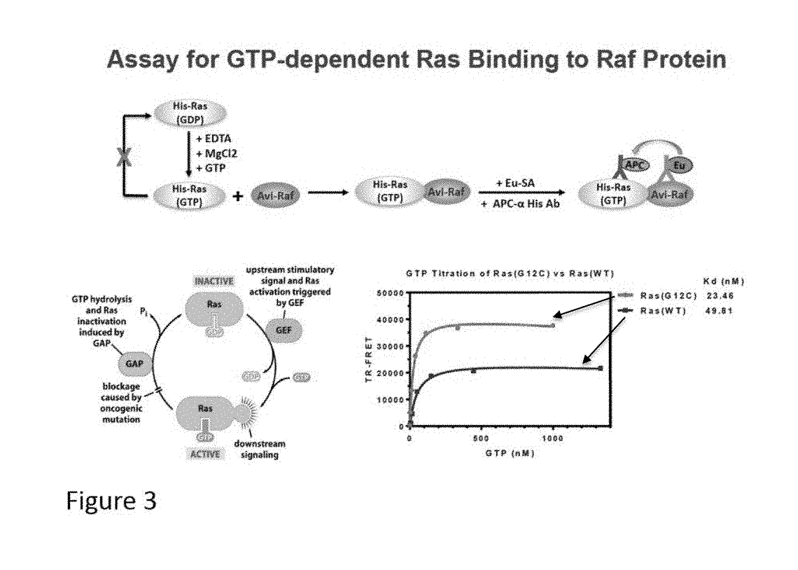

Exemplary K-Ras mutants are shown in FIGS. 2A and 2B, as well as Table 1 herein.

In various embodiments, the K-Ras mutants disclosed herein can be used to screen for new compounds as disclosed herein.

### Compounds

Compounds of any one of formula (A1)-(A38) are provided herein. The compounds described by Scaffolds (A1)-(A38) are contemplated to be useful in the practice of the invention, and consequently may find utility in pharmaceutical compositions for treatment of cancers and other diseases, especially where the cancer is associated with a K-Ras mutation, preferably K-Ras(G12C). The compounds are believed to be selective inhibitors of human K-Ras mutants. Molecular scaffolds for some respresentative embodiments of the invention are shown below, however, reference is made to the detailed description of these compounds found in the section “Illustrative Embodiments” at the end of this Detailed Description.

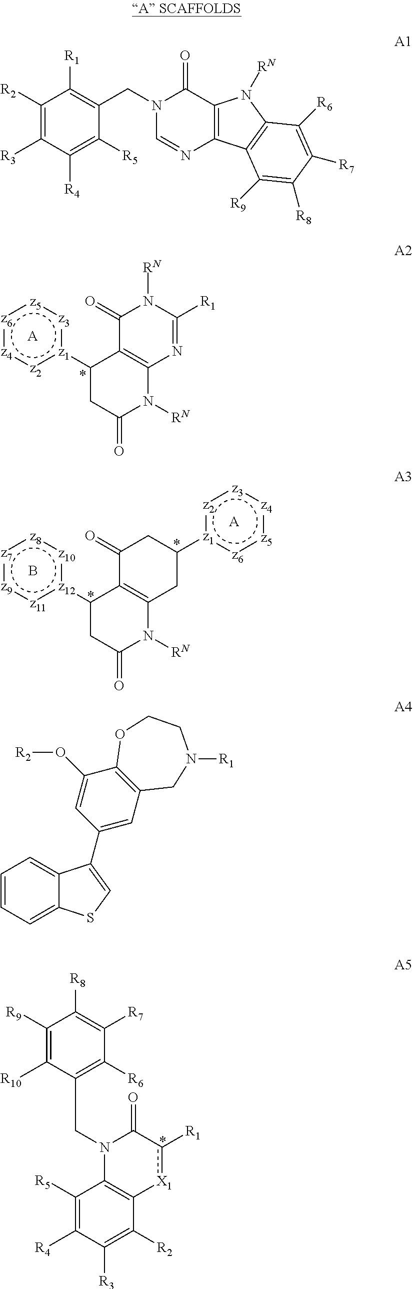

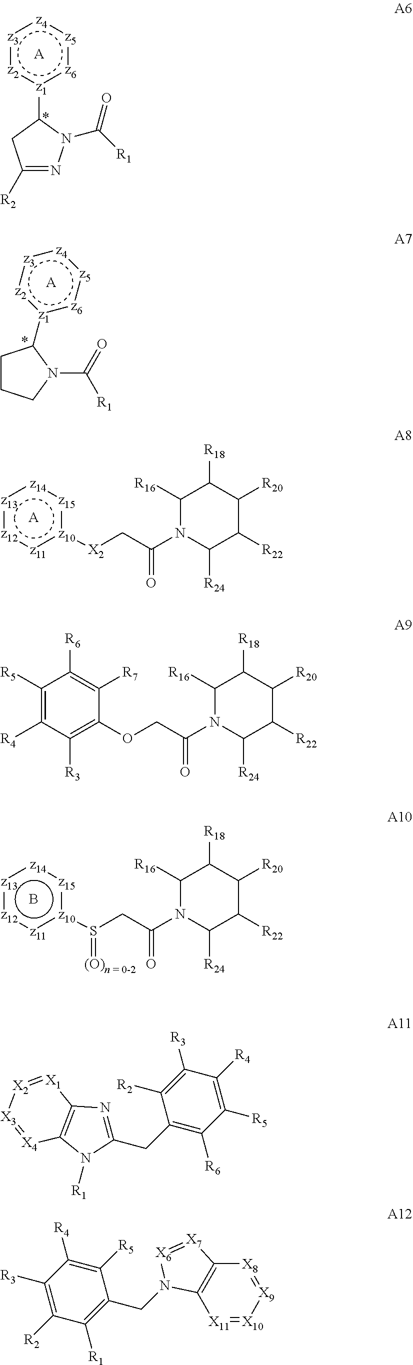

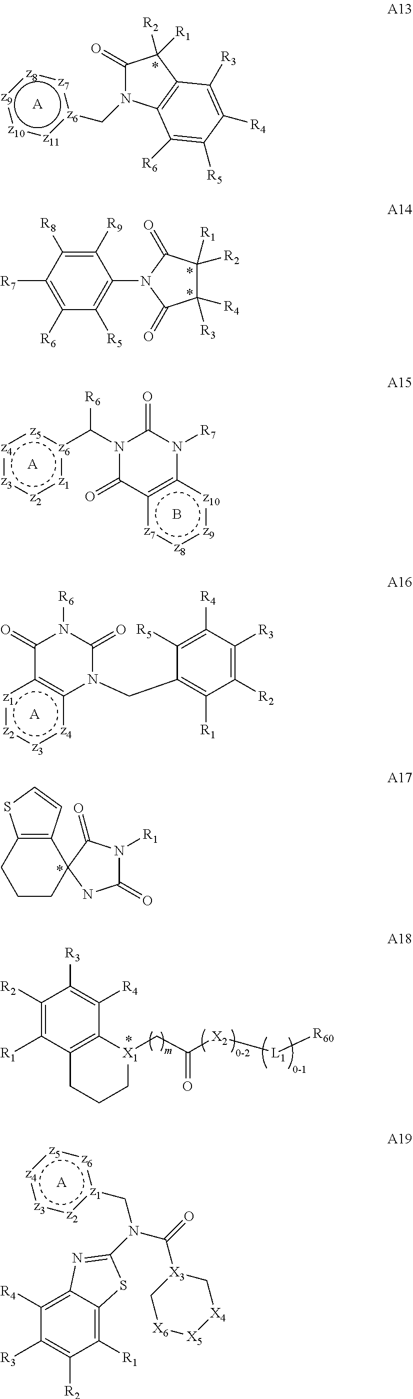

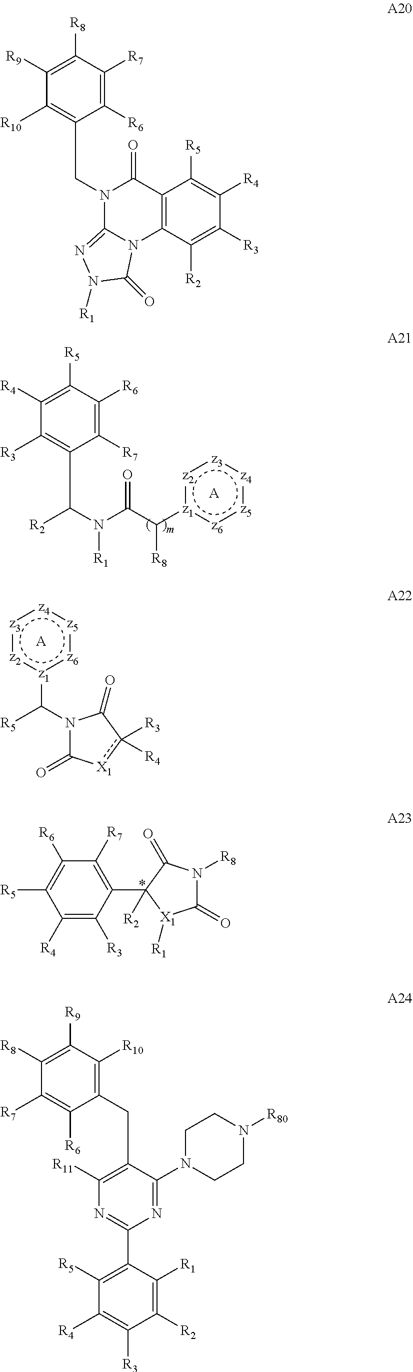

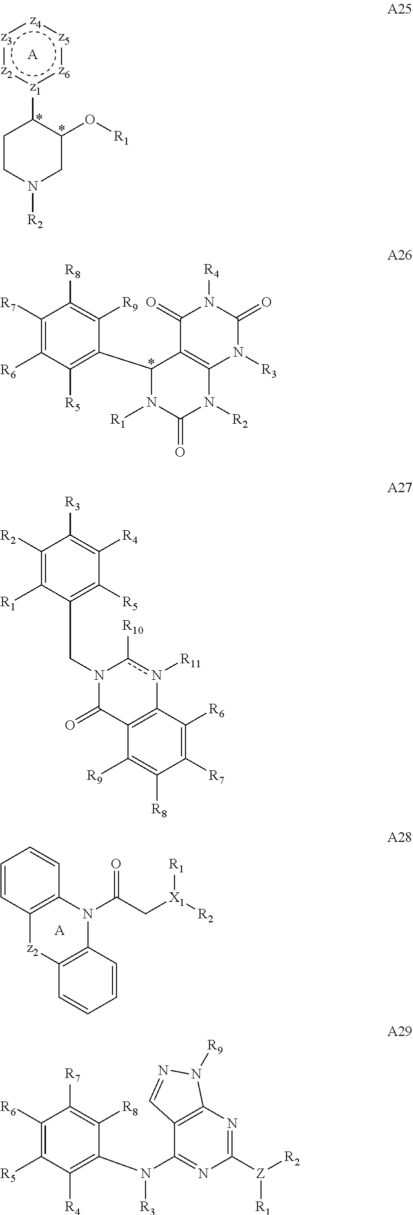

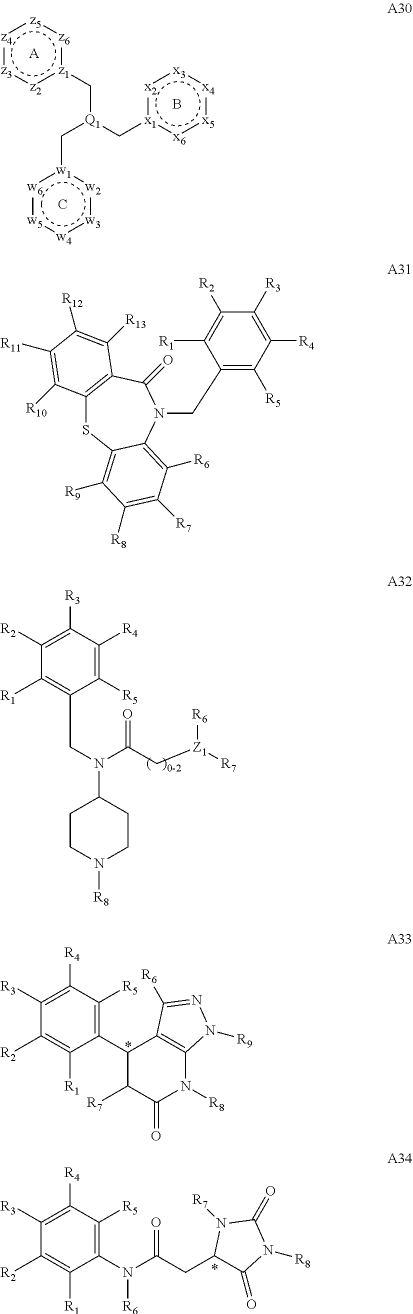

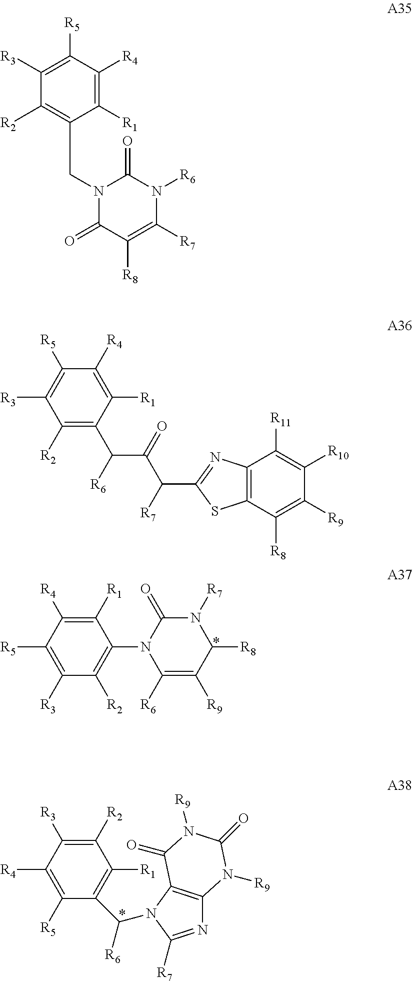

In any of the foregoing scaffolds, any “R” groups, e.g., R₁, R₂, R₃, R₄ R₅, R₆, etc., and R^{N}, R*, R^{L}, R′, R″, etc., may each independently selected from C₁₋₂₂ hydrocarbons, each optionally substituted with from 1-6 (or with 1-3) groups R and/or groups X and/or with from 1-12 (or from 1-10 or from 1-6 or from 1-3) heteroatoms, for example, selected from halogen (F, Cl, Br, I), N, O, S, and P. In scaffolds which contain more than one common “R” group (e.g., a scaffold or formula has two R₁ groups), it is independently selected at each occurrence. In some embodiments, any “R” groups may be hydrogen; halo; hydroxyl; C₁₋₆ (e.g., C₁₋₃) alkoxyl optionally substituted with 1 or more hydroxyl, oxo, cyano, amine, halo, R^{A} and/or R^{B}; C₁₋₆ (e.g., C₁₋₃) alkyl carbonyl optionally substituted with 1 or more hydroxyl, oxo, cyano, amine, halo, R^{A} and/or R^{B}; C₁₋₆ (e.g., C₁₋₃) alkoxycarbonyl optionally substituted with 1 or more hydroxyl, oxo, cyano, amine, halo, R^{A} and/or R^{B}; cyano; nitro; amine; R^{A} and R^{B}; wherein R^{A} at each occurrence is independently selected from C₁₋₆ (e.g., C₁₋₃) alkyl, C₂₋₆ (e.g., C₂₋₃) alkenyl and C₂₄ (e.g., C₂₋₃) alkynyl, each optionally substituted with 1 or more halo, hydroxyl, C₁₋₆ (e.g., C₁₋₃) alkoxyl, C₁₋₆ (e.g., C₁₋₃) thioalkoxyl, C₁₋₆ (e.g., C₁₋₃) alkyl carbonyl, C₁₋₆ (e.g., C₁₋₃) alkoxycarbonyl, oxo, cyano, nitro, and/or amine; wherein R^{B} at each occurrence is independently selected from C₃₋₁₂ cycloalkyl, C₂₋₆ heterocyclyl, C₆₋₁₂ aryl and C₃₋₁₂ heteroaryl, each optionally substituted with 1 or more of: halo; hydroxyl; C₁₋₆ alkyl optionally substituted with 1 or more C₃₋₁₂ cycloalkyl, C₂₋₆ heterocyclyl, C₆₋₁₂ aryl, C₃₋₁₂ heteroaryl, C₁₋₆ alkoxyl, C₁₋₆ carboxamide, amine, oxo, halo and/or hydroxyl; C₁₋₆ alkoxyl or C₁₋₆ thioalkoxyl, each optionally substituted with 1 or more C₃₋₁₂ cycloalkyl, C₂₋₆ heterocyclyl, C₆₋₁₂ aryl, C₃₋₁₂ heteroaryl, C₁₋₆ alkoxyl, C₁₋₆ carboxamide, amine, oxo, halo and/or hydroxyl; C₁₋₆ (e.g., C₁₋₃) alkyl carbonyl optionally substituted with 1 or more C₃₋₁₂ cycloalkyl, C₂₋₆ heterocyclyl, C₆₋₁₂ aryl, C₃₋₁₂ heteroaryl, C₁₋₆ alkoxyl, C₁₋₆ carboxamide, amine, oxo, halo and/or hydroxyl; C₁₋₆ carboxamide, C₁₋₆ carboxyl, or C₂₋₆ (e.g., C₂₋₃) alkoxycarbonyl, each optionally substituted with 1 or more C₃₋₁₂ cycloalkyl, C₂₋₆ heterocyclyl, C₆₋₁₂ aryl, C₃₋₁₂ heteroaryl, C₁₋₆ alkoxyl, C₁₋₆ carboxamide, amine, oxo, halo and/or hydroxyl; cyano; nitro; azide; amine; C₃₋₁₂ cycloalkyl; C₂₋₆ heterocyclyl; C₆₋₁₂ aryl; and/or C₃₋₁₂ heteroaryl; wherein each of the substituents C₃₋₁₂ cycloalkyl, C₂₋₆ heterocyclyl, C₆₋₁₂ aryl and C₃₋₂ heteroaryl is additionally optionally substituted with 1 or more halo, hydroxyl, C₁₋₆ alkyl, C₁₋₆ (e.g., C₁₋₃) alkoxyl, C₁₋₆ (e.g., C₁₋₃) thioalkoxyl, C₂₋₆ (e.g., C₁₋₃) alkoxycarbonyl, C₁₋₆ carboxamide, C₁₋₆ carboxyl, oxo, cyano, nitro and/or amine. Any two vicinal groups substituted in cyclic R^{B} groups may together form a fused ring to generate a polycyclic (i.e., bicyclic, tricyclic, etc.) R^{B} group. Any two geminal groups substituted in cyclic R^{B} groups may together form a spiro ring to generate a polycyclic R^{B} group. Any two non-geminal and non-vicinal groups substituted in cyclic R^{B} groups may together form a bridged ring to generate a polycyclic R^{B} group.

In any of the foregoing scaffolds, and as described herein, any ring subsitutent z₁, z₂, etc., Z₁, Z₂, etc., x₁, x₂, etc., X₁, X₂, etc., w₁, w₂, etc., may be selected from O, S, N, NH, NR, NRN, NR*, NX, C, CH, CR*, CR, CX, CH₂, C(R*)(R*), and C(R)(X), without limitation. The selections are made according to principles of bonding and valence known to those of skill in the art. Any rings disclosed herein may be aromatic, partially unsaturated, or saturated. Any rings may further have one or more additional rings fused thereto, or may be substituted with one or more groups R and/or X.

In various embodiments, any group R may be, without limitation, a group R^{A}, which may be C₆₋₁₂ aryl or C₃₋₁₂ heteroaryl, each optionally substituted with 1 or more of halo, hydroxyl; C₁₋₆ alkyl optionally substituted with 1 or more C₃₋₁₂ cycloalkyl, C₂₋₆ heterocyclyl, C₆₋₁₂ aryl, C₃₋₁₂ heteroaryl, C₁₋₆ alkoxyl, C₁₋₆ carboxamide, amine, oxo, halo and/or hydroxyl; C₁₋₆ alkoxyl or C₁₋₆ thioalkoxyl, each optionally substituted with 1 or more C₃₋₁₂ cycloalkyl, C₂₋₆ heterocyclyl, C₆₋₁₂ aryl, C₃₋₁₂ heteroaryl, C₁₋₆ alkoxyl, C₁₋₆ carboxamide, amine, oxo, halo and/or hydroxyl; C₁₋₆ (e.g., C₁₋₃) alkyl carbonyl optionally substituted with 1 or more C₃₋₁₂ cycloalkyl, C₂₋₆ heterocyclyl, C₆₋₁₂ aryl, C₃₋₁₂ heteroaryl, C₁₋₆ alkoxyl, C₁₋₆ carboxamide, amine, oxo, halo and/or hydroxyl; C₂₋₆ (e.g., C₂₋₃) alkoxycarbonyl optionally substituted with 1 or more C₃₋₁₂ cycloalkyl, C₂₋₆ heterocyclyl, C₆₋₁₂ aryl, C₃₋₁₂ heteroaryl, C₁₋₆ alkoxyl, C₁₋₆ carboxamide, amine, oxo, halo and/or hydroxyl; cyano; nitro; azide; amine; C₃₋₁₂ cycloalkyl; C₂₋₆ heterocyclyl; C₆₋₁₂ aryl; and/or C₃₋₁₂ heteroaryl.

Specific compounds believed to be selective inhibitors of human mutant K-Ras, and therefore useful in the practice of the invention, are provided herein. The compounds described herein, including in Appendix A, are believed to non-covalently bind K-Ras(G12C) and preferably modulate the binding of GDP or GTP to the K-Ras protein. It will be understood that each of the compounds of Appendix A are within the scope of the invention. Additionally, those compounds of Appendix A in which any hydrogen is replaced by a group R or X is also contemplated to be part of the invention. Those compounds of Appendix A in which any hydrogen (e.g., one, two, three, four, etc.) is replaced with lower alkyl are within the scope of the invention. Those compounds of Appendix A in which any hydrogen (e.g., one, two, three, four, etc.) is replaced with lower alkoxy is within the scope of the invention. Those compounds of Appendix A in which any hydrogen (e.g., one, two, three, four, etc.) is replaced with Cl, F, Br, and/or OH, are within the scope of the invention.

### Compound Forms and Salts

The compounds of the present disclosure may contain one or more asymmetric centers and thus occur as racemates and racemic mixtures, enantiomerically enriched mixtures, single enantiomers, individual diastereomers, and diastereomeric mixtures. All such isomeric forms of these compounds are expressly included in the present disclosure. The compounds of the present disclosure may also contain linkages (e.g., carbon-carbon bonds, carbon-nitrogen bonds such as amide bonds) wherein bond rotation is restricted about that particular linkage, e.g., restriction resulting from the presence of a ring or double bond. Accordingly, all cis/trans and E/Z isomers and rotational isomers are expressly included in the present disclosure. The compounds of the present disclosure may also be represented in multiple tautomeric forms, in such instances, the present disclosure expressly includes all tautomeric forms of the compounds described herein, even though only a single tautomeric form may be represented. All such isomeric forms of such compounds are expressly included in the present disclosure.

Optical isomers can be obtained in pure form by standard procedures known to those skilled in the art, and include, but are not limited to, diastereomeric salt formation, kinetic resolution, and asymmetric synthesis. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, N Y, 1962); Wilen, S. H. Tables of Resolving Agents and Optical Resolutions p. 268 (E. L. Eliel, Ed., Univ. of Notre Dame Press, Notre Dame, Ind. 1972), each of which is incorporated herein by reference in their entireties. It is also understood that the present disclosure encompass all possible regioisomers, and mixtures thereof, which can be obtained in pure form by standard separation procedures known to those skilled in the art, and include, but are not limited to, column chromatography, thin-layer chromatography, and high-performance liquid chromatography.

The compounds of the present disclosure include the compounds themselves, as well as their salts and their prodrugs, if applicable. A salt, for example, can be formed between an anion and a positively charged substituent (e.g., amino) on a compound described herein. Suitable anions include chloride, bromide, iodide, sulfate, nitrate, phosphate, citrate, methanesulfonate, trifluoroacetate, and acetate. Likewise, a salt can also be formed between a cation and a negatively charged substituent (e.g., carboxylate) on a compound described herein. Suitable cations include sodium ion, potassium ion, magnesium ion, calcium ion, and an ammonium cation such as tetramethylammonium ion. Examples of prodrugs include C₁₋₆ alkyl esters of carboxylic acid groups, which, upon administration to a subject, are capable of providing active compounds.

Pharmaceutically acceptable salts of the compounds of the present disclosure include those derived from pharmaceutically acceptable inorganic and organic acids and bases. As used herein, the term “pharmaceutically acceptable salt” refers to a salt formed by the addition of a pharmaceutically acceptable acid or base to a compound disclosed herein. As used herein, the phrase “pharmaceutically acceptable” refers to a substance that is acceptable for use in pharmaceutical applications from a toxicological perspective and does not adversely interact with the active ingredient.

Examples of suitable acid salts include acetate, adipate, alginate, aspartate, benzoate, benzenesulfonate, bisulfate, butyrate, citrate, camphorate, camphorsulfonate, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the present disclosure and their pharmaceutically acceptable acid addition salts. Salts derived from appropriate bases include alkali metal (e.g., sodium), alkaline earth metal (e.g., magnesium), ammonium and N-(alkyl)₄⁺ salts. The present disclosure also envisions the quaternization of any basic nitrogen-containing groups of the compounds disclosed herein. Water or oil-soluble or dispersible products may be obtained by such quaternization. Salt forms of the compounds of any of the formulae herein can be amino acid salts of carboxyl groups (e.g. L-arginine, -lysine, -histidine salts).

Lists of suitable salts are found in Remington's Pharmaceutical Sciences, 17th ed., Mack Publishing Company, Easton, Pa., 1985, p. 1418; Journal of Pharmaceutical Science, 66, 2 (1977); and “Pharmaceutical Salts: Properties, Selection, and Use A Handbook; Wermuth, C. G. and Stahl, P. H. (eds.) Verlag Helvetica Chimica Acta, Zurich, 2002 [ISBN 3-906390-26-8] each of which is incorporated herein by reference in their entireties.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

In addition to salt forms, the present disclosure provides compounds that are in a prodrug form. Prodrugs of the compounds described herein are those compounds that undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent. Prodrugs are often useful because, in some situations, they may be easier to administer than the parent drug. They may, for instance, be more bioavailable by oral administration than the parent drug. The prodrug may also have improved solubility in pharmacological compositions over the parent drug. A wide variety of prodrug derivatives are known in the art, such as those that rely on hydrolytic cleavage or oxidative activation of the prodrug. An example, without limitation, of a prodrug would be a compound of the present disclosure which is administered as an ester (the “prodrug”), but then is metabolically hydrolyzed to the carboxylic acid, the active entity. Additional examples include peptidyl derivatives of a compound of the present disclosure.

The present disclosure also includes various hydrate and solvate forms of the compounds.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present disclosure, whether radioactive or not, are intended to be encompassed within the scope of the present disclosure.

### Synthesis

The compounds can be prepared from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures. Those skilled in the art of organic synthesis will recognize that the nature and order of the synthetic steps presented may be varied for the purpose of optimizing the formation of the compounds described herein.

Synthetic chemistry transformations (including protecting group methodologies) useful in synthesizing the compounds described herein are known in the art and include, for example, those such as described in R. C. Larock, Comprehensive Organic Transformations, 2d. Ed., Wiley-VCH Publishers (1999); P. G. M. Wuts and T. W. Greene, Protective Groups in Organic Synthesis, 4th Ed., John Wiley and Sons (2007); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The processes described herein can be monitored according to any suitable method known in the art. For example, product formation can be monitored by spectroscopic means, such as nuclear magnetic resonance spectroscopy (e.g., ¹H or ¹³C), infrared spectroscopy (FT-IR), spectrophotometry (e.g., UV-visible), or mass spectrometry (MS), or by chromatography such as high performance liquid chromatography (HPLC) or thin layer chromatography (TLC).

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene, et al., Protective Groups in Organic Synthesis, 2d. Ed., Wiley & Sons, 1991, which is incorporated herein by reference in its entirety.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

Resolution of racemic mixtures of compounds can be carried out by any of numerous methods known in the art. An example method includes preparation of the Mosher's ester or amide derivative of the corresponding alcohol or amine, respectively. The absolute configuration of the ester or amide is then determined by proton and/or ¹⁹F NMR spectroscopy. An example method includes fractional recrystallization using a “chiral resolving acid” which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid, or the various optically active camphorsulfonic acids. Resolution of racemic mixtures can also be carried out by elution on a column packed with an optically active resolving agent (e.g., dinitrobenzoylphenylglycine). Suitable elution solvent compositions can be determined by one skilled in the art.

### Pharmaceutical Compositions

The term “pharmaceutically acceptable carrier” refers to a carrier or adjuvant that may be administered to a subject (e.g., a patient), together with a compound of the present disclosure, and which does not destroy the pharmacological activity thereof and is nontoxic when administered in doses sufficient to deliver a therapeutic amount of the compound.

Pharmaceutically acceptable carriers, adjuvants and vehicles that may be used in the compositions of the present disclosure include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts, or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

The compositions for administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, with each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules, losenges, or the like in the case of solid compositions. In such compositions, the compound is usually a minor component (from about 0.1 to about 50% by weight or preferably from about 1 to about 40% by weight) with the remainder being various vehicles or carriers and processing aids helpful for forming the desired dosing form.

The amount administered depends on the compound formulation, route of administration, etc. and is generally empirically determined in routine trials, and variations will necessarily occur depending on the target, the host, the route of administration, etc. Generally, the quantity of active compound in a unit dose of preparation may be varied or adjusted from about 1, 3, 10, or 30 to about 30, 100, 300, or 1000 mg, according to the particular application. In a particular embodiment, unit dosage forms are packaged in a multipack adapted for sequential use, such as blisterpack, comprising sheets of at least 6, 9, or 12 unit dosage forms. The actual dosage employed may be varied depending upon the requirements of the patient and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired.

The following are examples (Formulations 1-4) of capsule formulations.

### Capsule Formulations

| | Capsule Formulation | | | |
|---|---|---|---|---|
| | Formu- | Formu- | Formu- | Formu- |
| | lation 1 | lation 2 | lation 3 | lation 4 |
| | mg/capsule | mg/capsule | mg/capsule | mg/capsule |
| Compound | 100 | 400 | 400 | 200 |
| (solid | | | | |
| solution) | | | | |
| Silicon Dioxide | 0.625 | 2.5 | 3.75 | 1.875 |
| Magnesium | 0.125 | 0.5 | 0.125 | 0.625 |
| Stearate NF2 | | | | |
| Croscarmellose | 11.000 | 44.0 | 40.0 | 20.0 |
| Sodium NF | | | | |
| Pluronic F68 | 6.250 | 25.0 | 50.0 | 25.0 |
| NF | | | | |
| Silicon | 0.625 | 2.5 | 3.75 | 1.875 |
| Dioxide NF | | | | |
| Magnesium | 0.125 | 0.5 | 1.25 | 0.625 |
| Stearate NF | | | | |
| Total | 118.750 | 475.00 | 475.00 | 475.00 |
| Capsule Size | No. 4 | No. 0 | No. 0 | No. 2 |

### Preparation of Solid Solution

Crystalline compound (80 g/batch) and the povidone (NF K29/32 at 160 g/batch) are dissolved in methylene chloride (5000 mL). The solution is dried using a suitable solvent spray dryer and the residue reduced to fine particles by grinding. The powder is then passed through a 30 mesh screen and confirmed to be amorphous by X-ray analysis.

The solid solution, silicon dioxide and magnesium stearate are mixed in a suitable mixer for 10 minutes. The mixture is compacted using a suitable roller compactor and milled using a suitable mill fitted with 30 mesh screen. Croscarmellose sodium, Pluronic F68, and silicon dioxide are added to the milled mixture and mixed further for 10 minutes. A premix is made with magnesium stearate and equal portions of the mixture. The premix is added to the remainder of the mixture, mixed for 5 minutes, and the mixture encapsulated in hard shell gelatin capsule shells.

### Use

In one aspect, methods for treating (e.g., controlling, relieving, ameliorating, alleviating, or slowing the progression of) or methods for preventing (e.g., delaying the onset of or reducing the risk of developing) one or more diseases, disorders, or conditions associated with K-Ras in a subject in need thereof are featured. The methods include administering to the subject an effective amount of a compound of formula (A1)-(A38) (and/or a compound of any of the other formulae described herein) or a salt (e.g., a pharmaceutically acceptable salt) thereof as defined anywhere herein to the subject.

In another aspect, the use of a compound of formula (A1)-(A38) (and/or a compound of any of the other formulae described herein) or a salt (e.g., a pharmaceutically acceptable salt) thereof as defined anywhere herein in the preparation of, or for use as, a medicament for the treatment (e.g., controlling, relieving, ameliorating, alleviating, or slowing the progression of) or prevention (e.g., delaying the onset of or reducing the risk of developing) of one or more diseases, disorders, or conditions associated with K-Ras is featured.

In embodiments, the one or more diseases, disorders, or conditions can be cancer, including but not limited to neoplasm or malignant tumors found in mammals (e.g., humans), including leukemia, carcinomas, and sarcomas. Exemplary cancers that may be treated with a compound or method provided herein include cancer of the thyroid, endocrine system, brain, breast, cervix, colon, head and neck, liver, kidney, lung, non-small cell lung, melanoma, mesothelioma, ovary, sarcoma, stomach, uterus, Medulloblastoma, colorectal cancer, or pancreatic cancer. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

### Administration

The compounds and compositions described herein can, for example, be administered orally, parenterally (e.g., subcutaneously, intracutaneously, intravenously, intramuscularly, intraarticularly, intraarterially, intrasynovially, intrasternally, intrathecally, intralesionally, or by intracranial injection or infusion techniques), by inhalation spray, topically, rectally, nasally, buccally, vaginally, via an implanted reservoir, by injection, subdermally, intraperitoneally, transmucosally, or in an ophthalmic preparation, with a dosage ranging from about 0.01 mg/kg to about 1000 mg/kg (e.g., from about 0.01 to about 100 mg/kg, from about 0.1 to about 100 mg/kg, from about 1 to about 100 mg/kg, from about 1 to about 10 mg/kg), every 4 to 120 hours, or according to the requirements of the particular drug. The interrelationship of dosages for animals and humans (based on milligrams per meter squared of body surface) is described by Freireich et al., Cancer Chemother. Rep. 50, 219 (1966). Body surface area may be approximately determined from height and weight of the patient. See, e.g., Scientific Tables, Geigy Pharmaceuticals, Ardsley, N.Y., 537 (1970). In certain embodiments, the compositions are administered by oral administration or administration by injection. The methods herein contemplate administration of an effective amount of compound or compound composition to achieve the desired or stated effect. Typically, the pharmaceutical compositions of the present disclosure will be administered from about 1 to about 6 times per day or, alternatively, as a continuous infusion. Such administration can be used as a chronic or acute therapy.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of the present disclosure may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

In some embodiments, the compounds described herein can be coadministered with one or more other therapeutic agents. In certain embodiments, the additional agents may be administered separately, as part of a multiple dose regimen, from the compounds of the present disclosure (e.g., sequentially, e.g., on different overlapping schedules with the administration of one or more compounds of formula (A1)-(A38) (including any subgenera or specific compounds thereof)). In other embodiments, these agents may be part of a single dosage form, mixed together with the compounds of the present disclosure in a single composition. In still another embodiment, these agents can be given as a separate dose that is administered at about the same time that one or more compounds of formula (A1)-(A38) (including any subgenera or specific compounds thereof) are administered (e.g., simultaneously with the administration of one or more compounds of formula (A1)-(A38) (including any subgenera or specific compounds thereof)). When the compositions of the present disclosure include a combination of a compound of the formulae described herein and one or more additional therapeutic or prophylactic agents, both the compound and the additional agent can be present at dosage levels of between about 1 to 100%, and more preferably between about 5 to 95% of the dosage normally administered in a monotherapy regimen.

The compositions of the present disclosure may contain any conventional non-toxic pharmaceutically-acceptable carriers, adjuvants, or vehicles. In some cases, the pH of the formulation may be adjusted with pharmaceutically acceptable acids, bases, or buffers to enhance the stability of the formulated compound or its delivery form.

The compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution, and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The compositions of the present disclosure may be orally administered in any orally acceptable dosage form including, but not limited to, capsules, tablets, emulsions and aqueous suspensions, dispersions, and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The compositions of the present disclosure may also be administered in the form of suppositories for rectal administration. These compositions can be prepared by mixing a compound of the present disclosure with a suitable non-irritating excipient that is solid at room temperature but liquid at the rectal temperature and therefore will melt in the rectum to release the active components. Such materials include, but are not limited to, cocoa butter, beeswax, and polyethylene glycols.

Topical administration of the compositions of the present disclosure is useful when the desired treatment involves areas or organs readily accessible by topical application. For application topically to the skin, the composition should be formulated with a suitable ointment containing the active components suspended or dissolved in a carrier. Carriers for topical administration of the compounds of the present disclosure include, but are not limited to, mineral oil, liquid petroleum, white petroleum, propylene glycol, polyoxyethylene polyoxypropylene compound, emulsifying wax, and water. Alternatively, the composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol, and water. The compositions of the present disclosure may also be topically applied to the lower intestinal tract by rectal suppository formulation or in a suitable enema formulation.

In some embodiments, topical administration of the compounds and compositions described herein may be presented in the form of an aerosol, a semi-solid pharmaceutical composition, a powder, or a solution. By the term “a semi-solid composition” is meant an ointment, cream, salve, jelly, or other pharmaceutical composition of substantially similar consistency suitable for application to the skin. Examples of semi-solid compositions are given in Chapter 17 of The Theory and Practice of Industrial Pharmacy, Lachman, Lieberman, and Kanig, published by Lea and Febiger (1970) and in Remington's Pharmaceutical Sciences, 21st Edition (2005) published by Mack Publishing Company, which is incorporated herein by reference in its entirety.

Topically transdermal patches are also included in the present disclosure. Also within the present disclosure is a patch to deliver active chemotherapeutic combinations herein. A patch includes a material layer (e.g., polymeric, cloth, gauze, bandage) and the compound of the formulae herein as delineated herein. One side of the material layer can have a protective layer adhered to it to resist passage of the compounds or compositions. The patch can additionally include an adhesive to hold the patch in place on a subject. An adhesive is a composition, including those of either natural or synthetic origin, that when contacted with the skin of a subject, temporarily adheres to the skin. It can be water resistant. The adhesive can be placed on the patch to hold it in contact with the skin of the subject for an extended period of time. The adhesive can be made of a tackiness, or adhesive strength, such that it holds the device in place subject to incidental contact, however, upon an affirmative act (e.g., ripping, peeling, or other intentional removal) the adhesive gives way to the external pressure placed on the device or the adhesive itself, and allows for breaking of the adhesion contact. The adhesive can be pressure sensitive; that is, it can allow for positioning of the adhesive (and the device to be adhered to the skin) against the skin by the application of pressure (e.g., pushing, rubbing) on the adhesive or device.

The compositions of the present disclosure may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

A composition having the compound of the formulae herein and an additional agent (e.g., a therapeutic agent) can be administered using any of the routes of administration described herein. In some embodiments, a composition having the compound of the formulae herein and an additional agent (e.g., a therapeutic agent) can be administered using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). See Negrin et al., Biomaterials, 22(6):563 (2001). Timed-release technology involving alternate delivery methods can also be used in the present disclosure. For example, timed-release formulations based on polymer technologies, sustained-release techniques and encapsulation techniques (e.g., polymeric, liposomal) can also be used for delivery of the compounds and compositions delineated herein.

The present disclosure will be further described in the following examples. It should be understood that these examples are for illustrative purposes only and are not to be construed as limiting the present disclosure in any manner. For example, one of ordinary skill will be able to exercise routine experimentation only, following the examples below, to ascertain compounds that have efficacy in treating or preventing diseases, disorders, or conditions associated with K-Ras. One of ordinary skill will also be able to design and test additional compounds by, e.g., making one or more substitutions thereto, based on principles of medicinal chemistry and pharmaceutical chemistry, again, using routine experimentation only.

### EXAMPLES

### Example 1: In Silico Screen

Compounds that may bind to site A (also referred to as the “irreversible site”) were first identified by substructure searches of the eMolecules structural database to find reversible “analogs” of compounds covalently bound in mutant K-Ras X-ray structures. Next, high-throughput docking was performed with a processed version of the eMolecules structural database (prepared with 3D coordinates representing a sampling of relevant or reasonable protonation states, isomers, tautomers, and conformers), a computationally prepared K-Ras(G12C) protein model derived from a suitable X-ray structure, and Glide software (Schrödinger), followed by spot-checking with Gold software (The Cambridge Crystallographic Data Centre). Also, similarity searches of the eMolecules structural database identified additional analogs of selected compounds that were first discovered by substructure searches or high-throughput docking and later found to be active in assays described herein.

### Example 2: Engineered K-Ras Mutants

The human K-Ras protein (Accession Number P01116) was engineered to include an N-terminal His6 tag, followed by the TEV protease cleavage site (ENLYFQ↓G, highlighted in Table 1 below) immediately before the start codon encoding methionine (underlined in Table 1). The mutation sites are shown with a double underline (e. “ [symbol] ” in G12C).

TABLE 1
K-Ras Constructs Used

| | KRAS(1-169) | Sequence (including His6-tag and TEV cleavage site) |
|---|---|---|
| 1. | WT | 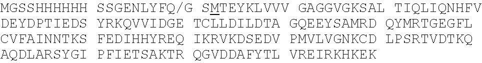 |
| 2. | G12C | 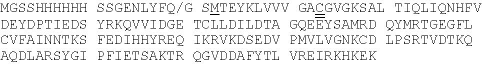 |
| 3. | G12C/S17V | 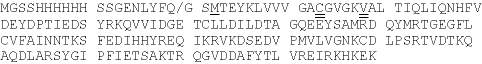 |
| 4. | G12C/T20I | 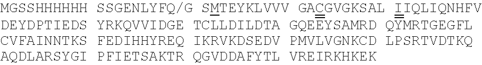 |
| 5. | G12C/T20F | 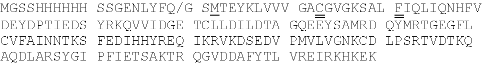 |
| 6. | G12C/I55F | 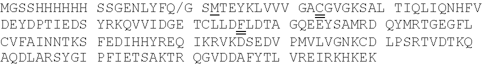 |
| 7. | G12C/D57E | 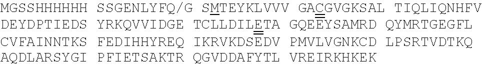 |
| 8. | G12C/D57F | 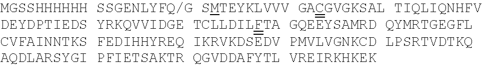 |
| 9. | G12C/D57R | 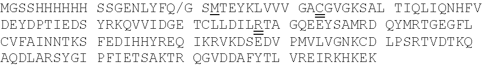 |
| 10. | G12C/T58A | 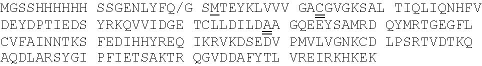 |
| 11. | G12C/T58V | 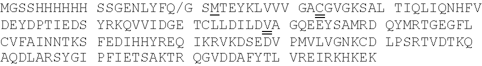 |
| 12. | G12C/T58F | 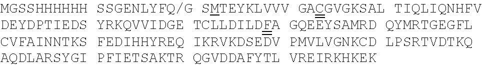 |
| 13. | G12C/G60A | 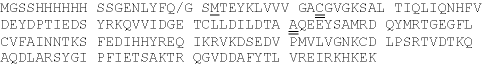 |
| 14. | G12C/G60W | 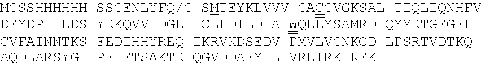 |
| 15. | G12C/Y71W | 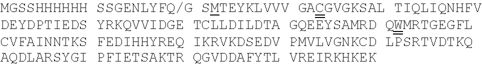 |
| 16. | G12V | 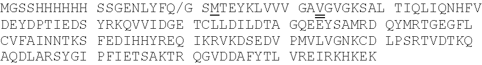 |

### Example 3: Purification of GDP-Bound His6-K-Ras(1-169) Proteins

The pET28b vectors harboring His6-K-Ras(1-169) mutants were transformed into E. coli BL21(DE3) (Novagen). Cultures (typically 2 L) derived form single colonies were grown at 37° C. in LB medium containing 50 μg/ml kanamycin until A600 reaches 0.6-0.8. The culture was chilled on ice for 30 min, then His6-K-Ras expression was induced with 1 mM isopropyl β-D-thiogalactoside (IPTG). Incubation was continued at 23° C. overnight. Cells were harvested by centrifugation using an SLA 3000 rotor at 5,000 rpm for 20 min, and cells were stored at −80° C.

All subsequent procedures were carried out at 4° C. Cell pellets were suspended in buffer A (50 mM Tris/HCl and 500 mM NaCl) and 1:20000 ratio of Benzonase® Nuclease (Sigma, 250 units/μL) was added. The suspensions were mixed gently for at least 30 min, and the cells were lysed by microfluidizer. The insoluble material was removed by centrifugation at 14,000 rpm in an SLA1500 rotor for 45 min. The soluble lysates were gently mixed with 4 ml of 50% slurry Ni-NTA agarose (Qiagen) beads, which had been equilibrated in buffer A. The resins were recovered by centrifugation using a Thermo Scientific Legend XTR centrifuge at 4,000 rpm for 5 min, and washed with 20-column volume (CV) of buffer A containing 25 mM imidazole. The cycle of centrifugation and resuspension of the resins was repeated three times. At third wash, transfer beads carefully over to disposable column. Bound proteins were eluted stepwise with 2 CV of 50, 100, 200, 300, and 500 mM imidazole in BufferA. The elution profiles were monitored by SDS-PAGE. Most of the bound His6-K-Ras(1-169) protein was recoved in the 50-100 mM imidazole eluates. Peak fractions containing His6-K-Ras(1-169) were pooled and concentrated using Amicon Ultra-15 (10 kDa MWCO) to achieve approximately 500 M (11 mg/ml).

To remove pre-bound GTP from His6-K-Ras(1-169), the protein was incubated with Incubate with 25 mM EDTA, 2 mM DTT, and 5 mM GDP for overnight at 4° C. The GDP exchanged His6-K-Ras(1-169) was filtered through 0.22 μm membrane and loaded onto Superdex 75 16/60, which had been equilibrated in 50 mM HEPES (pH 7.5), 200 mM NaCl, 10% glycerol, and 1 mM TCEP. Most of the bound His6-K-Ras(1-169) protein was eluted at retention volume of 73 ml, and the peak fractions were pooled and concentrated using Amicon Ultra-15 (10 kDa MWCO). Excess amount of GDP and EDTA was also removed by gel-filtration. Due to nucleotide binding property of His6-K-Ras(1-169), protein concentrations were determined by using the Bradford protein assay (Bio-Rad) with bovine serum albumin (BSA) as the standard.

### Example 4: Purification of GST-TEV-Avi-cRaf (55-132)

The pGEX vector harboring GST-Avi-cRaf (55-132) was transformed into E. coli that contains biotin ligase BirA. Culture derived form a single colony was grown at 37° C. in TB medium containing 100 μg/ml ampicillin. GST-cRaf (55-132) was induced with 0.5 mM IPTG and in vivo biotinylated in the presence of 50 μM biotin. Induction was carried out at 18° C. for overnight. Cells were harvested by centrifugation and stored at −80° C. The protein sequence for expression vector harboring GST-TEV-Avi-cRaf (55-132) is shown below with the TEV protease cleavage site underlined (ENLYFQG↓G):

MSPILGYWKI KGLVQPTRLL LEYLEEKYEE HLYERDEGDK
WRNKKFELGL EFPNLPYYID GDVKLTQSMA IIRYIADKHN
MLGGSPKERA EISMLEGAVL DIRYGVSRIA YSKDFETLKV
DFLSKLPEML KMFEDRLSHK TYLNGDHVTH PDFMLYDALD
VVLYMDPMSL DAFPKLVSFK KRIEAIPQID KYLKSSKYIA
WPLQGWQATF GGGDHPPKSD LVPRGSGSEN LYFQG↓GLNDI
FEAQKIEWRS NTIRVFLPNK QRTVVNVRNG MSLHDCLMKA
LKVRGLQPEC CAVFRLLHEH KGKKARLDWN TDAASLIGEE
LQVDFLD.

All subsequent procedures were carried out at 4° C. The cell pellet was resuspended in 50 mM HEPES (pH 8.0), 400 mM NaCl, 10% glycerol, 1 mM DTT, 20 units/mL benzonase (EMD Millipore, Cat No. 70746) and cOmplete™, EDTA Free Protease Inhbitor Cocktail Tablets (Roche Diagonotics, Cat No. 11873580001) as per manufacturers recommended concentrations. The suspension was lysed using Microfluidics microfluidizer and the supernatant was retained via centrifugation of lysis at 12,000 rpm for 40 min. The supernatant was filtered using 0.22 μm filter unit (EMD Millipore, Cat No. SCGPT02RE) before loading onto GSTrap HP (GE Healthcare, Cat No. 17-5282-02) using ÄKTA Purifier. The column was washed as per manufacturer recommended protocol and then eluted with 20 mM L-glutathione, reduced (Sigma Aldrich, Cat No. G4251) with the eluate collected in fractions. The fractions were analyzed using SDS-PAGE and fractions with the target protein were collected and pooled. Pooled fractions were concentrated using Vivacell 100, 30K MWCO (Sartorius, Cat No. VC1022) and loaded onto a Hiload Superdex 200 26/600 PG (GE Healthcare, Cat No. 28989336), pre-equilibrated with 50 mM HEPES (pH 8.0), 200 mM NaCl, 10% glycerol, and 1 mM DTT. SDS-PAGE was used to analyze the fractions for the target protein, and corresponding fractions were collected and pooled. The pooled fractions were concentrated using Vivacell 100, 30K MWCO (Sartorius, Cat No. VC1022) until 3.6 mg/mL.

### Example 5: K-Ras Mutant/Raf Binding Assay

### K-Ras Mutant/Raf Binding Assay Used for Single-Point Screen

K-Ras mutant activity was measured by its binding ability with c-Raf (a downstream effector molecule). The K-Ras mutants tested were K-Ras(WT), K-Ras(G12C), and K-Ras(G12C, T58A).

1.33 μM recombinant His-tag K-Ras mutant protein (GDP form) was prepared in Assay Buffer 1 (AB-1; 20 mM HEPES pH 7.5, 100 mM NaCl, 0.3 mM TCEP, and 10 mM EDTA). 1.5 μL per well of test compounds (10 mM stock in DMSO) was added into a 384-well polypropylene black plate (NUNC) followed by 30 μL per well of K-Ras/AB-1 solution. The plate was incubated at 4° C. overnight (15-20 hours) and then incubated at room temperature (RT, 20° C.) for 60 min.

5 μL per well of Assay Buffer 2 (AB-2; 50 mM Tris pH 7.4, 100 mM NaCl, 0.75 mM DTT, 1.46 mg/mL BSA, and 4% (v/v) DMSO) was added to the plate which was then centrifuged at 1,200 rpm (Eppendorf 5810R Plate centrifuge) for 1 min. 9.5 μL from each well in the plate was transferred to a new polypropylene plate containing 72.5 μL per well of Assay Buffer 3 (AB-3; 50 mM Tris pH 7.4, 100 mM NaCl, 0.75 mM DTT, 0.2 mg/mL BSA, and 4% (v/v) DMSO). 24 μL per well from the K-Ras/AB-3 dilution plate was transferred to three new empty polypropylene plates.

0.782 μM GST-TEV-Avi-cRaf, 5.6 nM Europium-labeled streptavidin (Eu-SA, Perkin Elmer), 230 nM allophycocyanin (APC)-conjugated anti His6 antibody (available from Columbia Biosciences), and GTP (400 nM for K-Ras(G12C, T58A); 120 nM for K-Ras(G12C) and K-Ras (WT)) was prepared in Assay Buffer 4 (AB-4; 50 mM Tris pH 7.4, 100 mM NaCl, 0.75 mM DTT, 0.4 mg/mL BSA, and 8 mM MgCl2). 4 μL per well of the Raf/Eu-SA/APC/GTP/AB-4 solution was added to the three plates. 4 μL per well of Assay Buffer 5 (AB-5; 50 mM Tris pH 7.4, 100 mM NaCl, 0.75 mM DTT, 80 mM EDTA, and 4% (v/v) DMSO) was also added to the three plates. The plates were centrifuged at 1,200 rpm for 1 min and then incubated at RT for 30 min. Assay signals were monitored by exciting the samples at 340 nm and reading emission fluorescence at 615 nm and 665 nm on an Envision reader.

Normalized time-resolved fluorescence resonance energy transfer (TR-FRET) assay signal (Rₙ) was calculated using the following formulas:

Rn=(E₆₁₅−B₆₁₅)·[(S₆₆₅−B₆₆₅)−C·(S₆₁₅−B₆₁₅)]/(S₆₁₅−B₆₁₅)

C=(E₆₆₅−B₆₆₅)/(E₆₁₅−B₆₁₅)

where E₆₁₅ and E₆₆₅ are the fluorescence intensities of 0.7 nM Eu-SA in AB-3 at 615 nm and 665 nm, respectively; B₆₁₅ and B₆₆₅ are the fluorescence intensities of AB-3 at 615 nm and 665 nm, respectively; S₆₁₅ and S₆₆₅ are the fluorescence intensities of the samples at 615 nm and 665 nm, respectively; and C is the cross-talk factor.

The percent inhibition of each compound was calculated based on wells initially containing 1.5 μL DMSO.

### K-Ras Mutant/Raf Binding Assay for Dose Response of Compounds

K-Ras mutant activity was measured by its binding ability with Raf (a downstream effector molecule). The K-Ras mutants tested were K-Ras(WT), K-Ras(G12C), K-Ras(G12V), and K-Ras(G12C, T58A).

1.33 μM recombinant His-tag K-Ras mutant protein (GDP form) was prepared in Assay Buffer 1 (AB-1; 20 mM HEPES pH 7.5, 100 mM NaCl, 0.3 mM TCEP, and 10 mM EDTA). Test compounds (10 mM stock in DMSO) were diluted 3-fold in series in DMSO for 7 concentrations. 1.2 μL per well of the test compound dilutions was added into a 384-well polypropylene black plate (NUNC) followed by 24 μL per well of K-Ras/AB-1 solution. The plate was incubated either at room temperature (RT, 20° C.) for 30 min or at 4° C. overnight followed by 1 hour at RT.

4 μL per well of Assay Buffer 2 (AB-2; 50 mM Tris pH 7.4, 100 mM NaCl, 0.75 mM DTT, 1.46 mg/mL BSA, and 4% (v/v) DMSO) was added to the plate which was then centrifuged at 1,200 rpm (Eppendorf 5810R Plate centrifuge) for 1 min. 9.5 μL from each well in the plate was transferred to a new polypropylene plate containing 72.5 μL per well of Assay Buffer 3 (AB-3; 50 mM Tris pH 7.4, 100 mM NaCl, 0.75 mM DTT, 0.2 mg/mL BSA, and 4% (v/v) DMSO). 24 μL per well from the K-Ras/AB-3 dilution plate was transferred to three new empty polypropylene plates.

0.782 μM GST-TEV-Avi-cRaf, 5.6 nM Europium-labeled streptavidin (Eu-SA, Perkin Elmer), 230 nM allophycocyanin (APC)-conjugated anti His6 antibody, and GTP (400 nM or 200 nM for K-Ras(G12C, T58A); 120 nM for K-Ras(G12C), K-Ras(G12V), and K-Ras (WT)) was prepared in Assay Buffer 4 (AB-4; 50 mM Tris pH 7.4, 100 mM NaCl, 0.75 mM DTT, 0.4 mg/mL BSA, and 8 mM MgCl₂). 4 μL per well of the Raf/Eu-SA/APC/GTP/AB-4 solution was added to the three plates. 4 μL per well of Assay Buffer 5 (AB-5; 50 mM Tris pH 7.4, 100 mM NaCl, 0.75 mM DTT, 80 mM EDTA, and 4% (v/v) DMSO) was also added to the three plates. The plates were centrifuged at 1,200 rpm for 1 min and then incubated at RT for 30 min. Assay signals were monitored by exciting the samples at 340 nm and reading emission fluorescence at 615 nm and 665 nm on an Envision reader.

Normalized time-resolved fluorescence resonance energy transfer (TR-FRET) assay signal (Rn) was calculated using the following formulas:

Rₙ=(E₆₁₅−B₆₁₅)·[(S₆₆₅−B₆₆₅)−C·(S₆₁₅−B₆₁₅)]/(S₆₁₅−B₆₁₅)

C=(E₆₆₅−B₆₆₅)/(E₆₁₅−B₆₁₅)

where E₆₁₅ and E₆₆₅ are the fluorescence intensities of 0.7 nM Eu-SA in AB-3 at 615 nm and 665 nm, respectively; B₆₁₅ and B₆₆₅ are the fluorescence intensities of AB-3 at 615 nm and 665 nm, respectively; S₆₁₅ and S₆₆₅ are the fluorescence intensities of the samples at 615 nm and 665 nm, respectively; and C is the cross-talk factor.

The percent inhibition of each compound well was calculated based on wells initially containing 1.2 μL DMSO. IC₅₀ values were calculated using either Prism (GraphPad) or ActivityBase (IDBS) software.

A total of 719 compounds were tested in various data sets using the proteins indicated herein. In some embodiments, a “primer” (e.g., benzimidazole, etc.) was added to serve as a positive allosteric enhancer of inhibitor activity.

The percent inhibition for each compound at each selected concentration in each data set is determined in triplicate, and the arithmetic average was used in single-concentration testing and for each point of a dose-response curve.

The sample concentration used for the assay measurements of inhibition is 35.7 μM for all compounds. The sample concentrations for dose-response determinations are 35.7, 11.9, 3.97, 1.32, 0.441, and 0.0490 μM.

### EQUIVALENTS

The present disclosure provides among other things K-Ras mutants, compounds and use thereof. While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

To date, over two hundred compounds were identified as assay hits and are represented by, e.g., formula (A1)-(A38). Without wishing to be bound by theory, formula (A1)-(A38) compounds are believed to bind to site A. In some embodiments, the compounds exhibited inhibition ≥20.0% or an IC₅₀ value ≤35.7 μM. In other embodiments, the compounds exhibited inhibition <20.0% but >0%. The compounds may exhibit enhanced inhibition in the presence of a primer.

### INCORPORATION BY REFERENCE

All publications, patents, and sequence database entries mentioned herein are hereby incorporated by reference in their entirety as if each individual publication or patent was specifically and individually indicated to be incorporated by reference.

### ILLUSTRATIVE EMBODIMENTS

Described below are illustrative embodiments EA-ELL of the invention. It will be understood that referencing an embodiment (e.g., EC, ED, EE, EF, etc.) will refer to all subembodiments described in connection with that embodiment. For example, refences to embodiment EC include EC1, EC2, EC3, EC4, EC5, EC6, EC7, EC8, etc. Any disclosed range of embodiments (e.g., EA-ELL) refer to each embodiment within the disclosed range (e.g., a range EY-ECC specifies embodiments EY, EZ, EAA, EBB, and ECC).

In the chemical structures shown below, atoms marked with an asterix “*” may be chiral centers. In some embodiments, chiral compounds of the invention may be in the “R” or “S” configuaration, or a racemic mixture thereof. Atoms marked with “*” may also be achiral (not chiral), depending on the attached substituents.

{EA1}. A non-naturally occurring mutant of human K-Ras protein, comprising a mutation selected from the group consisting of: S17V, T20I, T20F, I55F, D57E, D57F, D57R, T58A, T58V, T58F, G60A, G60W, and Y71W.
{EA2}. A non-naturally occurring mutant of human K-Ras protein, comprising a first mutation of G12C and a second mutation selected from the group consisting of: S17V, T20I, T20F, I55F, D57E, D57F, D57R, T58A, T58V, T58F, G60A, G60W, and Y71W.
{EA3}. A cDNA encoding the non-naturally occurring mutant of human K-Ras protein of Embodiments {EA1} or {EA2}.
{EA4}. A small primer molecule (e.g., benzimidazole,), which provides enhanced sensitivity in the testing and identification of potential inhibitors of mutant K-Ras.
{EB1}. A method for identifying compounds that selectively inhibit mutant K-Ras, comprising:
(a) providing a non-naturally occurring mutant of human K-Ras protein according to Embodiment {EA3}, or a functional fragment thereof,
(b) contacting said non-naturally occurring mutant of human K-Ras protein with Raf and a candidate compound;
(c) measuring inhibition of binding of said non-naturally occurring mutant of human K-Ras protein with Raf by the candidate compound as compared to a wildtype K-Ras control,
(d) measuring inhibition of binding of wildtype human K-Ras protein with Raf by the candidate compound;
   wherein, greater inhibition of binding of Raf with said non-naturally occurring mutant of human K-Ras protein compared to said wildtype human K-Ras protein indicates that the candidate compound is a selective inhitor of mutant K-Ras.
   {EB2}. A pharmaceutical composition comprising a pharmaceutically acceptable excipient and a compound or compounds that selectively inhibit mutant K-Ras.
   {EB3}. A method of treating cancer, comprising administering an effective amount of the compound or compounds that selectively inhibit mutant K-Ras, wherein the cancer is associated with a K-Ras mutation (e.g., K-Ras(G12C)).

### Scaffold A1—Embodiment C (“EC”)

{EC1}. A K-Ras inhibiting compound having a structure according to formula (A1), or a pharmaceutically acceptable salt thereof:

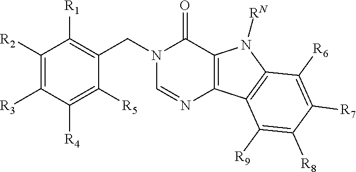

wherein, R₁, R₂, R₃, R₄, R₅, R₆, R₇, R₈, and R₉ are each independently selected from the group consisting of hydrogen, —X, —R, and -L₁-R;
X is independently selected at each occurrence from —F, —Cl, —Br, —I, —OH, —OR*, —NH₂, —NHR*, —N(R*)₂, —N(R*)₃⁺, —N(R*)—OH, —N(→O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂⁻, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —C(═NR*)—R*, —SCN, —NCS, —NSO, —SSR*, —N(R*)—C(═O)—N(R*)₂, —CH₃, —CH₂—CH₃, —CH₂H₂—CH₃, —C(H)(CH₂)₂, —C(CH₃)₃, —N(R*)—C(═S)—N(R*)₂, —S(O)₁₋₂—R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂;
R* is, independently at each occurrence, H or a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.);
R is independently selected at each occurrence from C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteratoms selected from O, S, N, P, F, Cl, Br, I, and combinations thereof;
L₁ is selected independently at each occurrence from —O—, —S—, —NH—, —N(R^{N})—, —(CH₂)₁₋₄—, —C═C—, —C═C—(CH₂)₁₋₃—, —C(O)—, —C(O)—(CH₂)₁₋₃—, —(CH₂)₁₋₃—C(O)—, —C(O)—O—(CH₂)₁₋₃—, —C(O)—N(R^{N})—, —N(R^{N})—C(O), —C(O)—N(R^{N})—(CH₂)₁₋₃—, —(CH₂)₁₋₃—C(O)—N(R^{N})—, —NH—S(O)₁₋₂—, —N(R^{N})—S(O)₁₋₂—, —S(O)₁₋₂—N(R^{N})—, —O—(CH₂)₁₋₃—, —(CH₂)₁₋₃—O—, —S—(CH₂)₁₋₃—, —(CH₂)₁₋₃—S—, —NH—(CH₂)₁₋₃—, —N(R^{N})—CH₂)₁₋₃—, —(CH₂)₁₋₃—N(R^{N})—, —S(═O)₁₋₂—, or —(OCH₂CH₂)₁₋₃—; and
R^{N} is hydrogen, methyl, ethyl, or propyl.
{EC2}. The K-Ras inhibiting compound according to Embodiment {EC1}, wherein R₁, R₂, R₄, and R₅ are all hydrogen.
{EC3}. The K-Ras inhibiting compound according to Embodiment {EC1}, wherein R₃ is selected from the group consisting of —OH, —SH, —NH₂; —N(R*)₂; —F, —Cl, —Br, —I, —CN, —CH₃, —CF₃, —CHF₂, —O—CF₃, —OCHF₂, —CH₂CH₃, —CH₂CH₂CH₃, —CH(CH₃)₂, —OCH₃, and —SCH₃.
{EC4}. The K-Ras inhibiting compound according to Embodiment {EC1}, wherein R₆, R₇, R₈, and R₉ are independently selected from the group consisting of hydrogen, —OH, —SH, —NH₂; —N(R*)₂; —F, —Cl, —Br, —I, —CF₃, —CHF₂, —CH₃, —CH₂CH₃, —CH₂CH₂CH₃, —CH(CH₃)₂, —OCH₃, —OCHF₂, and —OCF₃.
{EC5}. The K-Ras inhibiting compound according to Embodiment {EC1}, wherein R₃ is —F.
{EC6}. The K-Ras inhibiting compound according to Embodiment {EC1}, wherein R₃ is —OCH₃.
{EC7}. The K-Ras inhibiting compound according to Embodiment {EC1}, wherein one of R₆, R₇, R₈, and R₉ is —CH₃ or —OCH₃, and the remaining groups R₆, R₇, R₈, and R₉ are each hydrogen.
{EC8}. The K-Ras inhibiting compound according to Embodiments {EC1}-{EC7}, wherein R^{N} is hydrogen.

### Scaffold A2—Embodiment D (“ED”)

{ED1}. A compound for inhibiting K-Ras having formula (A2), or a pharmaceutically acceptable salt thereof:

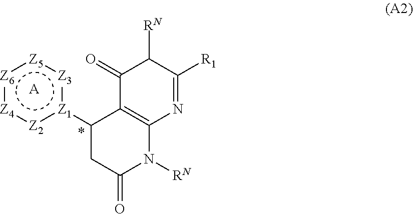

wherein, ring “A” is a five- or six-membered optionally aromatic ring, z₁ is C, CH, or N; and z₂-z₆ are independently selected from N, NH, NR^{N}, O, S, C═O, CH, CX, CR, CH₂, C(X)(X), C(R)(X), or C(R)(R); and wherein ring “A” may contain 0, 1, 2, or 3 double bonds; and in the case where ring “A” is a five-membered ring, z₄ is a bond (i.e., it is absent); and wherein any two vicinal substituents X and/or R and/or R^{N} may together form a 5- or 6-membered ring fused to ring “A”;
wherein, R₁ is selected from the group consisting of hydrogen, —X, —R, -L₁-R, -L₁-R^{B}—R^{Q}—R, —R^{Q}—X, -(L)₀₋₁-(R^{L})₀₋₁—(R^{Q})₀₋₁—X, -(L₁)₀₋₁-(R^{L})₀₋₁—(R^{Q})₀₋₁—R^{B} and -(L₁)₀₋₁-(R^{L})₀₋₁—(R^{Q})₀₋₁—R;
X is independently selected at each occurrence from —F, —Cl, —Br, —I, —OH, —OR*, —NH₂, —NHR*, —N(R*)₂, —N(R*)₃⁺, —N(R*)—OH, —N(→O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂⁻, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(O)₁₋₂—R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂;
R* is, independently at each occurrence, H or a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), or aryl-alkyl (e.g., toluyl);
R is independently selected at each occurrence from hydrogen or C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteratoms selected from O, S, N, P, F, Cl, Br, I, and combinations thereof;
R^{Q} is a cyclic group having the structure:

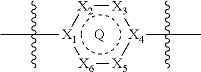

wherein ring “Q” is a five- or six-membered optionally aromatic ring, x₁ and x₄ are independently C, CH, CR or N; and x₂, x₃, x₅, and x₆, are independently selected from N, NH, NR^{N}, O, S, C═O, CH, CX, CR, CH₂, C(X)(X), C(R)(X), and C(R)(R); and in the case where ring “Q” is a five-membered ring, x₃ or x₅ is a bond (i.e., it is absent); and wherein any two vicinal substituents X and/or R and/or R^{N} may together form a 5- or 6-membered ring fused to ring “Q”;
R^{B} is independently selected at each occurrence from C₂₋₁₂ cyclic hydrocarbons (alicyclic or aromatic) and heterocycles (e.g., heteroaryl), optionally substituted with one or more (e.g., 1-3) groups X and/or with 1-10 heteratoms selected from O, S, N, P, F, Cl, Br, I, and combinations thereof;
L₁ is selected independently at each occurrence from —O—, —S—, —NH—, —N(R^{N})—, —(CH₂)₁₋₄—, —C═C—, —C═C—(CH₂)₁₋₃—, —C(O)—, —C(O)—(CH₂)₁₋₃—, —(CH₂)₁₋₃—C(O)—, —C(O)—O—(CH₂)₁₋₃—, —C(O)—N(R^{N})—, —N(R^{N})—C(O)—, —C(O)—N(R^{N})—(CH₂)₁₋₃—, —(CH₂)₁₋₃—C(O)—N(R^{N})—, —NH—S(O)₁₋₂—, —N(R^{N})—S(O)₁₋₂—, —S(O)₁₋₂—N(R^{N})—, —O—(CH₂)₁₋₃—, —(CH₂)₁₋₃—O—, —S—(CH₂)₁₋₃—, —(CH₂)₁₋₃—S—, —NH—(CH₂)₁₋₃—, —N(R^{N})—(CH₂)₁₋₃—, —(CH₂)₁₋₃—N(R^{N})—, —S(═O)₁₋₂—, or —(OCH₂CH₂)₁₋₃—; and
R^{N} is independently selected at each occurrence from hydrogen, methyl, ethyl, or propyl;
and wherein the chiral center indicate by “*” may be in the “R” or “S” configuaration, or a racemic mixture thereof.
{ED2}. The K-Ras inhibiting compound according to Embodiment {ED1}, wherein R₁ is —R^{B} or -L₁-R^{B}.
{ED3}. The K-Ras inhibiting compound according to Embodiment {ED2}, R^{B} has the form:

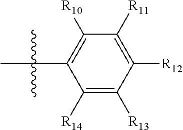

wherein R₁₀, R₁₁, R₁₂, R₁₃, and R₁₄ are independently selected at each occurrence from the group consisting of hydrogen, X, —R*, and —OR*.
{ED4}. The K-Ras inhibiting compound according to Embodiment {ED3}, wherein R₁₀, R₁₁, R₁₂, R₁₃, and R₁₄ are independently selected from the group consisting of hydrogen, —OH, —SH, —NH₂; —N(R*)₂; —F, —Cl, —Br, —I, —CH₃, —CH₂CH₃, —CH₂CH₃, —CH₂CH₂CH₃, —CH(CH₃)₂, —OCH(CH₃)₂, —OCH₃, —SCH₃, and —OCF₃.
{ED5}. The K-Ras inhibiting compound according to Embodiment {ED3}, wherein all of R₁₀, R₁₁, R₁₂, R₁₃, and R₁₄ are hydrogen.
{ED6}. The K-Ras inhibiting compound according to Embodiment {ED3}, wherein one of R₁₀, R₁₁, R₁₂, R₁₃, and R₁₄ is —Cl.
{ED7}. The K-Ras inhibiting compound according to any one of Embodiments {ED2}-{ED6}, wherein R₁ is -L₁-R^{B} where L₁ is —S—CH₂—.
{ED8}. The K-Ras inhibiting compound according to any one of Embodiments {ED1}-{ED7}, wherein ring “A” is a six-membered aromatic ring, z₁ is C, and at least one of z₂, z₃, z₄, z₅, and z₆ is CX.
{ED9}. The K-Ras inhibiting compound according to Embodiment {ED8}, wherein X is F.
{ED10}. The K-Ras inhibiting compound according to Embodiment {ED8}, wherein z₁ is C and at least two of z₂, z₃, z₄, z₅, and z₆ are CF.
{ED11}. The K-Ras inhibiting compound according to any one of Embodiments {ED1}-{ED10}, wherein R^{N} is H at each occurrence.
{ED12}. The K-Ras inhibiting compound according to Embodiment {ED1}, wherein ring “A” is may be selected from the group consisting of:

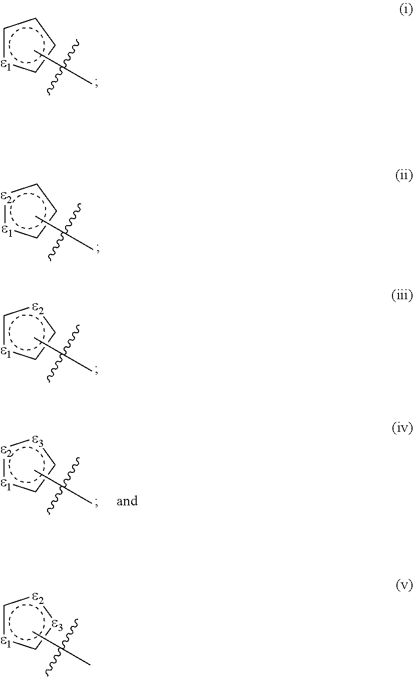

wherein ε₁, ε₂, and ε₃, are independently selected from N, NH, NR^{N}, NR*, —C(═O)—, S, and O; with the proviso that where the point of attachment is ε₁, ε₂, or ε₃, then that position represents N; and wherein carbon atoms which are not the point of attachment may be optionally substituted with a group X (e.g., F, Cl, Br, —SH, —OH, —OCH₃) or with a group R (e.g., —CH₃, —CH₂CH₃, etc.) which may in turn be substituted with one or more (e.g., 1-3) groups X and/or 1-10 heteratoms selected from O, S, N, P, F, Cl, Br, I, and combinations thereof; and wherein the dashed circles indicate that each ring may comprise zero, one, or two double bonds and may be aromatic, and wherein any two adjacent groups X, R^{N}, R*, and/or R may together form a 5- or 6-membered ring fused with ring “A.”
{ED13}. The K-Ras inhibiting compound according to Embodiment {ED1}, wherein ring “A” is a thiophen-3-yl radical of the form

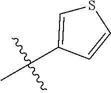

wherein, any available carbon atom is optionally substituted with a group X (e.g., F, Cl, Br, —SH, —OH, or —OCH₃) or with a group R (e.g., —CH₃, —CH₂CH₃, etc.).
{ED14}. The K-Ras inhibiting compound according to Embodiment {ED12}, wherein z₁ is C and z₂ is CX.
{ED15}. The K-Ras inhibiting compound according to Embodiment {ED12}, wherein z₁ is C and z₂ is CF and at least one of z₃, z₄, z₅, and z₆ is CF.
{ED16}. The K-Ras inhibiting compound according to Embodiment {ED12}, wherein z₁ is C and at least three of z₂, z₃, z₄, z₅, and z₆ are CF.
{ED17}. The K-Ras inhibiting compound according to Embodiment {ED1}, wherein R₁ is —R^{Q}—X.
{ED18}. The K-Ras inhibiting compound according to Embodiment {ED17}, wherein ring “Q” is a six-membered ring and x₂ is N.
{ED19}. The K-Ras inhibiting compound according to Embodiment {ED17}, wherein —X is —C(O)—NH₂.
{ED20}. The K-Ras inhibiting compound according to any one of Embodiments {ED1}-{ED19}, wherein R^{N} is hydrogen at each occurence.
{ED21}. The K-Ras inhibiting compound according to Embodiments {ED1}, wherein R₁ is a group of the form —S—(CH₂)₀₋₂—R^{B}, where R^{B} has the form:

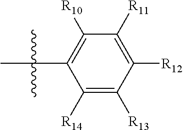

wherein R₁₀, R₁₁, R₁₂, R₁₃, and R₁₄ are independently selected at each occurrence from the group consisting of hydrogen, X, —R*, and —OR*; and
ring “A” is a phenyl ring, optionally substituted with 1-5 groups X, and where R^{N} is hydrogen at each occurrence.

### Scaffold A3—Embodiment E (“EE”)

{EE1}. A compound for inhibiting K-Ras having formula (A3), or a pharmaceutically acceptable salt thereof:

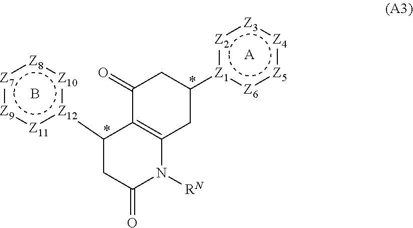

wherein ring “A” is a five- or six-membered optionally aromatic ring, z₁ is selected from C, CH, or N; and z₂-z₆ are selected from N, NH, NR^{N}, O, S, C═O, CH, CX, CR, CH₂, C(X)(X), C(R)(X), and C(R)(R); and wherein ring “A” may contain 0, 1, 2, or 3 double bonds; and in the case where ring “A” is a five-membered ring, z₄ is a bond (i.e., it is absent); and wherein any two vicinal substituents X and/or R and/or R^{N} may together form a 5- or 6-membered ring fused to ring “A”;
ring “B” is a five- or six-membered optionally aromatic ring, z₁₂ is selected from C, CH, or N; and z₇-z₁₁ are selected from N, NH, NR^{N}, O, S, C═O, CH, CX, CR, CH₂, C(X)(X), C(R)(X), and C(R)(R); and wherein ring “B” may contain 0, 1, 2, or 3 double bonds; and in the case where ring “B” is a five-membered ring, z₇ is a bond (i.e., it is absent); and wherein any two vicinal substituents X and/or R and/or R^{N} may together form a 5- or 6-membered ring fused to ring “B”;
X is independently selected at each occurrence from —F, —Cl, —Br, —I, —OH, —OR*, —NH₂, —NHR*, —N(R*)₂, —N(R*)₃⁺, —N(R*)—OH, —N(→O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(O)₁₋₂—R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —CH₃, —O—(CH₂)₁₋₄CH₃, —OC(H)(CH₂)₂—OC(CH₃)₃, —PR*₂, —O—P(═O)(OR*)₂, or —P(═O)(OR*)₂;
R* is, independently at each occurrence, H or a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.);
R is independently selected at each occurrence from hydrogen or C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteratoms selected from O, S, N, P, F, Cl, Br, I, and combinations thereof;
R^{N} is independently selected at each occurrence from hydrogen, methyl, ethyl, or propyl; and
wherein “*” indicates a chiral center which may be in the “R” or “S” configuration, or a racemic mixture thereof, and wherein the compound may be in the form of R,R or R,S or S,R or S,S diastereomers.
{EE2}. The K-Ras inhibiting compound according to Embodiment {EE}, wherein z₁ is C, and z₂-z₆ are, respectively, groups C—R₁₀, C—R₁₁, C—R₁₂, C—R₁₃, and C—R₁₄, such that ring “A” has the form:

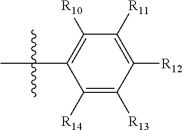

wherein R₁₀, R₁₁, R₁₂, R₁₃, and R₁₄ are independently selected at each occurrence from the group consisting of hydrogen, X, —R, —R*, and —OR*, wherein any two adjacent groups R and/or R* and/or —OR*, may together form a 5- or 6-membered ring fused to ring “A.”
{EE3}. The K-Ras inhibiting compound according to Embodiment {EE2}, wherein R₁₀, R₁₁, R₁₂, R₁₃, and R₁₄ are independently selected from the group consisting of hydrogen, —OH, —SH, —NH₂; —N(R*)₂; —F, —Cl, —Br, —I, —CH₃, —CH₂CH₃, —CH₂CH₃, —CH₂CH₂CH₃, —CH(CH₃)₂, —OCH₂CH₃, —OCH(CH₃)₂, —OCH₃, —SCH₃, and —OCF₃.
{EE4}. The K-Ras inhibiting compound according to Embodiment {EE3}, wherein all (or at least 1, 2, 3, or 4) of R₁₀, R₁₁, R₁₂, R₁₃, and R₁₄ are hydrogen.
{EE5}. The K-Ras inhibiting compound according to Embodiment {EE3}, wherein at least one one (e.g., 1, 2, 3, 4, or 5) of R₁₀, R₁₁, R₁₂, R₁₃, and R₁₄ is —OCH₃.
{EE6}. The K-Ras inhibiting compound according to Embodiment {EE3}, wherein at least one one (e.g., 1, 2, 3, 4, or 5) of R₁₀, R₁₁, R₁₂, R₁₃, and R₁₄ is —F.
{EE7}. The K-Ras inhibiting compound according to Embodiment {EE1}, wherein z₄ is a bond (i.e., it is absent), and z₁, z₂, z₃, z₅, and z₆ are, respectively, groups C—R₁₀, C—R₁₁, C—R₁₂, C—R₁₃, and C—R₁₄, such that ring “A” may be selected from the group consisting of:

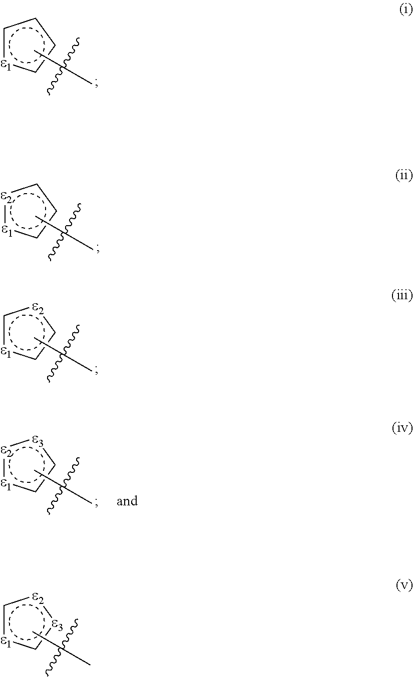

wherein ε₁, ε₂, and ε₃, are independently selected from N, NH, NR^{N}, NR*, —C(═O)—, S, and O; with the proviso that where the point of attachment is ε₁, ε₂, or ε₃, then that position represents N; and wherein carbon atoms which are not the point of attachment may be optionally substituted with a group X (e.g., F, Cl, Br, —SH, —OH, —OCH₃) or with a group R (e.g., —CH₃, —CH₂CH₃, etc.) which may in turn be substituted with one or more (e.g., 1-3) groups X and/or 1-10 heteratoms selected from O, S, N, P, F, Cl, Br, I, and combinations thereof; and wherein the dashed circles indicate that each ring may comprise zero, one, or two double bonds and may be aromatic, and wherein any two adjacent groups X, R^{N}, R*, and/or R may together form a 5- or 6-membered ring fused with ring “A.”
{EE8}. The K-Ras inhibiting compound according to Embodiment {EE7}, wherein ring “A” is a thiophen-2-yl radical of the form

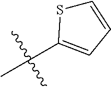

wherein, any available carbon atom is optionally substituted with a group X (e.g., F, Cl, Br, —SH, —OH, or —OCH₃) or with a group R (e.g., —CH₃, —CH₂CH₃, etc.).
{EE9}. The K-Ras inhibiting compound according to Embodiment {EE}, wherein z₁₂ is C, and z₇-z₁₁ are, respectively, groups C—R₁₆, C—R₁₇, C—R₁₅, C—R₁₄, and C—R₁₈, such that ring “B” has the form:

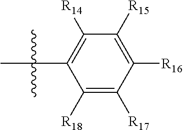

wherein R₁₄, R₁₅, R₁₆, R₁₇, and R₁₈ are independently selected at each occurrence from the group consisting of hydrogen, X, —R, —R*, and —OR*, wherein any two adjacent groups R and/or R* and/or —OR*, may together form a 5- or 6-membered ring fused to ring “A.”
{EE10}. The K-Ras inhibiting compound according to Embodiment {EE9}, wherein R₁₄, R₁₅, R₁₆, R₁₇, and R₁₈ are independently selected from the group consisting of hydrogen, —OH, —SH, —NH₂; —N(R*)₂; —F, —Cl, —Br, —I, —CH₃, —CH₂CH₃, —CH₂CH₃, —CH₂CH₂CH₃, —CH(CH₃)₂, —OCH₂CH₃, —OCH(CH₃)₂, —OCH₃, —SCH₃, and —OCF₃.
{EE11}. The K-Ras inhibiting compound according to Embodiment {EE9}, wherein all (or at least 1, 2, 3, or 4) of R₁₄, R₁₅, R₁₆, R₁₇, and R₁₈ are hydrogen.
{EE12}. The K-Ras inhibiting compound according to Embodiment {EE9}, wherein at least one one (e.g., 1, 2, 3, 4, or 5) of R₁₄, R₁₅, R₁₆, R₁₇, and R₁₈ is —OCH₃.
{EE13}. The K-Ras inhibiting compound according to Embodiment {EE9}, wherein at least one one (e.g., 1, 2, 3, 4, or 5) of R₁₄, R₁₅, R₁₆, R₁₇, and R₁₈ is —F.
{EE14}. The K-Ras inhibiting compound according to Embodiment {EE}, wherein z₇ is a bond (i.e., it is absent), such ring “B” may be selected from the group consisting of:

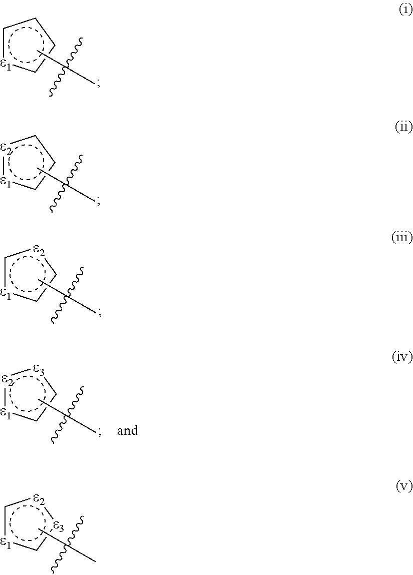

wherein ε₁, ε₂, and ε₃, are independently selected from N, NH, NR^{N}, NR*, —C(═O)—, S, and O; with the proviso that where the point of attachment is ε₁, ε₂, or ε₃, then that position represents N; and wherein carbon atoms which are not the point of attachment may be optionally substituted with a group X (e.g., F, Cl, Br, —SH, —OH, —OCH₃) or with a group R (e.g., —CH₃, —CH₂CH₃, etc.) which may in turn be substituted with one or more (e.g., 1-3) groups X and/or 1-10 heteratoms selected from O, S, N, P, F, Cl, Br, I, and combinations thereof; and wherein the dashed circles indicate that each ring may comprise zero, one, or two double bonds and may be aromatic, and wherein any two adjacent groups X, R^{N}, R*, and/or R may together form a 5- or 6-membered ring fused with ring “B.”
{EE15}. The K-Ras inhibiting compound according to Embodiment {EE9}, wherein ring “B” is a thiophen-2-yl radical of the form

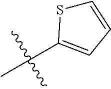

wherein, any available carbon atom is optionally substituted with a group X (e.g., F, Cl, Br, —SH, —OH, or —OCH₃) or with a group R (e.g., —CH₃, —CH₂CH₃, etc.).
{EE16}. The K-Ras inhibiting compound according to any of Embodiments {EE1}-{EE15}, wherein R^{N} is hydrogen at each occurence.

### Scaffold A4—Embodiment F (“EF”)

{EF1}. A compound for inhibiting K-Ras having formula (A4), or a pharmaceutically acceptable salt thereof:

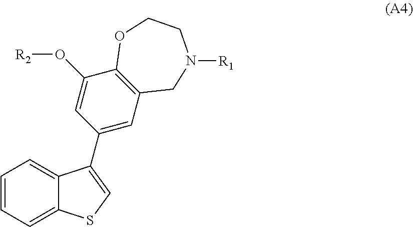

wherein, R₁ is selected from the group consisting of hydrogen, —R, —R^{B}—X, -L₁-X, -L₁-R, -L₁-R^{B}, -L₁-R^{L}—X, -(L₁)₀₋₁-(R^{L})₀₋₁—X, —(R^{L})₀₋₁-(L₁)₀₋₁-X, -(L₁)₀₋₁-(R_{L})₀₋₁—R, —(R^{L})₀₋₁-(L₁)₀₋₁-R, -(L₁)₀₋₁-(R^{L})₀₋₁—R^{B}, and —(R^{L})₀₋₁-(L₁)₀₋₁-R^{B}; where
R₂ is hydrogen or R; where
R is selected from hydrogen or C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteroatoms selected from O, S, N, P, F, Cl, Br, I, or combinations thereof;
L₁ is selected independently at each occurrence from —O—, —S—, —NH—, —N(R^{N})—, —(CH₂)₁₋₄—, —C═C—, —C═C—(CH₂)₁₋₃—, —C(O)—, —C(O)—CH₂)₁₋₃—, —(CH₂)₁₋₃—(O), —C(O)—O—(CH₂)₁₋₃—, —C(O)—N(R^{N})—, —N(R^{N})—C(O)—, —C(O)—N(R^{N})—(CH₂)₁₋₃—, —(CH₂)₁₋₃—C(O)—N(R^{N})—, —NH—S(O)₁₋₂—, —N(R^{N})—S(O)₁₋₂—, —S(O)₁₋₂—N(R^{N})—, —O—(CH₂)₁₋₃—, —(CH₂)₁₋₃—O—, —S—(CH₂)₁₋₃—, —(CH₂)₁₋₃—S—, —NH—(CH₂)₁₋₃—, —N(R^{N})—(CH₂)₁₋₃—, —(CH₂)₁₋₃—N(R^{N})—, —S(═O)₁₋₂—, or —(OCH₂CH₂)₁₋₃—;
R^{B} is a C₃₋₆ cyclic hydrocarbon (alicyclic or aromatic) or heterocycle (e.g., heteroaryl), optionally substituted with one or more (e.g., 1-5) groups X and/or with 1-6 heteroatoms selected from O, S, N, P, F, Cl, Br, I, and combinations thereof, where any two vicinal groups may together form a 5- or 6-membered fused ring with said cyclic hydrocarbon;
R^{L} is independently selected at each occurrence from C₁₋₆ linear or branched bivalent hydrocarbon radicals; optionally substituted with one or more (e.g., 1-5) groups X and/or with 1-6 heteroatoms selected from O, S, N, P, F, Cl, Br, I, and combinations thereof,
X is independently selected at each occurrence from —F, —Cl, —Br, —I, —OH, —OR*, —NH₂, —NHR*, —N(R*)₂, —N(R*)₃⁺, —N(R*)—OH, —N(→O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂⁻, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(O)₁₋₂—R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₄CH₃, —OC(H)(CH₂)₂—OC(CH₃)₃, —PR*₂, —O—P(═O)(OR*)₂, or —P(═O)(OR*)₂;
R^{N} is independently selected at each occurrence from hydrogen, methyl, ethyl, or propyl; and
R* is, independently at each occurrence, H or a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.), and wherein R* is optionally substituted with 1-5 groups X and/or 1-4 heteroatoms selected from O, S, N, P, F, Cl, Br, I, and combinations thereof.
{EF2}. The K-Ras inhibiting compound according to Embodiment {EF1}, wherein R₁ is -L₁-R, where L₁ is —(CH₂)₁₋₃—, —C(═O)—, or —(CH₂)₁₋₃—C(═O)—; and R is selected from C₁₋₁₂ linear or branched hydrocarbons, C₃₋₁₂ cyclic hydrocarbons (alicyclic or aromatic) or C₂₋₁₂ heterocycles (e.g., heteroaryl), optionally substituted with one or more (e.g., 1-5) groups X and/or with 1-10 heteroatoms selected from O, S, N, P, F, Cl, Br, I, and combinations thereof.
{EF3}. The K-Ras inhibiting compound according to Embodiment {EF2}, wherein L₁ of R₁ is a group —C(O)—.
{EF4}. The K-Ras inhibiting compound according to Embodiment {EF2}, wherein R of R₁ has the form —(CR′R″)₀₋₄—N(R*)₂, where R′ and R″ are independently selected at each occurrence from hydrogen, —OH, —OCH₃, —CH₃, —CH₂CH₃, and C₁₋₆ hydrocarbons optionally substituted with one or more (e.g., 1-5) groups X and/or with 1-10 heteroatoms selected from O, S, N, P, F, Cl, Br, I, and combinations thereof; where R′ or R″ may together with R* form a heterocyclic ring; and where groups R* may together, with the nitrogen atom to which they are attached form a 3-6 membered heterocyclic ring, or where a group R* may together with R′ or R″ form a 5- or 6-membered heterocyclic ring.
{EF5}. The K-Ras inhibiting compound according to Embodiment {EF1}, wherein R₂ is hydrogen, and R₁ is -L₁-R^{L}—X, were R^{L} is a group —CH₂— or —C(H)(CH₃)— and X is —NH₂ or a salt thereof.
{EF6}. The K-Ras inhibiting compound according to Embodiment {EF5}, wherein R₁ has a single chiral center in the “S” configuration.
{EF7}. The K-Ras inhibiting compound according to any one of Embodiments {EF1}-{EF6}, wherein said compound is a pharmaceutically acceptable salt.
{EF8}. The K-Ras inhibiting compound according to Embodiment {EF3}, wherein R of R₁ has the form —(CR′R″)₁₋₄—OR*, where R′ and R″ are independently selected at each occurrence from hydrogen, —CH₃, and —CH₂CH₃; where R′ or R″ may together with R* form a heterocyclic ring.
{EF9}. The K-Ras inhibiting compound according to Embodiment {EF3}, where R of R₁ is a ring “A” having the following structure:

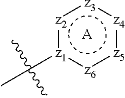

where ring “A” is a five- or six-membered, optionally aromatic ring, where z₁ is C, CH, or N; and z₂-z₆ are independently selected from N, NH, NR^{N}, O, S, C═O, CH, CX, CR*, CH₂, C(X)(X), C(R*)(X), or C(R*)(R*), and in the case where ring “A” is a five-membered ring, z₄ is a bond (i.e., it is absent); and wherein any two vicinal substituents X and/or R* and/or R^{N} may together form a 5- or 6-membered ring fused to ring “A.”
{EF10}. The K-Ras inhibiting compound according to Embodiment {EF6}, wherein A is 6-membered aromatic ring.
{EF11}. The K-Ras inhibiting compound according to Embodiment {EF7}, wherein z₁ is C and one or two of z₂-z₆ is N.
{EF12}. The K-Ras inhibiting compound according to Embodiment {EF1}, wherein R₁ is a group -L₁-R^{B}.
{EF13}. The K-Ras inhibiting compound according to Embodiment {EF1}, wherein R₁ is a group -L₁-R^{B}, where L₁ is —C(O)— and R^{B} has the structure:

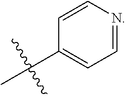 [SMILES: *c1ccncc1]

{EF14}. The K-Ras inhibiting compound according to any one of Embodiments {EF}-{EF12}, wherein R₂ is hydrogen.

### Scaffold A5—Embodiment G (“EG”)

{EG1}. A compound for inhibiting K-Ras having formula (A5), or a pharmaceutically acceptable salt thereof:

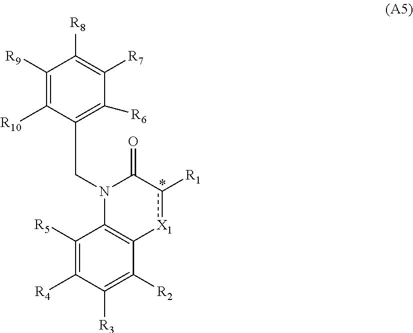

wherein the “dashed” bond may be a single or double bond;
X₁ may be CH, CR, CX, C═O, CH₂, C(X)(X), C(R)(R), N, NH, NR, NX, S, or O;
R₁ is selected from hydrogen, —R, -L₁-R, —R^{L}-L₁-R, —R^{L}-L₁-R^{L}—R, or —R^{L}-(L₁)₁₋₂-R^{B}—R^{L}-(L₁)₁₋₂-(R^{L})₀₋₁—R^{B};
R₂-R₁₀ are independently selected from hydrogen, —X, and —R;
R is selected from hydrogen and C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteroatoms selected from O, S, N, P, F, Cl, Br, I, and combinations thereof;
R^{L} is independently selected from each occurrence from C₁₋₆ linear or branched bivalent hydrocarbon radicals; optionally substituted with one or more (e.g., 1-5) groups X and/or with 1-6 heteroatoms selected from O, S, N, P, F, Cl, Br, I, and combinations thereof;
R^{B} is a C₃₋₆ cyclic hydrocarbon (alicyclic or aromatic) or heterocycle (e.g., heteroaryl), optionally substituted with one or more (e.g., 1-5) groups X and/or with 1-6 heteroatoms selected from O, S, N, P, F, Cl, Br, I, and combinations thereof, where any two vicinal groups may together form a 5- or 6-membered fused ring with said cyclic hydrocarbon;
X is independently selected at each occurrence from —F, —Cl, —Br, —I, —OH, —OR*, —NH₂, —NHR*, —N(R*)₂, —N(R*)₃⁺, —N(R*)—OH, —N(→O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂⁻, CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂H₂—CH₃, —C(H)(CH₂)₂, —C(CH₃)₃, —N(R*)—C(═S)—N(R*)₂, —S(O)₁₋₂—R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₄CH₃, —OC(H)(CH₂)₂—OC(CH₃)₃, —PR*₂, —O—P(═O)(OR*)₂, or —P(═O)(OR*)₂;
R* is independently selected at each occurrence from hydrogen or a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.); and
L₁ is selected independently at each occurrence from —O—, —S—, —NH—, —N(R^{N})—, —(CH₂)₁₋₄—, —C═C—, —C═C—(CH₂)₁₋₃—, —C(O)—, —C(O)—(CH₂)₁₋₃—, —(CH₂)₁₋₃—C(O)—, —C(O)—O—, —O—C(O)—, —C(O)—O—(CH₂)₁₋₃—, —C(O)—N(R^{N})—, —N(R^{N})—C(O)—, —C(O)—N(R^{N})—(CH₂)₁₋₃—, —(CH₂)₁₋₃—C(O)—N(R^{N})—, —NH—S(O)₁₋₂—, —N(R^{N})—S(O)₁₋₂—, —S(O)₁₋₂—N(R^{N})—, —O—(CH₂)₁₋₃—, —(CH₂)₁₋₃—O—, —S—(CH₂)₁₋₃—, —(CH₂)₁₋₃—S—, —NH—(CH₂)₁₋₃—, —N(R^{N})—(CH₂)₁₋₃—, —(CH₂)₁₋₃—N(R^{N})—, —S(═O)₁₋₂—; and —(OCH₂CH₂)₁₋₃—; where
R^{N} is independently selected at each occurrence from hydrogen, methyl, ethyl, or propyl.
{EG2}. The K-Ras inhibiting compound according to Embodiment {EG1} having the structure:

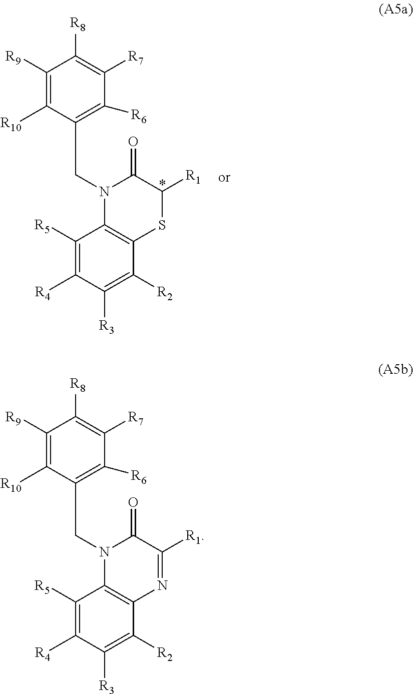

{EG3}. The K-Ras inhibiting compound according to Embodiment {EG1}, wherein R₁ is —R^{L}-(L₁)₁₋₂-R^{B}, where L₁ is selected at each occurrence from from —C(O)—N(H)— and —C(O)—N(H)—(CH₂)₁₋₃—.
{EG4}. The K-Ras inhibiting compound according to Embodiment {EG3}, wherein —R^{L}— of R₁ is —C(R^{N})₂—.
{EG5}. The K-Ras inhibiting compound according to Embodiment {EG3}, wherein R^{B} of R₁ is a six-membered ring having the following structure:

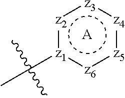

where ring “A” is a five- or six-membered, optionally aromatic ring, where z₁ is C, CH, or N; and z₂-z₆ are independently selected from N, NH, NR^{N}, O, S, C═O, CH, CX, CR*, CH₂, C(X)(X), C(R*)(X), or C(R*)(R*); and in the case where ring “A” is a five-membered ring, z₄ is a bond (i.e., it is absent); and wherein any two vicinal substituents X and/or R* and/or R^{N} may together form a 5- or 6-membered ring fused to ring “A.”
{EG6}. The K-Ras inhibiting compound according to Embodiment {EG3}, wherein ring “A” is aromatic.
{EG7}. The K-Ras inhibiting compound according to Embodiment {EG3}, wherein at least one of z₂, z₃, z₄, z₅, and z₆ are CX, where X is selected from —O—R*, —C(O)—N(R*)₂, —Cl or —F.
{EG8}. The K-Ras inhibiting compound according to Embodiment {EG3}, wherein at least two of z₂, z₃, z₄, z₅, and z₆ are CX, where X is selected from —O—R*, —C(O)—N(R*)₂, —Cl or —F.
{EG9}. The K-Ras inhibiting compound according to Embodiment {EG1}, wherein at least one of R₂-R₅ is —CF₃.
{EG10}. The K-Ras inhibiting compound according to Embodiment {EG1}, wherein R₃ is —CF₃ and R₂, R₄, and R₅ are hydrogen.
{EG11}. The K-Ras inhibiting compound according to Embodiment {EG1}, wherein at least one of R₆-R₁₀ is —Cl or —F.
{EG12}. The K-Ras inhibiting compound according to Embodiment {EG1}, wherein at least two of R₆-R₁₀ is —Cl or —F.
{EG13}. The K-Ras inhibiting compound according to Embodiment {EG1} having the structure:

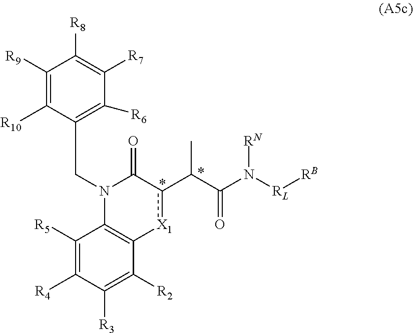

{EG14}. The K-Ras inhibiting compound according to Embodiment {EG13}, wherein the two specified stereocenters are in the (R,S), (R,R), (S,S), or (S,R) configurations.
{EG15}. The K-Ras inhibiting compound according to Embodiment {EG1} having the structure:

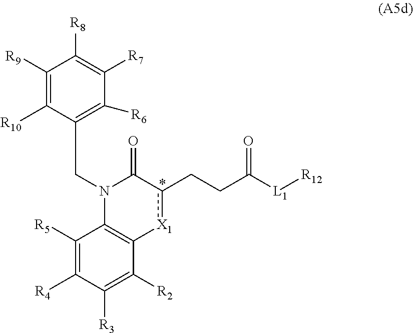

wherein R₁₂ is -(L₁)₀₋₁-(R^{L})₀₋₁-(L₁)₀₋₁-R^{B}.

### Scaffold A6—Embodiment H (“EH”)

{EH1}. A compound for inhibiting K-Ras having a structure of formula (A6), or a pharmaceutically acceptable salt thereof:

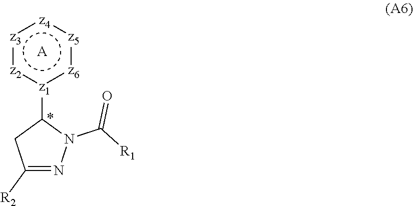

wherein ring “A” is a five- or six-membered, optionally aromatic, ring; where z₁ is C, CH, or N; and z₂-z₆ are independently selected from N, NH, NR^{N}, O, S, C═O, CH, CX, CR, CH₂, C(X)(X), C(R)(X), or C(R)(R); and wherein ring “A” may contain 0, 1, 2, or 3 double bonds; and in the case where ring “A” is a five-membered ring, z₄ is a bond (i.e., it is absent); and wherein any two vicinal substituents X and/or R and/or R^{N} may together form a 5- or 6-membered ring fused to ring “A”;
R₁ is selected from the group consisting of hydrogen, —X, —R, —R^{B}, —R^{Q}—R^{B}, -L₁-R, -(L₂)ₓ-(CR*₂)_{y}-(L₃)ₘ-(R^{Q})ₙ-(L₄)ₚ-(CR*₂)_{q}-(L₅)ᵣ-R, -(L₂)ₓ-(CR*₂)_{y}-(L₃)ₘ-(R^{Q})ₙ-(L₄)ₚ-(CR*₂)_{q}-(L₅)ᵣ-R^{B}, or -(L₂)ₓ-(CR*₂)_{y}-(L₃)ₘ-(R^{Q})ₙ-(L₄)ₚ-(CR*₂)_{q}-(L₅)ᵣ-X; where x, y, m, n, p, q, and r are integers independently selected from 0-3 (i.e., 0, 1, 2, or 3);
R₂ is selected from the group consisting of hydrogen, —X, —R, —R^{B}, —R^{Q}—R^{B}, -L₁-R, -(L₂)ₓ-(CR*₂)_{y}-(L₃)ₘ-(R^{Q})ₙ-(L₄)ₚ-(CR*₂)_{q}-(L₅)ᵣ-R, (L₂)ₓ-(CR*₂)_{y}-(L₃)ₘ-(R^{Q})ₙ-(L₄)ₚ-(CR*₂)_{q}-(L₅)ᵣ-R^{B}, or -(L₂)ₓ-(CR*₂)_{y}-(L₃)ₘ-(R^{Q})ₙ-(L₄)ₚ(CR*₂)_{q}-(L₅)ᵣ-X; where x, y, m, n, p, q, and r are integers independently selected from 0-3 (i.e., 0, 1, 2, or 3);
R is selected from hydrogen and C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteroatoms selected from O, S, N, P, F, Cl, Br, I, and combinations thereof;
R^{Q} is a monocyclic or fused bicyclic group having the structure:

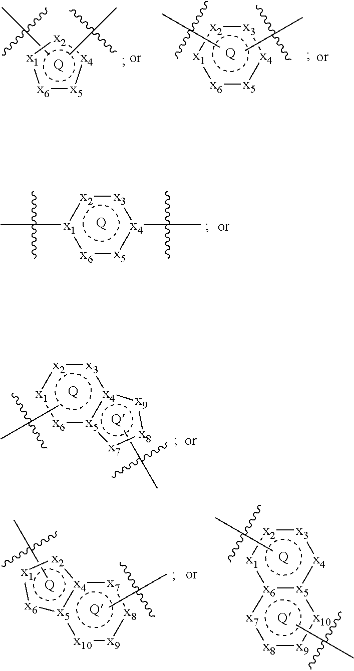

wherein ring Q and Q′ are independently five- or six-membered, optionally aromatic rings; x₁-x₁₀ are independently selected from N, NH, NR^{N}, O, S, C═O, C, CH CX, CR, CH₂, C(X)(X), C(R)(X), and C(R)(R); and in the case where ring “Q” is a five-membered ring, X₃ is a bond (i.e., it is absent); and wherein any two vicinal substituents X and/or R and/or R^{N} may together form an optionally substituted 5- or 6-membered ring fused to ring “Q” and/or ring “Q′”;
R^{B} is a C₃₋₆ cyclic hydrocarbon (alicyclic or aromatic) or heterocycle (e.g., heteroaryl), optionally substituted with one or more (e.g., 1-5) groups X and/or with 1-6 heteroatoms selected from O, S, N, P, F, Cl, Br, I, or combinations thereof, where any vicinal groups may together form a 5- or 6-membered fused ring with said cyclic hydrocarbon;
X is independently selected at each occurrence from —F, —Cl, —Br, —I, —OH, —OR*, —NH₂, —NHR*, —N(R*)₂, —N(R*)₃⁺, —N(R*)—OH, —N(→O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂⁻, CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(═O)₁₋₂—R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂; where
R* is independently selected at each occurrence from hydrogen and a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.); and
L₁-L₅ are selected independently at each occurrence from group L, where L is —O—, —S—, —NH—, —N(R^{N})—, —(CH₂)₁₋₄—, —C═C—, —C═C—(CH₂)₁₋₃—, —C(O)—, —C(O)—(CH₂)₁₋₃—, —C(O)—O—(CH₂)₁₋₃—, —C(O)—N(R^{N})—, —C(O)—N(R^{N})—(CH₂)₁₋₃—, —(CH₂)₁₋₃—C(O)—N(R^{N})—, —(CH₂)₀₋₃—NH—C(O)—O—, —(CH₂)₀₋₃—NH—(CH₂)₁₋₃—, —CH₂—NH—S(O)₁₋₂—, —N(R^{N})—S(O)₁₋₂—, —O—(CH₂)₁₋₃—, —O—(CH₂)₁₋₃—C(O)—O—, —S—(CH₂)₁₋₃—, —N(R^{N})—(CH₂)₁₋₃—, and —(OCH₂CH₂)₁₋₃—; where
R^{N} is independently selected at each occurrence from hydrogen, methyl, ethyl, or propyl, and pharmaceutically acceptable salts thereof.
{EH2}. The K-Ras inhibiting compound according to Embodiment {EH1}, wherein ring “A” is phenyl.
{EH3}. The K-Ras inhibiting compound according to Embodiment {EH1}, wherein ring “A” is five-membered.
{EH4}. The K-Ras compound structure according to Embodiment {EH1}, wherein ring “A” is thiophenyl, furanyl, or pyrrolyl.
{EH5}. The K-Ras compound structure according to Embodiment {EH1}, wherein ring “A” is napthyl.
{EH6}. The K-Ras compound structure according to Embodiment {EH1}, wherein ring “A” is substituted with at least one halogen.
{EH7}. The K-Ras compound structure according to Embodiment {EH1}, wherein ring “A” is substituted with at least one C₁₋₆ alkoxy.
{EH8}. The K-Ras inhibiting compound according to Embodiment {EH1}, wherein R₁ has the form —(CH₂)₀₋₁-L₃-R^{Q}—CH₂-L₄-R or —(CH₂)₀₋₁-L₃-R^{Q}—CH₂-L₄-R^{B}, where -L₃-R^{Q}—CH₂-L₄- has the structure:

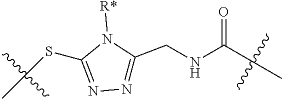

{EH9}. The K-Ras inhibiting compound according to Embodiment {EH1}, wherein two vicinal groups R on ring “A” together form a fused ring are —N═CH—CH═N—, where each nitrogen is attached to the adjacent carbon atom on ring A to form a fused ring.
{EH10}. The K-Ras inhibiting compound according to Embodiment {EH1}, having formula (A6a)

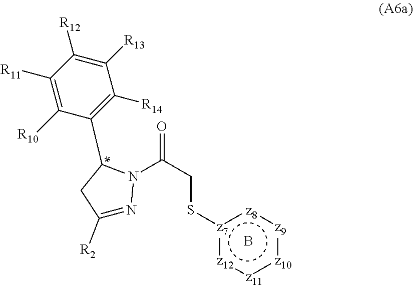

wherein ring “B” is a five- or six-membered optionally aromatic ring, z₇ is C, CH, or N; and z₈-z₁₂ are independently selected from N, NH, NR^{N}, O, S, C═O, CH, CX, CL₁R, CL₁X, C(L₄)ₚ(CR*₂)_{q}(L₅)ᵣR, C(L₄)ₚ(CR*₂)_{q}(L₅)ᵣX, CR, CH₂, C(X)(X), C(R)(X), or C(R)(R); and in the case where ring “B” is a five-membered ring, z₁₀ is a bond (i.e., it is absent); and wherein any two vicinal substituents X and/or R and/or R^{N} may together form a 5- or 6-membered ring fused to ring “B”; and
R₁₀-R₁₄ are independently selected from hydrogen, —X, —OR*, or —R*.
{EH11}. The K-Ras inhibiting compound according to Embodiment {EH1}, having the structure:

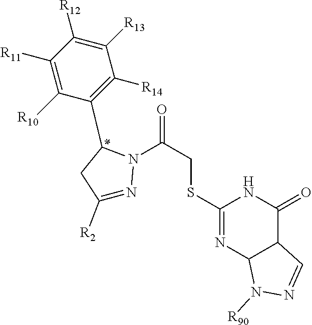

wherein R₉₀ is hydrogen, —R (e.g., lower alkyl, etc.), or —(CH₂)₀₋₂—X.
{EH12}. The K-Ras inhibiting compound according to Embodiment {EH6}, wherein ring “B” is a five-membered ring, selected from the group consisting of optionally substituted pyrazole, imidazole, triazole, and tetrazole, each optionally having a 5- or 6-membered aromatic ring fused thereto.
{EH13}. The K-Ras inhibiting compound according to Embodiment {EH7}, where z₈ is NH or NR; and z₁₁ and z₁₂ are N.
{EH14}. The K-Ras inhibiting compound according to Embodiment {EH8}, wherein z₉ is CR and R is —CH₂—NH—C(O)—CH₂—R^{B}
{EH15}. The K-Ras inhibiting compound according to Embodiment {EH6}, wherein R₂ is R^{B} or —R^{Q}—R^{B}.
{EH16}. The K-Ras inhibiting compound according to Embodiment {EH15}, wherein R^{B} of R₂ is a six-membered optionally substituted aromatic ring.
{EH17}. The K-Ras inhibiting compound according to Embodiment {EH15}; wherein R^{B} of R₂ is optionally substituted thiophenyl, furanyl, or pyrrolyl.
{EH18}. The K-Ras inhibiting compound according to Embodiment {EH1} having the formula (A6b):

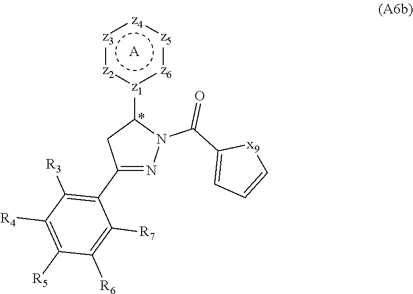

wherein, x₉ is selected from NH, NR^{N}, O, and S; and R₃-R₇ are independently selected from hydrogen, —X, -Q, —R, -L₁-R, or a group -(L₂)ₓ-(CR*₂)_{y}-(L₃)ₘ-(R^{Q})ₙ-(L₄)ₚ-(CR*₂)_{q}-(L₅)ᵣ-R;
{EH19} The K-Ras inhibiting compound according to Embodiment {EH18}, wherein x₉ is S or O.
{EH20}. The K-Ras inhibiting compound according to Embodiment {EH18}, wherein any one of R₃-R₇ has the form -L₂-(CR*₂)ₙ-L₃-R, where n is 0, 1, or 2.
{EH21}. The K-Ras inhibiting compound according to Embodiment {EH20}, wherein L₂ is O and L₃ is —C(O)—O—.
{EH22}. The K-Ras inhibiting compound according to Embodiment {EH21}, wherein one of R₃-R₇ is -L₂-(CR*₂)ₙ—C(O)—R^{B}
{EH23}. The K-Ras inhibiting compound according to Embodiment {EH18}, wherein one of R₃-R₇ is -L₂-CH₂—C(O)—R^{B}
{EH24}. The K-Ras inhibiting compound according to Embodiment {EH22} or {EH23}, wherein R^{B} of said one of R₃-R₇ is a five- or six-membered heterocyclic ring, having a nitrogen atom at the point of attachment.
{EH25}. The K-Ras inhibiting compound according to Embodiment {EH24}, wherein R^{B} of said one of R₃-R₇ is selected from piperidenyl, morpholinyl, and pyrroyl.
{EH26}. The K-Ras inhibiting compound according to Embodiment {EH18}, wherein R₃-R₇ are independently selected from the group consisting of hydrogen, —OH, —Cl, —F, —Br, —I, —CH₃, —CH₂—CH₃, —O—CH₃, —O—CH₂—CH₃, —NH₂, and —CN.
{EH27}. The K-Ras inhibiting compound according to Embodiment {EH1}, having a structure of formula (A6c):

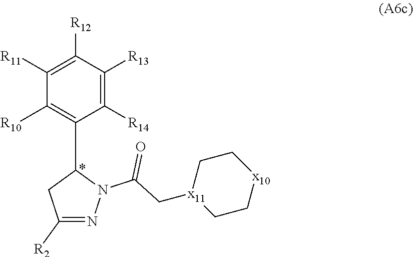

wherein x₁₀ is NH, NX, NR, CH₂, C(H)(R), C(H)(X), C(X)(X), C(R)(X), or C(R)(R); where any geminal groups may form a 5- or 6-membered spiro ring; and
x₁₁ may be C, N, or NH⁺.
{EH28}. The K-Ras inhibiting compound according to Embodiment {EH27}, wherein x₁₀ is C(H)(X), where X is —C(O)—NH₂.
{EH29}. The K-Ras inhibiting compound according to Embodiment {EH27}, wherein x₁₀ is C(R)(R), where each R together form a group —O—CH₂—CH₂—O—, wherein the oxygen atoms form an acetal at x₁₀.
{EH30}. The K-Ras inhibiting compound according to Embodiment {EH27}, wherein x₁₀ is N(R^{B}) or N(X).
{EH31}. The K-Ras inhibiting compound according to Embodiment {EH27}, wherein x₁₁ is NH⁺
{EH32}. The K-Ras inhibiting compound according to Embodiment {EH1}, wherein R₂ is phenyl.
{EH33}. The K-Ras inhibiting compound according to Embodiment {EH1}, wherein R₂ is napthyl.
{EH34}. The K-Ras inhibiting compound according to Embodiment {EH1}, wherein R₂ is thiophenyl.
{EH35}. The K-Ras inhibiting compound according to Embodiment {EH1}, wherein R₂ is thiophen-2-yl.
{EH36}. The K-Ras inhibiting compound according to Embodiment {EH1}, having a structure of formula (A6d):

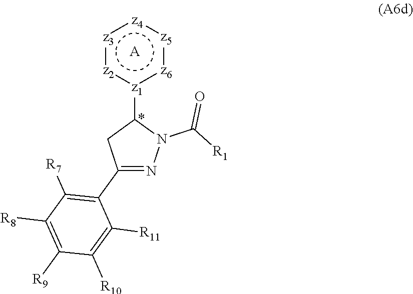

wherein R₇-R₁₁ are independently selected from hydrogen, F, Cl, Br, OH, OCH₃, OCH₂CH₃, OCH₂CH₂CH₃, OC(CH₃)₃, CH₃, CH₂CH₃, CH₂CH₂CH₃, or C(CH₃)₃.
{EH37}. The K-Ras inhibiting compound according to Embodiment {EH1}, having the structure:

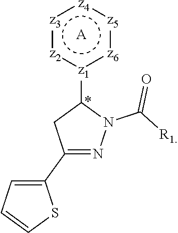

{EH38}. The K-Ras inhibiting compound according to Embodiment {EH1}, having the structure:

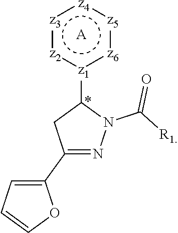

{EH39}. The K-Ras inhibiting compound according to Embodiment {EH1}, having the structure:

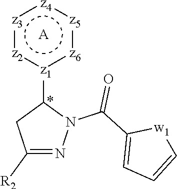

wherein w₁ is S or O.
{EH40}. The K-Ras inhibiting compound according to Embodiment {EH36}, wherein one of R₇-R₁₁ is not hydrogen.
{EH41}. The K-Ras inhibiting compound according to Embodiment {EH1}, wherein R₂ is hydrogen.
{EH42}. The K-Ras inhibiting compound according to Embodiment {EH1}, having a structure of formula (A6e):

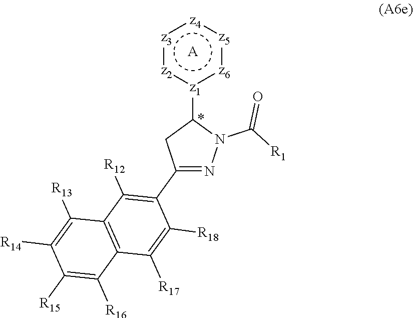

wherein R₁₂-R₁₈ are independently selected from hydrogen, F, Cl, Br, OH, OCH₃, OCH₂CH₃, OCH₂CH₂CH₃, OC(CH₃)₃, CH₃, CH₂CH₃, CH₂CH₂CH₃, or C(CH₃)₃.
{EH43}. The K-Ras inhibiting compound according to Embodiment {EH1}, having a structure of formula (A6f):

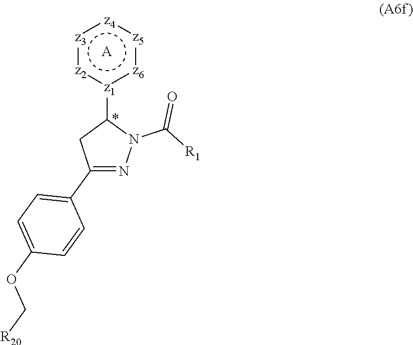

wherein R₂₀ is hydrogen, —CH₃, —X, —R, —C(O)—R, —C(O)—R^{B}, —C(O)—X or —N(R₂₁)(R₂₂), R₂₁ and R₂₂ are independently hydrogen or R;
and in the case where R₂₀ is —N(R₂₁)(R₂₂), R₂₁ and R₂₂ may together form a five- or six-membered saturated ring comprising N optionally subsititued with O.
{EH44}. The K-Ras inhibiting compound according to Embodiment {EH1}, having the structure:

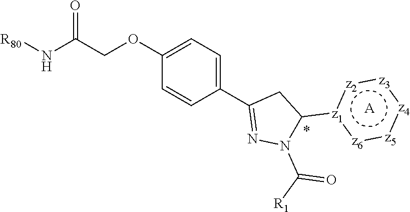

wherein R₈₀ is hydrogen, or —(CH₂)₀₋₃—X.
{EH45}. The K-Ras inhibiting compound according to Embodiment {EH1}, wherein R₈₀ is —(CH₂)₀₋₃—OH.
{EH46}. The K-Ras inhibiting compound according to Embodiment {EH1}, having a structure of formula (A6g):

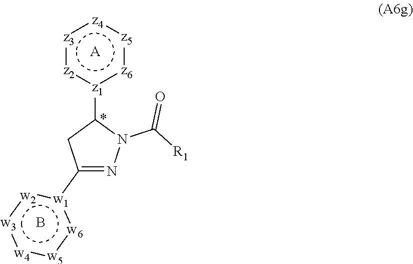

wherein ring “B” is a five- or six-membered, optionally aromatic, ring; where w₁ is C, CH, or N; and w₂-w₆ are independently selected from N, NH, NR^{N}, O, S, C═O, CH, CX, CR, CH₂, C(X)(X), C(R)(X), or C(R)(R); and wherein ring “A” may contain 0, 1, 2, or 3 double bonds; and in the case where ring “B” is a five-membered ring, w₄ is a bond (i.e., it is absent); and wherein any two vicinal substituents X and/or R and/or R^{N} may together form a 5- or 6-membered ring fused to ring “B”.
{EH47}. The K-Ras inhibiting compound according to Embodiment {EH46}, having a structure of formula (A6h):

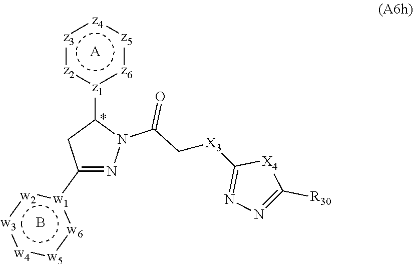

wherein X₃ is selected from CH₂, NH, O or S;
X₄ is NH, NR, N—R^{L}—R^{B}, N—R^{B}, O or S;
R₃₀ is R, R^{B}, —R^{L}-L₁-R, —R^{L}-L₁-R^{B}, —(R^{L})₀₋₁-(L₁)₀₋₂-(R^{L})₀₋₁—R^{B}, -(L₁)₀₋₁-(R^{L})₀₋₁-(L₁)₀₋₂₋(R^{L})₀₋₁—R^{B}, —(R^{L})₀₋₁-(L₁)₀₋₂-(R^{L})₀₋₁—R, -(L₁)₀₋₁-(R^{L})₀₋₁-(L₁)₀₋₂-(R^{L})₀₋₁—R, —(R^{L})₀₋₁-(L₁)_{0- 2}-(R^{L})₀₋₁—R₄₀, or -(L₁)₀₋₁-(R^{L})₀₋₁-(L₁)₀₋₂-(R^{L})₀₋₁-R₄₀; and R₄₀ is hydrogen, R, R^{B}, or X.
{EH48}. The K-Ras inhibiting compound according to Embodiment {EH47}, wherein R₃₀ has the structure:

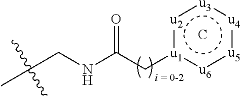

wherein ring “C” is a five- or six-membered, optionally aromatic, ring; where u₁ is C, CH, or N; and u₂-u₆ are independently selected from N, NH, NR^{N}, O, S, C═O, CH, CX, CR, CH₂, C(X)(X), C(R)(X), or C(R)(R); and wherein ring “A” may contain 0, 1, 2, or 3 double bonds; and in the case where ring “B” is a five-membered ring, w₄ is a bond (i.e., it is absent); and wherein any two vicinal substituents X and/or R and/or R^{N} may together form a 5- or 6-membered ring fused to ring “C”.
{EH49}. The K-Ras inhibiting compound according to Embodiment {EH48}, wherein “i” is zero.
{EH50}. The K-Ras inhibiting compound according to Embodiment {EH47}, wherein ring “C” is optionally substituted phenyl.
{EH51}. The K-Ras inhibiting compound according to Embodiment {EH47}, wherein ring “C” is mono or bisubstituted phenyl, and said phenyl is substituted with Cl, F, Br or combinations thereof.
{EH52}. The K-Ras inhibiting compound according to Embodiment {EH47}, wherein ring “C” is mono or bi substituted phenyl, and said phenyl is substituted with C₁₋₄ alkoxy.
{EH53}. The K-Ras inhibiting compound according to Embodiment {EH47}, wherein X₄ is N(CH₃) and R₃₀ has the structure:

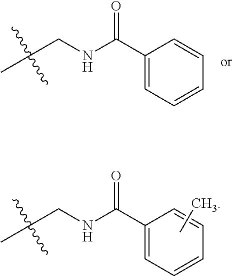

{EH54}. The K-Ras inhibiting compound according to Embodiment {EH1}, having the structure:

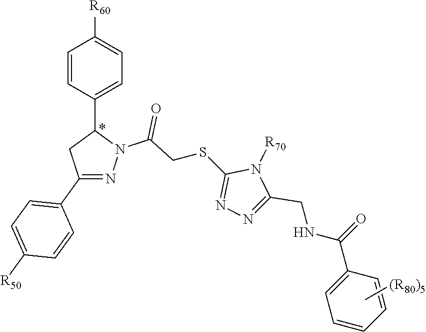

wherein R₅₀, R₆₀, and R₇₀ are independently selected from —R or —X and
R₈₀ is independently selected at each occurrence from hydrogen and —X or —R.
{EH55}. The K-Ras inhibiting compound according to Embodiment {EH54}, wherein R₄₀ and R₅₀ are independently —X (e.g., F, Cl, Br, etc.) and R₆₀ and R₇₀ are independently hydrogen or lower alkyl (e.g., methyl).
{EH56}. The K-Ras inhibiting compound according to Embodiment {EH1}, having the structure:

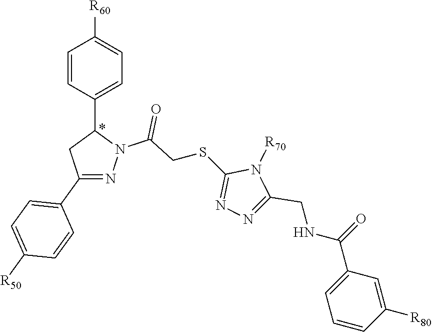

wherein R₅₀, R₆₀, and R₇₀ are independently selected from —R or —X and
R₈₀ is independently selected at each occurrence from hydrogen and —X, or —R.
{EH57}. The K-Ras inhibiting compound according to Embodiment {EH56}, wherein R₅₀ and R₆₀ are independently —X (e.g., Cl, Br, etc.) and R₇₀ and R₈₀ are independently lower alkyl (e.g., methyl).

### Scaffold A7—Embodiment I (“EI”)

{EI1}. A compound for inhibiting K-Ras having formula (A7), or a pharmaceutically acceptable salt thereof:

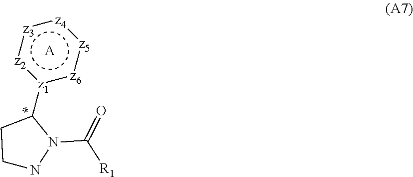

wherein ring “A” is a five- or six-membered optionally aromatic ring, z₁ is C, CH, or N; and z₂-z₆ are independently selected from N, NH, NR^{N}, O, S, C═O, CH, CX, CR, CH₂, C(X)(X), C(R)(X), C(R)(R); and wherein ring “A” may contain 0, 1, 2, or 3 double bonds; and in the case where ring “A” is a five-membered ring, z₄ is a bond (i.e., it is absent); and wherein any two vicinal substituents X and/or R and/or R^{N} may together form an optionally aromatic 5- or 6-membered ring fused to ring “A”;
R₁ is either (i) a C₁₋₁₂ hydrocarbon radical (e.g., alkyl, alkenyl, alkynyl, aryl, alkyl-aryl, aryl-alkyl, and combinations thereof) optionally substituted with one or more (e.g., 1-5) groups X and/or with 1-10 heteratoms selected from O, S, N, P, F, Cl, Br, I, and combinations thereof; or (ii) a group of the form —R^{Q}—R^{B}, —R^{Q}—X, L₁-R^{B}, —R^{Q}-L₁-X, -L₁-R^{Q}—X, R^{L}—R^{B}, -(L₁)ₓ-(CR*₂)_{y}-(L₂)_{z}-(R^{Q})ₘ-(L₃)ₙ-(CR*₂)ₚ-(L₄)_{q}-(R^{Q})ᵣ-(L₅)ₛ-R, or -(L₁)ₓ(CR*₂)_{y}-(L₂)_{z}-(R^{Q})ₘ-(L₃)ₙ-(CR*₂)ₚ-(L₄)_{q}(R^{Q})ᵣ-(L₅)ₛ-X; where x, y, z, m, n, p, q, r, and s are integers independently selected from 0 1, 2, or 3;
L₁-L₅ are each selected independently at each occurrence from —O—, —S—, —NH—, —N(R^{N})—, —(CH₂)₁₋₄—, —C═C—, —C═C—(CH₂)₁₋₃—, —C(O)—, —C(O)—(CH₂)₁₋₃—, —(CH₂)₁₋₃—C(O)—, —C(O)—O—(CH₂)₁₋₃—, —C(O)—N(R^{N})—, —N(R^{N})—C(O)—, —C(O)—N(R^{N})—(CH₂)₁₋₃—, —(CH₂)₁₋₃—C(O)—N(R^{N})—, —NH—S(O)₁₋₂—, —N(R^{N})—S(O)₁₋₂—, —S(O)₁₋₂—N(R^{N})—, —O—(CH₂)₁₋₃—, —(CH₂)₁₋₃—O—, —S—(CH₂)₁₋₃—, —(CH₂)₁₋₃—S—, —NH—(CH₂)₁₋₃—, —N(R^{N})—(CH₂)₁₋₃—, —(CH₂)₁₋₃—N(R^{N})—, —S(═O)₁₋₂—, or —(OCH₂CH₂)₁₋₃—;
R^{Q} is a cyclic group having the structure:

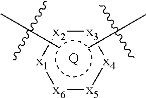

wherein ring “Q” is a five- or six-membered optionally aromatic ring, x₁ and x₄ are independently C, CH, CR or N; and x₂, x₃, x₅, and x₆, are independently selected from N, NH, NR^{N}, O, S, C═O, CH, CX, CR, CH₂, C(X)(X), C(R)(X), and C(R)(R); and in the case where ring “Q” is a five-membered ring, x₃ or x₅ is a bond (i.e., it is absent); and wherein any two vicinal substituents X and/or R and/or R^{N} may together form a 5- or 6-membered ring fused to ring “Q”;
R^{B} is a C₃₋₁₂ monocyclic or fused bicyclic hydrocarbon (alicyclic or aromatic) or heterocycle (e.g., heteroaryl), optionally substituted with one or more (e.g., 1-5) groups X, R*, and/or with 1-6 heteroatoms selected from O, S, N, P, F, Cl, Br, I, and combinations thereof;
X is independently selected at each occurrence from —F, —Cl, —Br, —I, —OH, —OR*, —NH₂, —NHR*, —N(R*)₂, —N(R*)₃⁺, —N(R*)—OH, —N(→O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂—, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(═O)₁₋₂—R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂;
R^{L} is independently selected at each occurrence from C₁₋₆ linear or branched bivalent hydrocarbon radicals; optionally substituted with one or more (e.g., 1-5) groups X and/or with 1-6 heteroatoms selected from O, S, N, P, F, Cl, Br, I, and combinations thereof;
R* is, independently at each occurrence, H or a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.);
R is independently selected at each occurrence from hydrogen and C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteratoms selected from O, S, N, P, F, Cl, Br, I, and combinations thereof;
R^{N} is independently selected at each occurrence from hydrogen, methyl, ethyl, or propyl; and
wherein “*” indicates a chiral center which may be in the “R” or “S” configuration, or a racemic mixture thereof, and wherein the compound may be in the form of R,R or R,S or S,R or S,S diastereomers.
{EI2}. The K-Ras inhibiting compound according to Embodiment {EI1}, wherein z₁ is C, and z₂-z₆ are, respectively, groups C—R₁₀, C—R₁₁, C—R₁₂, C—R₁₃, and C—R₁₄, such that ring “A” has the form:

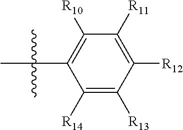

wherein, R₁₀, R₁₁, R₁₂, R₁₃, and R₁₄ are independently selected at each occurrence from the group consisting of hydrogen, X, —R, —R*, and —OR*, wherein any two adjacent groups R and/or R* and/or —OR*, may together form a 5- or 6-membered ring fused to ring “A.”
{EI3}. The K-Ras inhibiting compound according to Embodiment {EI2}, wherein R₁₀, R₁₁, R₁₂, R₁₃, and R₁₄ are independently selected from the group consisting of hydrogen, —OH, —SH, —NH₂; —N(R*)₂; —F, —Cl, —Br, —I, —CH₃, —CH₂CH₃, —CH₂CH₃, —CH₂CH₂CH₃, —CH(CH₃)₂, —OCH₂CH₃, —OCH(CH₃)₂, —OCH₃, —SCH₃, and —OCF₃.
{EI4}. The K-Ras inhibiting compound according to Embodiment {EI3}, wherein one of R₁₀, R₁₁, R₁₂, R₁₃, and R₁₄ is —CH₃, —OCH₃, or —OCH₂CH₃.
{EI5}. The K-Ras inhibiting compound according to Embodiment {EI3}, wherein one of R₁₀, R₁₁, R₁₂, R₁₃, and R₁₄ is —F.
{EI6}. The K-Ras inhibiting compound according to Embodiment {EI1}, wherein z₄ is a bond (i.e., it is absent), and z₁, z₂, z₃, z₅, and z₆ are, respectively, groups C—R₁₀, C—R₁₁, C—R₁₂, C—R₁₃, and C—R₁₄, such that ring “A” may be selected from the group consisting of:

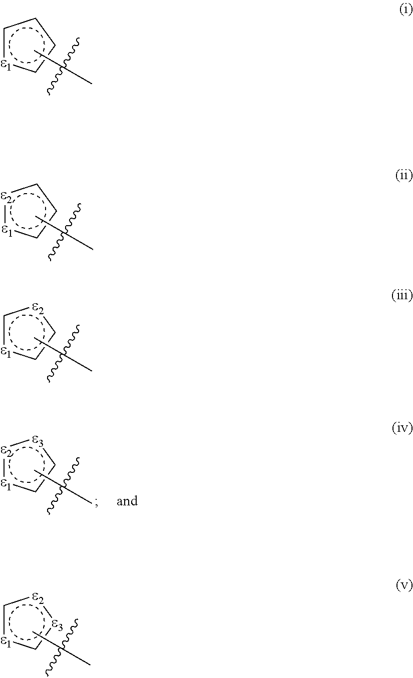

wherein ε₁, ε₂, and ε₃, are independently selected from N, NH, NR^{N}, NR*, —C(═O)—, S, and O; with the proviso that where the point of attachment is ε₁, ε₂, or ε₃, then that position represents N; and wherein carbon atoms which are not the point of attachment may be optionally substituted with a group X (e.g., F, Cl, Br, —SH, —OH, —OCH₃) or with a group R (e.g., —CH₃, —CH₂CH₃, etc.) which may in turn be substituted with one or more (e.g., 1-3) groups X and/or 1-10 heteratoms selected from O, S, N, P, F, Cl, Br, I, and combinations thereof; and wherein the dashed circles indicate that each ring may comprise zero, one, or two double bonds and may be aromatic, and wherein any two adjacent groups X, R^{N}, R*, and/or R may together form a 5- or 6-membered ring fused with ring “A,” and wherein a six-membered aromatic ring may be fused to any two adjacent groups z₁, z₂, z₃, z₅, and z₆.
{EI7}. The K-Ras inhibiting compound according to Embodiment {EI1}, wherein ring “A” is a thiophen-3-yl radical of the form:

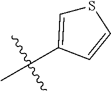

wherein, any available carbon atom is optionally substituted with a group X (e.g., F, Cl, Br, —SH, —OH, or —OCH₃) or with a group R (e.g., —CH₃, —CH₂CH₃, etc.).
{EI8}. The K-Ras inhibiting compound according to Embodiment {EI1}, wherein ring “A” is a pyrazole radical of the form:

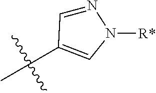

wherein, any available carbon atom is optionally substituted with a group X (e.g., F, Cl, Br, —SH, —OH, or —OCH₃) or with a group R (e.g., —CH₃, —CH₂CH₃, etc.).
{EI9}. The K-Ras inhibiting compound according to Embodiment {EI1}, wherein R₁ is a group of the form —R^{Q}—CR*₂—NH—C(O)—NH₂.
{EI10}. The K-Ras inhibiting compound according to Embodiment {EI1}, wherein R₁ is a group of the form —R^{Q}-L₃-CR*₂—C(O)—NH₂.
{EI11}. The K-Ras inhibiting compound according to Embodiment {EI1}, wherein R₁ is a group of the form —R^{Q}—R^{B}, where R^{B} is a five-membered heteroaromatic ring (e.g., pyrrole, imidazole, triazole, tetrazole, etc.).
{EI12}. The K-Ras inhibiting compound according to Embodiment {EI1}, wherein R₁ is a group of the form —(CR*₂)₁₋₃—R^{B}, where R^{B} is five- or six-membered aryl or heteroaromatic ring (e.g., pyrrole, imidazole, triazole, tetrazole, etc.), optionally fused to a five- or six-membered aryl (e.g., phenyl, pyridyl, etc.) or heteroaromatic ring.
{EI13}. The K-Ras inhibiting compound according to Embodiment {EI1}, wherein R₁ is a group of the form —(CR*₂)ₘ—R^{Q}-(L₃)ₙ-(CR*₂)ₚ—C(O)—NH₂, where m, n, and p are integers independently selected from 0-3 (i.e., 0, 1, 2, or 3), R^{Q} has the structure:

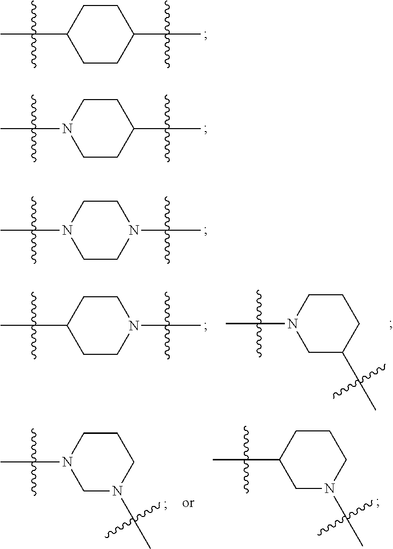

and where L₃ is selected from —O—, —S—, —NR^{N}—, —C(O)—, —NHC(O)—, —C(O)NH—, —OC(O)—, - and —C(O)O—.
{EI14}. The K-Ras inhibiting compound according to Embodiment {EI13}, wherein m, n and p are independently is 0 or 1.
{EI15}. The K-Ras inhibiting compound according to Embodiment {EI1}, wherein x, y, z, m, n, p, q, r, and s are integers independently selected from 0 or 1.
{EI16}. The K-Ras inhibiting compound according to Embodiment {EI1}, wherein R₁ is a group of the form —(CR*₂)ₘ—R^{Q}-(L₃)ₙ-(CR*₂)ₚ—C(O)—NH₂, where m, n, and p are integers independently selected from 0-3 (i.e., 0, 1, 2, or 3), wherein R^{Q} is a six-membered aromatic aryl;
and where L₃ is selected from —O—, —S—, —NR^{N-}, —C(O)—, —NHC(O)—, —C(O)NH—, —OC(O)—, - and —C(O)O—.
{EI17}. The K-Ras inhibiting compound according to Embodiment {EI13}, wherein m, n and p are independently is 0 or 1.
{EI18}. The K-Ras inhibiting compound according to Embodiment {EI1} having the structure:

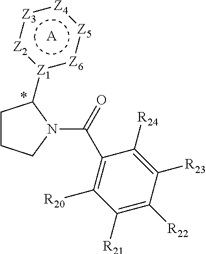

wherein R₂₀-R₂₄ are independently selected from hydrogen, —X, —(CH₂)₀₋₃—X, or —(CH₂)₀₋₃-L₁-X.
{EI19}. The K-Ras inhibiting compound according to Embodiment {EI18}, wherein X is —NH₂.
{EI20}. The K-Ras inhibiting compound according to Embodiment {EI18}, wherein L₁ is —NH—C(O)—.

### Scaffolds A8-A10—Embodiment J (“EJ”)

{EJ1}. A compound for inhibiting K-Ras having formula A(x), or a pharmaceutically acceptable salt thereof:

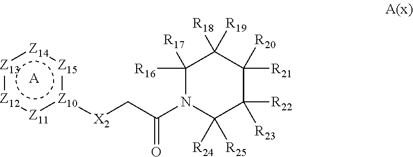

ring “A” is a five- or six-membered optionally aromatic ring; z₁₀ is C, CH, or N; and z₁₁-z₁₅ are independently selected from N, NH, NR^{N}, O, S, C═O, CH, CX, CR, CH₂, C(X)(X), C(R)(X), or C(R)(R); and wherein ring “A” may contain 0, 1, 2, or 3 double bonds; and in the case where ring “A” is a five-membered ring, z₁₃ is a bond (i.e., it is absent); and wherein any two vicinal substituents X and/or R and/or R^{N} may together form a 5- or 6-membered ring fused to ring “A”;
X₂ is selected from the group consisting of —O—, —S—, —S(O)₁₋₂—, —N(R₈)—, —C(O)—, —CH₂—, —C(CH₃)(H)—, and —C(R₈)(R₉)—; where R₈ and R₉ are independently selected from hydrogen, —X, —R, and -L₁-R; where R₈ and R₉ may together form a [3-6]-membered ring, and where R₈ or R₉ may together with z₁₁ or z₁₅ form a 5- or 6-membered fused ring;
R₁₆-R₂₅ are independently selected from hydrogen, R, X, or R₂₅; wherein at least one of R₁₆-R₂₅ is a group R₂₆;
R₂₆ is selected from X, -L₁-R^{B}, -L₁-R, -L₁-R^{L}—R^{B}, -L₁-R^{L}—X, -L₁-R^{L}-L₁-R, -L₁-R^{L}-L₁-R^{B}, -L₁-R^{L}-L₁-X, -L₁(R^{L})₀₋₁₋C(R*)(R*)X, -L (R^{L})₀₋₁—C(R*)(R^{B})(X), -L₁-(R^{L})₀₋₁—C(R*)(R*)(R*);
R^{B} is a C₃₋₁₂ monocyclic or fused bicyclic hydrocarbon (alicyclic or aromatic) or heterocycle (e.g., heteroaryl), optionally substituted with one or more (e.g., 1-5) groups X, R*, and/or with 1-6 heteroatoms selected from O, S, N, P, F, Cl, Br, I, and combinations thereof;
R^{L} at each occurrence is a C₁₋₆ linear or branched bivalent hydrocarbon radical; optionally substituted with one or more (e.g., 1-5) groups X and/or with 1-6 heteroatoms selected from O, S, N, P, F, Cl, Br, I, and combinations thereof;
X is independently selected at each occurrence from —F, —Cl, —Br, —I, —OH, —OR*, —NH₂, —NHR*, —N(R*)₂, —N(R*)₃⁺, —N(R*)—OH, —N(*O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂⁻, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(═O)₁₋₂—R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂;
R* is independently selected at each occurrence from hydrogen and a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.);
R is selected from hydrogen or C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteroatoms selected from O, S, N, P, F, Cl, Br, I, and combinations thereof; and
L₁ is selected independently at each occurrence from —O—, —S—, —NH—, —N(R^{N})—, —(CH₂)₁₋₄—, —C═C—, —C═C—(CH₂)₁₋₃—, —C(O)—, —C(O)—(CH₂)₁₋₃—, —(CH₂)₁₋₃—C(O)—, —C(O)—O—(CH₂)₁₋₃—, —C(O)—N(R^{N})—, —N(R^{N})—C(O)—, —C(O)—N(R^{N})—(CH₂)₁₋₃—, —(CH₂)₁₋₃—C(O)—N(R^{N})—, —NH—S(O)₁₋₂—, —N(R^{N})—S(O)₁₋₂—, —S(O)₁₋₂—N(R^{N})—, —O—(CH₂)₁₋₃—, —(CH₂)₁₋₃—O—, —S—(CH₂)₁₋₃—, —(CH₂)₁₋₃—S—, —NH—(CH₂)₁₋₃—, —N(R^{N})—(CH₂)₁₋₃—, —(CH₂)₁₋₃—N(R^{N})—, —S(═O)₁₋₂—; and —(OCH₂CH₂)₁₋₃—; and
R^{N} is independently selected at each occurrence from hydrogen, methyl, ethyl, or propyl.
{EJ2}. The K-Ras inhibiting compound according to Embodiment {EJ1}, wherein each of R₁₆-R₁₉ is hydrogen.
{EJ3}. The K-Ras inhibiting compound according to Embodiment {EJ1}, having formula A(xa), or a pharmaceutically acceptable salt thereof:

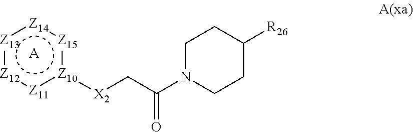

{EJ4}. The K-Ras inhibiting compound according to Embodiment {EJ1} having the structure:

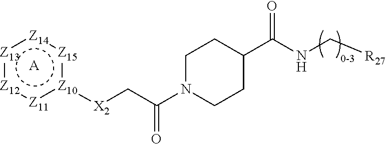

wherein R₂₇ is selected from —R, —R^{B}, —CH(R^{B})(R), —CH(R^{B})(X), or —X.
{EJ5}. The K-Ras inhibiting compound according to Embodiment {EJ1} having the structure:

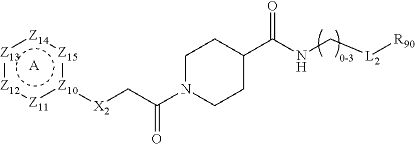

wherein R₉₀ is —R, —X, or —R^{B}; and
L₂ is —O—, —C(O)—NH—, or —NH—C(O)—.
{EJ6}. The K-Ras inhibiting compound according to Embodiment {EJ1} having the structure:

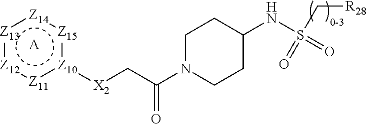

wherein R₂₈ is hydrogen, or lower alkyl (e.g., methyl, etc.).
{EJ7}. The K-Ras inhibiting compound according to Embodiment {EJ3}, wherein R₂₆ has the form —NH-L₁-R.
{EJ8}. The K-Ras inhibiting compound according to Embodiment {EJ3}, wherein R₂₆ has the form —NH—C(O)—R.
{EJ9}. The K-Ras inhibiting compound according to Embodiment {EJ3}, wherein R₂₆ has the form —NH—S(O)₂—R.
{EJ10}. The K-Ras inhibiting compound according to Embodiment {EJ3}, wherein R₂₆ has the form —C(O)-L₁-R.
{EJ11}. The K-Ras inhibiting compound according to Embodiment {EJ3}, wherein R₂₆ has the form —C(O)—NH—R.
{EJ12}. The K-Ras inhibiting compound according to Embodiment {EJ1} or {EJ11}, wherein R₂₆ has the form -(L₁)₀₋₁-(CH₂)₀₋₂—R^{B} or form -(L₁)₀₋₁-(CH₂)₀₋₂-R.
{EJ13}. The K-Ras inhibiting compound according to Embodiment {EJ12}, wherein R^{B} of R₂₆ is phenyl.
{EJ14}. The K-Ras inhibiting compound according to Embodiment {EJ12}, wherein R^{B} of R₂₆ is pyridinyl.
{EJ15}. The K-Ras inhibiting compound according to Embodiment {EJ12}, wherein R^{B} of R₂₆ is pyrrolyl or furanyl.
{EJ16}. The K-Ras inhibiting compound according to Embodiment {EJ12}, wherein R^{B} of R₂₆ is phenyl substituted with Cl, F, Br, —CH₃, —CH₂—CH₃, —CH₂—CH₂—CH₃, or —C(CH₃)₃.
{EJ17}. The K-Ras inhibiting compound according to Embodiment {EJ12}, wherein R^{B} of R₂₆ is piperidinyl.
{EJ18}. The K-Ras inhibiting compound according to Embodiment {EJ1}, having a structure of Formula (A8):

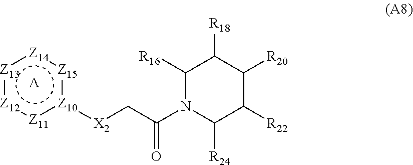

{EJ19}. The K-Ras inhibiting compound according to Embodiment {EJ18}, wherein ring “A” is a six-membered aromatic ring.
{EJ20}. The K-Ras inhibiting compound according to Embodiment {EJ18}, wherein at least one of z₁₂ and z₁₄ is independently selected from N, NR, or NX.
{EJ21}. The K-Ras inhibiting compound according to Embodiment {EJ18}, wherein z₁₃ is CR₁₃, z₁₂ or z₁₄ is CR₁₂, where R₁₃ and R₁₂ are R, and R₁₂ and R₁₃ together form a five-membered fused aromatic ring with ring A.
{EJ22}. The K-Ras inhibiting compound according to Embodiment {EJ18}, wherein R₁ and R₂ are C₁₋₄ hydrocarbons each with at least one substitution selected from S, O, and N, and where R₁ and R₂ may together form a 5-membered spiro ring.
{EJ23}. The K-Ras inhibiting compound according to Embodiment {EJ3}, wherein R₈ or R₉ and the vicinal group of z₁₁ or z₁₅ form a 5- or 6-membered fused ring form a five- or six-membered fused ring optionally substituted with O, N, and S.{EJ24}. The K-Ras inhibiting compound according to Embodiment {EJ1}, having a structure of Formula (A8a):

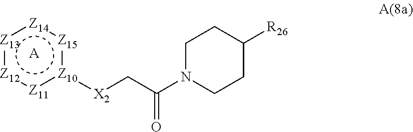

{EJ25}. The K-Ras inhibiting compound according to Embodiment {EJ1}, having a structure of Formula (A9):

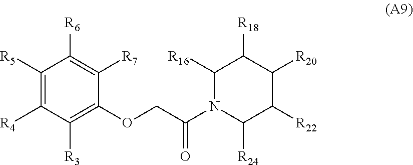

wherein R₃-R₇ are independently selected from hydrogen, —X, —R, and -L₁-R; where any two vicinal groups R₃, R₄, R₅, R₆, and R₇ may together form a 5- or 6-membered fused ring.
{EJ26}. The K-Ras inhibiting compound according to Embodiment {EJ1}, having a structure of Formula (A10):

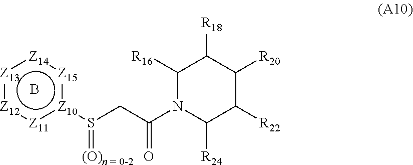

wherein, ring “B” is a five- or six-membered aromatic ring; z₁₀ is C or N; and z₁₁-z₁₅ are independently selected from N, NH, NR^{N}, O, S, C═O, CH, CX, CR, CH₂, C(X)(X), C(R)(X), and C(R)(R); and in the case where ring “B” is a five-membered ring, z₁₃ is a bond (i.e., it is absent); and wherein any two vicinal substituents X and/or R and/or R^{N} may together form a 5- or 6-membered ring fused to ring “B”; and
where the value of integer “n” indicates a thioether (in the case n=0), a sulfoxide (in the case n=1), or sulfone (in the case n=2).
{EJ27}. The K-Ras inhibiting compound according to Embodiment {EJ26}, wherein ring “B” is a six-membered ring.
{EJ28}. The K-Ras inhibiting compound according to Embodiment {EJ26}, where in at least one of z₁₁-z₁₅ is N.
{EJ29}. The K-Ras inhibiting compound according to Embodiment {EJ26}, wherein two vicinal substituents on ring “B” form a five-membered aromatic fused ring.
{EJ30}. The K-Ras inhibiting compound according to Embodiment {EJ12}, wherein said vicinal substituents of ring “B” are C₁₋₄ hydrocarbons, and at least one of R₁ or R₂ has at least one substitution selected from S, O, and N.
{EJ31}. The K-Ras inhibiting compound according to Embodiments {EJ1}-{EJ30}, wherein Z₃ is —C(R₁)(R₂).
{EJ32}. The K-Ras inhibiting compound according to Embodiment {EJ31}, wherein R₁ and/or R₂ is -L₁-R or -L₁-(CR*₂)₁₋₂-(L₁)₀₋₁-R; where L₁ is selected from the group consisting of —O—, —C(═O)—, —NH—, —NR^{N}—, —C(O)—NR^{N}—, —NR^{N}—C(O)—, —NR^{N}—S(O)₂—.
{EJ33}. The K-Ras inhibiting compound according to Embodiments {EJ1}-{EJ32}, wherein R has the form —(CR′R″)₁₋₃—R^{A}, where R′ and R″ are independently selected at each occurrence selected from hydrogen, —OH, —OCH₃, —CH₃, and —CH₂CH₃; where any two geminal or vicinal R′ and/or R″ may together form an alicyclic ring; and where R^{A} is a C₃₋₆ linear or branched hydrocarbon, optionally substituted with 1-3 groups X and/or with 1-6 heteroatoms selected from O, S, N, P, F, Cl, Br, I, and combinations thereof.
{EJ34}. The K-Ras inhibiting compound according to Embodiments {EJ1}-{E26}, wherein R is has the form —(CR′R″)₁₋₃—R^{B}, where R′ and R″ are independently selected at each occurrence from hydrogen, —OH, —OCH₃, —CH₃, and —CH₂CH₃; where R′ or R″ may together form an alicyclic ring; and where R^{B} is a C₃₋₆ cyclic hydrocarbon (alicyclic or aromatic) or heterocycle (e.g., heteroaryl), optionally substituted with one or more groups X and/or with 1-6 heteroatoms selected from O, S, N, P, F, Cl, Br, I, and combinations thereof.
{EJ35}. The K-Ras inhibiting compound according to Embodiments {EJ1}-{EJ32}, wherein L₁ is —C(O)—NH— or —NH—C(O)—.
{EJ36}. The K-Ras inhibiting compound according to Embodiment {EJ1}, wherein L₁ is —NH—S(O)₂— and R is —CH₃.
{EJ37}. The K-Ras inhibiting compound according to Embodiment {EJ1}, wherein R₁ is L₁-R, where L₁ is —NH—C(O)—, and R₁ and R₂ together form a five-membered alicyclic ring.
{EJ38}. The K-Ras inhibiting compound according to Embodiment {EJ1}, wherein z₁₁ or z₁₅ is CR or CX, and R₈ together with the geminal group of z₁₁ or z₁₅ forms a five- or six-membered fused ring optionally substituted with from 1-5 heteroatoms selected from O, N, and S.
{EJ39}. The K-Ras inhibiting compound according to Embodiments {EJ1}-{E38}, wherein two vicinal groups on ring “A” together form a fused 5- or 6-membered fused ring optionally substituted with 1-5 heteroatoms selected from O, N, and S.
{EJ40}. The K-Ras inhibiting compound according to Embodiments {EJ1}-{EJ39}, wherein z₁₁-z₁₅ are independently selected from CH and CX, where X is independently selected at each occurrence from the group consisting of hydrogen, —Cl, —F, —CH₃, —OCH₃, —O—CH₂CH₃, and —CN.
{EJ41}. The K-Ras inhibiting compound according to Embodiment {EJ40}, wherein one of z₁₁-z₁₅ is CX, where X is is independently selected from the group consisting of —Cl, —F, —CH₃, —O—CH₃, —O—CH₂—CH₃, and —CN.
{EJ42}. The K-Ras inhibiting compound according to Embodiment {EJJ40}, wherein two of z₁₁-z₁₅ are CX, where X is independently selected from the group consisting of —Cl, —F, —CH₃, —O—CH₃, —O—CH₂—CH₃, and —CN.

### Scaffold A11—Embodiment K (“EK”)

{EK1}. A compound for inhibiting K-Ras having formula (A11), or a pharmaceutically acceptable salt thereof:

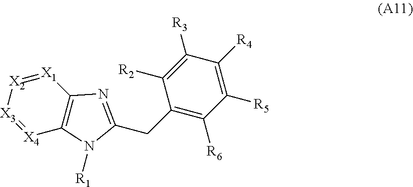

X₁, X₂, X₃, and X₄ are independently selected from CH or N;
R₁ is -L₁-R^{Q}—R^{B}, or —R^{Q}-L₁-R^{B}; and
R₂-R₆ are independently selected from hydrogen, —R, or —X; where
X is independently selected at each occurrence from —F, —Cl, —Br, —I, —OH, —OR*, —NH₂, —NHR*, —N(R*)₂, —N(R*)₃⁺, —N(R*)—OH, —N(*O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂⁻, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(═O)₁₋₂—R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂;
R* is independently selected at each occurrence from hydrogen and a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.);
R is selected from hydrogen or C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteroatoms selected from O, S, N, P, F, Cl, Br, I, and combinations thereof;
R^{Q} has the structure:

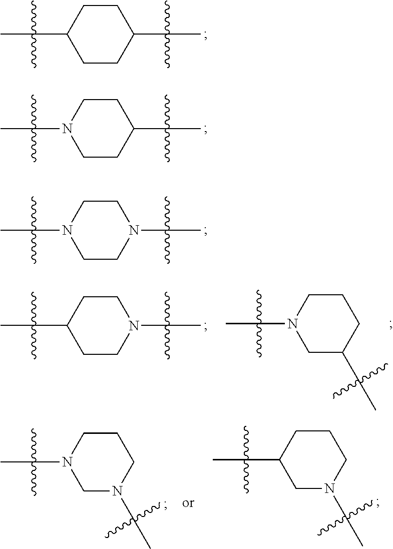

R^{B} is a C₃₋₁₂ monocyclic or fused bicyclic hydrocarbon (alicyclic or aromatic) or heterocycle (e.g., heteroaryl), optionally substituted with one or more (e.g., 1-5) groups X, R*, and/or with 1-6 heteroatoms selected from O, S, N, P, F, Cl, Br, I, and combinations thereof;
L₁ is selected independently at each occurrence from —O—, —S—, —NH—, —N(R^{N})—, —(CH₂)₁₋₄—, —C═C—, —C═C—(CH₂)₁₋₃—, —C(O)—, —C(O)—(CH₂)₁₋₃—, —(CH₂)₁₋₃—C(O)—, —C(O)—O—(CH₂)₁₋₃—, —C(O)—N(R^{N})—, —N(R^{N})—C(O)—, —C(O)—N(R^{N})—(CH₂)₁₋₃—, —(CH₂)₁₋₃—C(O)—N(R^{N})—, —NH—S(O)₁₋₂—, —N(R^{N})—S(O)₁₋₂—, —S(O)₁₋₂—N(R^{N})—, —O—(CH₂)₁₋₃—, —(CH₂)₁₋₃—O—, —S—(CH₂)₁₋₃—, —(CH₂)₁₋₃—S—, —NH—(CH₂)₁₋₃—, —N(R^{N})—(CH₂)₁₋₃—, —(CH₂)₁₋₃—N(R^{N})—, —S(═O)₁₋₂—; and —(OCH₂CH₂)₁₋₃—; and
R^{N} is independently selected at each occurrence from hydrogen, methyl, ethyl, or propyl.
{EK2}. The K-Ras inhibiting compound according to Embodiment {EK1}, wherein R₁ is -L₁-R^{Q}—R^{B} or —R^{Q}-L₁-R^{B}.
{EK3}. The K-Ras inhibiting compound according to Embodiment {EK2}, wherein R^{Q} of R₁ is

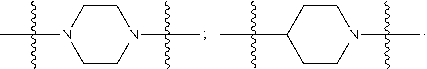 [SMILES: *N1CCN(*)CC1 ; ";" ; *C1CCN(*)CC1 ; "."]

{EK4}. The K-Ras inhibiting compound according to Embodiment {EK2}, wherein L₁ of R₁ is —C(O)—, or —(CH₂)₁₋₃—C(O)—.
{EK5}. The K-Ras inhibiting compound according to Embodiment {EK2}, wherein R^{B} of R₁ is a six-membered or a five-membered aromatic ring optionally substituted with 1-2 heteroatoms selected from F, Cl, N, S, or O.
{EK6}. The K-Ras inhibiting compound according to Embodiment {EK2}, wherein R^{B} of R₁ is a five-membered ring fused to a six-membered ring, optionally substituted one with one or more (e.g., 1-5) groups R^{N}.
{EK7}. The K-Ras inhibiting compound according to Embodiment {EK6}, wherein R^{B} of R₁ has the structure:

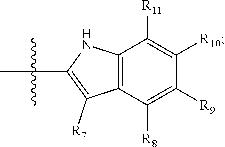

where R₇-R₁₁ are independently selected from hydrogen, methyl, and ethyl.
{EK8}. The K-Ras inhibiting compound according to Embodiment {EK7}, wherein at least one of R₇-R₁₁ are methyl.
{EK9}. The K-Ras inhibiting compound according to Embodiment {EK7}, wherein at least two of R₇-R₁₁ are methyl.
{EK10}. The K-Ras inhibiting compound according to Embodiment {EK7}, wherein R₇ is hydrogen.
{EK11}. The K-Ras inhibiting compound according to Embodiment {EK7}, wherein R₈ and R₁₀ are each methyl, and R₇, R₉, R₁₁ and R₁₂ are each hydrogen.
{EK12}. The K-Ras inhibiting compound according to Embodiment {EK7}, having the structure:

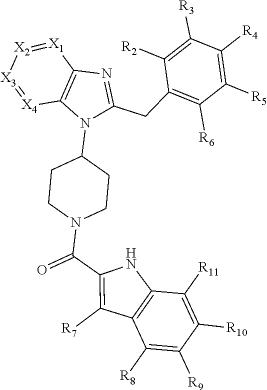

{EK13}. The K-Ras inhibiting compound according to Embodiment {EK12}, wherein R₇ is hydrogen.
{EK14}. The K-Ras inhibiting compound according to Embodiment {EK12} or {EK13}, wherein R₈ and R₁₀ are each methyl; and
   R₉, R₁₁ and R₁₂ are each hydrogen.
{EK15}. The K-Ras inhibiting compound according to any one of Embodiments {EK1}-{EK14}, wherein R₂-R₆ are independently selected from hydrogen, —Cl, —F, —Br, methyl, ethyl, and propyl.
{EK16}. The K-Ras inhibiting compound according to any one of Embodiments {EK1}-{EK14}, wherein at least one of R₂-R₆ is not hydrogen.
{EK17}. The K-Ras inhibiting compound according to any one of Embodiments {EK1}-{EK14}, wherein X₁—X₄ are each CH.
{EK18}. The K-Ras inhibiting compound according to any one of Embodiments {EK1}-{EK14}, wherein at least one of X₁—X₄ is N.
{EK19}. The K-Ras inhibiting compound according to any one of Embodiments {EK1}-{EK14}, wherein R^{B} is selected from

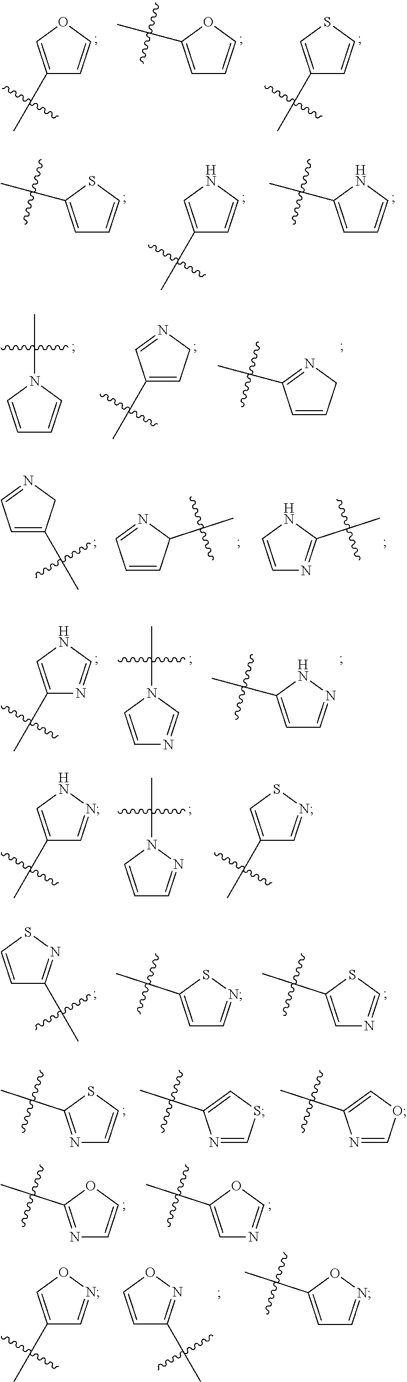

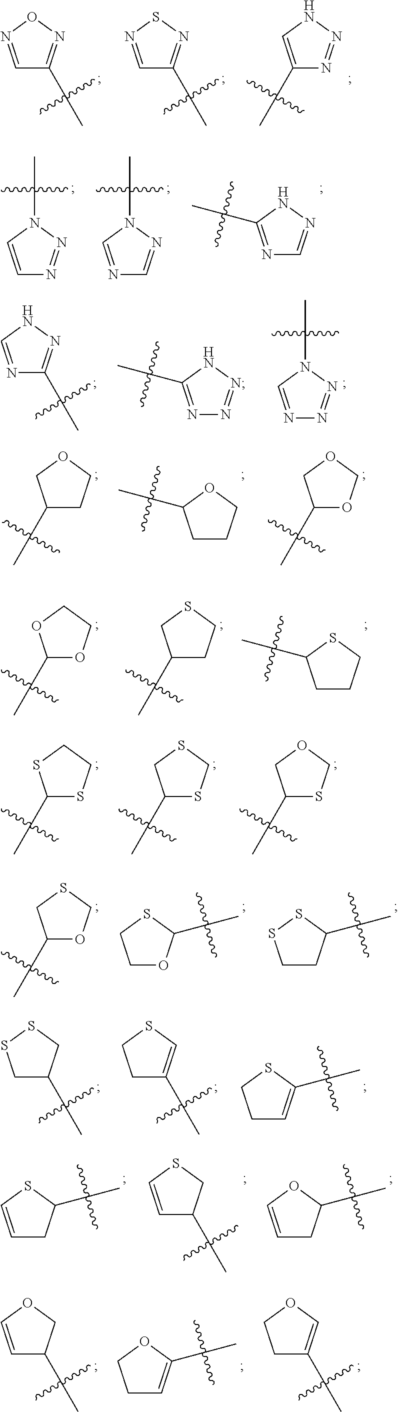

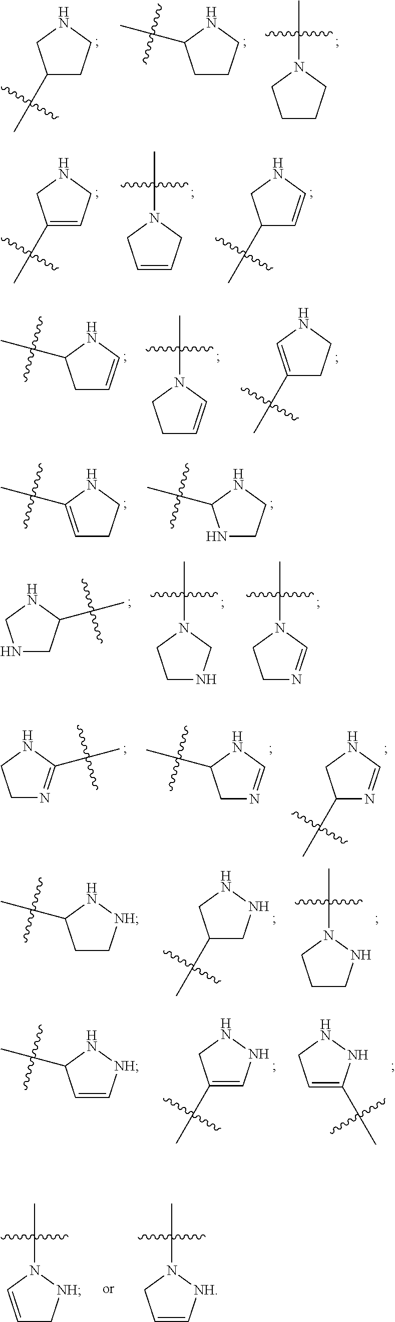

### Scaffold A12—Embodiment L (“EL”)

{EL1}. A compound for inhibiting K-Ras having formula (A12), or a pharmaceutically acceptable salt thereof:

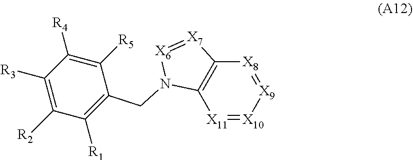

wherein X₆ is N or CR₆; X₇ is N or CR₇; X₈ is N or CR₈; X₉ is N or CR₉; X₁₀ is N or CR₁₀; X₁₁ is N or CR₁₁;
R₁-R₅ are selected from hydrogen, —R, —OR, and —X;
R₆, R₇, and R₈ are independently selected from hydrogen, —X, —R, —R^{B}, —R^{Q}—R, —R^{Q}—X, —R^{Q}-(L₁)₀₋₁-R^{B}, —R^{Q}-(L₁)₀₋₁-R, -L₁-R, -L₁-R^{B}, —R^{Q}-L₁-R, -L₁-X, —C(R*₂)₀₋₂-L₁-(CR*₂)₁₋₃—R^{B}, - L₁-(CR*₂)₁₋₃—R, —R^{L}—R, —R^{L}—X, —R^{L}-(L₁)₀₋₁-R^{B}, —R^{L}-(L₁)₀₋₁-R, -L₁-R, -L₁-R^{B}, —R^{L}-L₁-R, and —C(R₁₂)(R₁₃); and vicinal groups R₆ and R₇ may together form a 5- or 6-membered fused ring;
R₁₂ is hydrogen, R, X, or —R^{Q}-(L₁)₀₋₁-R^{B};
R₁₃ is R, X, or —(CR*₂)₀₋₃-(L₁)₀₋₁-(CR*₂)₀₋₄—R^{B}; R₉-R₁₁ are independently selected from hydrogen, —X, —R, -L₁-X, or -L₁-R; where
X is independently selected at each occurrence from —F, —Cl, —Br, —I, —OH, —OR*, —NH₂, —NHR*, —N(R*)₂, —N(R*)₃⁺, —N(R*)—OH, —N(*O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂⁻, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(═O)₁₋₂—R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂;
R^{L} at each occurrence is a C₁₋₆ linear, branched, or cyclic bivalent hydrocarbon radical; optionally substituted with one or more (e.g., 1-5) groups X and/or with 1-6 heteroatoms selected from O, S, N, P, F, Cl, Br, I, and combinations thereof;
L₁ is selected independently at each occurrence from —O—, —S—, —NH—, —N(R^{N})—, —(CH₂)₁₋₄—, —C═C—, —C═C—(CH₂)₁₋₃—, —C(O)—, —C(O)—(CH₂)₁₋₃—, —C(O)—O—(CH₂)₁₋₃—, —C(O)—N(R^{N})—, —C(O)—N(R^{N})—(CH₂)₁₋₃—, —(CH₂)₁₋₃—C(O)—N(R^{N})—, —NH—S(O)₁₋₂—, —N(R^{N})—S(O)₁₋₂—, —O—(CH₂)₁₋₃—, —S(O)₁₋₂—, —S—(CH₂)₁₋₃—, —NH—(CH₂)₁₋₃—, —N(R^{N})—(CH₂)₁₋₃—, and —(OCH₂CH₂)₁₋₃—;
R^{N} is independently selected at each occurrence from hydrogen, methyl, ethyl, or propyl;
R* is independently selected at each occurrence from hydrogen and a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.);
R is selected from hydrogen or C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteroatoms selected from O, S, N, P, F, Cl, Br, I, and combinations thereof;
R^{B} is a C₃₋₆ cyclic hydrocarbon (alicyclic or aromatic) or heterocycle (e.g., heteroaryl), optionally substituted with one or more groups X and/or with 1-6 heteroatoms selected from O, S, N, P, F, Cl, Br, I, and combinations thereof, where any vicinal groups may form a 5- or 6-membered ring fused with said cyclic hydrocarbon; and
R^{Q} is a cyclic group having the structure:

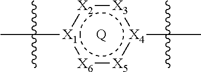

wherein ring “Q” is a five- or six-membered optionally aromatic ring, x₁ and x₄ are independently C, CH, CR or N; and x₂, x₃, x₅, and x₆ are independently selected from N, NH, NR^{N}, O, S, C═O, CH, CX, CR, CH₂, C(X)(X), C(R)(X), and C(R)(R); and in the case where ring “Q” is a five-membered ring, x₃ or x₅ is a bond (i.e., it is absent); and wherein any two vicinal substituents X and/or R and/or R^{N} may together form a 5- or 6-membered ring fused to ring “Q”.
{EL2} The K-Ras inhibiting compound according to Embodiment {EL1}, wherein R₁₂ and/or R₁₃ is hydrogen.
{EL3} The K-Ras inhibiting compound according to Embodiment {EL1}, wherein one or more of R₁-R₅ and R₈-R₁₁ are independently selected from the group consisting of hydrogen, —OH, —Cl, —F, —Br, —I, —CH₃, —CH₂—CH₃, —O—CH₃, —O—CH₂—CH₃, and —CN.
{EL4}. The K-Ras inhibiting compound according to Embodiment {EL1}, wherein R₈-R₁₁ are independently selected from hydrogen and -L₁-R; wherein L₁ is selected from —NH—C(O)—, —NH—S(O)₂—, and —C(O)—.
{EL5}. The K-Ras inhibiting compound according to Embodiment {EL1}, wherein x₉ is CR₉, and R₉ is -L₁-R; where L₁ is selected from —NH—C(O)—, —NH—S(O)₂—, and —C(O)—.
{EL6}. The K-Ras inhibiting compound according to Embodiment {EL5}, wherein R in the group -L₁-R of R₉ is lower alkyl (e.g., methyl, ethyl, propyl, isopropyl, isobutyl, etc.).
{EL7}. The K-Ras inhibiting compound according to Embodiment {EL5}, wherein L₁ is —NH—C(O)—, and R is a hydrogen or C₁₋₆ alkyl.
{EL8}. The K-Ras inhibiting compound according to Embodiment {EL5}, wherein L₁ is —NH—S(O)₂— and R is R^{B}
{EL9}. The K-Ras inhibiting compound according to Embodiment {EL1}, wherein one of R₆ and R₇ is —R-L₁-(CR*₂)₁₋₃—R^{B}.
{EL10}. The K-Ras inhibiting compound according to Embodiment {EL9}, wherein L₁ is independently selected at each occurrence from —C(O)—NH— and —NH—.
{EL11}. The K-Ras inhibiting compound according to Embodiment {EL10)}, wherein R₆ or R₇ is —C(R*₂)-L₁-(CR*₂)₁₋₃—R^{B}, L₁ is —NH— or —C(O)—NH—, and R^{B} is an optionally substituted five-membered heterocyclic ring.
{EL12} The K-Ras inhibiting compound according to Embodiment {EL11}, wherein R^{B} of R₆ or R₇ is pyrrolidinyl.
{EL13}. The K-Ras inhibiting compound according to Embodiment {EL12}, wherein R₆ or R₇ is —X, where X is —C(═O)—OR* or —C(═O)—OH.
{EL14}. The K-Ras inhibiting compound according to Embodiment {EL12}, wherein R₆ or R₇ is-S—CH₂—C(O)—NH₂.
{EL15}. The K-Ras inhibiting compound according to Embodiment {EL1}, wherein R₆ or R₇ is —R^{Q}-L₁-R^{B}; where R^{Q} is a six-membered heterocyclic ring comprising nitrogen at the point of attachment to L₁.
{EL16}. The K-Ras inhibiting compound according to Embodiment {EL15}, wherein L₁ of R₆ or R₇ is —SO₂—.
{EL17}. The K-Ras inhibiting compound according to any one of Embodiments {EL8}-{EL11}, {EL15}, or {EL16}, wherein R^{B} of R₆ or R₇ is optionally substituted phenyl, pyrollyl, thiophenyl, furanyl, or imidazolyl.
{EL18}. The K-Ras inhibiting compound according to Embodiment {EL1}, wherein R₆ or R₇ is C(R₁₂)(R₁₃); where R₁₃ is —R^{Q}—O—CH₂—R^{B}.
{EL19}. The K-Ras inhibiting compound according to Embodiment {EL18}, wherein R₁₂ is C(R*₂)—C(O)—NH—(CR*₂)₁₋₂—R^{B}.
{EL20}. The K-Ras inhibiting compound according to Embodiment {EL18} or {EL19}, wherein R^{B} of R₁₂ and R₁₃ is independently selected from pyrrolyl, piperidinyl, pyridinyl, morpholinyl, or phenyl.
{EL21}. The K-Ras inhibiting compound according to Embodiment {EL1}, wherein R₆ or R₇ together make a fused ring, such that the compound has the structure:

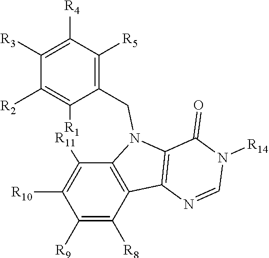

where R₁₄ is selected from hydrogen, —X, —R, and —(CR*₂)₀₋₃-(L₁)₀₋₁-(CR*₂)₀₋₃—R^{B}.
{EL22}. The K-Ras inhibiting compound according to Embodiment {EL1} having the structure:

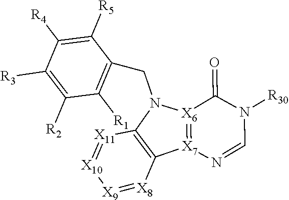

wherein R₃₀ is —(CH₂)₀₋₃-R^{B}
{EL23}. The K-Ras inhibiting compound according to Embodiment {EL1} having the structure:

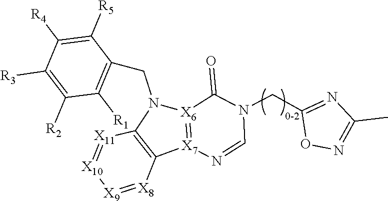

{EL24}. The K-Ras inhibiting compound according to Embodiment {EL1}, wherein the compound has the structure:

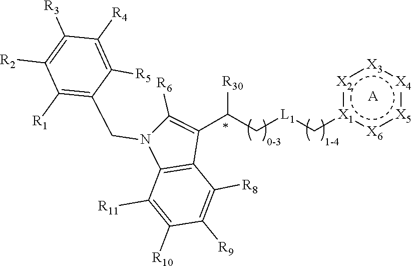

wherein ring “A” is a five- or six-membered, optionally aromatic ring, x₁ is C, CH, CR or N; x₂-x₆ are independently selected from N, NH, NR^{N}, O, S, C═O, CH, CX, CR, CH₂, C(X)(X), C(R)(X), or C(R)(R); and in the case where ring “A” is a five-membered ring, x₄ is a bond (i.e., it is absent); and wherein any two vicinal substituents X and/or R and/or R^{N} may together form a 5- or 6-membered ring fused to ring “A”; and
R₃₀ is hydrogen or lower alkyl (e.g., C₁₋₆).
{EL25}. The K-Ras inhibiting compound according to Embodiment {EL24}, wherein ring “A” is selected from the group consisting of:

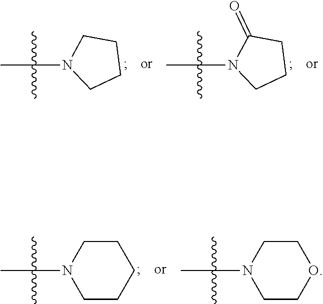

{EL26}. The K-Ras inhibiting compound according to Embodiment {EL1}, wherein the compound has the structure:

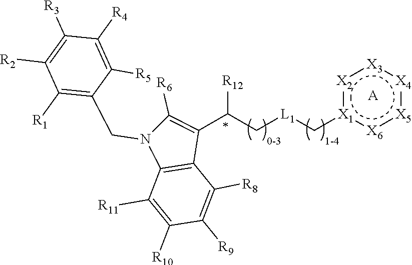

wherein ring “A” is a five- or six-membered, optionally aromatic ring, x₁ is C, CH, CR or N; x₂-x₆ are independently selected from N, NH, NR^{N}, O, S, C═O, CH, CX, CR, CH₂, C(X)(X), C(R)(X), or C(R)(R); and in the case where ring “A” is a five-membered ring, x₄ is a bond (i.e., it is absent); and wherein any two vicinal substituents X and/or R and/or R^{N} may together form a 5- or 6-membered ring fused to ring “A”.
{EL27}. The K-Ras inhibiting compound according to Embodiment {EL26} wherein R₁₂ is —R^{Q}-L₁-R^{B}.
{EL28}. The K-Ras inhibiting compound according to Embodiment {EL27}, wherein the compound has the structure:

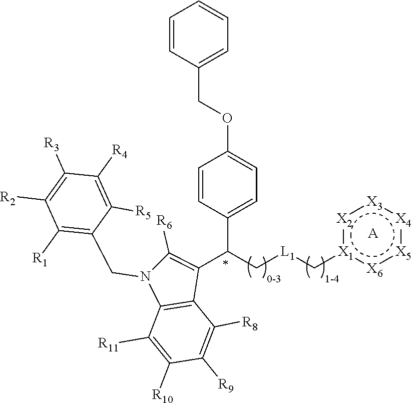

wherein ring “A” is a five- or six-membered, optionally aromatic ring, x₁ is C, CH, CR or N; x₂-x₆ are independently selected from N, NH, NR^{N}, O, S, C═O, CH, CX, CR, CH₂, C(X)(X), C(R)(X), or C(R)(R); and in the case where ring “A” is a five-membered ring, x₄ is a bond (i.e., it is absent); and wherein any two vicinal substituents X and/or R and/or R^{N} may together form a 5- or 6-membered ring fused to ring “A”.
{EL29}. The K-Ras inhibiting compound according to Embodiment {EL28}, wherein the compound has the structure:

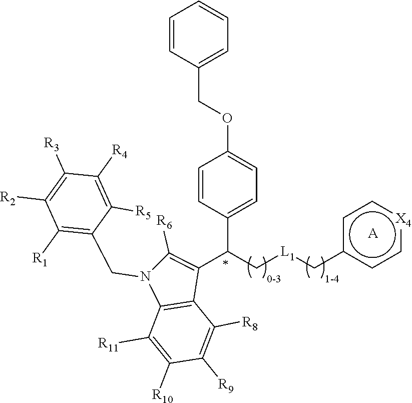

wherein ring “A” is a six-membered aromatic ring, x₄ is selected from N, or CH.
{EL30}. The K-Ras inhibiting compound according to Embodiment {EL29}, having the structure:

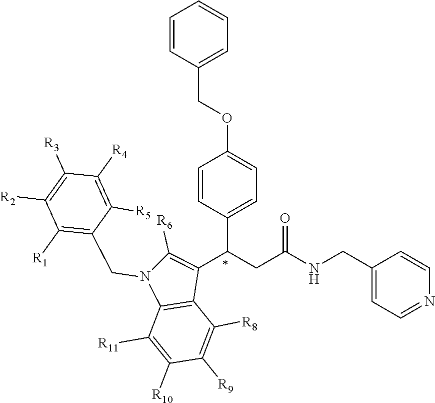

{EL31}. The K-Ras inhibiting compound according to any one of Embodiments {EL1} or {EL24}-{EL30}, wherein R₈-R₁₁ are each hydrogen.
{EL32}. The K-Ras inhibiting compound according to any one of Embodiments {EL1}-{EL31}, wherein at least one of R₁-R₅ are C₁₋₇ alkoxy (e.g., methoxy, ethoxy, etc.) and the rest are hydrogen.
{EL33}. The K-Ras inhibiting compound according to Embodiment {EL1}, wherein at least one of X₆—X₁₁ are N.
{EL34}. The K-Ras inhibiting compound according to Embodiment {EL1} wherein X₆ and X₁₁ are each N or X₇ is N.
{EL35}. The K-Ras inhibiting compound according to Embodiment {EL1}, wherein R₈ is -L₁-X, where -L₁- is —C(O)—O—(CH₂)₁₋₃—C(O)— and —X is —NH₂.
{EL36}. The K-Ras inhibiting compound according to Embodiment {EL1}, having the structure:

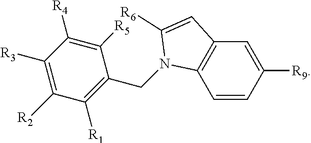

{EL37}. The K-Ras inhibiting compound according to Embodiment {EL36}, wherein at least one of R₁-R₅ is —X (e.g., Cl, F, etc.) and the rest are hydrogen.
{EL38}. The K-Ras inhibiting compound according to Embodiment {EL36} or {EL37}, wherein R₆ and R₉ are independently selected from hydrogen and a group -L₁-(CH₂)₀₋₂-R₆₀, where R₆₀ is independently selected at each occurrence from hydrogen, R (e.g., lower alkyl, including methyl, ethyl, propyl, isopropyl, butyl, isobutyl, tert-butyl etc.), or R_{B} (e.g., phenyl).
{EL39}. The K-Ras inhibiting compound according to Embodiment {EL38}, wherein L₁ is —C(O)—NH— or —NH—C(O)—.
{EL40}. The K-Ras inhibiting compound according to Embodiment {EL1} having the structure:

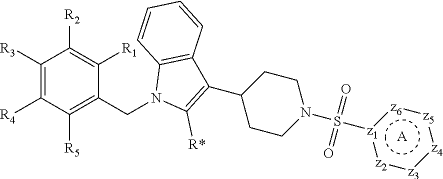

ring “A” is a five- or six-membered optionally aromatic ring; z₁₀ is C, CH, or N; and z₁₁-z₁₅ are independently selected from N, NH, NR^{N}, O, S, C═O, CH, CX, CR, CH₂, C(X)(X), C(R)(X), or C(R)(R); and wherein ring “A” may contain 0, 1, 2, or 3 double bonds; and in the case where ring “A” is a five-membered ring, z₁₃ is a bond (i.e., it is absent); and wherein any two vicinal substituents X and/or R and/or R^{N} may together form a 5- or 6-membered ring fused to ring “A”.

### Scaffold A13—Embodiment M (“EM”)

{EM1}. A compound for inhibiting K-Ras having a structure of formula (A13), or a pharmaceutically acceptable salt thereof:

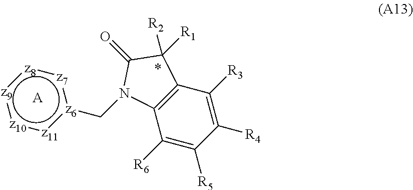

wherein, ring “A” is a five- or six-membered aromatic ring, z₆ is C; z₇ is CR₇, N, NR₇, O, or S; z₈ is CR₈, N, NR₈, O, or S; z₉ is CR₉, N, NR₉, O, or S; z₁₀ is CR₁₀, N, NR₁₀, O, or S; z₁₁ is CR₁₁, N, NR₁₁, O, or S; and wherein ring “A” may contain 0, 1, 2, or 3 double bonds; and in the case where ring “A” is a five-membered ring, z₉ is a bond (i.e., it is absent); and wherein any two vicinal substituents X and/or R and/or R^{N} may together form a 5- or 6-membered ring fused to ring “A”;
wherein, R₁ and R₂ are independently selected from hydrogen, —X, —R, -L₁-X, —R^{B}, -L₁-R, or -L₁-R^{B}; where R₁ and R₂ may together form a 5- or 6-membered spiro ring;
R₃₋₁₁ are independently selected from hydrogen, —X, —R, —OR, or —N(R^{N})R; where any two vicinal groups R₃, R₄, R₅, and R₆ may together form an optionally aromatic 5- or 6-membered fused ring; and where any two vicinal groups R₇, R₈, R₉, R₁₀, and R₁₁ may together form an optionally aromatic 5- or 6-membered ring fused to ring “A;”
X is independently selected at each occurrence from —F, —Cl, —Br, —I, —OH, —OR*, —NH₂, —NHR*, —N(R*)₂, —N(R*)₃⁺, —N(R*)—OH, —N(→O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂—, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(═O)₁₋₂—R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂;
R* is independently selected at each occurrence from hydrogen and a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.);
R and R′ are independently selected from hydrogen or C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteroatoms selected from O, S, N, P, F, Cl, Br, I, and combinations thereof;
R^{B} is a C₃₋₆ cyclic hydrocarbon (alicylic or aromatic) or heterocycle (e.g., heteroaryl), optionally substituted with one or more groups X and or with 1-6 heteroatoms selected from O, S, N, P, F, Cl, B, I, and combinations thereof; and wherein R^{B} may further comprise an additional 5- or 6-membered optionally aromatic ring fused thereto;
L₁ is selected independently at each occurrence from —O—, —S—, —NH—, —N(R^{N})—, —(CH₂)₁₋₄—, —C═C—, —C═C—(CH₂)₁₋₃—, —C(O)—, —C(O)—(CH₂)₁₋₃—, —C(O)—O—(CH₂)₁₋₃—, —C(O)—N(R^{N})—, —C(O)—N(R^{N})—(CH₂)₁₋₃—, —(CH₂)₁₋₃—C(O)—N(R^{N})—, —NH—S(O)₁₋₂—, —N(R^{N})—S(O)₁₋₂—, —O—(CH₂)₁₋₃—, —S(O)₁₋₂—, —S—(CH₂)₁₋₃—, —NH—(CH₂)₁₋₃—, —N(R^{N})—(CH₂)₁₋₃—, and —(OCH₂CH₂)₁₋₃—; where
R^{N} is independently selected at each occurrence from hydrogen, methyl, ethyl, or propyl.
{EM2}. The K-Ras inhibiting compound according to Embodiment {EM1}, wherein R₃₋₁ are independently selected from the group consisting of hydrogen, —Cl, —F, —Br, —I, —CH₃, —CH₂—CH₃, —O—CH₃, —O—CH₂—CH₃, —NH₂, and —CN.
{EM3}. The K-Ras inhibiting compound according to Embodiment {EM1}, where any two vicinal groups R₃, R₄, R₅, and R₆ together form a 5- or 6-membered fused ring.
{EM4}. The K-Ras inhibiting compound according to Embodiment {EM1}, where any two vicinal groups R₇, R₈, R₉, R₁₀, and R₁₁ together form a 5- or 6-membered fused aromatic ring.
{EM5}. The K-Ras inhibiting compound according to Embodiment {EM1}, wherein R₁ or R₂ is selected from hydrogen; —F; —Cl; —Br; —I; —OH; —NH₂; and —CN.
{EM6}. The K-Ras inhibiting compound according to Embodiment {EM5}, wherein R₁ or R₂ is —OH.
{EM7}. The K-Ras inhibiting compound according to Embodiment {EM1}, wherein R₁ and R₂ together form a 5- or 6-membered spiro ring.
{EM8}. The K-Ras inhibiting compound according to Embodiment {EM7}, wherein R₁ or R₂ is -L₁-R or —R′-L₁-X, where L₁ is —N(R^{N})—C(O)—, and R′ is —(CH₂)₁₋₃—.
{EM9}. The K-Ras inhibiting compound according to Embodiment {EM8}, wherein X of R₁ or R₂ is selected from the group consisting of —CH₃, —CH₂—CH₃, —O—CH₃, —O—CH₂—CH₃, —NH₂ and —CN.
{EM10}. The K-Ras inhibiting compound according to Embodiment {EM7}, where R₁ is -L₁-R^{B}, where L₁ is —NH—C(O)—, and R^{B} is a C₃₋₆ cyclic hydrocarbon (alicyclic or aromatic) or heterocycle (e.g., heteroaryl), optionally substituted with one or more groups R, X and/or with 1-6 heteroatoms selected from O, S, N, P, F, Cl, Br, I, and combinations thereof, and where R₂ is —NH—, and where R^{B} may together with or R₂ form a six-membered ring.
{EM11}. The K-Ras inhibiting compound according to Embodiment {EM8}, wherein R₁ is —(CH₂)—C(O)—R^{B}, where R^{B} has the form:

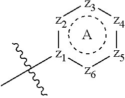

where ring “A” is a five- or six-membered optionally aromatic ring, z₁ is C, CH, or N; and z₂-z₆ are independently selected from N, NH, NR^{N}, O, S, C═O, CH, CX, CR, CH₂, C(X)(X), C(R)(X), or C(R)(R); and in the case where ring “A” is a five-membered ring, z₄ is a bond (i.e., it is absent); and wherein any two vicinal substituents X and/or R and/or R^{N} may together form a 5- or 6-membered ring fused to ring “A.”
{EM12}. The K-Ras inhibiting compound according to Embodiment {EM11}, wherein R^{B} of R₁ is a six-membered aromatic ring; where z₂₋₆ are independently CH or CX; where X is selected from the group consisting of —OH, —O—(CH₂)₀₋₃—CH₃, —NH₂, —F, —Cl, —Br, —I, and —(CH₂)₀₋₃—CH₃.
{EM13}. The K-Ras inhibiting compound according to Embodiment {EM11}, wherein R^{B} of R₁ is a six-membered aromatic ring, where z₂₋₆ are independently CH or CR″, where R″ is a C₃₋₆ cyclic hydrocarbon (alicyclic or aromatic) or heterocycle (e.g., heteroaryl), optionally substituted with one or more groups X and/or with 1-6 heteroatoms selected from O, S, N, P, F, Cl, Br, I, and combinations thereof.
{EM14}. The K-Ras inhibiting compound according to Embodiment {EM11}, wherein z₄ is a bond, and ring “A” is be selected from the group consisting of:

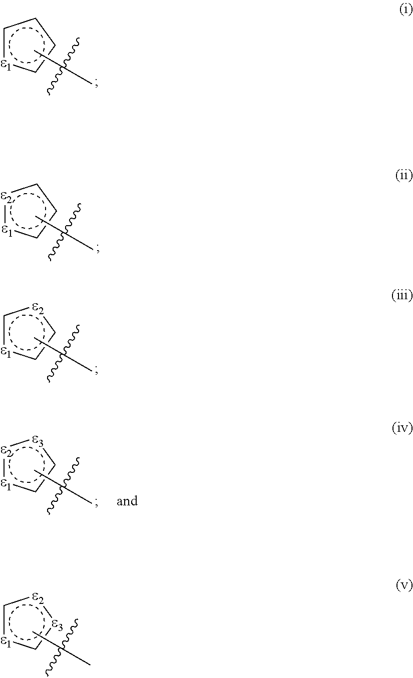

wherein ε₁, ε₂, and ε₃, are independently selected from N, NH, NR^{N}, NR*, —C(═O)—, S, and O; with the proviso that where the point of attachment is ε₁, ε₂, or ε₃, then that position represents N; and wherein carbon atoms which are not the point of attachment may be optionally substituted with a group X (e.g., F, Cl, Br, —SH, —OH, —OCH₃) or with a group R (e.g., —CH₃, —CH₂CH₃, etc.) which may in turn be substituted with one or more (e.g., 1-3) groups X and/or 1-10 heteroatoms selected from O, S, N, P, F, Cl, Br, I, and combinations thereof; and wherein the dashed circles indicate that each ring may comprise zero, one, or two double bonds and may be aromatic, and wherein any two adjacent groups X, R^{N}, R*, and/or R may together form a 5- or 6-membered ring fused with ring “A.”
{EM15}. The K-Ras inhibiting compound according to Embodiment {EM14}, wherein ring “A” is selected from the group consisting of furanyl, pyrrolyl, and thiophenyl radicals; where any available carbon atom is optionally substituted with —(CH₂)₀₋₂—CH₃.
{EM16}. The K-Ras inhibiting compound according to Embodiment {EM1}, wherein the compound has the structure:

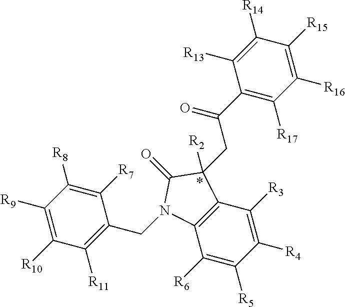

wherein, R₁₃-R₁₇ are independently selected from hydrogen, X, R*, R, and R^{B}.
{EM17}. The K-Ras inhibiting compound according to Embodiment {EM16}, wherein one of R₁₃-R₁₇ is a 5-membered heterocycle (e.g., imidazole).
{EM18}. The K-Ras inhibiting compound according to Embodiment {EM16}, wherein at least one of (e.g., one of) R₁₃-R₁₇ is F.
{EM19}. The K-Ras inhibiting compound according to Embodiment {EM16}, wherein at least one of R₁₃-R₁₇ is Cl.
{EM20}. The K-Ras inhibiting compound according to Embodiment {EM16}, wherein at least one of R₁₃-R₁₇ is —CH₃, —CH₂CH₃, or —CH(CH₃)₂.
{EM21}. The K-Ras inhibiting compound according to Embodiment {EM16}, wherein at least one of R₁₃-R₁₇ is —OH.
{EM22}. The K-Ras inhibiting compound according to Embodiment {EM16}, wherein at least one of R₁₃-R₁₇ is —OCH₃, —OCH₂CH₃, or —OCH(CH₃)₂.
{EM23}. The K-Ras inhibiting compound according to Embodiment {EM16}, wherein at least one of (e.g., one of) R₁₃-R₁₇ is —NH₂ or —NR^{N}₂.
{EM24}. The K-Ras inhibiting compound according to any one of Embodiments {EM1}-{EM4}, or {EM10}-{EM23}, wherein R₂ is —OH.
{EM25}. The K-Ras inhibiting compound according to any one of Embodiments {EM16}-{EM24}, wherein at least one of (e.g., one of) R₇-R₁₁ is lower alkyl (e.g., methyl).
{EM26}. The K-Ras inhibiting compound according to any one of Embodiments {EM16}-{EM25}, wherein at least one of (e.g., one of) R₃-R₆ is lower alkyl (e.g., methyl).
{EM27}. The K-Ras inhibiting compound according to Embodiment {EM16}, wherein two adjacent groups R₁₃-R₁₇ together form a divalent radical —O(CH₂)₁₋₂O—.
{EM28}. The K-Ras inhibiting compound according to Embodiment {EM1}, having the structure:

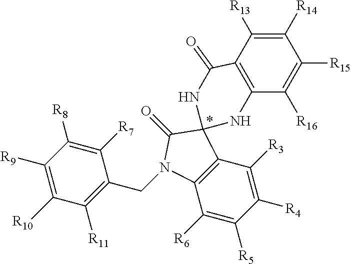

wherein, R₁₃-R₁₇ are independently selected from hydrogen, X, R*, R, and R^{B}.
{EM29}. The K-Ras inhibiting compound according to Embodiment {EM1}, having the structure:

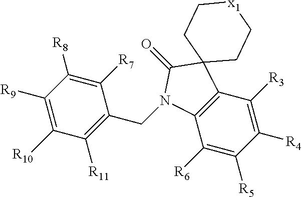

wherein x₁ has the form CH—X, CH—R*, CH—R, N—X, N—R*, or N—R.
{EM30}. The K-Ras inhibiting compound according to Embodiment {EM29}, where x₁ is N—X, where X is —C(O)—NR^{N}₂ or —C(O)—NH₂.
{EM31}. The K-Ras inhibiting compound according to Embodiment {EM1}, where R₁ is R^{B}
{EM32}. The K-Ras inhibiting compound according to Embodiment {EM1}, wherein ring “A” is a six-membered ring.
{EM33}. The K-Ras inhibiting compound according to Embodiment {EM1}, wherein ring “A” is a five-membered ring and z₇ or z₁₁ is S.
{EM34}. The K-Ras inhibiting compound according to Embodiment {EM1}, wherein R₁ is OH.

### Scaffold A14—Embodiment N (“EN”)

{EN1}. A compound for inhibiting K-Ras having a structure of formula (A14), or a pharmaceutically acceptable salt thereof:

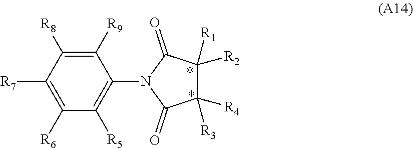

wherein R₁-R₄ are independently selected from hydrogen, —X, —R, —R^{B}, -L₁-R^{B}, —R^{Q}—R, —R^{Q}—X, -L₁-R^{Q}—X, -L₁-R^{Q}—R, or -L₁-R; where R₁ and R₂ or R₃ and R₄ may together form a 5- or 6-membered spiro ring; and any two vicinal groups R₁, R₂, R₃, and R₄ may together form a 5- or 6-membered fused ring;
R₅₋₉ are independently selected from hydrogen, —X, or —R; where any two vicinal groups R₅, R₆, R₇, R₈, and R₉ may together form a 5- or 6-membered fused ring;
X is independently selected at each occurrence from —F, —Cl, —Br, —I, —OH, —OR*, —NH₂, —NHR*, —N(R*)₂, —N(R*)₃⁺, —N(R*)—OH, —N(→O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂⁻, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(═O)₁₋₂—R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂;
R* is independently selected at each occurrence from hydrogen and a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.);
R^{B} is a C₃₋₆ cyclic hydrocarbon (alicylic or aromatic) or heterocycle (e.g., heteroaryl), optionally substituted with one or more groups X and or with 1-6 heteroatoms selected from O, S, N, P, F, Cl, B, I, and combinations thereof; and wherein R^{B} may further comprise an additional 5- or 6-membered optionally aromatic ring fused thereto;
R is selected from hydrogen or C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteroatoms selected from O, S, N, P, F, Cl, Br, I, and combinations thereof; and
L₁ is selected independently at each occurrence from —O—, —S—, —NH—, —N(R^{N})—, —(CH₂)₁₋₄—, —C═C—, —C═C—(CH₂)₁₋₃—, —C(O)—, —C(O)—(CH₂)₁₋₃—, —C(O)—O—(CH₂)₁₋₃—, —C(O)—N(R^{N})—, —C(O)—N(R^{N})—(CH₂)₁₋₃—, —(CH₂)₁₋₃—C(O)—N(R^{N})—, —NH—S(O)₁₋₂—, —N(R^{N})—S(O)₁₋₂—, —O—(CH₂)₁₋₃—, —S(O)₁₋₂—, —S—(CH₂)₁₋₃—, —NH—(CH₂)₁₋₃—, —N(R^{N})—(CH₂)₁₋₃—, or —(OCH₂CH₂)₁₋₃—; where
R^{N} is independently selected at each occurrence from hydrogen, methyl, ethyl, or propyl.
{EN2}. The K-Ras inhibiting compound according to Embodiment {EN1}, wherein R₅-R₉ are independently selected from the group consisting of hydrogen, —Cl, —F, —Br, —I, —CH₃, —CH₂CH₃, —OH, —O—CH₃, —O—CH₂CH₃, —NH₂, —NR^{N}₂, and —CN.
{EN3}. The K-Ras inhibiting compound according to Embodiment {EN1},

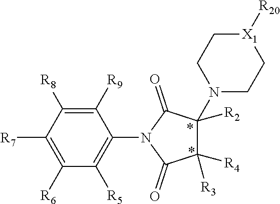

where X₁ is N or CH, and R₂₀ is —X, —R, —R^{B}, —R^{Q}—X, —R^{Q}-L₁-R, and -L₁-R where R^{Q} is a cyclic group having the structure:

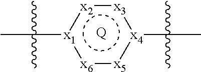

wherein ring “Q” is a five- or six-membered optionally aromatic ring, x₁ and x₄ are independently C, CH, CR or N; and x₂, x₃, x₅, and x₆, are independently selected from N, NH, NR^{N}, O, S, C═O, CH, CX, CR, CH₂, C(X)(X), C(R)(X), and C(R)(R); and in the case where ring “Q” is a five-membered ring, x₃ or x₅ is a bond (i.e., it is absent); and wherein any two vicinal substituents X and/or R and/or R^{N} may together form a 5- or 6-membered ring fused to ring “Q”.
{EN4}. The K-Ras inhibiting compound according to Embodiment {EN3}, where R₂₀ is —C(O)OH, —C(O)O—R*, —C(O)NH₂, or —C(O)NR^{N}₂.
{EN5}. The K-Ras inhibiting compound according to Embodiment {EN1}, wherein at least one vicinal group R₁, R₂, R₃ and R₄ together forms a 6-membered ring, such that the compound has the structure:

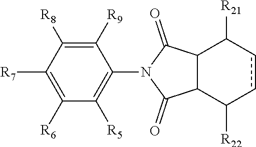

where R₂₁ and R₂₂ are independently groups X, R, R^{B}, and R^{Q}-(L₁)-R.
{EN6}. The K-Ras inhibiting compound according to Embodiment {EN1}, wherein R₁ is a group R^{B}.
{EN7}. The K-Ras inhibiting compound according to Embodiment {EN1}, wherein R^{N} and R together with N form a heterocyclic ring fused with R^{B}.
{EN8}. The K-Ras inhibiting compound according to Embodiment {EN1}, wherein one of R₁-R₄ has the form -L₁-(CH₂)₀₋₃—R^{B}, where L₁ is —NH-n and R^{B} is of the form:

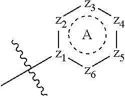

where ring “A” is a five- or six-membered optionally aromatic ring, z₁ is C, CH, or N; and z₂-z₆ are independently selected from N, NH, NR^{N}, O, S, C═O, CH, CX, CR, CH₂, C(X)(X), C(R)(X), or C(R)(R) and in the case where ring “A” is a five-membered ring, z₄ is a bond (i.e., it is absent); and wherein any two vicinal substituents X and/or R and/or R^{N} may together form a 5- or 6-membered ring fused to ring “A.”
{EN9}. The K-Ras inhibiting compound according to Embodiment {EN1}, wherein ring “A” is a six-membered aromatic ring and at least one z₂-z₆ is of the form CX, where X is selected from the group consisting of —Cl, —F, —Br, —I, —OH, —O—CH₃, —O—CH₂—CH₃, —NH₂, —CN, —S(O)₂—OH, and —S(O)₂—N(H)₂.
{EN10}. The K-Ras inhibiting compound according to Embodiment {EN1}, wherein R₁ has the form:

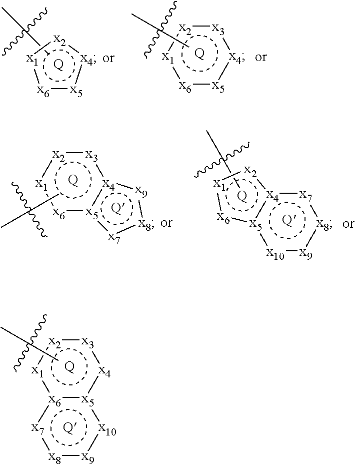

where x₁-x₁₀ are independently selected from O, S, N, NH, NX, NR, C═O, CH, CX, and CR, where X is selected from the group consisting of —Cl, —F, —Br, —I, —OH, —O—CH₃, —O—CH₂—CH₃, —C(CH₃)(CH₃), —OC(CH₃)(CH₃), —NH₂, —NR^{N}₂, —CN, —C(O)OR*, —C(O)OH, —C(O)NH₂—C(O)H, —S(O)₂—OH, and —S(O)₂—N(H)₂.
{EN11}. The K-Ras inhibiting compound according to Embodiment {EN10}, wherein R₁ has the form

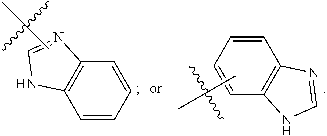

{EN12}. The K-Ras inhibiting compound according to Embodiment {EN1}, having the structure:

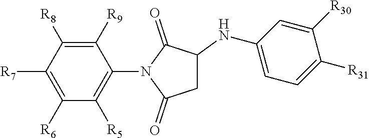

wherein R₃₀ and R₃₁ are independently selected from hydrogen, —R (e.g., lower alkyl, methyl, ethyl), —X, and -L₁-X.
{EN13}. The K-Ras inhibiting compound according to Embodiment {EN12}, wherein R₃₀ or R₃₁ is -L₁-X.
{EN14}. The K-Ras inhibiting compound according to Embodiment {EN12}, wherein R₃₀ or R₃₁ is —S(O)₂—X.
{EN15}. The K-Ras inhibiting compound according to Embodiment {EN12}, wherein R₃₀ or R₃₁ is -L₁-NH₂.
{EN16}. The K-Ras inhibiting compound according to Embodiment {EN12}, wherein R₃₀ or R₃₁ is —S(O)₂—NH₂.

### Scaffold A15—Embodiment O (“EO”)

{EO1}. A compound for inhibiting K-Ras having a structure of formula (A15), or a pharmaceutically acceptable salt thereof:

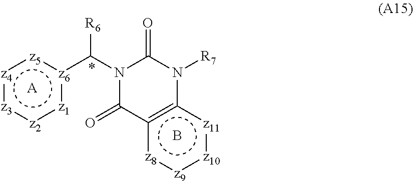

wherein ring “A” is a five- or six-membered optionally aromatic ring; and z₁ is independently selected from N, NR₁, O, S, C═O, CR₁, or C(R₁)₂; z₂ is independently selected from N, NR₂, O, S, C═O, CR₂, or C(R₂)₂; z₃ is independently selected from N, NR₃, O, S, C═O, CR₃, or C(R₃)₂; z₄ is independently selected from N, NR₄, O, S, C═O, CR₄, and C(R₄)₂; z₅ is independently selected from N, NR₅, O, S, C═O, CR₅, C(R₅)₂; z₆ is selected from C, CH, or N, and in the case where ring “A” is a five-membered ring, z₃ is a bond (i.e., it is absent);
ring “B” is a five- or six-membered optionally aromatic ring; and z₈ is indepdently selected from from N, NR₈, O, S, C═O, CR₈, or C(R₈)₂; z₉ is indepdently selected from from N, NR₉, O, S, C═O, CR₉, or C(R₉)₂; z₁₀ is indepdently selected from from N, NR₁₀, O, S, C═O, CR₁₀, or C(R₁₀)₂; z₁₁ is indepdently selected from from N, NR₁₁, O, S, C═O, CR₁₁, or C(R₁₁)₂; and in the case where ring “B” is a five-membered ring, z₁₁ is a bond (i.e., it is absent); and wherein any two vicinal or geminal substituents may together form an additional 5- or 6-membered ring;
R₁₋₅ are independently selected at each occurence from hydrogen, —R, and —X;
R₇ is selected from hydrogen, —X, —R, -L₁-R, —R^{B}, —R^{Q}—R, —R^{L}—R^{B}, -L₁-R^{B}, or —(R^{L})ₙ-(L₁)ₘ-(R^{L})ₚ-(L₂)_{q}-R, —(R^{L})ₙ-(L₁)ₘ-(R^{L})ₚ-(L₂)_{q}-R^{B}; where n, m, p, and q are integers independently selected from 0-2 (i.e., 0, 1, and 2);
R₈-R₁₁ are independently selected at each occurrence from hydrogen, —R, —R^{B}, —X, -L₁-X, -L₁-R, or -L₁-R^{B};
where R^{L} are independently selected at each occurrence from C₁₋₆ (or C₁₋₃) hydrocarbons (e.g., alkyl); and
R is selected from hydrogen or C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteroatoms selected from O, S, N, P, F, Cl, Br, I, and combinations thereof;
X is independently selected at each occurrence from —F, —Cl, —Br, —I, —OH, —OR*, —NH₂, —NHR*, —N(R*)₂, —N(R*)₃⁺, —N(R*)—OH, —N(*O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂⁻, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(═O)₁₋₂—R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂;
R* is independently selected at each occurrence from hydrogen and a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.);
R^{B} is a C₃₋₆ cyclic hydrocarbon (alicylic or aromatic) or heterocycle (e.g., heteroaryl), optionally substituted with one or more groups X and or with 1-6 heteroatoms selected from O, S, N, P, F, Cl, B, I, and combinations thereof; and wherein R^{B} may further comprise an additional 5- or 6-membered optionally aromatic ring fused thereto; and wherein R^{B} may further comprise an additional 5- or 6-membered optionally aromatic ring spiro thereto;
R^{Q} is a cyclic group having the structure:

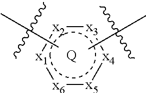

wherein ring “Q” is a six-membered ring, x₁ is C, CH, CR or N; x₄ is C; and x₂, x₃, x₅, and x₆, are independently selected from N, NH, NR^{N}, O, S, C═O, CH, CX, CR, CH₂, C(X)(X), C(R)(X), and C(R)(R); and in the case where ring “Q” is a five-membered ring, x₃ or x₅ is a bond (i.e., it is absent); and wherein any two vicinal substituents X and/or R and/or R^{N} may together form a 5- or 6-membered ring fused to ring “Q”. R is selected from C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteroatoms selected from O, S, N, P, F, Cl, Br, I, and combinations thereof; and
L₁ and L₂ are selected independently at each occurrence from groups L, where L is —O—, —S—, —NH—, —N(R^{N})—, —(CH₂)₁₋₄—, —C═C—, —C═C—(CH₂)₁₋₃—, —C(O)—, —C(O)—(CH₂)₁₋₃—, —C(O)—O—(CH₂)₁₋₃—, —C(O)—N(R^{N})—, —C(O)—N(R^{N})—(CH₂)₁₋₃₋, —(CH₂)₁₋₃—C(O)—N(R^{N})—, —NH—S(O)₁₋₂—, —N(R^{N})—S(O)₁₋₂—, —O—(CH₂)₁₋₃—, —S(O)₁₋₂—, —S—(CH₂)₁₋₃—, —NH—(CH₂)₁₋₃—, —N(R^{N})—(CH₂)₁₋₃—, and —(OCH₂CH₂)₁₋₃—; where
R^{N} is independently selected at each occurrence from hydrogen, methyl, ethyl, or propyl.
{EO2}. The K-Ras inhibiting compound according to Embodiment {EO1}, wherein R₁-R₅ are independently selected at each occurrence from the group consisting of hydrogen, —Cl, —F, —Br, —I, —CH₃, —CH₂—CH₃, —O—CH₃, —O—CH₂—CH₃, —NH₂, —NR^{N}₂, —COOR*, —COOH, —CONH₂, and —CN.
{EO3}. The K-Ras inhibiting compound according to Embodiment {EO1}, wherein R₇ is —R, —CH₂—C(O)—NH—R, or —C(O)—NH—R.
{EO4}. The K-Ras inhibiting compound according to Embodiment {EO3}, wherein R is R^{B}, where R^{B} is a ring “C” having the form

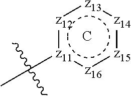

where ring “C” is a five- or six-membered optionally aromatic ring, z₁₁ is C, CH, or N; and z₁₂-z₁₆ are independently selected from N, NH, NR^{N}, O, S, C═O, CH, CX, CR, CH₂, C(X)(X), C(R)(X), or C(R)(R); and in the case where ring “C” is a five-membered ring, z₁₄ is a bond (i.e., it is absent); and wherein any two vicinal substituents X and/or R and/or R^{N} may together form a 5- or 6-membered ring fused to ring “C.”
{EO5}. The K-Ras inhibiting compound according to Embodiment {EO4}, wherein ring “C” is a six-membered aromatic ring.
{EO6}. The K-Ras inhibiting compound according to Embodiment {EO5}, wherein at least one z₁₂-z₁₆ is of the form C(X), where X is selected from the group consisting of —Cl, —F, —Br, —I, —OH, —O—CH₃, —O—CH₂—CH₃, —NH₂, —NR^{N}₂, and —CN.
{EO7}. The K-Ras inhibiting compound according to Embodiment {EO5}, wherein at least two z₁₂-z₁₆ is of the form C(X), where X is selected from the group consisting of —Cl, —F, —Br, —I, —OH, —O—CH₃, —O—CH₂—CH₃, —NH₂, —NR^{N}₂, and —CN.
{EO8}. The K-Ras inhibiting compound according to Embodiment {EO5}, wherein at least one z₁₂-z₁₆ is of the form C(R), where R is a C₃₋₆ linear or branched hydrocarbon, optionally substituted with one or more groups X and/or with 1-6 heteroatoms selected from O, S, N, P, F, Cl, Br, I, and combinations thereof.
{EO9}. The K-Ras inhibiting compound according to Embodiments {EO4}-{EO8}, wherein R is of the form —R.
{EO10}. The K-Ras inhibiting compound according to Embodiments {EO4}-{EO8}, wherein R₇ is of the form —CH₂-L₁-R.
{EO11}. The K-Ras inhibiting compound according to Embodiment {EO1}, wherein ring “B” is a six-membered aromatic ring.
{EO12}. The K-Ras inhibiting compound according to Embodiment {EO11}, wherein at least one z₇-z₁₀ is of the form C(X), where X is selected from the group consisting of —Cl, —F, —Br, —I, —CH₃, —OH, —O—CH₃, —O—CH₂—CH₃, —NH₂, —NR^{N}₂, —CN, —C(O)OH, —C(O)OR*, and —C(O)—NH₂.
{EO13}. The K-Ras inhibiting compound according to Embodiment {EO11}, wherein at least two of z₇-z₁₀ are C(X).
{EO14}. The K-Ras inhibiting compound according to Embodiment {EO11}, wherein at least one z₇-z₁₀ is of the form C(R), where R is a C₃₋₆ linear or branched hydrocarbon, optionally substituted with one or more groups X and/or with 1-6 heteroatoms selected from O, S, N, P, F, Cl, Br, I, and combinations thereof.
{EO15}. The K-Ras inhibiting compound according to Embodiment {EO11}, wherein at least one of z₈-z₁₁ is NH or N.
{EO16}. The K-Ras inhibiting compound according to Embodiment {EO11}, wherein at least two of z₈-z₁₁ are N.
{EO17}. The K-Ras inhibiting compound according to Embodiment {EO1}, wherein ring “B” is a five-membered aromatic ring.
{EO18}. The K-Ras inhibiting compound according to Embodiment {EO17}, wherein at least one of z₈-z₁₀ is selected from the group consisting of N, NH, N(R), and S.
{EO19}. The K-Ras inhibiting compound according to Embodiment {EO17}, wherein at least two of z₈-z₁₀ is independently selected at each occurrence from the group consisting of N, NH, N(R), and S.
{EO20}. The K-Ras inhibiting compound according to Embodiment {EO17}, wherein z₈ or z₁₀ is N; and the other of z₈ or z₁₀ is N(R′); where R′ is of the form —(CH₂)ᵢ—R^{B}; where i is an integer from 0-2 (e.g., i=1) and R^{B} has the form:

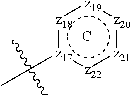

where ring “D” is a six-membered aromatic ring; z₁₇ is C, CH, or N; and z₁₈-z₂₂ are independently selected from N, NH, NR^{N}, O, S, C═O, CH, CX, CR, CH₂, C(X)(X), C(R)(X), and C(R)(R); and wherein any two vicinal substituents X and/or R and/or R^{N} may together form a 5- or 6-membered ring fused to ring “D.”
{EO21}. The K-Ras inhibiting compound according to Embodiment {EO20}, wherein at least one of z₁₈-z₂₂ is of the form CX, where X is selected from —Cl, —F, —Br, —I, —CF₃, —O—CH₃, —O—CH₂—CH₃, —NH₂, —CN, —C(O)—OH, and —O—CH₃.
{EO22}. The K-Ras inhibiting compound according to Embodiment {EO20}, wherein at least two z₁₈-z₂₂ are of the form CX, where X is selected from —Cl, —F, —Br, —I, —O—CH₃, —O—CH₂—CH₃, —NH₂, —CN, —C(O)—OH, and —O—CH₃.
{EO23}. The K-Ras inhibiting compound according to Embodiment {EO20}, wherein at least one z₁₈-z₂₂ is of the form CR^{A}, where R^{A} is a C₃₋₆ linear or branched hydrocarbon, optionally substituted with one or more groups X and/or with 1-6 heteroatoms selected from O, S, N, P, F, Cl, Br, I, and combinations thereof.
{EO24}. The K-Ras inhibiting compound according to Embodiment {EO20}, wherein at z₇-z₉ are selected independently from N, S, O, C(R), and C(-L₁-R); where L₁ is of the form —C(O)—N(R^{N})—R′, where R^{N} and R′ may together form a cyclic ring, where R′ is of the form —(CH₂)₁₋₃—R″, where R″ is a C₃₋₆ cyclic hydrocarbon (alicyclic or aromatic) or heterocycle (e.g., heteroaryl), optionally substituted with one or more groups X and/or with 1-6 heteroatoms selected from O, S, N, P, F, Cl, Br, I, and combinations thereof.
{EO25}. The K-Ras inhibiting compound according to Embodiment {EO1}, having the structure:

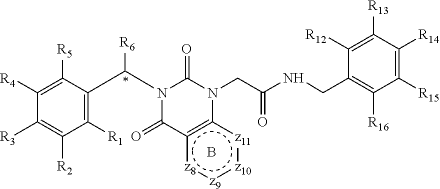

where R₁₂-R₁₆ are independently selected at each occurrence from hydrogen, —X (e.g., Cl, F, etc.), —R, and —OR.
{EO26}. The K-Ras inhibiting compound according to any one of Embodiments {EO1}-{EO25}, wherein ring “B” is pyrrole, thiphene, or imidazole.
{EO27}. The K-Ras inhibiting compound according to Embodiment {EO1}, having the structure:

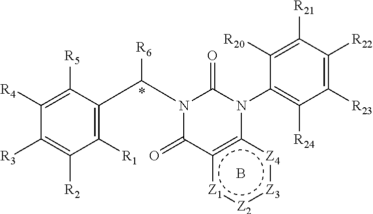

where R₂₀-R₂₄ are independently selected at each occurrence from hydrogen, —X, —R, and —OR.
{EO28}. The K-Ras inhibiting compound according to any one of Embodiments {EO1}-{EO27}, wherein ring “B” is a six-membered aromatic ring.
{EO29}. The K-Ras inhibiting compound according to any one of Embodiments {EO1}-{EO27}, wherein ring “A” is a five-membered aromatic ring.
{EO30}. The K-Ras inhibiting compound according to Embodiment {EO29}, wherein one or two of z₁-z₅ is selected from N, O, or S.
{EO31}. The K-Ras inhibiting compound according to Embodiments {EO1}, having the structure:

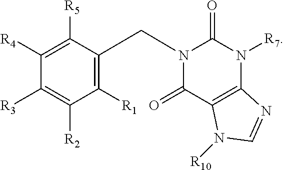

{EO32}. The K-Ras inhibiting compound according to Embodiments {EO31}, wherein R₁₀ is -L₁-R^{B}.
{EO33}. The K-Ras inhibiting compound according to Embodiments {EO31}, wherein R₁₀ is —CH₂—R^{B}.
{EO34}. The K-Ras inhibiting compound according to Embodiments {EO1}, having the structure:

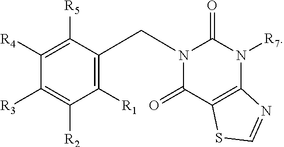

{EO35}. The K-Ras inhibiting compound according to Embodiments {EO34}, wherein R₇ is —CH₂-L₁-CH₂—R^{B}.
{EO36}. The K-Ras inhibiting compound according to Embodiments {EO34}, wherein R₇ is —CH₂—C(O)—NH—CH₂—R^{B}.

### Scaffold A16—Embodiment P (“EP”)

{EP1}. A compound for inhibiting K-Ras having a structure of formula (A16), or a pharmaceutically acceptable salt thereof:

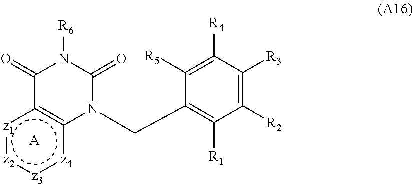

wherein R₁-R₅ are independently selected from hydrogen, —R, or —X;
R₆ is independently selected from hydrogen, —R, —X, —R^{B}, —R^{Q}—X, —R^{Q}—R, —(CH₂)₀₋₃-L₁-(CH₂)₀₋₃—R, —(CH₂)₀₋₃—R^{Q}—(CH₂)₀₋₃—R, —R^{Q}—(CH₂)₀₋₃-L₁-(CH₂)₀₋₃—R, —R^{B}, —R^{B}, —R^{B}, —R^{Q}, —R^{Q}—R^{B}, —(CH₂)₀₋₃-L₁-(CH₂)₀₋₃—R^{B}, —(CH₂)₀₋₃—R^{Q}—(CH₂)₀₋₃—R^{B}, or —R^{Q}—(CH₂)₀₋₃-L₁-(CH₂)₀₋₃-R^{B};
ring “A” is a five- or six-membered optionally aromatic ring; and z₁-z₄ are independently selected from N, NH, NR^{N}, O, S, C═O, CH, CX, CL₁X, CL₁R, NR, NX, CR, CH₂, C(X)(X), C(R)(X), C(R)(R), C(H)(L₁X) or C(H)(L₁R); and in the case where ring “A” is a five-membered ring, z₄ is a bond (i.e., it is absent); and wherein ring “A” may contain 0, 1, 2, or 3 double bonds; and wherein any two vicinal substituents X and/or R and/or R^{N} may together form a 5- or 6-membered ring fused to ring “A”, optionally substituted with one, two, or three groups X; and wherein any two geminal substituents X and/or R and/or R^{N} may together form a 5- or 6-membered spiro ring to ring “A;”
X is independently selected at each occurrence from —F, —Cl, —Br, —I, —OH, —OR*, —NH₂, —NHR*, —N(R*)₂, —N(R*)₃⁺, —N(R*)—OH, —N(*O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂—, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(═O)₁₋₂—R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂;
R* is independently selected at each occurrence from hydrogen and a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.);
R is selected from hydrogen or C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteroatoms selected from O, S, N, P, F, Cl, Br, I, and combinations thereof,
R^{B} is a C₃₋₆ cyclic hydrocarbon (alicyclic or aromatic) or heterocycle (e.g., heteroaryl), optionally substituted with one or more (e.g., 1-5) groups X and/or with 1-6 heteroatoms selected from O, S, N, P, F, Cl, Br, I, and combinations thereof, where any vicinal groups can form a 5- or 6-membered fused ring with said cyclic hydrocarbon;
R^{Q} is a cyclic group having the structure:

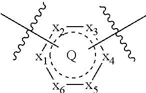

wherein ring “Q” is a five- or six-membered optionally aromatic ring; x₁ and x₄ are independently C, CH, CR or N; and x₂, x₃, x₅, and x₆, are independently selected from N, NH, NR^{N}, O, S, C═O, CH, CX, CR, CH₂, C(X)(X), C(R)(X), and C(R)(R); and in the case where ring “Q” is a five-membered ring, x₃ or x₅ is a bond (i.e., it is absent); and wherein any two vicinal substituents X and/or R and/or R^{N} may together form a 5- or 6-membered ring fused to ring “Q”; and
L₁ is selected independently at each occurrence from —O—, —S—, —NH—, —N(R^{N})—, —(CH₂)₁₋₄—, —C═C—, —C═C—(CH₂)₁₋₃—, —C(O)—, —C(O)—(CH₂)₁₋₃—, —C(O)—O—(CH₂)₁₋₃—, —C(O)—N(R^{N})—, —C(O)—N(R^{N})—(CH₂)₁₋₃—, —(CH₂)₁₋₃—C(O)—N(R^{N})—, —(CH₂)₀₋₃—NH—C(O)—, —(CH₂)₀₋₃—NH—C(O)—O—, —NH—S(O)₁₋₂—, —N(R^{N})—S(O)₁₋₂—, —O—(CH₂)₁₋₃—, —S(O)₁₋₂—, —S—(CH₂)₁₋₃—, —NH—(CH₂)₁₋₃—, —N(R^{N})—(CH₂)₁₋₃—, and —(OCH₂CH₂)₁₋₃—; where
R^{N} is independently selected at each occurrence from hydrogen, methyl, ethyl, or propyl.
{EP2}. The K-Ras inhibiting compound according to Embodiment {EP1}, wherein R₁-R₅ are independently selected from the group consisting of hydrogen, —Cl, —OH, —F, —Br, —I, —CH₃, —CH₂—CH₃, —O—CH₃, —O—CH₂—CH₃, —C(O)OH, —C(O)OR*, —NH₂, and —CN.
{EP3}. The K-Ras inhibiting compound according to Embodiment {EP1}, wherein R₆ is —R^{B}, —R^{Q}—R, or —R^{Q}—(CH₂)₀₋₃-L₁-(CH₂)₀₋₂-R.
{EP4}. The K-Ras inhibiting compound according to Embodiment {EP1}, wherein R₆ is —R^{Q-}(CH₂)₂—C(O)—NH—CH₂—R^{B} or R^{Q}—(CH₂)₂—NH—C(O)—CH₂—R^{B}.
{EP5}. The K-Ras inhibiting compound according to Embodiment {EP1}, wherein ring “A” is thiophenyl.
{EP6}. The K-Ras inhibiting compound according to Embodiment {EP1} having the structure:

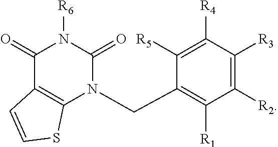

{EP7}. The K-Ras inhibiting compound according to Embodiment {EP1} having the structure:

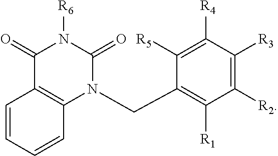

{EP8}. The K-Ras inhibiting compound according to Embodiment {EP3}, wherein R^{Q} and/or R^{B} is optionally substituted phenyl.
{EP9}. The K-Ras inhibiting compound according to Embodiment {EP8}, wherein R₆ is —R^{Q-}(CH₂)₀₋₃-L₁-(CH₂)₀₋₂—R, where R^{Q} is phenyl and L₁ is —C(O)—NH— or —C(O)—.
{EP10}. The K-Ras inhibiting compound according to Embodiment {EP9}, wherein R is R^{B}, where R^{B} is optionally substituted phenyl, furanyl, cyclopropyl, and cyclopentyl, wherein vicinal groups in R^{B} may together form a 5- or 6-membered fused ring.
{EP11}. The K-Ras inhibiting compound according to Embodiment {EP1}, wherein ring “A” is a 6-membered aromatic ring.
{EP12}. The K-Ras inhibiting compound according to Embodiment {EP11}, wherein z₁-z₄ are CH.
{EP13}. The K-Ras inhibiting compound according to Embodiment {EP11}, wherein z₁ and/or z₄ is N.
{EP14}. The K-Ras inhibiting compound according to Embodiment {EP1}, wherein ring “A” is a 5-membered aromatic ring.
{EP15}. The K-Ras inhibiting compound according to Embodiment {EP14}, wherein z₁ and/or z₃ is independently S or N.
{EP16}. The K-Ras inhibiting compound according to Embodiment {EP14}, wherein z₁ is S; and z₂ and z₃ are independently selected from C(R), CH, or CHL₁R.
{EP17}. The K-Ras inhibiting compound according to Embodiment {EP16}, wherein the substituents of z₂ and z₃ together form a five or six membered ring.
{EP18}. The K-Ras inhibiting compound according to Embodiment {EP16}, wherein L₁ is —NH—CO—.
{EP19}. The K-Ras inhibiting compound according to Embodiment {EP1}, wherein vicinal R groups at z₂ and z₃ together form a five- or six-membered ring, optionally containing from 1-6 heteroatoms selected from N, O, S, P, F, Cl, Br, and I, and/or optionally comprising from 1-3 groups X.
{EP20}. The K-Ras inhibiting compound according to Embodiment {EP19}, with the structure:

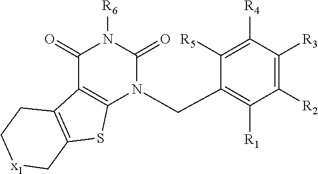

wherein x₁ is selected from CH₂, CR*₂, C(H)(L₁-R*), NH, NR*, or N(L₁-R*), where L₁ is —C(O)— or —C(O)O—.
{EP21}. The K-Ras inhibiting compound according to Embodiment {EP14} or {EP20}, where x₁ is N—C(O)—R* or N—C(O)—OR*.
{EP22}. The K-Ras inhibiting compound according to Embodiment {EP20}, having the structure:

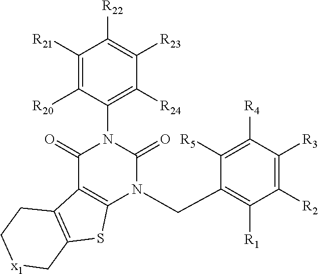

wherein R₁-R₅ and R₂₀-R₂₄ are independently selected at each occurrence from hydrogen and —X (e.g., Cl or F).
{EP23}. The K-Ras inhibiting compound according to Embodiment {EP22}, wherein x₁ is N—C(O)—X₁₀—R, where X₁₀ is O, S, NH, or CH₂.
{EP24}. The K-Ras inhibiting compound according to Embodiment {EP20}-{EP23}, wherein R* of x₁ is a C₁₋₆ alkyl.
{EP25}. The K-Ras inhibiting compound according to Embodiment {EP1}, with the structure:

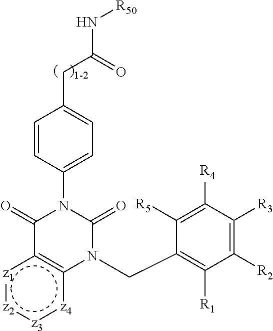

wherein R₅₀ is selected from (i) C₁₋₆ hydrocarbons, (ii) —R, (iii) —(CH₂)₁₋₃—R, and (iv) —(CH₂)₁₋₃—R^{B}, wherein R₅₀ is optionally substituted with 1-6 heteroatoms selected from N, O, S, P, F, Cl, Br, and I, and/or optionally comprising from 1-3 groups X.
{EP26}. The K-Ras inhibiting compound according to Embodiment {EP25}, with the structure:

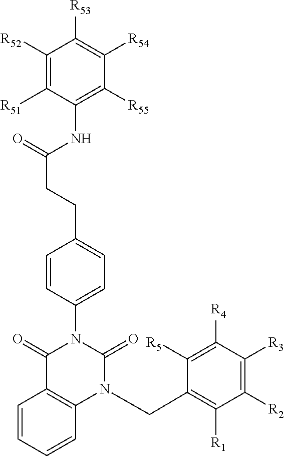

wherein R₁-R₅ and R₅₁-R₅₅ are independently selected from hydrogen-X (e.g., Cl or F), lower alkyl (e.g., methyl, ethyl), or lower alkoxy (e.g., methoxy, ethoxy).
{EP27}. The K-Ras inhibiting compound according to Embodiment {EP26}, wherein one of R₁-R₅ is methoxy, one or two of R₅₁-R₅₅ are independently Cl and/or F, and the rest are hydrogen.

### Scaffold A17—Embodiment Q (“EQ”)

{EQ1}. A compound for inhibiting K-Ras having formula (A17), or a pharmaceutically acceptable salt thereof:

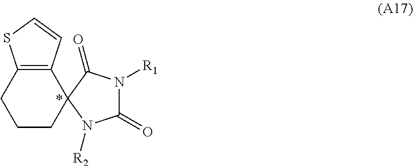

wherein R₁ is selected from hydrogen, —R, -L₁-R, -L₁-X, —(CH₂)₀₋₂-L₁-(R^{Q})₀₋₁—(CH₂)₀₋₂—X, or —(CH₂)₀₋₂-L₁-(R^{Q})₀₋₁—(CH₂)₀₋₂-L₁-R, —R^{B}, -L₁-R^{B}, or —(CH₂)₀₋₂-L₁-(R)₀₋₁—(CH₂)₀₋₂-L₁-R^{B};
R₂ is selected from hydrogen, methyl, ethyl, propyl, or isopropyl.
X is independently selected at each occurrence from —F, —Cl, —Br, —I, —OH, —OR*, —NH₂, —NHR*, —N(R*)₂, —N(R*)₃⁺, —N(R*)—OH, —N(*O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂⁻, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(═O)₁₋₂—R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂;
R* is independently selected at each occurrence from hydrogen and a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.);
R^{Q} is a cyclic group having the structure:

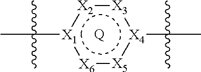

wherein ring “Q” is a five- or six-membered optionally aromatic ring, x₁ and x₄ are independently C, CH, CR or N; and x₂, x₃, x₅, and x₆, are independently selected from N, NH, NR^{N}, O, S, C═O, CH, CX, CR, CH₂, C(X)(X), C(R)(X), and C(R)(R); and in the case where ring “Q” is a five-membered ring, x₃ or x₅ is a bond (i.e., it is absent); and wherein any two vicinal substituents X and/or R and/or R^{N} may together form a 5- or 6-membered ring fused to ring “Q”;
R is selected from hydrogen or C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteroatoms selected from O, S, N, P, F, Cl, Br, I, and combinations thereof;
R^{B} is a C₃₋₆ cyclic hydrocarbon (alicyclic or aromatic) or heterocycle (e.g., heteroaryl), optionally substituted with one or more (e.g., 1-5) groups X and/or with 1-6 heteroatoms selected from O, S, N, P, F, Cl, Br, I, and combinations thereof, where any two vicinal groups may together form a 5- or 6-membered fused ring with said cyclic hydrocarbon; and
L₁ is selected independently at each occurrence from —O—, —S—, —NH—, —N(R^{N})—, —(CH₂)₁₋₄—, —C═C—, —C═C—(CH₂)₁₋₃—, —C(O)—, —C(O)—(CH₂)₁₋₃—, —C(O)—O—(CH₂)₁₋₃—, —C(O)—N(R^{N})—, —C(O)—N(R^{N})—(CH₂)₁₋₃—, —(CH₂)₁₋₃—C(O)—N(R^{N})—, —(CH₂)₀₋₃—NH—C(O)—O—, —NH—S(O)₁₋₂—, —N(R^{N})—S(O)₁₋₂—, —O—(CH₂)₁₋₃—, —S(O)₁₋₂—, —S—(CH₂)₁₋₃—, —NH—(CH₂)₁₋₃—, —N(R^{N})—(CH₂)₁₋₃—, or —(OCH₂CH₂)₁₋₃—; where
R^{N} is independently selected at each occurrence from hydrogen, methyl, ethyl, or propyl.
{EQ2}. The K-Ras inhibiting compound according to Embodiment {EQ1}, wherein R₁ is -L₁-C(O)—NH₂.
{EQ3}. The K-Ras inhibiting compound according to Embodiment {EQ1}, wherein R₁ is —(CH₂)₁₋₄—X.
{EQ4}. The K-Ras inhibiting compound according to Embodiment {EQ1}, wherein R₁ is —(CH₂)₁₋₃-L₁-R^{B}; where R^{B} has the form

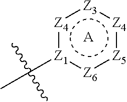

where ring “A” is a five- or six-membered optionally aromatic ring; z₁ is C, CH, or N; and z₂-z₆ are independently selected at each occurence from N, NH, NR^{N}, O, S, C═O, CH, CX, CR, CH₂, C(X)(X), C(R)(X), and C(R)(R); and in the case where ring “A” is a five-membered ring, z₄ is a bond (i.e., it is absent); and wherein any two vicinal substituents X and/or R and/or R^{N} may together form a 5- or 6-membered ring fused to ring “A.”
{EQ5}. The K-Ras inhibiting compound according to Embodiment {EQ4}, wherein ring “A” is a six-membered aromatic ring.
{EQ6}. The K-Ras inhibiting compound according to Embodiment {EQ4}, wherein at least one z₂-z₆ is of the form C(R′), where R′ is of the form —(CH₂)₁₋₃—NH—C(O)—R*, where R* is methyl, ethyl, or propyl.
{EQ7}. The K-Ras inhibiting compound according to Embodiment {EQ1}, wherein the compound has a structure:

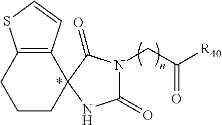

where R₄₀ is a group —X, —R, —R^{B}, —R^{Q}—R, —R—R^{B}, where R₄₀ is a C₃-C₆ optionally aromatic cyclic hydrocarbon, where R and R^{B} are optionally substituted with one or more groups X or with 1-6 heteroatoms selected from O, S, N, P, F, and Cl, and where n is an integer from 0-4 (e.g., 1, 2, 3, or 4).
{EQ8}. The K-Ras inhibiting compound according to Embodiment {EQ7}, wherein R₄₀ is a group —C(O)NH₂.
{EQ9}. The K-Ras inhibiting compound according to Embodiment {EQ7}, wherein R₄₀ is a group —R^{Q}—NHC(O)CH₃, where R^{Q} is phenyl.
The K-Ras inhibiting compound according to Embodiment {EQ1}, wherein the compound has a structure:

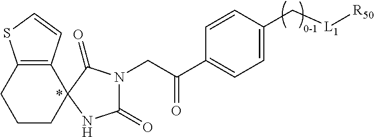

wherein R₅₀ is hydrogen, X, lower alkyl (e.g., —CH₃; —CH₂—CH₃; —CH₂—CH₂—CH₃, —C(CH₃)₃, R, or R^{B}

### Scaffold A18—Embodiment R (“ER”)

{ER1}. A compound for inhibiting K-Ras having formula (A18), or a pharmaceutically acceptable salt thereof:

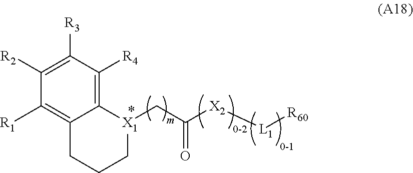

wherein “m” is zero or one;
X₁ is N or CH;
X₂ is independently at each occurence NH or CH₂;
R₁₋₄ are independently selected from hydrogen, —R, —R^{B}, and —X;
R₆₀ is R_{B} or C(R₅)(R₆)(R₇);
R₅₋₆ are independently selected from hydrogen, —R, —X, -L₁-R, -L₁-R^{B}, -L₁-C(R^{B})(X), and -L₁-X; where groups R₅ and R₆ may together form a five- or six-membered ring, optionally including one or more heteroatoms selected from O, N, or S, and/or one or more (e.g., one, two, three, etc.) groups X, R, Cl, F, or Br;
R₇ is hydrogen, —R, —R^{B}, or —X;
X is independently selected at each occurrence from —F, —Cl, —Br, —I, —OH, —OR*, —NH₂, —NHR*, —N(R*)₂, —N(R*)₃⁺, —N(R*)—OH, —N(*O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂—, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(═O)₁₋₂—R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂;
R* is independently selected at each occurrence from hydrogen and a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.);
R^{B} is a C₃₋₆ cyclic hydrocarbon (alicyclic or aromatic) or heterocycle (e.g., heteroaryl), optionally substituted with one or more (e.g., 1-5) groups R and/or X and/or with 1-6 heteroatoms selected from O, S, N, P, F, Cl, Br, I, and combinations thereof, where any vicinal groups can form a 5- or 6-membered fused ring;
R is selected from hydrogen or C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteroatoms selected from O, S, N, P, F, Cl, Br, I, and combinations thereof; and
L₁ is selected independently at each occurrence from —O—, —S—, —NH—, —N(R^{N})—, —(CH₂)₁₋₄—, —C═C—, —C═C—(CH₂)₁₋₃—, —C(O)—, —C(O)—(CH₂)₁₋₃—, —C(O)—O—(CH₂)₁₋₃—, —C(O)—N(R^{N})—, —N(R^{N})—C(O)—, —C(O)—N(R^{N})—(CH₂)₁₋₃—, —(CH₂)₁₋₃—C(O)—N(R^{N})—, —(CH₂)₀₋₃—NH—C(O)—O—, —NH—S(O)₁₋₂—, —N(R^{N})—S(O)₁₋₂—, —O—(CH₂)₁₋₃—, —S(O)₁₋₂—, —S—(CH₂)₁₋₃—, —NH—(CH₂)₁₋₃—, —N(R^{N})—(CH₂)₁₋₃₋, and —(OCH₂CH₂)₁₋₃—; where
R^{N} is independently selected at each occurrence from hydrogen, methyl, ethyl, or propyl.
{ER2}. The K-Ras inhibiting compound according to Embodiment {ER1}, wherein R₁₋₄ are independently selected from the group consisting of hydrogen, —Cl, —F, —Br, —I, —OH, —CH₃, —CH₂CH₃, —OCH₃, —OCH₂CH₃, —C(O)OH, —NH₂, —NR^{N}₂, and —CN.
{ER3}. The K-Ras inhibiting compound according to Embodiment {ER1}, wherein R₅ and/or R₆ are/is —R^{B}; where R^{B} is independently a C₃₋₆ cyclic hydrocarbon (alicyclic or aromatic) or heterocycle (e.g., heteroaryl), optionally substituted with one or more groups X and/or with 1-6 heteroatoms selected from O, S, N, P, F, Cl, Br, I, and combinations thereof.
{ER4}. The K-Ras inhibiting compound according to Embodiment {ER3}, wherein R^{B} of R₅ and/or R₆ is a five-membered aromatic ring.
{ER5}. The K-Ras inhibiting compound according to Embodiment {ER4}, wherein R^{B} R₅ and/or R₆ is thiophenyl, furanyl, or pyrrolyl.
{ER6}. The K-Ras inhibiting compound according to Embodiment {ER4}, wherein R₅ and/or R₆ are/is -L₁-R or -L₁-X; where L₁ is independently at each occurrence —S—, —NH—C(O)—, or —(CH₂)—NH—C(O)—.
{ER7}. The K-Ras inhibiting compound according to Embodiment {ER6}, wherein R of R₅ and/or R₆ is R^{B}; where R^{B} is independently at each occurrence a C₃₋₆ cyclic hydrocarbon (alicyclic or aromatic) or heterocycle (e.g., heteroaryl), optionally substituted with one or more groups X and/or with 1-6 heteroatoms selected from O, S, N, P, F, Cl, Br, I, and combinations thereof.
{ER8}. The K-Ras inhibiting compound according to Embodiment {ER7}, wherein R^{B} of R₅ and/or R₆ is a five-membered aromatic ring.
{ER9}. The K-Ras inhibiting compound according to Embodiment {ER7}, wherein R^{B} is selected from the group consisting of:

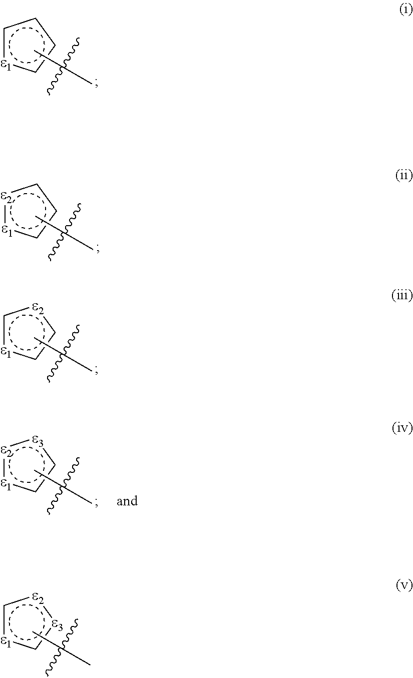

wherein ε₁, ε₂, and ε₃, are independently selected from N, NH, NR^{N}, NR*, —C(O)—, S, and O; with the proviso that where the point of attachment is ε₁, ε₂, or ε₃, then that position represents N; and wherein carbon atoms which are not the point of attachment may be optionally substituted with a group X (e.g., F, Cl, Br, —SH, —OH, —OCH₃) or with a group R (e.g., —CH₃, —CH₂CH₃, etc.) which may in turn be substituted with one or more (e.g., 1-3) groups X and/or 1-10 heteroatoms selected from O, S, N, P, F, Cl, Br, I, and combinations thereof; and wherein the dashed circles indicate that each ring may comprise zero, one, or two double bonds and may be aromatic, and wherein any two adjacent groups X, R^{N}, R*, and/or R may together form a 5- or 6-membered ring fused with the ring.
{ER10}. The K-Ras inhibiting compound according to Embodiment {ER8}, wherein R^{B} of R₅ and/or R₆ is thiophenyl, furanyl, or pyrrolyl.
{ER11}. The K-Ras inhibiting compound according to Embodiment {ER6}, wherein R₅ or R₆ is -L₁-X, where L₁ is —NH—C(O)— and X is —NH₂.
{ER12}. The K-Ras inhibiting compound according to Embodiment {ER6}, wherein R₅ or R₆ is -L₁-R, where L₁ is —NH—C(O)— and R is —CH₃.
{ER13}. The K-Ras inhibiting compound according to Embodiment {ER6}, wherein R₅ or R₆ is -L₁-R^{B}, where L₁ is —NH—C(O)— and R^{B} is an 5- or 6-membered aromatic heterocycle (e.g., furanyl, thiophenyl, etc.).
{ER14}. The K-Ras inhibiting compound according to Embodiment {ER6}, wherein R₅ or R₆ is -L₁-R^{L}-L₂-R, where L₁ and L₂ are —NH—C(O)—; R^{L} is an optionally substituted (with 1-3 groups X and/or 1-5 heteroatoms) divalent C₁₋₁₂ hydrocarbon radical.
{ER15}. The K-Ras inhibiting compound according to Embodiment {ER14}, wherein R^{L} is a group —(CH₂)ₚ—C(R*)(R^{B})—, where “p” is an integer from 0-3.
{ER16}. The K-Ras inhibiting compound according to Embodiment {ER1}, wherein R₅ is -L₁-X, and R₆ is —R^{B}, respectively; where R^{B} is a C₃₋₆ cyclic hydrocarbon (alicyclic or aromatic) or heterocycle (e.g., heteroaryl), optionally substituted with one or more groups X and/or with 1-6 heteroatoms selected from O, S, N, P, F, Cl, Br, I, and combinations thereof; and L₁ is —NH—C(O)—.
{ER17}. The K-Ras inhibiting compound according to Embodiment {ER1}, wherein R₅ is of the form -L₁-R′; where R′ is —C(R^{B})(X); where R^{B} a C₃₋₆ cyclic hydrocarbon (alicyclic or aromatic) or heterocycle (e.g., heteroaryl), optionally substituted with one or more groups X and/or with 1-6 heteroatoms selected from O, S, N, P, F, Cl, Br, I, and combinations thereof.
{ER18}. The K-Ras inhibiting compound according to Embodiment {ER1}, having the structure:

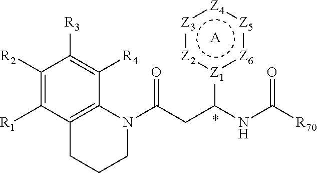

where z₁ is C, CH, or N; and z₂-z₆ are independently selected from N, NH, NR^{N}, O, S, C═O, CH, CX, CR, CH₂, C(X)(X), C(R)(X), and C(R)(R); and in the case where ring “A” is a five-membered ring, z₄ is a bond (i.e., it is absent); and wherein any two vicinal substituents X and/or R and/or R^{N} may together form an optionally aromatic 5- or 6-membered ring fused to ring “A”;
R₇₀ is hydrogen, group X, or a C₁₋₁₂ hydrocarbon optionally substituted with one or more groups X and/or with 1-6 heteroatoms selected from O, S, N, P, F, Cl, Br, I, and combinations thereof.
{ER19}. The K-Ras inhibiting compound according to Embodiment {ER18}, where R₇₀ is methyl.
{ER20}. The K-Ras inhibiting compound according to Embodiment {ER18}, where R₇₀ is —NR^{N}₂.
{ER21}. The K-Ras inhibiting compound according to Embodiment {ER18}, where ring “A” is a five membered ring.
{ER22}. The K-Ras inhibiting compound according to Embodiment {ER21}, wherein ring “A” has the form:

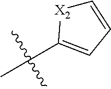

wherein, X₂ is S or O; and any available carbon atom is optionally substituted with a group X (e.g., F, Cl, Br, —SH, —OH, or —OCH₃) or with a group R (e.g., —CH₃, —CH₂CH₃, etc.).
{ER23}. The K-Ras inhibiting compound according to Embodiment {ER22}, wherein X₂ is S.
{ER24}. The K-Ras inhibiting compound according to Embodiment {ER1}, wherein R₅ and R₆ together form a six-membered ring.
{ER25}. The K-Ras inhibiting compound according to Embodiment {ER24}, wherein said six-membered ring is alicyclic.
{ER26}. The K-Ras inhibiting compound according to Embodiment {ER24}, wherein said six-membered ring has substituted at with at least one heteroatom selected from N, O, or S.
{ER27}. The K-Ras inhibiting compound according to Embodiment {ER1}, wherein R₇ is —C(O)—NH₂.
{ER28}. The K-Ras inhibiting compound according to Embodiment {ER1}, wherein X₁ is N.
{ER29}. The K-Ras inhibiting compound according to Embodiment {ER1}, wherein X₁ is CH.

### Scaffold A19—Embodiment S (“ES”)

{ES1}. A compound for inhibiting K-Ras having formula (A19), or a pharmaceutically acceptable salt thereof:

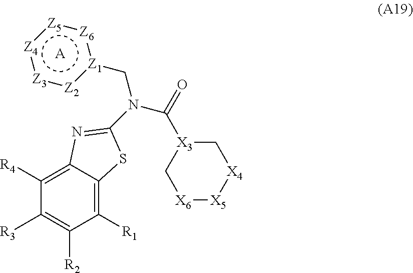

where ring “A” is a five- or six-membered optionally aromatic ring, z₁ is C, CH, or N; and z₂-z₆ are independently selected from N, NH, NR^{N}, O, S, C═O, CH, CX, NR, NX, CR, CH₂, C(X)(X), C(R)(X), C(R)(R), and C(H)(L₁R); and wherein ring “A” may contain 0, 1, 2, or 3 double bonds; and in the case where ring “A” is a five-membered ring, z₄ is a bond (i.e., absent); and wherein any two vicinal substituents X and/or R and/or R^{N} may together form a 5- or 6-membered ring fused to ring “A”;
X₃ is selected from N or CH;
X₄—X₆ may each be absent (i.e. it is a bond) or independently selected from NH, NR, NX, C═O, N-L₁-R, CH-L₁-R, CH₂, CHX, or CHR; wherein if any two vicinal positions X₄, X₅ and/or X₆ are absent then the two said vicinal positions are together a single bond; and if X₄, X₅, and X₆ are each absent, they together are a single bond;
wherein R₁-R₅ are independently selected from hydrogen, —R, —OR, and —X; and where any two vicinal groups R₁-R₄ may together form a 5- or 6-membered fused ring;
X is independently selected at each occurrence from —F, —Cl, —Br, —I, —OH, —OR*, —NH₂, —NHR*, —N(R*)₂, —N(R*)₃⁺, —N(R*)—OH, —N(*O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂⁻, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(═O)₁₋₂—R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂;
R^{N} is independently selected at each occurrence from hydrogen, methyl, ethyl, or propyl;
R* is independently selected at each occurrence from hydrogen and a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.);
L₁ is selected independently at each occurrence from —O—, —S—, —NH—, —N(R^{N})—, —(CH₂)₁₋₄—, —C═C—, —C═C—(CH₂)₁₋₃—, —C(O)—, —C(O)—(CH₂)₁₋₃—, —C(O)—O—(CH₂)₁₋₃—, —C(O)—N(R^{N})—, —N(R^{N})—C(O)—, —C(O)—N(R^{N})—(CH₂)₁₋₃₋, —(CH₂)₁₋₃—C(O)—N(R^{N})—, —(CH₂)₀₋₃—NH—C(O)—O—, —NH—S(O)₁₋₂—, —N(R^{N})—S(O)₁₋₂—, —O—(CH₂)₁₋₃—, —S(O)₁₋₂—, —S—(CH₂)₁₋₃—, —NH—(CH₂)₁₋₃—, —N(R^{N})—(CH₂)₁₋₃—, or —(OCH₂CH₂)₁₋₃—; and
R is selected from hydrogen or C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteroatoms selected from O, S, N, P, F, Cl, Br, I, and combinations thereof.
{ES2}. The K-Ras inhibiting compound according to Embodiment {ES1}, wherein one or more of R₁-R₄ are independently selected from the group consisting of hydrogen, —OH, —Cl, —F, —Br, —I, —NH₂, NR^{N}₂, —CH₃, —CH₂CH₃, —OCH₃, —OCH₂CH₃, —C(O)OR*, —C(O)NR^{N}₂, and —CN.
{ES3}. The K-Ras inhibiting compound according to Embodiment {ES1}, wherein one or more of R₁-R₄ are hydrogen or —CH₃.
{ES4}. The K-Ras inhibiting compound according to Embodiment {ES1}, wherein one or more of R₂, R₃, and R₄ are —CH₃.
{ES5}. The K-Ras inhibiting compound according to Embodiment {ES1}, wherein ring A is aromatic and at least one of z₂, z₃, z₄, or z₅ is N.
{ES6}. The K-Ras inhibiting compound according to Embodiment {ES1}, wherein X₄ is CH, and X₃ and X₅ are CH₂.
{ES7}. The K-Ras inhibiting compound according to Embodiment {ES1}, wherein X₃ and/or X₅ is a bond.
{ES8}. The K-Ras inhibiting compound according to Embodiment {ES1}, wherein X₃ is NR.
{ES9}. The K-Ras inhibiting compound according to Embodiment {ES1}, wherein X₆ is C═O.
{ES10}. The K-Ras inhibiting compound according to Embodiment {ES8}, wherein R of X₃ comprises a six-membered aromatic ring.
{ES11}. The K-Ras inhibiting compound according to Embodiment {ES1)}, wherein X₅ is N-L₁-R, and L₁ is —C(O)—.
{ES12}. The K-Ras inhibiting compound according to Embodiment {ES11}, wherein R of X₅ is a C₁₋₄ branched hydrocarbon radical.

### Scaffold A20—Embodiment T (“ET”)

{ET1}. A compound for inhibiting K-Ras having formula (A20), or a pharmaceutically acceptable salt thereof:

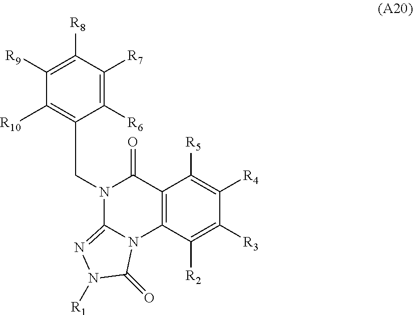

wherein R₁ is selected from —X, —R, -L₁-R^{B}, or —R^{B};
R₂, R₄, and R₅ are independently selected from hydrogen, —X, —R, or —R^{B}
R₃ is independently selected from hydrogen, —X, —R, —R^{B}, -L₁-X, -L₁-R, -L₁-(CH₂)₀₋₂—R^{B}, or -L₁-R^{B};
R₆-R₁₀ are independently selected from —R or —X;
X is independently selected at each occurrence from —F, —Cl, —Br, —I, —OH, —OR*, —NH₂, —NHR*, —N(R*)₂, —N(R*)₃⁺, —N(R*)—OH, —N(*O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂⁻, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(═O)₁₋₂—R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂;
L₁ is selected independently at each occurrence from —O—, —S—, —NH—, —N(R^{N})—, —(CH₂)₁₋₄—, —C═C—, —C═C—(CH₂)₁₋₃—, —C(O)—, —(CH₂)₁₋₃—C(O)—, —C(O)—(CH₂)₁₋₃—, —C(O)—O—(CH₂)₁₋₃—, —C(O)—N(R^{N})—, —N(R^{N})—C(O)—, —C(O)—N(R^{N})—(CH₂)₁₋₃—, —(CH₂)₁₋₃—C(O)—N(R^{N})—, —(CH₂)₀₋₃—NH—C(O)—O—, —NH—S(O)₁₋₂—, —N(R^{N})—S(O)₁₋₂—, —O—(CH₂)₁₋₃—, —S(O)₁₋₂—, —S—(CH₂)₁₋₃—, —NH—(CH₂)₁₋₃—, —N(R^{N})—(CH₂)₁₋₃—, Or —(OCH₂CH₂)₁₋₃—;
R* is independently selected at each occurrence from hydrogen or a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.);
R is selected from hydrogen or C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteroatoms selected from O, S, N, P, F, Cl, Br, I, and combinations thereof;
R^{N} is hydrogen, methyl, ethyl, or propyl;
R^{B} is a C₃₋₆ cyclic hydrocarbon (alicyclic or aromatic) or heterocycle (e.g., heteroaryl), optionally substituted with one or more (e.g., 1-5) groups R and/or X and/or with 1-6 heteroatoms selected from O, S, N, P, F, Cl, Br, I, and combinations thereof, where any vicinal groups can form a 5- or 6-membered fused ring.
{ET2}. The K-Ras inhibiting compound according to Embodiment {ET1}, wherein R₁ is a group —CH₂—R^{B}, where R^{B} is a six-membered aromatic hydrocarbon optionally substituted with a group selected from —F, —Cl, —CH₃, —CH₂—CH₃, or —OH.
{ET3}. The K-Ras inhibiting compound according to Embodiment {ET1}, wherein R₂-R₅ are independently selected from hydrogen and -L₁-R, and R is R*.
{ET4}. The K-Ras inhibiting compound according to Embodiment {ET1}, wherein R₂, R₄, and R₅ are each hydrogen.
{ET5}. The K-Ras inhibiting compound according to Embodiment {ET1}, wherein R₃ is -L₁-R.
{ET6}. The K-Ras inhibiting compound according to Embodiment {ET5}, wherein R of R₃ is a C₄ branched hydrocarbon.
{ET7}. The K-Ras inhibiting compound according to Embodiment {ET5}, wherein L₁ of R₃ is —C(O)—NH—.
{ET8}. The K-Ras inhibiting compound according to Embodiment {ET1}, wherein R₆-R₁₀ are each independently selected from hydrogen, —F, —Cl, —CH₃, —CH₂—CH₃, or —OH.

### Scaffold A21—Embodiment U (“EU”)

{EU1}. A compound for inhibiting K-Ras having formula (A21), or a pharmaceutically acceptable salt thereof:

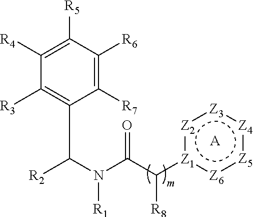

wherein m is 0 or 1;
ring “A” is a five- or six-membered optionally aromatic ring; z₁ is selected from C, CH, or N; and z₂-z₆ are selected from N, NR, NH, NR^{N}, NR^{B}, O, S, C═O, CH, CX, CR, CR^{B}, CH₂, CHR^{B}, C(X)(X), C(R)(X), or C(R)(R); and in the case where ring “A” is a five-membered ring, z₄ is a bond (i.e., it is absent); and wherein any two vicinal substituents X and/or R and/or R^{N} may together form a 5- or 6-membered ring fused to ring “A”;
R₁ and R₂ are independently selected from hydrogen or R;
R₃-R₇ are independently selected from hydrogen, R, OR, or X; where any two vicinal substituents X and/or OR and/or R may together form a 5- or 6-membered fused ring;
R₈ is independently at each occurence hydrogen, methyl, or ethyl;
X is independently selected at each occurrence from —F, —Cl, —Br, —I, —OH, —OR*, —NH₂, —NHR*, —N(R*)₂, —N(R*)₃⁺, —N(R*)—OH, —N(→O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂—, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(═O)₁₋₂—R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂;
R^{L} at each occurrence is a C₁₋₆ linear or branched bivalent hydrocarbon radical; optionally substituted with one or more (e.g., 1-5) groups X and/or with 1-6 heteroatoms independently selected from O, S, N, P, F, Cl, Br, or I;
L₁ is selected independently at each occurrence from —O—, —S—, —NH—, —N(R^{N})—, —(CH₂)₁₋₄—, —C═C—, —C═C—(CH₂)₁₋₃—, —C(O)—, —C(O)—(CH₂)₁₋₃—, —S(O)₂—, —C(O)—O—(CH₂)₁₋₃—, —C(O)—N(R^{N})—, —N(R^{N})—C(O)—, —C(O)—N(R^{N})—(CH₂)₁₋₃—, —(CH₂)₁₋₃—C(O)—N(R^{N})—, —(CH₂)₀₋₃—NH—C(O)—, —NH—S(O)₁₋₂—, —N(R^{N})—S(O)₁₋₂⁻, —O—(CH₂)₁₋₃—, —S(O)₁₋₂—, —S—(CH₂)₁₋₃—, —NH—(CH₂)₁₋₃—, —N(R^{N})—(CH₂)₁₋₃—, and —(OCH₂CH₂)₁₋₃—;
R^{B} is independently at each occurence a monocyclic or fused bicyclic group having the structure:

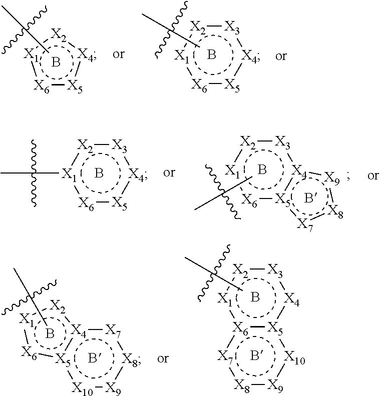

wherein ring B and B′ are independently five- or six-membered, optionally aromatic rings; x₁-x₁₀ are independently selected from N, NH, NR^{N}, O, S, S(═O)₂, C═O, C, CH, CX, CR, CH₂, C(X)(X), C(R)(X), and C(R)(R); and in the case where ring “B” is a five-membered ring, x₃ is a bond (i.e., it is absent); and wherein any two vicinal substituents X and/or R and/or R^{N} may together form an optionally substituted 5- or 6-membered ring fused to ring “B” and/or “B′”;
R^{N} is independently selected at each occurrence from hydrogen, methyl, ethyl, or propyl;
R* is independently selected at each occurrence from hydrogen and a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.); and
R is selected from hydrogen or C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteroatoms selected from O, S, N, P, F, Cl, Br, I, and combinations thereof.
{EU2}. The K-Ras inhibiting compound according to Embodiment {EU1}, wherein R₁ and R₂ are independently selected from hydrogen, methyl, ethyl, propyl, isopropyl, or C₃-C₅ alicyclic hydrocarbons.
{EU3}. The K-Ras inhibiting compound according to Embodiment {EU1}, wherein one or more of R₃-R₇ are independently selected from the group consisting of hydrogen, —OH, —Cl, —F, —NH₂, —CH₃, —CH₂CH₃, and —OCH₃.
{EU4}. The K-Ras inhibiting compound according to Embodiment {EU3}, wherein two vicinal groups selected from R₃ and R₄, R₄ and R₅, R₅ and R₆, or R₆ and R₇ are each OCH₃, and wherein said two vicinal groups form a fused ring.
{EU5}. The K-Ras inhibiting compound according to Embodiment {EU1}, wherein at least one of z₂, z₃, z₄, z₅, or z₆ is selected from CHX, NX, CL₁X, or CX.
{EU6}. The K-Ras inhibiting compound according to Embodiment {EU5}, wherein X of z₂, z₃, z₄, z₅, and/or z₆ is independently selected at each occurrence from —F, —Cl, —OH, —C(O)—NH₂, —C(O)OR*, —CO₂⁻, —OR*, —NH₂ or —N(R*)₂,
{EU7}. The K-Ras inhibiting compound according to Embodiment {EU5}, wherein L₁ of z₂, z₃, z₄, z₅, and/or z₆ is independently selected at each occurrence is selected from —S—, —O—, —NH—, —(CH₂)₁₋₄, —(CH₂)₀₋₃—NH—C(O)—, or —S(O)₂—.
{EU8}. The K-Ras inhibiting compound according to Embodiment {EU1}, wherein at least one of z₂, z₃, z₄, z₅, or z₆ is selected from NR^{c}, N—R^{L}—R^{B}, N-L₁-R^{B}, C—R^{B}, C—R^{L}—R^{B}, C-L₁-R^{B}, CHR^{B}, CH—R^{L}—R^{B}, or CH-L₁-R^{B}, and L₁ is —C(O)—NH—.
{EU9}. The K-Ras inhibiting compound according to Embodiment {EU8}, wherein R^{L} of z₂, z₃, z₄, z₅, and/or z₆ is —(CH₂)₁₋₃—.
{EU10}. The K-Ras inhibiting compound according to Embodiment {EU8}, wherein R^{B} of z₂, z₃, z₄, z₅, and/or z₆ has the structure

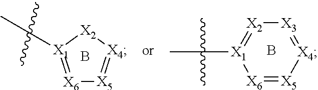

where x₂ is selected from O, S, NH, CX, CH₂, CH, N, or CX, and x₃-x₆ are independently selected from CH, N, and CX.
{EU11}. The K-Ras inhibiting compound according to Embodiment {EU10}, wherein X is selected from —F, —Cl, —Br, —NH₂, —SH, —CN, or —OH.
{EU12}. The K-Ras inhibiting compound according to Embodiment {EU8}, wherein R^{B} of z₂, z₃, z₄, z₅, or z₆ is aromatic and has the structure

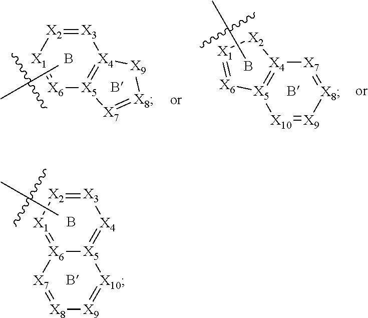

where x₁, x₃-x₈, and x₁₀ are independently selected from N or CH; and x₂ and x₉ are independently selected from N, NH, CH, SO₂, S, or O.
{EU13}. The K-Ras inhibiting compound according to Embodiment {EU12}, wherein at least one of x₁-x₁₀ is N.
{EU14}. The K-Ras inhibiting compound according to Embodiment {EU1} having the structure:

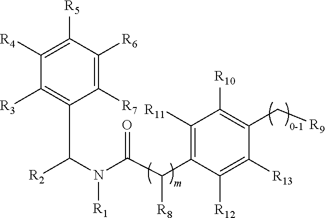

wherein R₉ is —X, or -L₁-X; and R₁₀-R₁₃ are independently H, R or X;
{EU15}. The K-Ras inhibiting compound according to Embodiment {EU14}, wherein R₉ is —NH—C(O)—NH₂.
{EU16}. The K-Ras inhibiting compound according to Embodiment {EU14}, wherein R₉ is —NH₂.
{EU17}. The K-Ras inhibiting compound according to Embodiment {EU1} having the structure:

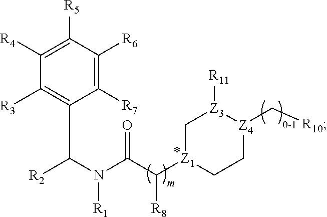

wherein z₁, z₃ and z₄ are independently CH or N;
R₁₀ is hydrogen or —X; and
R₁₁ is hydrogen or R^{B}.
{EU18}. The K-Ras inhibiting compound according to Embodiment {EU17}, wherein z₁ and z₄ are each CH; z₃ is N, R₁₀ is H, and R₁₁ is R^{B}.
{EU19}. The K-Ras inhibiting compound according to Embodiment {EU18}, wherein R^{B} is

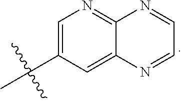 [SMILES: *c1cnc2nccnc2c1]

{EU20}. The K-Ras inhibiting compound according to Embodiment {EU18}, wherein R^{B} is

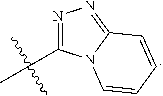 [SMILES: *c1nnc2ccccn12]

{EU21}. The K-Ras inhibiting compound according to Embodiment {EU18}, wherein R^{B} is

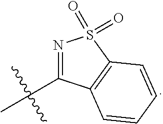 [SMILES: *C1=NS(=O)(=O)c2ccccc21]

### Scaffold A22—Embodiment V (“EV”)

{EV1}. A compound for inhibiting K-Ras having formula (A22), or a pharmaceutically acceptable salt thereof:

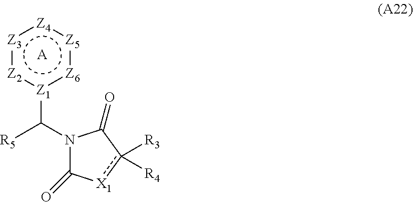

wherein the “dashed” bond may be a single or double bond;
X₁ is selected from NH, NR₁, CH, CR₁, N, S, O, C═O, CHR₁, or CH₂;
ring “A” is a five- or six-membered, optionally aromatic ring; where z₁ is C, CH, or N; and z₂-z₆ are independently selected from N, NH, NR^{N}, O, S, C═O, CH, CX, CR, CH₂, C(X)(X), C(R)(X), or C(R)(R); and in the case where ring “A” is a five-membered ring, z₄ is a bond (i.e., it is absent); and wherein ring “A” may contain 0, 1, 2, or 3 double bonds; and wherein any two vicinal substituents X and/or R and/or R^{N} may together form a 5- or 6-membered ring fused to ring “A”;
R₁ is selected from hydrogen, —R, —X, -L₁-R^{B}, -L₁-X, or -L₁-R;
R₃ and R₄ are independently selected from hydrogen, —R, —X, -L₁-X, -L₁-R, —R^{Q}—X, -L₁-R^{Q}—X, -L₁-R^{Q}—R, or —R^{L}-(L₁)₀₋₂-R^{B}, and wherein R₃ and R₄ may together form a five-, six, or seven-membered spiro ring; and wherein R₃ and R₄ may together form a functional group comprising a double bond selected from ═O, or ═S; and when the bond between X₁ and carbon is double, R₄ is absent;
R₅ is hydrogen, methyl, ethyl, or propyl;
wherein R₁ and R₃ or R₄ may together form a five- or six-membered fused ring; and
R₃ and R₄ may together form a spiro ring; where
X is independently selected at each occurrence from —F, —Cl, —Br, —I, —OH, —OR*, —NH₂, —NHR*, —N(R*)₂, —N(R*)₃⁺, —N(R*)—OH, —N(*O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂⁻, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(═O)₁₋₂—R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂;
R* is independently selected at each occurrence from hydrogen or a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.);
R is selected from hydrogen or C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteroatoms selected from O, S, N, P, F, Cl, Br, I, and combinations thereof;
R^{B} is a C₃₋₁₂ hydrocarbon (alicyclic or aromatic) or heterocycle (e.g., heteroaryl), optionally substituted with one or more (e.g., 1-5) groups R* and/or X and/or with 1-6 heteroatoms selected from O, S, N, P, F, Cl, Br, I, and combinations thereof, where any vicinal groups can form a 5- or 6-membered fused ring;
R^{Q} is a cyclic group having the structure:

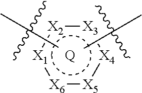

wherein ring “Q” is a five- or six-membered optionally aromatic ring, where x₁-x₆ are independently selected from N, NH, NR^{N}, O, S, C═O, CH, CX, CR, CH₂, C(X)(X), C(R)(X), and C(R)(R); with the proviso that the points of attachment are not O, S, or C═O, and in the case where ring “Q” is a five-membered ring, x₄ is a bond (i.e., it is absent); and wherein any two vicinal substituents X and/or R and/or R^{N} may together form a 5- or 6-membered ring fused to ring “Q”;
R^{L} at each occurrence is a C₁₋₈ linear or branched bivalent hydrocarbon radical; optionally substituted with one or more (e.g., 1-5) groups X and/or with 1-6 heteroatoms selected from O, S, N, P, F, Cl, Br, I, and combinations thereof; and
L₁ is selected independently at each occurrence from —O—, —S—, —NH—, —N(R^{N})—, —(CH₂)₁₋₄—, —C═C—, —C═C—(CH₂)₁₋₃—, —C(O)—, —C(O)—(CH₂)₁₋₃—, —NH—C(O)—, —C(O)—NH—, —C(O)—O—(CH₂)₁₋₃—, —C(O)—N(R^{N})—, —N(R^{N})—C(O)—, —C(O)—N(R^{N})—(CH₂)₁₋₃—, —(CH₂)₁₋₃—C(O)—N(R^{N})—, —(CH₂)₀₋₃—NH—C(O)—O—, —(CH₂)₀₋₃—NH—(CH₂)₁₋₃—, —CH₂—NH—S(O)₁₋₂—, —N(R^{N})—S(O)₁₋₂—, —O—(CH₂)₁₋₃—, —S(O)₁₋₂—, —S—(CH₂)₁₋₃—, —N(R^{N})—(CH₂)₁₋₃—, and —(OCH₂CH₂)₁₋₃—; where
R^{N} is independently selected at each occurrence from hydrogen, methyl, ethyl, or propyl.
{EV2}. The K-Ras inhibiting compound according to Embodiment {EV1} having the structure:

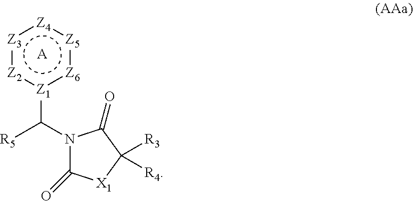

{EV3}. The K-Ras inhibiting compound according to Embodiment {EV1} having the structure:

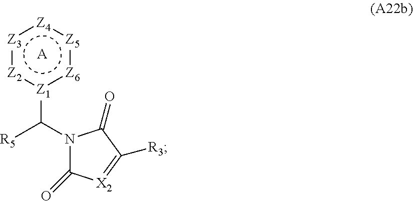

wherein X₂ is selected from CH, CR₁, or N.
{EV4}. The K-Ras inhibiting compound according to Embodiment {EV1}, wherein ring “A” is a six-membered aromatic ring and z₁-z₆ are each independently selected from CH, CX, N, and CR.
{EV5}. The K-Ras inhibiting compound according to Embodiment {EV4}, wherein at least one of z₁-z₆ is CX, and X is selected from the group consisting of —F, —Cl, —Br, —OR^{N}, and —R^{N}.
{EV6}. The K-Ras inhibiting compound according to Embodiment {EV1}, wherein ring “A” is a five-membered aromatic ring and z₁-z₆ are each independently selected from CH, CX, N, O, S, and CR.
{EV7}. The K-Ras inhibiting compound according to Embodiment {EV1}, wherein R₄ is hydrogen or R, and R₃ is selected from —R^{L}-(L₁)₀₋₂-R^{B}, or —R^{L}-(L₁)₀₋₂-X.
{EV8}. The K-Ras inhibiting compound according to Embodiment {EV7}, wherein L₁ of R₄ is selected from —NH—, —S(O₂)—, —NH—C(O)—, —NH—C(O)—(CH₂)—, —S—, or —C(O)—.
{EV9}. The K-Ras inhibiting compound according to Embodiment {EV7}, wherein R₃ and R₄ together form a spiro C₅-C₇ alicyclic ring optionally substituted with one or more heteroatoms selected from N, S, and O.
{EV10}. The K-Ras inhibiting compound according to Embodiment {EV7}, wherein R₁ or R₂ is R, where R₃ or R₄ and R₁ or R₂ together form a fused C₅-C₇ alicyclic ring optionally substituted with one or more heteroatoms selected from N, S, and O.
{EV11}. The K-Ras inhibiting compound according to Embodiment {EV7}, wherein R^{B} is a five-membered aromatic heterocycle.
{EV12}. The K-Ras inhibiting compound according to Embodiment {EV11}, wherein said five-membered aromatic heterocycle is selected from the group consisting of pyrrolyl, furanyl, imidazolyl, pyrazolyl, oxazolyl, thiazolyl, and thiophenyl.
{EV13}. The K-Ras inhibiting compound according to Embodiment {EV11}, wherein two vicinal groups of said five-membered aromatic heterocycle are substituted with R groups such that said two vicinal groups together form a fused ring to said five-membered aromatic heterocycle.
{EV14}. The K-Ras inhibiting compound according to Embodiment {EV13}, wherein said fused ring is a six-membered aromatic ring.
{EV15}. The K-Ras inhibiting compound according to Embodiment {EV7}, wherein R^{B} is a six-membered aromatic ring.
{EV16}. The K-Ras inhibiting compound according to Embodiment {EV15}, wherein said aromatic ring is substituted with or more groups —X and/or heteroatoms independently selected from —OCH₃, —CH₃, —F, —Cl, N, O, or S.
{EV17}. The K-Ras inhibiting compound according to Embodiment {EV16}, wherein two vicinal groups are each substituted with —OCH₃, and said two vicinal groups form a fused ring with said aromatic ring.
{EV18}. The K-Ras inhibiting compound according to Embodiment {EV1}, wherein R₃ and/or R₄ is selected from the group consisting of —R^{Q}—X, and -L₁-R^{Q}—X.
{EV19}. The K-Ras inhibiting compound according to Embodiment {EV18}, wherein R^{Q} is a six-membered ring optionally substituted with one or more N.
{EV20}. The K-Ras inhibiting compound according to Embodiment {EV18}, wherein L₁ is —NH—.
{EV21}. The K-Ras inhibiting compound according to Embodiment {EV18}, wherein —X is selected from —CO₂⁻, —C(O)—NH₂, or —C(O)—N(R*)₂, —F, —Cl, —NH₂, —OH, or —OR*.
{EV22}. The K-Ras inhibiting compound according to Embodiment {EV1} having the structure:

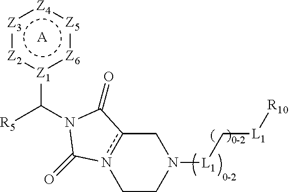

wherein R₁₀ is selected from R, R^{B} or X.
{EV23}. The K-Ras inhibiting compound according to Embodiment {EV22} having the structure

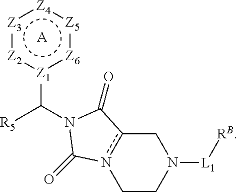

{EV24}. The K-Ras inhibiting compound according to Embodiment {EV23} wherein L₁ is SO₂.
{EV25}. The K-Ras inhibiting compound according to Embodiment {EV23} wherein R^{B} is thiophenyl.
{EV26}. The K-Ras inhibiting compound according to Embodiment {EV23} wherein R^{B} is 2-thiophenyl.
{EV27}. The K-Ras inhibiting compound according to Embodiment {EV22} having the structure:

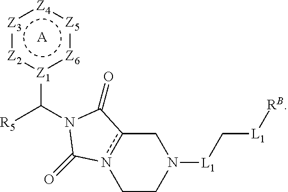

{EV28}. The K-Ras inhibiting compound according to Embodiment {EV27}, wherein L₁ is independently selected at each occurnce from —C(O)— and —S—.
{EV29}. The K-Ras inhibiting compound according to Embodiment {EV22} having the structure:

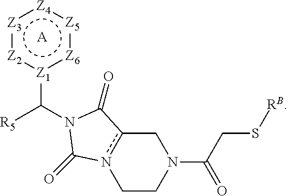

{EV30}. The K-Ras inhibiting compound according to Embodiment {EV27}, wherein R^{B} is pyridinyl.
{EV31}. The K-Ras inhibiting compound according to Embodiment {EV27}, wherein R^{B} is 4-pyridinyl.
{EV32}. The K-Ras inhibiting compound according to Embodiment {EV1} having the structure:

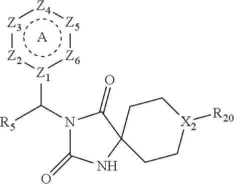

wherein X₂ is selected from N, CH, CHCH₂, CH₂CH, or CH₂CH₂ such that when X₂ is CH₂CH₂, R₂₀ is absent; and
R₂₀ is selected from hydrogen, —R, —R^{B}, —X, —R^{L}—R^{B}, or -L₁-R^{B}
{EV33}. The K-Ras inhibiting compound according to Embodiment {EV32} having the structure:

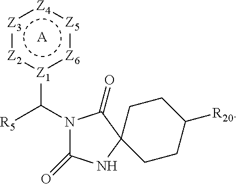

{EV34}. The K-Ras inhibiting compound according to Embodiment {EV33} wherein R₂₀ is hydrogen, methyl, ethyl, propyl, or isopropyl.
{EV35}. The K-Ras inhibiting compound according to Embodiment {EV32} having the structure:

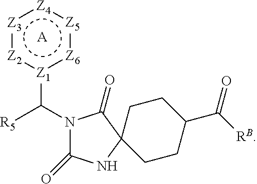

{EV36}. The K-Ras inhibiting compound according to Embodiment {EV32} having the structure:

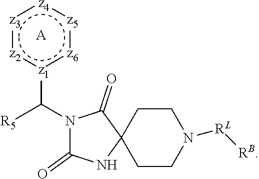

{EV37}. The K-Ras inhibiting compound according to Embodiment {EV32} having the structure:

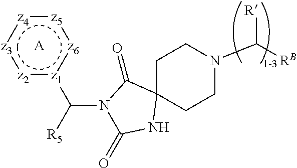

wherein R′ is independently selected at each occurrence from hydrogen or methyl.
{EV38}. The K-Ras inhibiting compound according to Embodiment {EV36}, wherein R^{B} is a five membered optionally aromatic ring substituted with N, O, or S.
{EV39}. The K-Ras inhibiting compound according to Embodiment {EV36}, wherein R^{B} is

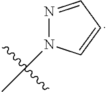 [SMILES: *n1cccn1]

{EV40}. The K-Ras inhibiting compound according to Embodiment {EV1} having the structure:

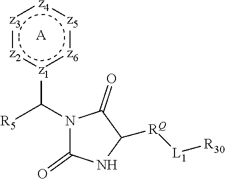

wherein R₃₀ is selected from hydrogen, —X, R or R^{B}.
{EV41}. The K-Ras inhibiting compound according to Embodiment {EV40}, wherein R^{Q} is selected from

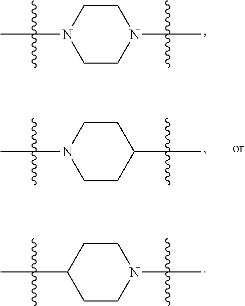 [SMILES: *N1CCN(*)CC1 ; *C1CCN(*)CC1 ; *C1CCN(*)CC1]

{EV42}. The K-Ras inhibiting compound according to Embodiment {EV40}, wherein L₁ is —C(O)—.
{EV43}. The K-Ras inhibiting compound according to Embodiment {EV40}, wherein R₃₀ is —X and —X is NH₂ or N(R*)(R*).

### Scaffold A23—Embodiment W (“EW”)

{EW1}. A compound for inhibiting K-Ras having formula (A23), or a pharmaceutically acceptable salt thereof:

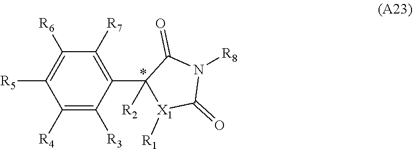

wherein, X₁ is CH or N;
R₁ and R₂ are independently selected from hydrogen, —R*, —R, and —X;
R₃-R₇ are independently selected from hydrogen, —R*, —R, and —X; and R₂ and R₃ may together form a 5- or 6-membered fused and spiro ring optionally substituted with N, O, or S or a group X;
R₈ is hydrogen, —R, —X, —R^{B}, —R^{Q}—X, —R^{Q}—R, -L₁-X, -L₁-R^{B}, or -L₁-R^{Q}—X, -L₁-R^{Q}—R, -L₁-R^{Q}—R^{B}, —(CH₂)₁₋₂-L₁-R^{Q}—X, —(CH₂)₁₋₂-L₁-R^{Q}—R, or —(CH₂)₁₋₂₋L₁-R^{Q}—R^{B}; where
X is independently selected at each occurrence from —F, —Cl, —Br, —I, —OH, —OR*, —NH₂, —NHR*, —N(R*)₂, —N(R*)₃⁺, —N(R*)—OH, —N(*O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂⁻, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(═O)₁₋₂—R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂;
L₁ is selected independently at each occurrence from —O—, —S—, —NH—, —N(R^{N})—, —(CH₂)₁₋₄—, —C═C—, —C═C—(CH₂)₁₋₃—, —C(O)—, —(CH₂)₁₋₃—C(O)—, —C(O)—(CH₂)₁₋₃—, —C(O)—O—(CH₂)₁₋₃—, —C(O)—N(R^{N})—, —N(R^{N})—C(O)—, —C(O)—N(R^{N})—(CH₂)₁₋₃—, —(CH₂)₁₋₃—C(O)—N(R^{N})—, —(CH₂)₀₋₃—NH—C(O)—O—, —NH—S(O)₁₋₂—, —N(R^{N})—S(O)₁₋₂₋, —O—(CH₂)₁₋₃—, —S(O)₁₋₂—, —S—(CH₂)₁₋₃—, —NH—(CH₂)₁₋₃—, —N(R^{N})—(CH₂)₁₋₃—, Or —(OCH₂CH₂)₁₋₃—;
R^{Q} is a cyclic group having the structure:

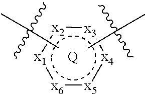

wherein ring “Q” is a five- or six-membered optionally aromatic ring, x₁-x₆ x₂, x₃, X₅, and x₆, are independently selected from N, NH, NR^{N}, O, S, C═O, CH, CX, CR, CH₂, C(X)(X), C(R)(X), and C(R)(R); and each x₁-x₆ comprising a linking bond is independently selected from C, CH, CR, or N; and in the case where ring “Q” is a five-membered ring, x₃ or x₅ is a bond (i.e., it is absent); and wherein any two vicinal substituents X and/or R and/or R^{N} may together form a 5- or 6-membered ring fused to ring “Q”;
R* is independently selected at each occurrence from hydrogen and a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.);
R^{B} is a C₃₋₆ cyclic hydrocarbon (alicyclic or aromatic) or heterocycle (e.g., heteroaryl), optionally substituted with one or more (e.g., 1-5) groups R and/or X and/or with 1-6 heteroatoms selected from O, S, N, P, F, Cl, Br, I, and combinations thereof, where any vicinal groups can form a 5- or 6-membered fused ring;
R is selected from hydrogen or C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteroatoms selected from O, S, N, P, F, Cl, Br, I, and combinations thereof; and
R^{N} is hydrogen, methyl, ethyl, or propyl.
{EW2}. The compound for inhibiting K-Ras of Embodiment {EW1}, wherein at least one of R₁-R₇ is methyl, ethyl, or propyl.
{EW3}. The compound for inhibiting K-Ras of Embodiment {EW2}, wherein R₂ and R₃. together form a five- or six-membered fused spiro ring.
{EW4}. The compound for inhibiting K-Ras of Embodiment {EW1}, X is N and R₁ is H.
{EW5}. The compound for inhibiting K-Ras of Embodiment {EW1}, wherein R₈ is —X, -L₁-X, or -L₁-R^{Q}—X, where R^{Q} is selected from cyclic groups having the structure

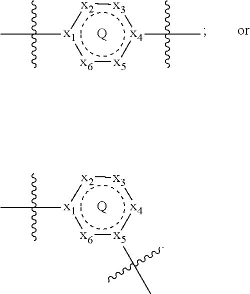

{EW6}. The compound for inhibiting K-Ras of Embodiment {EW5}, wherein ring “Q” is alicyclic and x₁ is N.
{EW7}. The compound for inhibiting K-Ras of Embodiment {EW5}, wherein ring “Q” is aromatic.
{EW8}. The compound for inhibiting K-Ras of Embodiment {EW5}, wherein —X of R₈ is selected from —Cl, —F, —OH, —NH, —COOR*, —C(O)—NH₂, —CF₃, or —CCl₃.
{EW9}. The compound for inhibiting K-Ras of Embodiment {EW5}, wherein -L₁- of R₈ is selected from —(CH₂)₁₋₄—, —(CH₂)₁₋₃—C(O)—, —C(O)—(CH₂)₁₋₃—, or —C(O)—O—(CH₂)₁₋₃—.
{EW10}. The compound for inhibiting K-Ras of Embodiment {EW5}, wherein R₈ is —R^{B} or -L₁-R^{B}.
{EW11}. The compound for inhibiting K-Ras of Embodiment {EW10}, wherein —R^{B} of R₈ is a six-membered aromatic optionally substituted with one or more groups selected from —Cl, —F, —OH, —NH, —R*, —COOR*, —C(O)—NH₂, —CF₃, or —CCl₃.
{EW12}. The compound for inhibiting K-Ras of Embodiment {EW10}, wherein —R^{B} of R₈ is a five-membered ring.
{EW13}. The compound for inhibiting K-Ras of Embodiment {EW12}, wherein —R^{B} of R₈ is selected from pyrrolyl, furanyl, thiophenyl, imidazolyl, pyrazolyl, oxazolyl, isoxazolyl, thiazolyl, or isothiazolyl, and wherein —R^{B} is optionally substituted with one or more groups —R*.
{EW14}. The compound for inhibiting K-Ras of Embodiment {EW10}, wherein -L₁- of R₈ is selected from from —(CH₂)₁₋₄—, —(CH₂)₁₋₃—C(O)—, —C(O)—(CH₂)₁₋₃—, or —C(O)—O—(CH₂)₁₋₃—.
{EW15}. The compound for inhibiting K-Ras of Embodiment {EW1} having the structure:

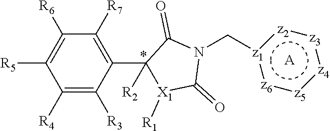

wheein ring “A” is a five- or six-membered optionally aromatic ring; z₁₀ is C, CH, or N; and z₁₁-z₁₅ are independently selected from N, NH, NR^{N}, O, S, C═O, CH, CX, CR, CH₂, C(X)(X), C(R)(X), or C(R)(R); and wherein ring “A” may contain 0, 1, 2, or 3 double bonds; and in the case where ring “A” is a five-membered ring, z₁₃ is a bond (i.e., it is absent); and wherein any two vicinal substituents X and/or R and/or R^{N} may together form a 5- or 6-membered ring fused to ring “A”.
{EW16}. The compound for inhibiting K-Ras of Embodiment {EW15}, wherein ring “A” is substituted phenyl.
{EW17}. The compound for inhibiting K-Ras of Embodiment {EW15}, wherein ring “A” is trifluoromethylphenyl.
{EW18}. The compound for inhibiting K-Ras of Embodiment {EW15}, wherein ring “A” is five-membered.
{EW19}. The compound for inhibiting K-Ras of Embodiment {EW15} having the structure:

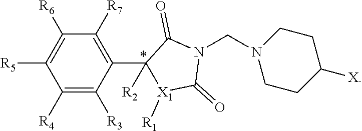

{EW20}. The compound for inhibiting K-Ras of Embodiment {EW15} having the structure:

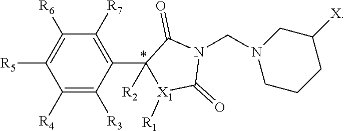

{EW21}. The compound for inhibiting K-Ras of Embodiment {EW15} having the structure:

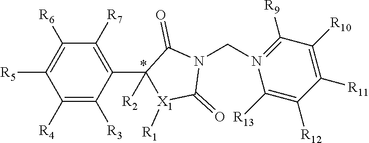

wherein R₉-R₁₃ are independently selected from hydrogen, —R, or —X.
{EW22}. The compound for inhibiting K-Ras of Embodiment {EW15} having the structure:

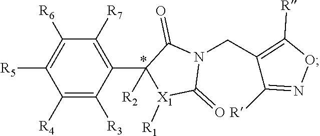

wherein R′ and R″ are independently selected from hydrogen, methyl, ethyl, propyl, and isopropyl.

### Scaffold A24—Embodiment X (“EX”)

{EX1}. A compound for inhibiting K-Ras having formula (A24), or a pharmaceutically acceptable salt thereof:

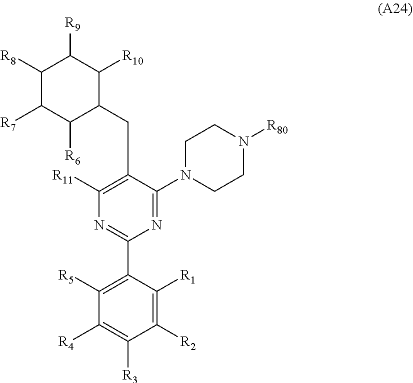

wherein, R₁-R₁₀ are independently selected from hydrogen, —R*, —R, and —X;
R₁₁ is hydrogen, methyl, or ethyl;
R₈₀ is hydrogen, —X, —R, —R^{B}, —R^{B}—X, -L₁-X, -L₁-R, -L₁-R^{B}, -(L₁)₀₋₁-R^{Q}-(L₁)₀₋₁-R, -(L₁)₀₋₁-R^{Q}-(L₁)₀₋₁-X, or -(L₁)₀₋₁-R^{B};
X is independently selected at each occurrence from —F, —Cl, —Br, —I, —OH, —OR*, —NH₂, —NHR*, —N(R*)₂, —N(R*)₃⁺, —N(R*)—OH, —N(*O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂⁻, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(═O)₁₋₂—R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂;
R* is independently selected at each occurrence from hydrogen and a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.);
L₁ is selected from —O—, —S—, —S(O)₂—, —S(O)₂ NR^{N}—, —NR^{N}—S(O)₂—, —NR^{N}—S(O)—, —S(O)—N(R^{N})—, —NH—, —N(R^{N})—, —(CH₂)₁₋₄—, —C═C—, —C═C—(CH₂)₁₋₃—, —C(O)—, —C(O)—(CH₂)₁₋₃—, —C(O)—O—(CH₂)₁₋₃—, —C(O)—N(R^{N})—, —N(R^{N})—C(O)—, —C(O)—N(R^{N})—(CH₂)₁₋₃—, —(CH₂)₁₋₃—C(O)—N(R^{N})—, —(CH₂)₀₋₃—NH—C(O)—O—, —O—(CH₂)₁₋₃—, —S(O)₁₋₂—, —S—(CH₂)₁₋₃—, —NH—(CH₂)₁₋₃—, —N(R^{N})—(CH₂)₁₋₃—, —(CH₂CH₂O)₁₋₃—; and —(OCH₂CH₂)₁₋₃—;
R^{B} is a C₃₋₆ cyclic hydrocarbon (alicyclic or aromatic) or heterocycle (e.g., heteroaryl), optionally substituted with one or more (e.g., 1-5) groups R and/or X and/or with 1-6 heteroatoms selected from O, S, N, P, F, Cl, Br, I, and combinations thereof, where any vicinal groups can form a 5- or 6-membered fused ring;
R^{Q} is a cyclic group having the structure:

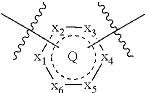

wherein ring “Q” is a five- or six-membered optionally aromatic ring, x₁-x₆ x₂, x₃, x₅, and x₆, are independently selected from N, NH, NR^{N}, O, S, C═O, CH, CX, CR, CH₂, C(X)(X), C(R)(X), and C(R)(R); and each x₁-x₆ comprising a linking bond is independently selected from C, CH, CR, or N; and in the case where ring “Q” is a five-membered ring, x₃ or x₅ is a bond (i.e., it is absent); and wherein any two vicinal substituents X and/or R and/or R^{N} may together form a 5- or 6-membered ring fused to ring “Q”;
R is selected from hydrogen or C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteroatoms selected from O, S, N, P, F, Cl, Br, I, and combinations thereof; and
R^{N} is hydrogen, methyl, ethyl, or propyl.
{EX2}. The compound for inhibiting K-Ras of Embodiment {EX1}, wherein the compound has the structure:

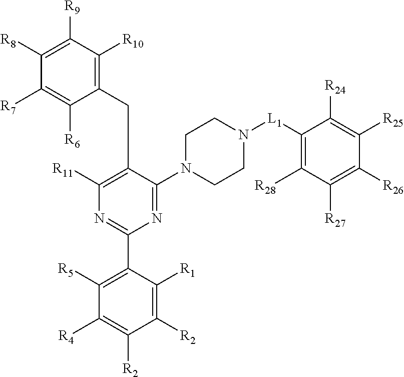

where R₂₄-R₂₈ are independently selected from hydrogen, R*, R, X, and R^{B}, and where any two adjacent groups R₂₄-R₂₈ may together form a five- or six-membered fused ring.
{EX3}. The compound for inhibiting K-Ras of Embodiment {EX2}, wherein R₆-R₁₀ are hydrogen.
{EX4}. The compound for inhibiting K-Ras of Embodiment {EX2}, wherein R₁₁ is methyl.
{EX5}. The compound for inhibiting K-Ras of Embodiment {EX2}, wherein one of R₁-R₅ is —CF₃.
{EX6}. The compound for inhibiting K-Ras of Embodiment {EX2}, wherein one of R₁-R₅ is —CF₃.
{EX7}. The compound for inhibiting K-Ras of Embodiment {EX2}, wherein L₁ is —S(O)₂—.
{EX8}. The compound for inhibiting K-Ras of Embodiment {EX2}, wherein R₂₄ and R₂₅ (or R₂₇ and R₂₈) together form a divalent radical —CH═CH—CH═CH—, such that a six-membered aromatic ring fused to the ring to which they are attached is formed.
{EX9}. The compound for inhibiting K-Ras of Embodiment {EX2}, wherein one or more of R₂₄-R₂₈ are independently selected from hydrogen, —OH, —SH, —Cl, —F, —Br, —I, —NH₂, —NR^{N}₂, —CH₃, —CH₂CH₃, —OCH₃, —OCH₂CH₃, —C(O)OR*, —C(O)NR^{N}₂, —NR^{N}—C(O)R*, —CF₃, —NO₂, and —CN.
{EX10}. The compound for inhibiting K-Ras of Embodiment {EX2}, wherein L₁ is —S(O)₂—, R₁₁ is methyl, R₆-R₁₀ are hydrogen; and one of R₁-R₅ is selected from hydrogen, —OH, —SH, —Cl, —F, —Br, —I, —NH₂, NR^{N}₂, —CH₃, —CH₂CH₃, —OCH₃, —OCH₂CH₃, —C(O)OR*, —C(O)NR^{N}₂, —NR^{N}—C(O)R*, —CF₃, —NO₂, and —CN.

### Scaffold A25—Embodiment Y (“EY”)

{EY1}. A compound for inhibiting K-Ras having formula (A25), or a pharmaceutically acceptable salt thereof:

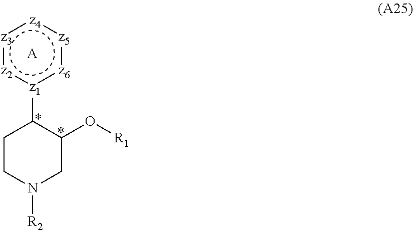

wherein ring “A” is a five- or six-membered, optionally aromatic, ring; where z₁ is C, CH, or N; and z₂-z₆ are independently selected from N, NH, NR^{N}, O, S, C═O, CH, CX, CR, CH₂, C(X)(X), C(R)(X), or C(R)(R); and wherein ring “A” may contain 0, 1, 2, or 3 double bonds; and in the case where ring “A” is a five-membered ring, z₄ is a bond (i.e., it is absent); and wherein any two vicinal substituents X and/or R and/or R^{N} may together form a 5- or 6-membered ring fused to ring “A”;
R₁ is hydrogen, or lower alkyl;
R₂ is selected from —R, —X, —R^{B}, -L₁-X, -L₁-R, -L₁-R^{B}, -L₁-L₁-X, -L₁-L₁-R, -L₁-L₁-X, -L₁-L₁-R, -L₁-L₁-R^{B}, -L₁-R^{Q}—R, -L₁-R^{Q}—X, -L₁-R^{Q}—R, -L₁-R^{Q}—R^{B}, -L₁-R^{L}—X, -L₁-R^{L}—R, -L₁-R^{L}-L₁-R^{B}, or -L₁-R^{L}—R^{B}; where
X is independently selected at each occurrence from —F, —Cl, —Br, —I, —OH, —OR*, —NH₂, —NHR*, —N(R*)₂, —N(R*)₃⁺, —N(R*)—OH, —N(*O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂—, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(═O)₁₋₂—R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂;
R^{B} is a monocyclic or fused bicyclic group having the structure:

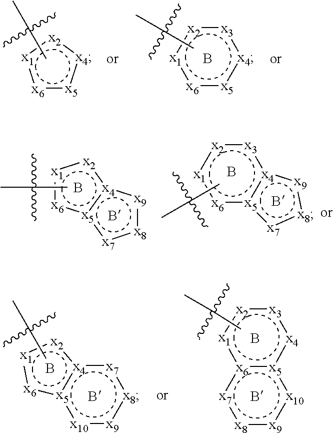

wherein ring B and B′ are independently five- or six-membered, optionally aromatic rings; x₁-x₁₀ are independently selected from N, NH, NR^{N}, O, S, S(═O)₂, C═O, C, CH, CX, CR, CH₂, C(X)(X), C(R)(X), and C(R)(R); and in the case where ring “B” is a five-membered ring, x₃ is a bond (i.e., it is absent); and wherein any two vicinal substituents X and/or R and/or R^{N} may together form an optionally substituted 5- or 6-membered ring fused to ring “B” and/or “B′”;
L₁ is selected independently at each occurrence from —O—, —S—, —NH—, —N(R^{N})—, —(CH₂)₁₋₄—, —C═C—, —C═C—(CH₂)₁₋₃—, —C(O)—, —(CH₂)₁₋₃—C(O)—, —C(O)—(CH₂)₁₋₃—, —C(O)—O—(CH₂)₁₋₃—, —C(O)—N(R^{N})—, —N(R^{N})—C(O)—, —C(O)—N(R^{N})—(CH₂)₁₋₃—, —(CH₂)₁₋₃—C(O)—N(R^{N})—, —(CH₂)₀₋₃—NH—C(O)—, —(CH₂)₀₋₃—NH—C(O)—O—, —NH—S(O)₁₋₂—, —N(R^{N})—S(O)₁₋₂—, —O—(CH₂)₁₋₃—, —S(O)₁₋₂—, —S—(CH₂)₁₋₃—, —NH—(CH₂)₁₋₃—, —N(R^{N})—(CH₂)₁₋₃₋, and —(OCH₂CH₂)₁₋₃—;
R^{Q} is a cyclic group having the structure:

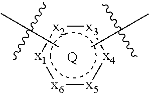

wherein ring “Q” is a five- or six-membered optionally aromatic ring x₁-x₆ are independently selected from N, NH, NR^{N}, O, S, C═O, CH, CX, CR, CH₂, C(X)(X), C(R)(X), and C(R)(R); each x₁-x₆ comprising a linking bond is independently selected from C, CH, CR, or N; and in the case where ring “Q” is a five-membered ring, x₃ or x₅ is a bond (i.e., it is absent); and wherein any two vicinal substituents X and/or R and/or R^{N} may together form a 5- or 6-membered ring fused to ring “Q”;
R^{L} at each occurrence is a C₁₋₆ linear, branched, or cyclic bivalent hydrocarbon radical; optionally substituted with one or more (e.g., 1-5) groups X and/or with 1-6 heteroatoms selected from O, S, N, P, F, Cl, Br, I, and combinations thereof;
R* is independently selected at each occurrence from hydrogen and a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.);
R is selected from hydrogen or C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteroatoms selected from O, S, N, P, F, Cl, Br, I, and combinations thereof; and
R^{N} is hydrogen, methyl, ethyl, or propyl.
{EY2}. The K-Ras inhibiting compound according to Embodiment {EY1}, wherein ring “A” is a five-membered aromatic ring selected from pyrrolyl, furanyl, thiophenyl, imidazolyl, pyrazolyl, oxazolyl, isoxazolyl, thiazolyl, or isothiazolyl, and wherein z₂-z₆ are each optionally substituted with a group —R*.
{EY3}. The K-Ras inhibiting compound according to Embodiment {EY1}, wherein ring “A” is a six-membered aromatic ring.
{EY4}. The K-Ras inhibiting compound according to Embodiment {EY3}, wherein z₂-z₆ are each optionally substituted with a group independently selected from —OH, —CH₃, —CH₂—CH₃, —CH═CH₂, —OCH₃, —O—CH₂—CH₃, —F, or —Cl.
{EY5}. The K-Ras inhibiting compound according to Embodiment {EY3}, wherein at least one of z₃-z₅ is substituted with a group independently selected from —OH, —CH₃, —CH₂—CH₃, —CH═CH₂, —OCH₃, —O—CH₂—CH₃, —F, or —Cl.
{EY6}. The K-Ras inhibiting compound according to Embodiment {EY3}, wherein at z₄ and z₃ or z₄ and z₅ are substituted with a group independently selected from —CH₃, —CH₂—CH₃, —CH═CH₂, —OCH₃, or —O—CH₂—CH₃.
{EY7}. The K-Ras inhibiting compound according to Embodiment {EY6}, wherein z₄ and z₃ or z₄ and z₅ together form a five- or six-membered fused ring.
{EY8}. The K-Ras inhibiting compound according to Embodiment {EY1}, wherein R₂ is selected from -L₁-X, -L₁-R, -L₁-R^{B}, -L₁-L₁-X, -L₁-L₁-R, -L₁-L₁-X, -L₁-L₁-R, -L₁-L₁-R^{B}, -L₁-R^{Q}—R, -L₁-R^{Q}—X, -L₁-R^{Q}—R, -L₁-R^{Q}—R^{B}, -L₁-R^{L}—X, -L₁-R^{L}—R, or -L₁-R^{L}—R^{B}, where L₁ is independently selected from —C(O)—, —(CH₂)₁₋₃—C(O)—, —C(O)—(CH₂)₁₋₃—, —S—, —NH—, or —NH—C(O)—.
{EY9}. The K-Ras inhibiting compound according to Embodiment {EY8} having the structure:

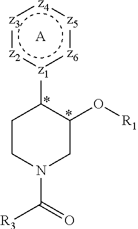

wherein R₃ is selected from —X, —R, —R^{B}, -L₁-X, -L₁-R-L₁-R^{B}, —R^{Q}—R, —R^{Q}—X, —R^{Q}—R, —R^{Q-}R^{B}, —R^{L}—X, —R^{L}—R, or —R^{L}—R^{B}
{EY10}. The K-Ras inhibiting compound according to Embodiment {EY9}, wherein R₃ is —R^{B}, —R^{Q}—R^{B}, or —R^{L}-R^{B}
{EY11}. The K-Ras inhibiting compound according to Embodiment {EY10}, wherein R^{B} is selected from

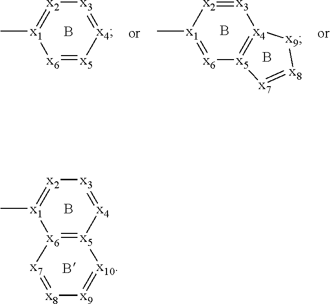

{EY12}. The K-Ras inhibiting compound according to Embodiment {EY11}, wherein x₉ is NH and/or x₇ and/or x₄ and/or x₃ is N.
{EY13}. The K-Ras inhibiting compound according to Embodiment {EY11}, wherein x₂ and x₅ are both N.
{EY14}. The K-Ras inhibiting compound according to Embodiment {EY11}, wherein x₅ or x₃ O.
{EY15}. The K-Ras inhibiting compound according to Embodiment {EY11}, wherein at least one of x₁-x₁₀ is CX, C═O, or CR, where X is selected from —NH₂, —OH, —Cl, or —F; and R is selected from methyl, ethyl or propyl.
{EY16}. The K-Ras inhibiting compound according to Embodiment {EY11}, wherein two of x₁-x₁₀ is CX, C═O, or CR, where X is selected from —NH₂, —OH, —Cl, —CN, or —F; and R is selected from methyl, ethyl or propyl.
{EY17}. The K-Ras inhibiting compound according to Embodiment {EY11}, wherein three of x₁-x₁₀ is CX, C═O, or CR, where X is selected from —NH₂, —OH, —Cl, —CN, or —F; and R is selected from methyl, ethyl or propyl.
{EY18}. The K-Ras inhibiting compound according to Embodiment {EY10}, wherein R^{B} is selected from

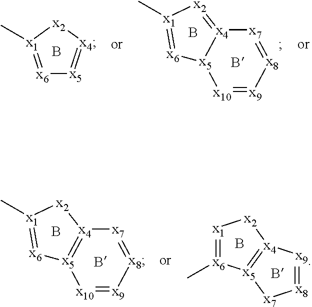

{EY19}. The K-Ras inhibiting compound according to Embodiment {EY18}, wherein one of x₁-x₁₀ is N, NH, NR, or NX, where X is selected from —NH₂, —OH, —Cl, —CN, or —F; and R is selected from methyl, ethyl or propyl.
{EY20}. The K-Ras inhibiting compound according to Embodiment {EY18}, wherein two of x₁-x₁₀ are independently N, NH, NR, or NX, where X is selected from —NH₂, —OH, —Cl, —CN, or —F; and R is selected from methyl, ethyl or propyl.
{EY21}. The K-Ras inhibiting compound according to Embodiment {EY18}, wherein three of x₁-x₁₀ are independently N, NH, NR, or NX, where X is selected from —NH₂, —OH, —Cl, —CN, or —F; and R is selected from methyl, ethyl or propyl.
{EY22}. The K-Ras inhibiting compound according to Embodiment {EY18}, wherein four of x₁-x₁₀ are independently N, NH, NR, or NX, where X is selected from —NH₂, —OH, —Cl, —CN, or —F; and R is selected from methyl, ethyl or propyl.
{EY23}. The K-Ras inhibiting compound according to Embodiment {EY18}, wherein at least one of x₁-x₁₀ is CX, C═O, or CR, where X is selected from —NH₂, —OH, —Cl, or —F; and R is selected from methyl, ethyl or propyl.
{EY24}. The K-Ras inhibiting compound according to Embodiment {EY18}, wherein two of x₁-x₁₀ is CX, C═O, or CR, where X is selected from —NH₂, —OH, —Cl, —CN, or —F; and R is selected from methyl, ethyl or propyl.
{EY25}. The K-Ras inhibiting compound according to Embodiment {EY10}, wherein R^{B} is selected from

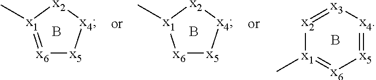

{EY26}. The K-Ras inhibiting compound according to Embodiment {EY25}, wherein one of x₂ or x₄ is C═O.
{EY27}. The K-Ras inhibiting compound according to Embodiment {EY25}, wherein at least one of x₁-x₆ is selected from N, NH, NR, or O, where R is methyl, ethyl, or propyl.
{EY28}. The K-Ras inhibiting compound according to Embodiment {EY25}, wherein at least one of x₁-x₆ is CX, or CR, where X is selected from —NH₂, —OH, —Cl, —CN, or —F; and R is selected from methyl, ethyl, or propyl.
{EY29}. The K-Ras inhibiting compound according to Embodiment {EY10}, wherein R^{Q} is a six-membered aromatic optionally substituted with N or S.
{EY30}. The K-Ras inhibiting compound according to Embodiment {EY10}, wherein R^{L} is a linear C₂-C₆ hydrocarbon optionally substituted with N or S.
{EY31}. The K-Ras inhibiting compound according to Embodiment {EY9}, wherein R₃ is selected from —R^{L}—X or -L₁-X.
{EY32}. The K-Ras inhibiting compound according to Embodiment {EY31}, wherein R^{L} is a cyclic C₃-C₅ hydrocarbon.
{EY33}. The K-Ras inhibiting compound according to Embodiment {EY31}, wherein L₁ is —(CH₂)₀₋₃—NH—CO—.
{EY34}. The K-Ras inhibiting compound according to Embodiment {EY31}, wherein X is selected from —NH₂, —OH, —Cl, —CN, or —F.
{EY35}. The K-Ras inhibiting compound according to Embodiment {EY1} having the structure:

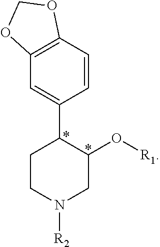

{EY36}. The K-Ras inhibiting compound according to Embodiment {EY1} having the structure:

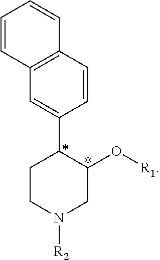

{EY37}. The K-Ras inhibiting compound according to Embodiment {EY1}, wherein R₁ is hydrogen.

### Scaffold A26—Embodiment Z (“EZ”)

{EZ1}. A compound for inhibiting K-Ras having formula (A26), or a pharmaceutically acceptable salt thereof:

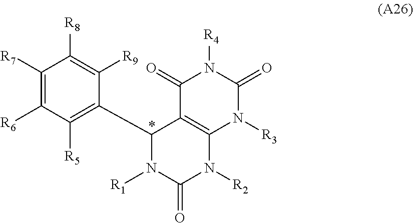

wherein R₁-R₄ are independently selected from hydrogen or R;
R₅-R₉ are independently selected from hydrogen, R, or X; where
X is independently selected at each occurrence from —F, —Cl, —Br, —I, —OH, —OR*, —NH₂, —NHR*, —N(R*)₂, —N(R*)₃⁺, —N(R*)—OH, —N(*O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂⁻, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(═O)₁₋₂—R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂;
R* is independently selected at each occurrence from hydrogen and a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.);
R is selected from hydrogen or C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteroatoms selected from O, S, N, P, F, Cl, Br, I, and combinations thereof; and
{EZ2}. The K-Ras inhibiting compound according to Embodiment {EZ1}, wherein R₁-R₄ are independently hydrogen or methyl.
{EZ3}. The K-Ras inhibiting compound according to Embodiment {EZ1}, wherein R₅-R₉ are independently selected from hydrogen, —Cl, —F, —OH, or NH₂.
{EZ4}. The K-Ras inhibiting compound according to Embodiment {EZ1}, wherein R₇ is —X and R₅, R₆, R₈, and R₉ are each hydrogen.
{EZ5}. The K-Ras inhibiting compound according to Embodiment {EZ4}, wherein R₇ is independently selected from hydrogen, —Cl, —F, —OH, or NH₂.
{EZ6}. The K-Ras inhibiting compound according to Embodiment {EZ4}, wherein R₇ is Cl.
{EZ7}. The K-Ras inhibiting compound according to Embodiment {EZ4}, wherein R₇ is F.

### Scaffold A27—Embodiment AA (“EAA”)

{EAA1}. A compound for inhibiting K-Ras having formula (A27) or (A27), or a pharmaceutically acceptable salt thereof:

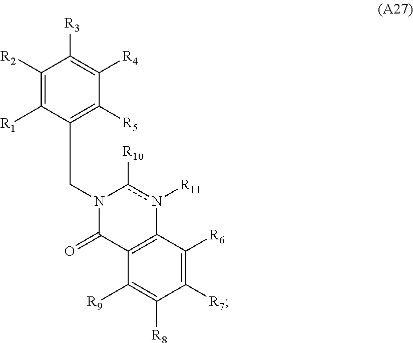

wherein the “dashed” bond is a double or single bond;
R₁-R₉ are independently selected from hydrogen, —R, —OR, —X, or —R^{B}; where any two vicinal groups R₁-R₉ may together form a fused ring;
R₁₀ and R₁₁ are independently selected from hydrogen, —X, —R^{B}, -L₁-X, -L₁-R^{B}, -L₁-L₂-R^{B}, -L₁-L₂-R^{L}—R^{B}, -L₁-R^{L}-L₂-R^{B}, or a group R₂₇;
R₂₇ is selected from -L₁-R^{L}-L₁-R^{B}, -L₁-R^{L}-L₁-X, R^{L}-L₁-R^{B}, -L₁-R^{L}-L₁-R, —R^{L}-L₁-R, where R₁₀ and R₁₁ may together form a fused ring, wherein said fused ring is optionally aromatic and may contain a substituted R₂₇; where
X is independently selected at each occurrence from —F, —Cl, —Br, —I, —OH, —OR*, —NH₂, —NHR*, —N(R*)₂, —N(R*)₃⁺, —N(R*)—OH, —N(*O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂⁻, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(═O)₁₋₂—R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂;
R* is independently selected at each occurrence from hydrogen and a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.);
R is selected from hydrogen or C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteroatoms selected from O, S, N, P, F, Cl, Br, I, and combinations thereof;
R^{L} at each occurrence is a C₁₋₆ linear or branched bivalent hydrocarbon radical; optionally substituted with one or more (e.g., 1-5) groups X and/or with 1-6 heteroatoms selected from O, S, N, P, F, Cl, Br, I, and combinations thereof;
R^{B} is a C₃₋₆ cyclic hydrocarbon (alicylic or aromatic) or heterocycle (e.g., heteroaryl), optionally substituted with one or more groups X and or with 1-6 heteroatoms selected from O, S, N, P, F, Cl, B, I, and combinations thereof; and wherein R^{B} may further comprise an additional 5- or 6-membered optionally aromatic ring fused thereto;
L₁ and L₂ are selected independently at each occurrence from —O—, —S—, —NH—, —N(R^{N})—, —(CH₂)₁₋₄—, —C═C—, —C═C—(CH₂)₁₋₃—, —C(O)—, —(CH₂)₁₋₃—C(O)—, —C(O)—(CH₂)₁₋₃—, —C(O)—O—(CH₂)₁₋₃—, —C(O)—N(R^{N})—, —N(R^{N})—C(O)—, —C(O)—N(R^{N})—(CH₂)₁₋₃—, —(CH₂)₁₋₃—C(O)—N(R^{N})—, —(CH₂)₀₋₃—NH—C(O)—, —(CH₂)₀₋₃—NH—C(O)—O—, —NH—S(O)₁₋₂—, —N(R^{N})—S(O)₁₋₂—, —O—(CH₂)₁₋₃—, —S(O)₁₋₂—, —S—(CH₂)₁₋₃—, —NH—(CH₂)₁₋₃—, —N(R^{N})—(CH₂)₁₋₃—, and —(OCH₂CH₂)₁₋₃—; and
R^{N} is hydrogen, methyl, ethyl, or propyl.
{EAA2}. The K-Ras inhibiting compound according to Embodiment {EAA1} having the structure:

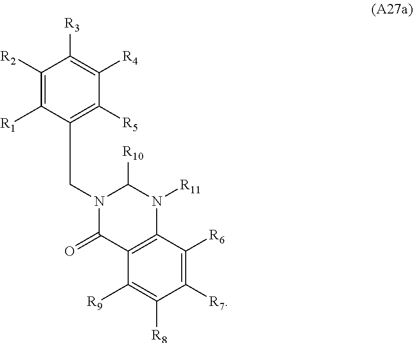

{EAA3}. The K-Ras inhibiting compound according to Embodiment {EAA1} having the structure:

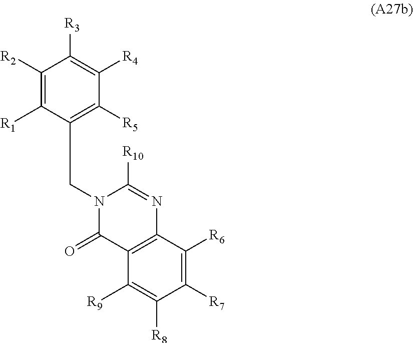

{EAA4}. The K-Ras inhibiting compound according to Embodiment {EAA1}, having the structure:

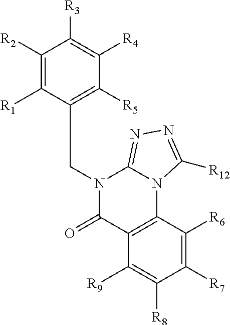

wherein R₁₂ is —R, —R^{B}, —R^{Q}—X, -(L₁)₀₋₃-R, -(L₁)₀₋₃-R^{B}, or -(L₁)₀₋₃-X.
{EAA5}. The K-Ras inhibiting compound according to Embodiment {EAA1}, wherein R₁-R₉ is selected from the group consisting of hydrogen, —F, —Cl, —CF₃, —CCl₃, —OH, —CH₃, —CH₂—CH₃, —CH₂—CH₂—CH₃, —OCH₃, —OCH₂—CH₃, or —OCH₂—CH₂—CH₃.
{EAA6}. The K-Ras inhibiting compound according to Embodiment {EAA5}, wherein two vicinal groups R₁-R₉ together form a fused ring.
{EAA7}. The K-Ras inhibiting compound according to Embodiment {EAA1}, wherein at least one of R₁-R₉ is a six-membered ring optionally substituted with —F, —Cl, —CF₃, —CCl₃, —OH, —CH₃, —CH₂—CH₃, —CH₂—CH₂—CH₃, —OCH₃, —OCH₂—CH₃, —OCH₂—CH₂—CH₃, N, or O.
{EAA8}. The K-Ras inhibiting compound according to Embodiment {EAA1}, wherein R₁₀ is a group X, and X is —NH—NH₂, where and R₁₀ and R₁₁ form a five-membered aromatic fused ring.
{EAA9}. The K-Ras inhibiting compound according to Embodiment {EAA1}, wherein R₁₁ of R₁₁ is -L₁-R^{B}, -L₁-L₂-R_{B}, -L₁-L₂-R^{L}—R^{B}, -L₁-L₁-R^{L}-L₂-R^{B}, -L₁-L₂-R_{L}-L₂-L₂-X, where L₁ is independently selected at each occurrence from —(CH₂)₁₋₄—, and R^{B} is a six-membered aromatic ring with at least one substitution with N, —F, —Cl, —CF₃, —CCl₃, —OH, —CH₃, —CH₂—CH₃, —CH₂—CH₂—CH₃, —OCH₃, —OCH₂—CH₃, or —OCH₂—CH₂—CH₃.
{EAA10}. The K-Ras inhibiting compound according to Embodiment {EAA9}, wherein L₂ of R₁₁ is independently selected at each occurrence from —C(O)—NH₂—, —NH—, —S—, —(CH₂)₁₋₃—C(O)—N(R^{N})—, —C(O)—N(R^{N})—(CH₂)₁₋₃₋, or —C(O)—, where R^{N} is hydrogen.
{EAA11}. The K-Ras inhibiting compound according to Embodiment {EAA9}, wherein R^{L} of R₁₁ is —C(R^{N})—.
{EAA12}. The K-Ras inhibiting compound according to Embodiment {EAA9}, wherein —X of R₁₁ is —NH₂.
{EAA13}. The K-Ras inhibiting compound according to Embodiment {EAA1} having the structure:

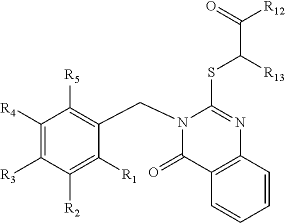

wherein R₁₂ is selected from —X, —R, —R^{B}, -L₁-X, -L₁-R, or -L₁-R^{B}; and
R₁₃ is selected from hydrogen or lower alkyl.
{EAA14}. The K-Ras inhibiting compound according to Embodiment {EAA13}, wherein R₁₂ is —R^{B} or -L₁-R^{B} and R^{B} is a five membered ring substituted with N or O.
{EAA15}. The K-Ras inhibiting compound according to Embodiment {EAA13}, wherein R₁₂ is -L₁-R^{B} and L₁ is —NH— or —NH—CH₂—.

### Scaffold A28—Embodiment BB (“EBB”)

{EBB1}. A compound for inhibiting K-Ras having formula (A28), or a pharmaceutically acceptable salt thereof:

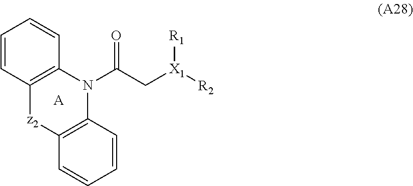

wherein ring “A” is a six- or seven-membered ring, z₂ is —CH₂CH₂— or —S—;
X₁ is selected from CH, O, or N, and in the case where X₁ is O, R₂ is absent;
R₁ and R₂ are independently selected from -L₁-R, -L₁-R^{Q}—X, -L₁-R^{Q}-L₁-R^{B}, -L₁-R^{Q}-L₁-X, -L₁-L₁-X, or -L₁-L₁-R; where R₁ and R₂ may together form a five- or six-membered ring, optionally including from 1-3 heteroatoms and/or groups X;
X is independently selected at each occurrence from —F, —Cl, —Br, —I, —OH, —OR*, —NH₂, —NHR*, —N(R*)₂, —N(R*)₃⁺, —N(R*)—OH, —N(*O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂⁻, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(═O)₁₋₂₋R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂;
R* is independently selected at each occurrence from hydrogen or a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.);
R is selected from hydrogen or C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteroatoms selected from O, S, N, P, F, Cl, Br, I, and combinations thereof,
R^{B} is a C₃₋₆ cyclic hydrocarbon (alicylic or aromatic) or heterocycle (e.g., heteroaryl), optionally substituted with one or more groups X and or with 1-6 heteroatoms selected from O, S, N, P, F, Cl, B, I, and combinations thereof; and wherein R^{B} may further comprise an additional 5- or 6-membered optionally aromatic ring fused thereto;
R^{Q} is a cyclic group having the structure:

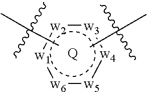

wherein ring “Q” is a five- or six-membered optionally aromatic ring, w₁-w₆ are independently selected from N, C, NH, NR^{N}, O, S, C═O, CH, CX, CR, CH₂, C(X)(X), C(R)(X), and C(R)(R); and in the case where ring “Q” is a five-membered ring, x₃ or x₅ is a bond (i.e., it is absent); and wherein any two vicinal substituents X and/or R and/or R^{N} may together form a 5- or 6-membered ring fused to ring “Q”;
L₁ is selected independently at each occurrence from —O—, —S—, —NH—, —N(R^{N})—, —(CH₂)₁₋₄—, —C═C—, —C═C—(CH₂)₁₋₃—, —C(O)—, —(CH₂)₁₋₃—C(O)—, —C(O)—(CH₂)₁₋₃—, —C(O)—O—(CH₂)₁₋₃—, —C(O)—N(R^{N})—, —N(R^{N})—C(O)—, —C(O)—N(R^{N})—(CH₂)₁₋₃—, —(CH₂)₁₋₃—C(O)—N(R^{N})—, —(CH₂)₀₋₃—NH—C(O)—, —(CH₂)₀₋₃—NH—C(O)—O—, —NH—S(O)₁₋₂—, —N(R^{N})—S(O)₁₋₂—, —S(O)₁₋₂—, —O—(CH₂)₁₋₃—, —S—(CH₂)₁₋₃—, —NH—(CH₂)₁₋₃—, —N(R^{N})—(CH₂)₁₋₃₋, and —(OCH₂CH₂)₁₋₃—; and R^{N} is hydrogen, methyl, ethyl, or propyl.
{EBB2}. The K-Ras inhibiting compound according to Embodiment {EBB1}, wherein L₁ is independently selected at each occurrence from —(CH₂)₁₋₃, —C(O)—, or —S(O)₂—.
{EBB3}. The K-Ras inhibiting compound according to Embodiment {EBB1}, wherein R^{Q} has the structure

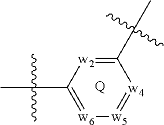

where w₂ and w₄-w₆ are independently selected from CH, CX, or CR.
{EBB4}. The K-Ras inhibiting compound according to Embodiment {EBB1}, wherein X is —NH₂ or —OH.
{EBB5}. The K-Ras inhibiting compound according to Embodiment {EBB1} having the structure:

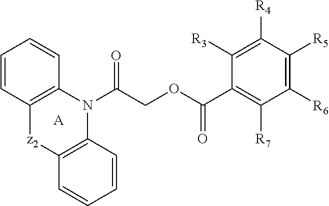

wherein R₃-R₇ are independently selected from hydrogen, —R, or —X;
{EBB6}. The K-Ras inhibiting compound according to Embodiment {EBB5}, wherein one of R₃-R₅ is not hydrogen.
{EBB7}. The K-Ras inhibiting compound according to Embodiment {EBB5}, wherein two of R₃-R₅ are not hydrogen.
{EBB8}. The K-Ras inhibiting compound according to Embodiment {EBB5}, wherein three of R₃-R₅ are not hydrogen.
{EBB9}. The K-Ras inhibiting compound according to Embodiment {EBB5}, wherein one of R₄ or R₆ is —SO₂—NH₂.
{EBB10}. The K-Ras inhibiting compound according to Embodiment {EBB5}, wherein one of R₄ or R₆ is —OH.
{EBB11}. The K-Ras inhibiting compound according to Embodiment {EBB5}, wherein one of R₃-R₅ is lower alkyl.
{EBB12}. The K-Ras inhibiting compound according to Embodiment {EBB5}, wherein two of R₃-R₅ are independently selected from lower alkyl.
{EBB13}. The K-Ras inhibiting compound according to Embodiment {EBB5}, wherein one of R₃-R₅ is methyl.
{EBB14}. The K-Ras inhibiting compound according to Embodiment {EBB5}, wherein two of R₃-R₅ are methyl.

### Scaffold A29—Embodiment CC (“ECC”)

{ECC1}. A compound for inhibiting K-Ras having formula (A29), or a pharmaceutically acceptable salt thereof:

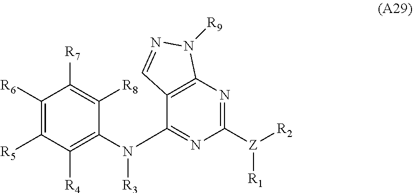

wherein R₁ and R₂ are independently selected from hydrogen, —X, —R, or —R^{L}—X, —R^{L}—R, -L₁-(R^{L})₀₋₁—X, -L₁-R, -L₁-R^{B}, —R^{L}—R^{B}, -L₁-(R^{L})₀₋₁—R^{B}, and R₁ and R₂ may together form a five- or six-membered ring optionally substituted with N, S, O, or X;
R₃ is selected from hydrogen or methyl;
R₄-R₇ are independently selected from hydrogen, —X, or —R;
R₉ is hydrogen or R^{B};
Z is selected from H, CH, or N; where
X is independently selected at each occurrence from —F, —Cl, —Br, —I, —OH, —OR*, —NH₂, —NHR*, —N(R*)₂, —N(R*)₃⁺, —N(R*)—OH, —N(*O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂⁻, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(═O)₁₋₂₋R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂;
R^{L} at each occurrence is a C₁₋₆ linear or branched bivalent hydrocarbon radical; optionally substituted with one or more (e.g., 1-5) groups X and/or with 1-6 heteroatoms selected from O, S, N, P, F, Cl, Br, I, and combinations thereof;
R^{B} is a C₃₋₁₂ hydrocarbon (alicyclic or aromatic) or heterocycle (e.g., heteroaryl), optionally substituted with one or more (e.g., 1-5) groups R* and/or X and/or with 1-6 heteroatoms selected from O, S, N, P, F, Cl, Br, I, and combinations thereof, where any vicinal groups can form a 5- or 6-membered fused ring;
R* is independently selected at each occurrence from hydrogen and a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.); and
R is selected from hydrogen or C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteroatoms selected from O, S, N, P, F, Cl, Br, I, and combinations thereof.
{ECC2}. The K-Ras inhibiting compound according to Embodiment {ECC1}, wherein R₁-R₈ are selected from hydrogen, —F, —Cl, —CF₃, —CCl₃, —OH, —CH₃, —CH₂—CH₃, —CH₂—CH₂—CH₃, —OCH₃, —OCH₂—CH₃, or —OCH₂—CH₂—CH₃.
{ECC3}. The K-Ras inhibiting compound according to Embodiment {ECC1}, wherein two of R₄-R₈ are R and the other of R₄-R₈ are each hydrogen, where R is selected from —CH₃, —CH₂—CH₃, or —CH₂—CH₂—CH₃.
{ECC4}. The K-Ras inhibiting compound according to Embodiment {ECC1}, wherein R₁ and R₂ are independently methyl, ethyl, or propyl, optionally substituted with OH.
{ECC5}. The K-Ras inhibiting compound according to Embodiment {ECC1}, wherein R₉ is R^{B}, and R^{B} is a six-membered aromatic ring.

### Scaffold A30—Embodiment DD (“EDD”)

{EDD1}. A compound for inhibiting K-Ras having formula (A30), or a pharmaceutically acceptable salt thereof:

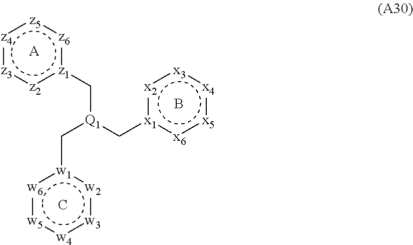

wherein ring “A” is a five- or six-membered, optionally aromatic, ring; where z₁ is C, CH, or N; and z₂-z₆ are independently selected from N, NH, NR^{N}, O, S, C═O, CH, CX, C(L₁R), C(L₁X), CR, CH₂, C(X)(X), C(R)(X), or C(R)(R); and wherein ring “A” may contain 0, 1, 2, or 3 double bonds; and in the case where ring “A” is a five-membered ring, z₄ is a bond (i.e., it is absent); and wherein any two vicinal substituents X and/or R and/or R^{N} may together form a 5- or 6-membered ring fused to ring “A”;
ring “B” is a five- or six-membered, optionally aromatic, ring; where x₁ is C, CH, or N; and x₂-x₆ are independently selected from N, NH, NR^{N}, O, S, C═O, CH, CX, C(L₁R), C(L₁X), CR, CH₂, C(X)(X), C(R)(X), or C(R)(R); and wherein ring “B” may contain 0, 1, 2, or 3 double bonds; and in the case where ring “B” is a five-membered ring, x₄ is a bond (i.e., it is absent); and wherein any two vicinal substituents X and/or R and/or R^{N} may together form a 5- or 6-membered ring fused to ring “B”;
ring “C” is a five- or six-membered, optionally aromatic, ring; where w₁ is C, CH, or N; and w₂-w₆ are independently selected from N, NH, NR^{N}, O, S, C═O, CH, CX, C(L₁R), C(L₁X), CR, CH₂, C(X)(X), C(R)(X), or C(R)(R); and wherein ring “C” may contain 0, 1, 2, or 3 double bonds; and in the case where ring “C” is a five-membered ring, w₄ is a bond (i.e., it is absent); and wherein any two vicinal substituents X and/or R and/or R^{N} may together form a 5- or 6-membered ring fused to ring “C”;
Q₁ is CH, CR, or N (preferably N); where
X is independently selected at each occurrence from —F, —Cl, —Br, —I, —OH, —OR*, —NH₂, —NHR*, —N(R*)₂, —N(R*)₃⁺, —N(R*)—OH, —N(*O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂—, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(═O)₁₋₂—R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂;
R* is independently selected at each occurrence from hydrogen or a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.);
R is selected from hydrogen or C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteroatoms selected from O, S, N, P, F, Cl, Br, I, and combinations thereof;
L₁ is selected independently at each occurrence from —O—, —S—, —NH—, —N(R^{N})—, —(CH₂)₁₋₄—, —C═C—, —C═C—(CH₂)₁₋₃—, —C(O)—, —(CH₂)₁₋₃—C(O)—, —C(O)—(CH₂)₁₋₃—, —C(O)—O—(CH₂)₁₋₃—, C(O)—N(R^{N})—, —N(R^{N})—C(O)—, —C(O)—N(R^{N})—(CH₂)₁₋₃—, —(CH₂)₁₋₃—C(O)—N(R^{N})—, —(CH₂)₀₋₃—NH—C(O)—, —(CH₂)₀₋₃—NH—C(O)—O—, —NH—S(O)₁₋₂—, —N(R^{N})—S(O)₁₋₂—, —O—(CH₂)₁₋₃—, —S(O)₁₋₂—, —S—(CH₂)₁₋₃—, —NH—(CH₂)₁₋₃—, —N(R^{N})—(CH₂)₁₋₃₋, and —(OCH₂CH₂)₁₋₃—; and
R^{N} is hydrogen, methyl, ethyl, or propyl.
{EDD2}. The K-Ras inhibiting compound according to Embodiment {EDD1}, wherein ring “A,” ring “B,” and ring “C” are each aromatic.
{EDD3}. The K-Ras inhibiting compound according to Embodiment {EDD2}, wherein z₄, w₄, and x₄ are each independently selected from N, CX, CL₁X, CL₁R, or CH, where X is selected from —F, —N(R*), —Cl, —CF₃, —CCl₃, —OH, —OCH₃, —OCH₂—CH₃, or —OCH₂—CH₂—CH₃, and R is selected from —CH₃, —CH₂—CH₃, or —CH₂—CH₂—CH₃.
{EDD4}. The K-Ras inhibiting compound according to Embodiment {EDD2}, wherein L₁ is —S(O)₂— or —C(O)—.
{EDD5}. The K-Ras inhibiting compound according to Embodiment {EDD2}, wherein at least one of ring “A,” ring “B,” or ring “C” is five-membered having the structure

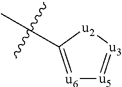

wherein u₂ is selected from NH, NR^{N}, O, S, C═O, CH₂, C(X)(X), C(R)(X), or C(R)(R), and u₃, u₅, and u₆ are independently selected from N, CH, CX, C(L₁R), C(L₁X), or CR.
{EDD6}. The K-Ras inhibiting compound according to Embodiment {EDD5}, wherein u₂ is selected from O, S, and NH, and u₆ is N.
{EDD7}. The K-Ras inhibiting compound according to Embodiment {EDD5}, wherein u₅ or u₆ is CL₁X or CL₁R, where X is selected from —F, —N(R*)—Cl, —CF₃, —CCl₃, —OH, —OCH₃, —OCH₂—CH₃, or —OCH₂—CH₂—CH₃, R is selected from —CH₃, —CH₂—CH₃, or —CH₂—CH₂—CH₃, and L₁ is —C(O)—, or —S(O₂)—.
{EDD8}. The K-Ras inhibiting compound according to Embodiment {EDD1}, wherein Q₁ is N.
{EDD9}. The K-Ras inhibiting compound according to Embodiment {EDD1}, wherein said compound is an acid addition salt.
{EDD10}. The K-Ras inhibiting compound according to Embodiment {EDD1} having the structure:

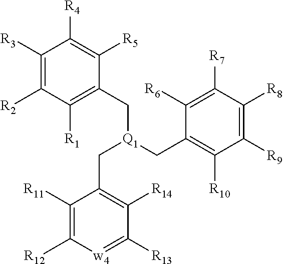

wherein R₁-R₁₄ are independently selected from hydrogen, —X, or -L₁-X, or -L₁-R.
{EDD11}. The K-Ras inhibiting compound according to Embodiment {EDD10}, wherein one of R₁-R₁₄ is -L₁-R, where L₁ is —S(O)₂—NH— or —C(O)—NH—.
{EDD12}. The K-Ras inhibiting compound according to Embodiment {EDD10}, wherein one of R₁-R₁₄ is -L₁-R′, where L₁ is —S(O)₂—NH— or —C(O)—NH— and R′ is lower alkyl.
{EDD13}. The K-Ras inhibiting compound according to Embodiment {EDD1} having the structure:

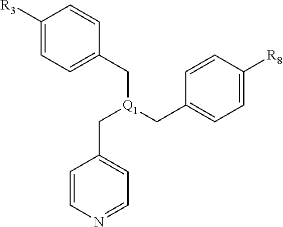

wherein R₃ and R₈ are independently selected from hydrogen, —X, or -L₁-X, or -L₁-R.
{EDD14}. The K-Ras inhibiting compound according to Embodiment {EDD13}, wherein one of R₃ or R₈ is selected from hydrogen, —Cl, —F, or —OH and the other of R₃ or R₈ is -L₁-X or -L₁-R.
{EDD15}. The K-Ras inhibiting compound according to Embodiment {EDD13}, wherein one of R₃ or R₈ is -L₁-R′, where R′ is lower alkyl and L₁ is —S(O)₂—NH— or —C(O)—NH—.
{EDD16}. The K-Ras inhibiting compound according to Embodiment {EDD1} having the structure:

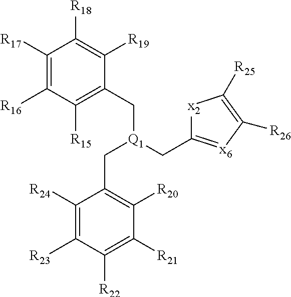

wherein R₁₅-R₂₄ are independently selected from hydrogen, —R, or —X;
R₂₅ and R₂₆ are independently selected from hydrogen, —X, or -L₁-R;
x₂ is selected from O, NH, or S; and
x₆ is selected from N or CH.
{EDD17}. The K-Ras inhibiting compound according to Embodiment {EDD16}, wherein R₂₅ is hydrogen.
{EDD18}. The K-Ras inhibiting compound according to Embodiment {EDD16}, wherein R₂₆ is -L₁-R, where L₁ is —S(O)₂—NH— or —C(O)—NH—.
{EDD19}. The K-Ras inhibiting compound according to Embodiment {EDD16}, wherein R₂₆ is -L₁-R′, where R′ is lower alkyl and L₁ is —S(O)₂—NH— or —C(O)—NH—.

### Scaffold A31—Embodiment EE (“EEE”)

{EEE1}. A compound for inhibiting K-Ras having formula (A31), or a pharmaceutically acceptable salt thereof:

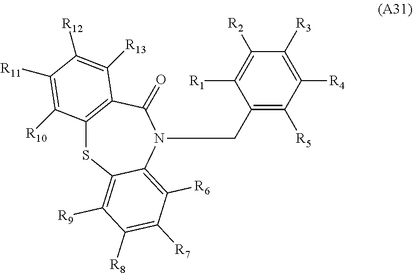

wherein R₁-R₁₃ are independently selected from hydrogen, —R*, or —X; where
X is independently selected at each occurrence from —F, —Cl, —Br, —I, —OH, —OR*, —NH₂, —NHR*, —N(R*)₂, —N(R*)₃⁺, —N(R*)—OH, —N(*O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂⁻, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(═O)₁₋₂—R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂;
and
R* is independently selected at each occurrence from hydrogen and a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.).
{EEE2}. The K-Ras inhibiting compound according to Embodiment {EEE1}, wherein R₁-R₁₃ are independently selected from hydrogen, —F, —Cl, —Br, —CO₂⁻, —NH₂, —CN, —CF₃, —O—CF₃, or —OH.
{EEE3}. The K-Ras inhibiting compound according to Embodiment {EEE2}, wherein one of R₁-R₁₃ is selected from —F, —Cl, —Br, —CO₂⁻, —NH₂, —CN, —CF₃, —O—CF₃, or —OH, and the other of R₁-R₁₃ are each hydrogen.
{EEE4}. The K-Ras inhibiting compound according to Embodiment {EEE2}, wherein two of R₁-R₁₃ are independently selected from —F, —Cl, —Br, —CO₂⁻, —NH₂, —CN, —CF₃, —O—CF₃, or —OH, and the other of R₁-R₁₃ are each hydrogen.

### Scaffold A32—Embodiment FF (“EFF”)

{EFF1}. A compound for inhibiting K-Ras having formula (A32), or a pharmaceutically acceptable salt thereof:

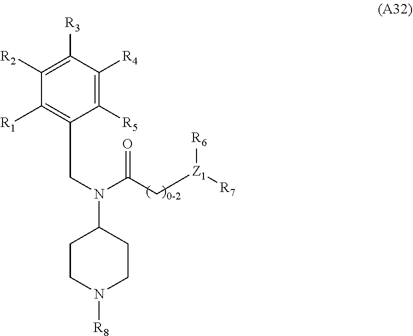

wherein R₁-R₅ are independently selected from hydrogen or —X,
R₆ and R₇ are independently selected from hydrogen, —R, —X, —R^{L}—X, —R^{L}—R, —R^{B}, -L₁-R, —R^{L}—R, or -L₁-R^{B}
R₈ are independently selected from hydrogen, —R, R^{B}, -L₁-R, or -L₁-R^{B}; and
Z is O, N, or CH, and when Z₁ is O, then R₆ is absent; where
X is independently selected at each occurrence from —F, —Cl, —Br, —I, —OH, —OR*, —NH₂, —NHR*, —N(R*)₂, —N(R*)₃⁺, —N(R*)—OH, —N(*O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂⁻, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(═O)₁₋₂—R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂;
R^{B} is a C₃₋₁₂ hydrocarbon (alicyclic or aromatic) or heterocycle (e.g., heteroaryl), optionally substituted with one or more (e.g., 1-5) groups R* and/or X and/or with 1-6 heteroatoms selected from O, S, N, P, F, Cl, Br, I, and combinations thereof, where any vicinal groups can form a 5- or 6-membered fused ring;
R^{L} is independently selected at each occurrence from C₁₋₆ linear or branched bivalent hydrocarbon radicals; optionally substituted with one or more (e.g., 1-5) groups X and/or with 1-6 heteroatoms selected from O, S, N, P, F, Cl, Br, I, and combinations thereof;
R is selected from hydrogen or C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteroatoms selected from O, S, N, P, F, Cl, Br, I, and combinations thereof;
R* is independently selected at each occurrence from hydrogen and a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.); and
R^{N} is hydrogen, methyl, ethyl, or propyl.
{EFF2}. The K-Ras inhibiting compound according to Embodiment {EFF1}, wherein R₁-R₅ are independently selected from hydrogen, —F, —Cl, —Br, —CO₂⁻, —NH₂, —CN, —CF₃, —O—CF₃, or —OH.
{EFF3}. The K-Ras inhibiting compound according to Embodiment {EFF2}, wherein one of R₁-R₅ is selected from —F, —Cl, —Br, —CO₂⁻, —NH₂, —CN, —CF₃, —O—CF₃, or —OH, and the other of R₁-R₅ are each hydrogen.
{EFF4}. The K-Ras inhibiting compound according to Embodiment {EFF1}, wherein at least one of R₆-R₈ is -L₁-R^{B}, where L₁ is —S(O₂)—.
{EFF5}. The K-Ras inhibiting compound according to Embodiment {EFF4}, wherein R^{B} is a six-membered aromatic hydrocarbon optionally substituted with —CH₃, —CH₂—CH₃, —CH₂—CH₂—CH₃, —F, —Cl, or —Br.
{EFF6}. The K-Ras inhibiting compound according to Embodiment {EFF1} having the structure:

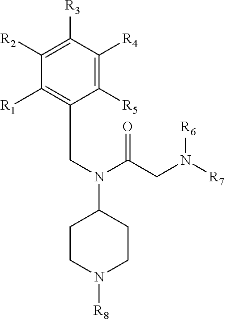

wherein R₇ and R₈ are -L₁-R^{B} and R₆ is R^{B}
{EFF7}. The K-Ras inhibiting compound according to Embodiment {EFF6} having the structure:

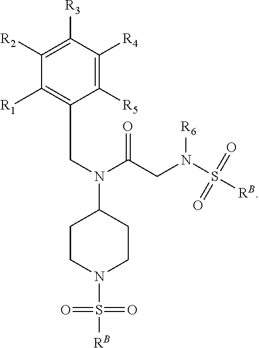

### Scaffold A33—Embodiment GG (“EGG”)

{EGG1}. A compound for inhibiting K-Ras having formula (A33), or a pharmaceutically acceptable salt thereof:

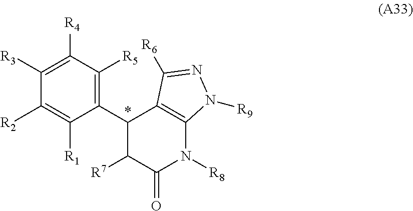

wherein R₁-R₅ are independently selected from hydrogen, —X, —R, or -L₁-X;
R₆-R₈ are independently selected from hydrogen, —R, or —X;
R₉ is hydrogen, —R, —X, or —R^{B}, —R^{L}—X, -L₁-R, -L₁-R^{B}, —R^{Q}—R, —R^{Q}—R^{B}, or R^{Q}—X; where
X is independently selected at each occurrence from —F, —Cl, —Br, —I, —OH, —OR*, —NH₂, —NHR*, —N(R*)₂, —N(R*)₃⁺, —N(R*)—OH, —N(*O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂⁻, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(═O)₁₋₂—R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂;
L₁ is selected independently at each occurrence from —O—, —S—, —NH—, —N(R^{N})—, —(CH₂)₁₋₄—, —C═C—, —C═C—(CH₂)₁₋₃—, —C(O)—, —(CH₂)₁₋₃—C(O)—, —C(O)—(CH₂)₁₋₃—, —C(O)—O—(CH₂)₁₋₃—, —C(O)—N(R^{N})—, —N(R^{N})—C(O)—, —C(O)—N(R^{N})—(CH₂)₁₋₃—, —(CH₂)₁₋₃—C(O)—N(R^{N})—, —(CH₂)₀₋₃—NH—C(O)—, —(CH₂)₀₋₃—NH—C(O)—O—, —NH—S(O)₁₋₂—, —N(R^{N})—S(O)₁₋₂—, —O—(CH₂)₁₋₃—, —S(O)₁₋₂—, —S—(CH₂)₁₋₃—, —NH—(CH₂)₁₋₃—, —N(R^{N})—(CH₂)₁₋₃₋, and —(OCH₂CH₂)₁₋₃—;
R^{B} is a C₃₋₁₂ hydrocarbon (alicyclic or aromatic) or heterocycle (e.g., heteroaryl), optionally substituted with one or more (e.g., 1-5) groups R* and/or X and/or with 1-6 heteroatoms selected from O, S, N, P, F, Cl, Br, I, and combinations thereof, where any vicinal groups can form a 5- or 6-membered fused ring;
R is selected from hydrogen or C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteroatoms selected from O, S, N, P, F, Cl, Br, I, and combinations thereof;
R^{Q} is a cyclic group having the structure:

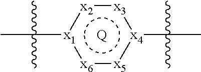

wherein ring “Q” is a five- or six-membered optionally aromatic ring, x₁ and x₄ are independently C, CH, CR or N; and x₂, x₃, x₅, and x₆, are independently selected from N, NH, NR^{N}, O, S, C═O, CH, CX, CR, CH₂, C(X)(X), C(R)(X), and C(R)(R); and in the case where ring “Q” is a five-membered ring, x₃ or x₅ is a bond (i.e., it is absent); and wherein any two vicinal substituents X and/or R and/or R^{N} may together form a 5- or 6-membered ring fused to ring “Q;”
R^{L} is independently selected at each occurrence from C₁₋₆ linear or branched bivalent hydrocarbon radicals; optionally substituted with one or more (e.g., 1-5) groups X and/or with 1-6 heteroatoms selected from O, S, N, P, F, Cl, Br, I, and combinations thereof;
R* is independently selected at each occurrence from hydrogen and a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.); and
R^{N} is hydrogen, methyl, ethyl, or propyl.
{EGG2}. The K-Ras inhibiting compound according to Embodiment {EGG1}, wherein R₁-R₅ are independently selected from hydrogen, —X and -L₁-X, where -L₁ is —O—.
{EGG3}. The K-Ras inhibiting compound according to Embodiment {EGG2}, wherein X is selected from —Cl, —F, —Br, —CF₃, —CHF₂, or —OH.
{EGG4}). The K-Ras inhibiting compound according to Embodiment {EGG2}, wherein at least one of R₁-R₅ is selected from —X and -L₁-X, where -L₁ is —O—.
{EGG5}. The K-Ras inhibiting compound according to Embodiment {EGG1}, wherein R₆ is X, where X is selected from —Cl, —F, —Br, —CF₃, —CHF₂, —O—CF₃, —O—CHF₂, or —OH.
{EGG6}. The K-Ras inhibiting compound according to Embodiment {EGG1}, wherein R₉ is R^{B}, and R^{B} is a six-membered ring.
{EGG7}. The K-Ras inhibiting compound according to Embodiment {EGG6}, wherein R^{B} is a saturated heterocycle substituted with N, O, or S.

### Scaffold A34—Embodiment HH (“EHH”)

{EHH1}. A compound for inhibiting K-Ras having formula (A34), or a pharmaceutically acceptable salt thereof:

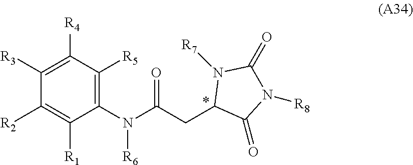

wherein R₁-R₆ are each independently selected from hydrogen, —R, or —X;
R₇ and R₈ are each independently selected from hydrogen, —R, —R^{L}—R^{B}, —R^{B}, or —X; where
X is independently selected at each occurrence from —F, —Cl, —Br, —I, —OH, —OR*, —NH₂, —NHR*, —N(R*)₂, —N(R*)₃⁺, —N(R*)—OH, —N(*O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂⁻, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(═O)₁₋₂—R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂;
R^{L} at each occurrence is a C₁₋₆ linear or branched bivalent hydrocarbon radical; optionally substituted with one or more (e.g., 1-5) groups X and/or with 1-6 heteroatoms selected from O, S, N, P, F, Cl, Br, I, and combinations thereof;
R^{B} is a C₃₋₁₂ hydrocarbon (alicyclic or aromatic) or heterocycle (e.g., heteroaryl), optionally substituted with one or more (e.g., 1-5) groups R* and/or X and/or with 1-6 heteroatoms selected from O, S, N, P, F, Cl, Br, I, and combinations thereof, where any vicinal groups can form a 5- or 6-membered fused ring;
R is selected from hydrogen or C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteroatoms selected from O, S, N, P, F, Cl, Br, I, and combinations thereof; and
R* is independently selected at each occurrence from hydrogen and a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.).
{EHH2}. The K-Ras inhibiting compound according to Embodiment {EHH1}, wherein R₁-R₅ are independently selected from hydrogen —Cl, —F, —Br, —CF₃, —CHF₂, —NH₂, —O—CF₃, —O—CHF₂, or —OH.
{EHH3}. The K-Ras inhibiting compound according to Embodiment {EHH1}, wherein R₈ and R₉ are independently selected from hydrogen, —R_{B}, or —R_{L}—R_{B}, where R_{B} is a six-membered aromatic hydrocarbon optionally substituted with one or more groups selected from methyl, ethyl, propyl, —Cl, —F, —Br, —CF₃, —CHF₂, —NH₂, —O—CF₃, —O—CHF₂, or —OH.
{EHH4}. The K-Ras inhibiting compound according to Embodiment {EHH1} having the structure:

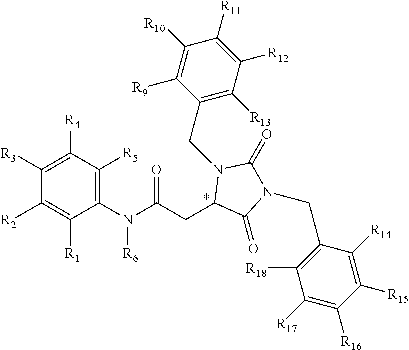

wherein R₉-R₁₈ are independently selected from hydrogen, —R, or —X.
{EHH5}. The K-Ras inhibiting compound according to Embodiment {EHH1} having the structure:

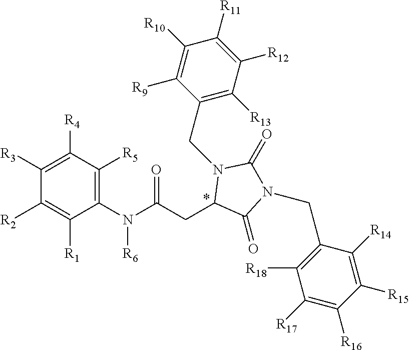

wherein R₉-R₁₈ are independently selected from hydrogen, or lower alkyl (e.g., methyl, etc.).

### Scaffold A35—Embodiment II (“EII”)

{EII1}. A compound for inhibiting K-Ras having formula (A35), or a pharmaceutically acceptable salt thereof:

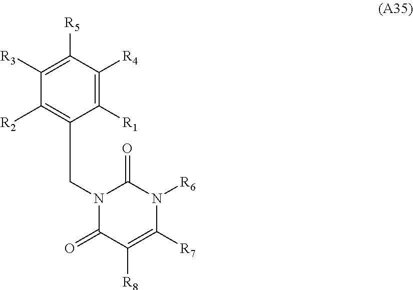

wherein R₁-R₅ are each independently selected from hydrogen, —R, or —X;
R₇-R₈ are each independently selected from hydrogen, —R, or —X;
R₇ is hydrogen, —R, —R^{B}, —X, —R^{Q}—X, —R^{Q}—R, -L₁-(R^{L})₀₋₁—X, -L₁-(R^{L})₀₋₁—R, or -L₁-(R^{L})₀₋₁—R^{B}; where
X is independently selected at each occurrence from —F, —Cl, —Br, —I, —OH, —OR*, —NH₂, —NHR*, —N(R*)₂, —N(R*)₃⁺, —N(R*)—OH, —N(*O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂⁻, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(═O)₁₋₂—R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂;
R is selected from hydrogen or C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteroatoms selected from O, S, N, P, F, Cl, Br, I, and combinations thereof;
R^{Q} is a cyclic group having the structure:

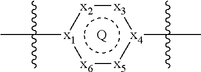

wherein ring “Q” is a five- or six-membered optionally aromatic ring, x₁ and x₄ are independently C, CH, CR or N; and x₂, x₃, x₅, and x₆, are independently selected from N, NH, NR^{N}, O, S, C═O, CH, CX, CR, CH₂, C(X)(X), C(R)(X), and C(R)(R); and in the case where ring “Q” is a five-membered ring, x₃ or x₅ is a bond (i.e., it is absent); and wherein any two vicinal substituents X and/or R and/or R^{N} may together form a 5- or 6-membered ring fused to ring “Q”;
R^{B} is a C₃₋₁₂ hydrocarbon (alicyclic or aromatic) or heterocycle (e.g., heteroaryl), optionally substituted with one or more (e.g., 1-5) groups R* and/or X and/or with 1-6 heteroatoms selected from O, S, N, P, F, Cl, Br, I, and combinations thereof, where any vicinal groups can form a 5- or 6-membered fused ring;
R^{L} is independently selected at each occurrence from C₁₋₆ linear or branched bivalent hydrocarbon radicals; optionally substituted with one or more (e.g., 1-5) groups X and/or with 1-6 heteroatoms selected from O, S, N, P, F, Cl, Br, I, and combinations thereof;
R* is independently selected at each occurrence from hydrogen and a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.); and
R^{N} is hydrogen, methyl, ethyl, or propyl.
{EII2}. The K-Ras inhibiting compound according to Embodiment {EII1}, wherein R₁-R₅ are independently selected from hydrogen —Cl, —F, —Br, —CF₃, —CHF₂, —NH₂, —O—CF₃, —O—CHF₂, or —OH.
{EII3}. The K-Ras inhibiting compound according to Embodiment {EII1}, wherein R₆-R₈ are independently selected from hydrogen and —R^{Q}—X, where R^{Q} is a six-membered alicyclic heterocycle.
{EII4}. The K-Ras inhibiting compound according to Embodiment {EII3}, wherein said six-membered alicyclic heterocycle is selected from the group consisting of piperidinyl, oxanyl, thianyl, piperazinyl, morpholynyl, or thiomorpholynyl.
{EII5}. The K-Ras inhibiting compound according to Embodiment {EII1} or {EII3}, wherein R₆-R₈ are independently selected from hydrogen and —R^{Q}—X, where X is —C(O₂)R*.
{EII6}. The K-Ras inhibiting compound according to Embodiment {EII1} having the structure:

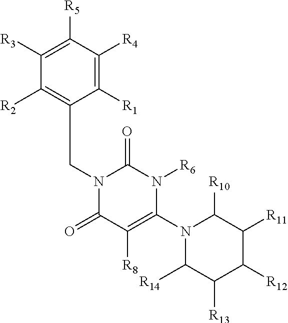

wherein R₁₀-R₁₄ are selected from hydrogen and X.
{EII7}. The K-Ras inhibiting compound according to Embodiment {EII6}, wherein one of R₁₀-R₁₄ is X.
{EII8}. The K-Ras inhibiting compound according to Embodiment {EII6}, wherein one of R₁₀-R₁₄ is —C(O)OR*.
{EII9}. The K-Ras inhibiting compound according to Embodiment {EII6}, wherein one of R₁₀-R₁₄ is —C(O)OR′, where R′ is hydrogen or lower alkyl.

### Scaffold A36—Embodiment JJ (“EJJ”)

{EJJ1}. A compound for inhibiting K-Ras having formula (A36), or a pharmaceutically acceptable salt thereof:

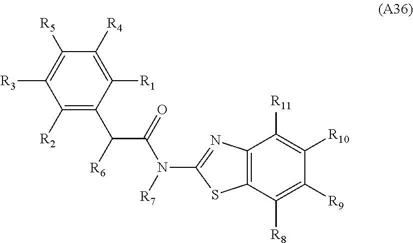

wherein R₁-R₆ are each independently selected from hydrogen, —R, or —X;
R₇ is selected from hydrogen, —R, —X, —R^{B}, —R^{L}—R, —R^{L}—R^{B}, or —R^{L}—X;
R₈-R₁₁ are independently selected from hydrogen, —R, —X, -L₁-X, and -L₁-R; where
X is independently selected at each occurrence from —F, —Cl, —Br, —I, —OH, —OR*, —NH₂, —NHR*, —N(R*)₂, —N(R*)₃⁺, —N(R*)—OH, —N(*O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂⁻, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(═O)₁₋₂—R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂;
R is selected from hydrogen or C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteroatoms selected from O, S, N, P, F, Cl, Br, I, and combinations thereof;
R^{L} is independently selected at each occurrence from C₁₋₆ linear or branched bivalent hydrocarbon radicals; optionally substituted with one or more (e.g., 1-5) groups X and/or with 1-6 heteroatoms selected from O, S, N, P, F, Cl, Br, I, and combinations thereof;
R^{B} is a C₃₋₁₂ hydrocarbon (alicyclic or aromatic) or heterocycle (e.g., heteroaryl), optionally substituted with one or more (e.g., 1-5) groups R* and/or X and/or with 1-6 heteroatoms selected from O, S, N, P, F, Cl, Br, I, and combinations thereof, where any vicinal groups can form a 5- or 6-membered fused ring;
L₁ is selected independently at each occurrence from —O—, —S—, —NH—, —N(R^{N})—, —(CH₂)₁₋₄—, —C═C—, —C═C—(CH₂)₁₋₃—, —C(O)—, —(CH₂)₁₋₃—C(O)—, —C(O)—(CH₂)₁₋₃—, —C(O)—O—(CH₂)₁₋₃—, —C(O)—N(R^{N})—, —N(R^{N})—C(O)—, —C(O)—N(R^{N})—(CH₂)₁₋₃—, —(CH₂)₁₋₃—C(O)—N(R^{N})—, —(CH₂)₀₋₃—NH—C(O)—, —(CH₂)₀₋₃—NH—C(O)—O—, —NH—S(O)₁₋₂—, —N(R^{N})—S(O)₁₋₂—, —O—(CH₂)₁₋₃—, —S(O)₁₋₂—, —S—(CH₂)₁₋₃—, —NH—(CH₂)₁₋₃—, —N(R^{N})—(CH₂)₁₋₃₋, and —(OCH₂CH₂)₁₋₃—;
R* is independently selected at each occurrence from hydrogen and a C₁₋₁₀ (e.g., C₁-s or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.); and
R^{N} is hydrogen, methyl, ethyl, or propyl.
{EJJ2}. The K-Ras inhibiting compound according to Embodiment {EJJ1}, wherein R₁-R₅ are independently selected from hydrogen, methyl, ethyl, propyl, methoxy, ethoxy, propoxy, —Cl, —F, —Br, —CF₃, —CHF₂, —NH₂, —O—CF₃, —O—CHF₂, or —OH.
{EJJ3}. The K-Ras inhibiting compound according to Embodiment {EJJ1}, wherein R₈-R₁₁ are independently selected from hydrogen or -L₁-R, where L₁ is —S(O₂)—, and R is methyl, ethyl, or propyl.
{EJJ4}. The K-Ras inhibiting compound according to Embodiment {EJJ1}, wherein R₇ is —R^{L}R^{B}, where R^{B} is a five-membered aromatic hydrocarbon ring.
{EJJ5}. The K-Ras inhibiting compound according to Embodiment {EJJ4}, wherein said five-membered aromatic hydrocarbon ring is selected from pyrrolyl, furanyl, or thiophenyl.
{EJJ6}. The K-Ras inhibiting compound according to Embodiment {EJJ1}, wherein R^{L} is a C₁-C₆ linear bivalent hydrocarbon radical.

### Scaffold A37—Embodiment K (“EKK”)

{EKK1}. A compound for inhibiting K-Ras having formula (A37), or a pharmaceutically acceptable salt thereof:

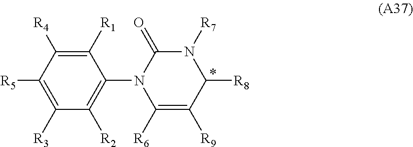

wherein R₁-R₅ are each independently selected from hydrogen, —R (e.g., lower alkyl, methyl, etc.), or —X;
R₆ is selected from from hydrogen, —R (e.g., lower alkyl, methyl, etc.), or —X;
R₇ is selected from from hydrogen, —R (e.g., lower alkyl, methyl, etc.), or —X;
R₈ and R₉ are independently selected from hydrogen, —R, —X, -L₁-X, -L₁-R, —R^{B}, -L₁-R^{B}, or —R^{Q}—X; where
X is independently selected at each occurrence from —F, —Cl, —Br, —I, —OH, —OR*, —NH₂, —NHR*, —N(R*)₂, —N(R*)₃⁺, —N(R*)—OH, —N(*O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂⁻, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(═O)₁₋₂₋R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂;
R^{Q} is a cyclic group having the structure:

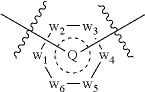

wherein ring “Q” is a five- or six-membered optionally aromatic ring, w₁-w₆ are independently selected from N, C, NH, NR^{N}, O, S, C═O, CH, CX, CR, CH₂, C(X)(X), C(R)(X), and C(R)(R); and in the case where ring “Q” is a five-membered ring, x₃ or x₅ is a bond (i.e., it is absent); and wherein any two vicinal substituents X and/or R and/or R^{N} may together form a 5- or 6-membered ring fused to ring “Q”;
R is selected from hydrogen or C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteroatoms selected from O, S, N, P, F, Cl, Br, I, and combinations thereof;
L₁ is selected independently at each occurrence from —O—, —S—, —NH—, —N(R^{N})—, —(CH₂)₁₋₄—, —C═C—, —C═C—(CH₂)₁₋₃—, —C(O)—, —(CH₂)₁₋₃—C(O)—, —C(O)—(CH₂)₁₋₃—, —C(O)—O—(CH₂)₁₋₃—, —C(O)—N(R^{N})—, —N(R^{N})—C(O)—, —C(O)—N(R^{N})—(CH₂)₁₋₃—, —(CH₂)₁₋₃—C(O)—N(R^{N})—, —(CH₂)₀₋₃—NH—C(O)—, —(CH₂)₀₋₃—NH—C(O)—O—, —NH—S(O)₁₋₂—, —N(R^{N})—S(O)₁₋₂—, —O—(CH₂)₁₋₃—, —S(O)₁₋₂—, —S—(CH₂)₁₋₃—, —NH—(CH₂)₁₋₃—, —N(R^{N})—(CH₂)₁₋₃₋, and —(OCH₂CH₂)₁₋₃—;
R* is independently selected at each occurrence from hydrogen and a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.); and
R^{N} is hydrogen, methyl, ethyl, or propyl.
{EKK2}. The K-Ras inhibiting compound according to Embodiment {EKK1}, wherein R₁-R₇ are independently selected from hydrogen, methyl, ethyl, propyl, methoxy, ethoxy, propoxy, —Cl, —F, —Br, —CF₃, —CHF₂, —NH₂, —O—CF₃, —O—CHF₂, or —OH.
{EKK3}. The K-Ras inhibiting compound according to Embodiment {EKK1}, wherein R₈ and R₉ are independently selected from hydrogen, —X, or —R^{Q}—X, where X is independently selected from —CN or —COOR*.
{EKK4}. The K-Ras inhibiting compound according to Embodiment {EKK3}, wherein R^{Q} is a six-membered aromatic hydrocarbon having the structure

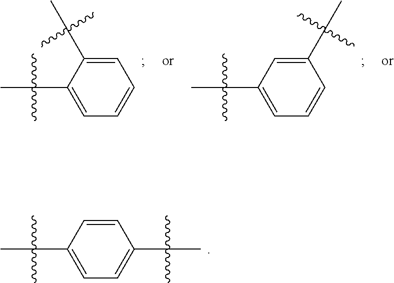 [SMILES: *c1ccccc1* ; *c1cccc(*)c1 ; *c1ccc(*)cc1]

{EKK5}. The K-Ras inhibiting compound according to Embodiment {EKK1} having the structure

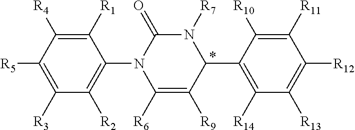

wherein R₁₀-R₁₄ are independently selected from hydrogen, —R, or —X.
{EKK6}. The K-Ras inhibiting compound according to Embodiment {EKK5}, wherein one of R₁₀-R₁₄ is not hydrogen.
{EKK7}. The K-Ras inhibiting compound according to Embodiment {EKK5}, wherein one of R₁₀-R₁₄ is —CN.

### Scaffold A38—Embodiment LL (“ELL”)

{ELL1}. A compound for inhibiting K-Ras having formula (A38), or a pharmaceutically acceptable salt thereof:

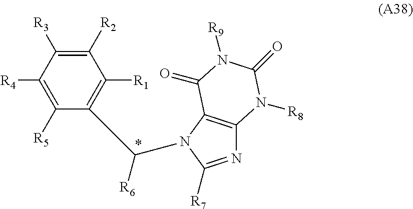

wherein R₁-R₇ are each independently selected from hydrogen, —R, or —X;
R₈ and R₉ are independently selected from hydrogen, —R, —X, —R^{B}, —R^{L}—R, —R^{L}—R^{B}, —R^{L}-(L₁)₀₋₁-R^{B}, —R^{L}-(L₁)₀₋₁-R, —R^{L}—X, —R^{L}—R^{Q}—R, or —R^{L}—R^{Q}—X; where
X is independently selected at each occurrence from —F, —Cl, —Br, —I, —OH, —OR*, —NH₂, —NHR*, —N(R*)₂, —N(R*)₃⁺, —N(R*)—OH, —N(*O)(R*)₂, —O—N(R*)₂, —N(R*)—O—R*, —N(R*)—N(R*)₂, —C═N—R*, —N═C(R*)₂, —C═N—N(R*)₂, —C(═NR*)(—N(R*)₂), —C(H)(═N—OH), —SH, —SR*, —CN, —NC, —CHF₂, —CCl₃, —CF₂Cl, —CFCl₂, —C(═O)—R*, —CHO, —CO₂H, —C(O)CH₃, —CO₂⁻, —CO₂R*, —C(═O)—S—R*, —O—(C═O)—H, —O—(C═O)—R*, —S—C(═O)—R*, —(C═O)—NH₂, —C(═O)—N(R*)₂, —C(═O)—NHNH₂, —O—C(═O)—NHNH₂, —C(═S)—NH₂, —(C═S)—N(R*)₂, —N(R*)—CHO, —N(R*)—C(═O)—R*, —C(═NR)—OR*, —O—C(═NR*)—R*, —SCN, —NCS, —NSO, —SSR*, —N(R*)—C(═O)—N(R*)₂, —CH₃, —CH₂—CH₃, —CH₂—CH₂—CH₃, —C(H)(CH₂)₂, —C(CH₃)₃, —N(R*)—C(═S)—N(R*)₂, —S(═O)₁₋₂₋R*, —O—S(═O)₂—R*, —S(═O)₂—OR*, —N(R*)—S(═O)₂—R*, —S(═O)₂—N(R*)₂, —O—SO₃, —O—S(═O)₂—OR*, —O—S(═O)—OR*, —O—S(═O)—R*, —S(═O)—OR*, —S(═O)—R*, —NO, —NO₂, —NO₃, —O—NO, —O—NO₂, —N₃, —N₂—R*, —N(C₂H₄), —Si(R*)₃, —CF₃, —O—CF₃, —O—CHF₂, —O—CH₃, —O—(CH₂)₁₋₆CH₃, —OC(H)(CH₂)₂—OC(CH₃)₃, —PR*₂, —O—P(═O)(OR*)₂, or —P(═O)(OR*)₂;
R^{L} is independently selected at each occurrence from C₁₋₆ linear or branched bivalent hydrocarbon radicals; optionally substituted with one or more (e.g., 1-5) groups X and/or with 1-6 heteroatoms selected from O, S, N, P, F, Cl, Br, I, and combinations thereof;
R^{B} is a C₃₋₁₂ hydrocarbon (alicyclic or aromatic) or heterocycle (e.g., heteroaryl), optionally substituted with one or more (e.g., 1-5) groups R* and/or X and/or with 1-6 heteroatoms selected from O, S, N, P, F, Cl, Br, I, and combinations thereof, where any vicinal groups can form a 5- or 6-membered fused ring;
R^{Q} is a cyclic group having the structure:

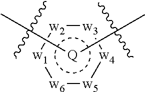

wherein ring “Q” is a five- or six-membered optionally aromatic ring, w₁-w₆ are independently selected from N, C, NH, NR^{N}, O, S, C═O, CH, CX, CR, CH₂, C(X)(X), C(R)(X), and C(R)(R); and in the case where ring “Q” is a five-membered ring, x₃ or x₅ is a bond (i.e., it is absent); and wherein any two vicinal substituents X and/or R and/or R^{N} may together form a 5- or 6-membered ring fused to ring “Q”;
L₁ is selected independently at each occurrence from —O—, —S—, —NH—, —N(R^{N})—, —(CH₂)₁₋₄—, —C═C—, —C═C—(CH₂)₁₋₃—, —C(O)—, —(CH₂)₁₋₃—C(O)—, —C(O)—(CH₂)₁₋₃—, —C(O)—O—(CH₂)₁₋₃—, —C(O)—N(R^{N})—, —N(R^{N})—C(O)—, —C(O)—N(R^{N})—(CH₂)₁₋₃—, —(CH₂)₁₋₃—C(O)—N(R^{N})—, —(CH₂)₀₋₃—NH—C(O)—, —(CH₂)₀₋₃—NH—C(O)—O—, —NH—S(O)₁₋₂—, —N(R^{N})—S(O)₁₋₂—, —O—(CH₂)₁₋₃—, —S(O)₁₋₂—, —S—(CH₂)₁₋₃—, —NH—(CH₂)₁₋₃—, —N(R^{N})—(CH₂)₁₋₃₋, and —(OCH₂CH₂)₁₋₃—;
R is selected from hydrogen or C₁₋₁₂ hydrocarbons (e.g., alkyl, alkenyl, alkynyl, aryl, alkyl-aryl, aryl-alkyl, and combinations thereof), optionally substituted with one or more (e.g., 1-5) groups X and/or with 1-10 heteroatoms selected from O, S, N, P, F, Cl, Br, I, and combinations thereof; and
R* is independently selected at each occurrence from hydrogen and a C₁₋₁₀ (e.g., C₁₋₈ or C₁₋₆ or C₁₋₄) hydrocarbon (e.g., alkyl, alkenyl, alkynyl, aryl (e.g., phenyl), alkyl-aryl (e.g., benzyl), aryl-alkyl (e.g., toluyl), etc.).
{ELL2}. The K-Ras inhibiting compound according to Embodiment {ELL1}, wherein R₁-R₇ are independently selected from hydrogen, methyl, ethyl, propyl, methoxy, ethoxy, propoxy, —Cl, —F, —Br, —CF₃, —CHF₂, —NH₂, —O—CF₃, —O—CHF₂, or —OH.
{ELL3}. The K-Ras inhibiting compound according to Embodiment {ELL1}, wherein R₈ and R₉ are independently selected from hydrogen, —R^{B}, and —R^{L}—R^{B}, where R^{B} is a six-membered aromatic hydrocarbon optionally subsititued with one or more heteroatoms selected from S, N, F, Cl, or Br.
{ELL4}. The K-Ras inhibiting compound according to Embodiment {ELL1}, wherein R₈ and R₉ are independently selected from —R^{B} and —R^{L}—R^{B}, where R^{B} is a six-membered aromatic hydrocarbon optionally subsititued with one or more heteroatoms selected from S, N, F, Cl or Br.

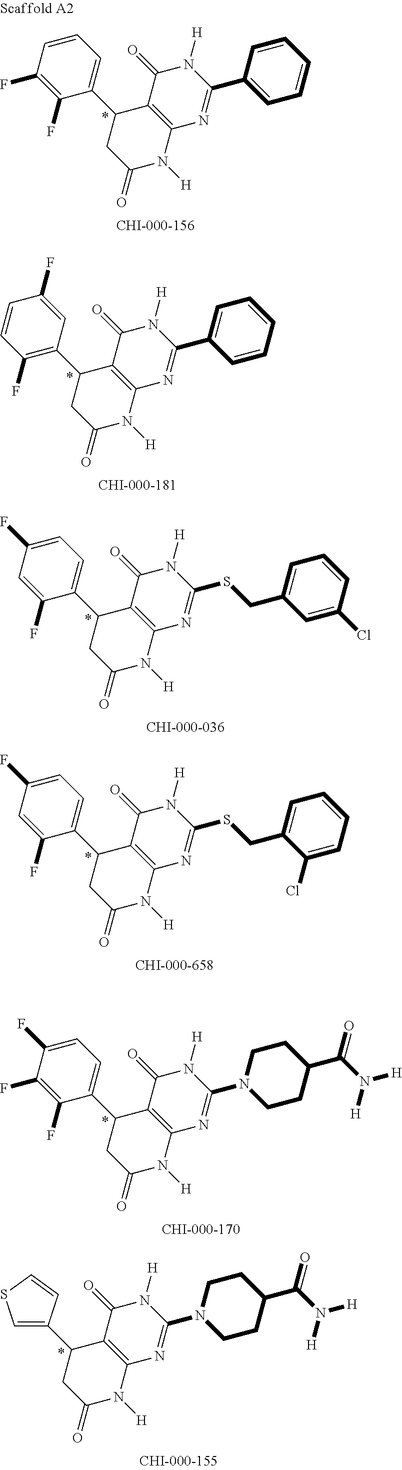

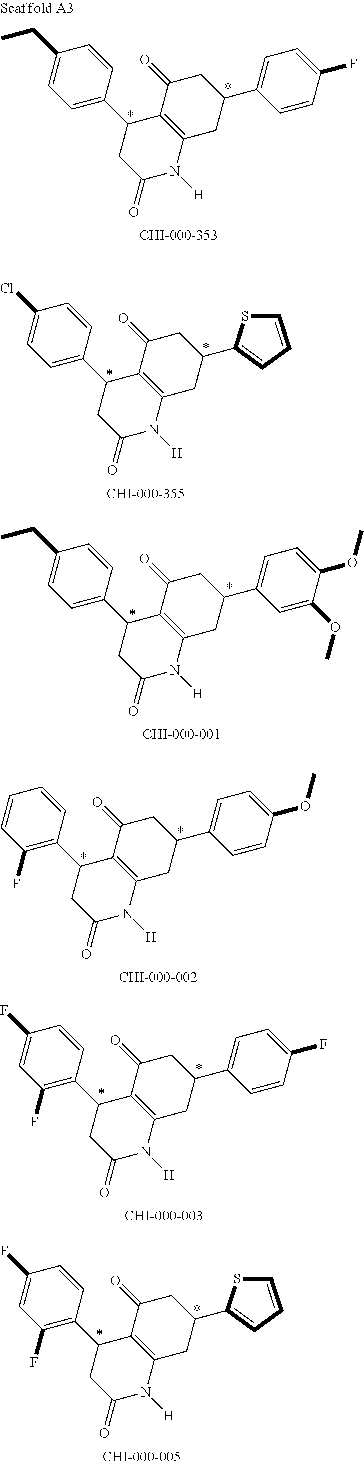

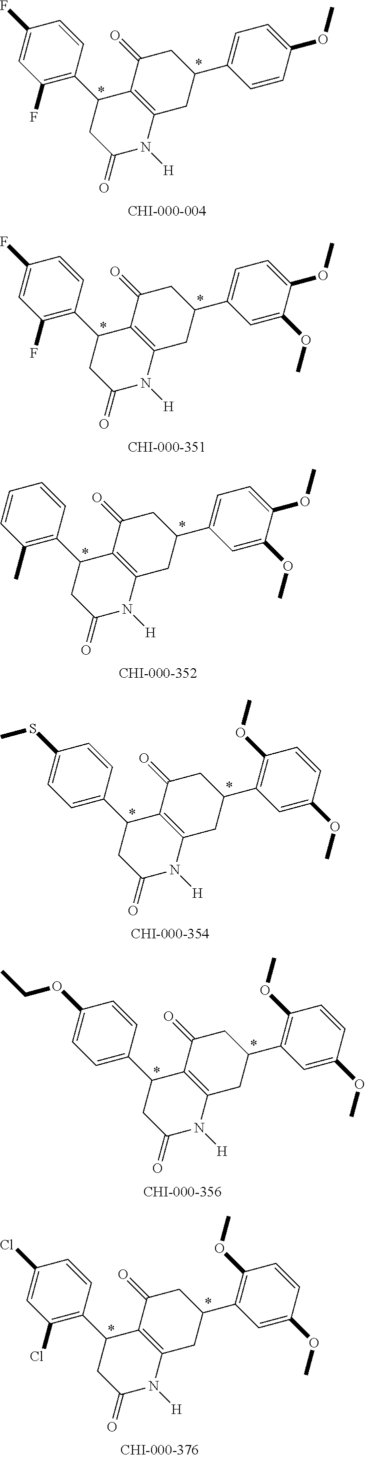

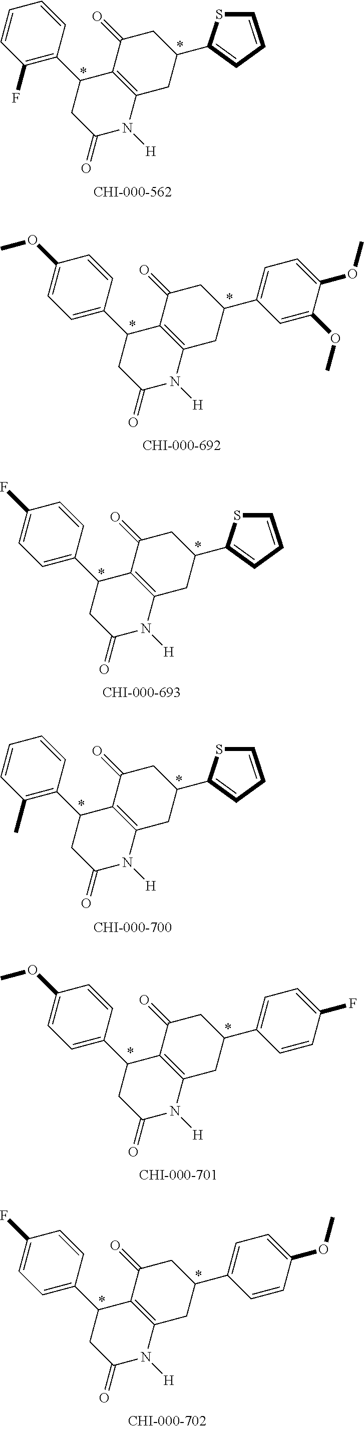

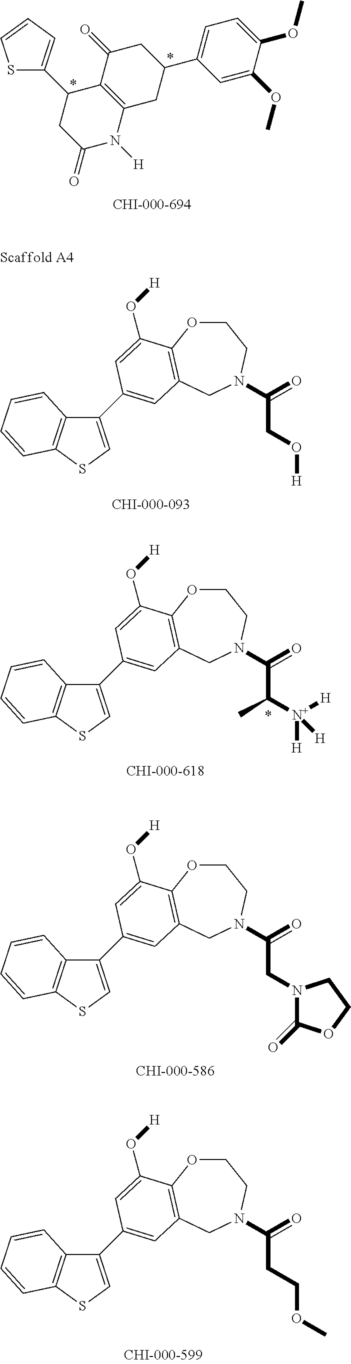

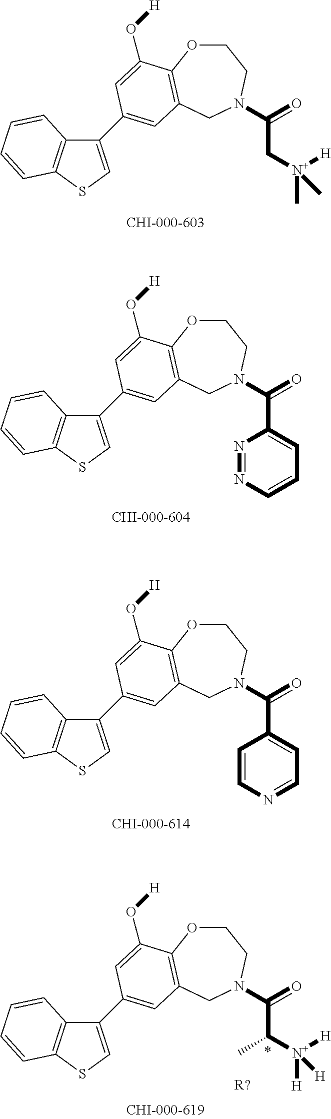

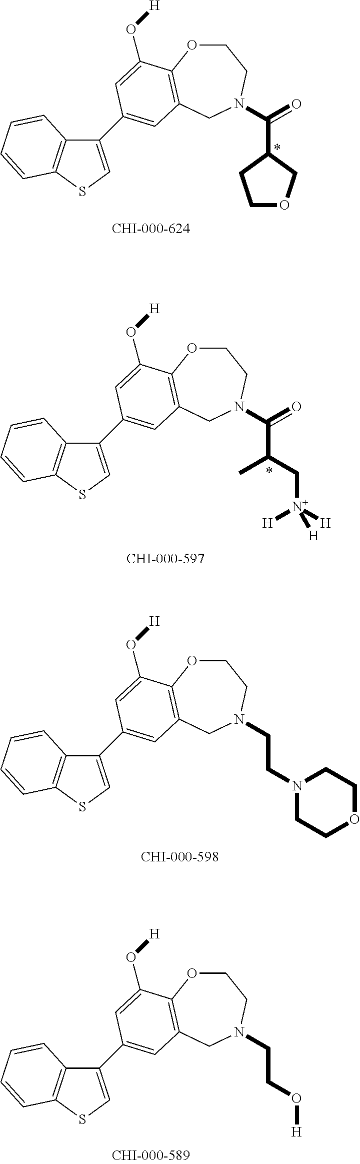

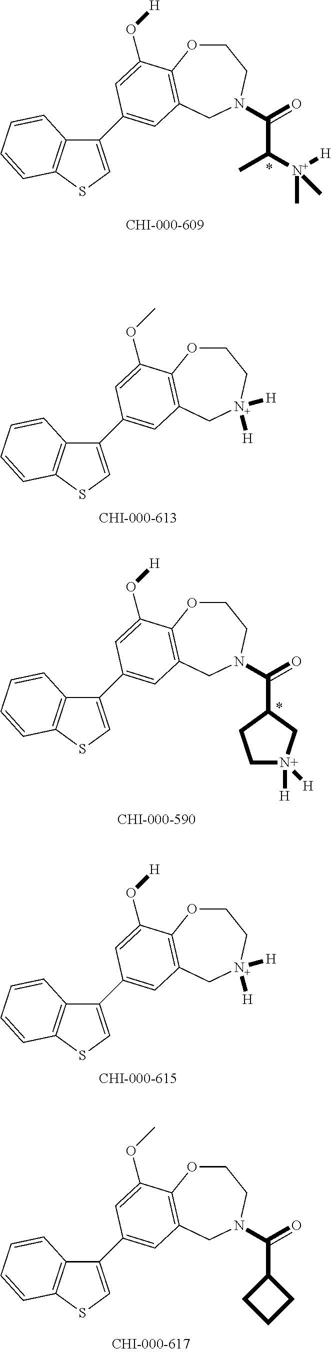

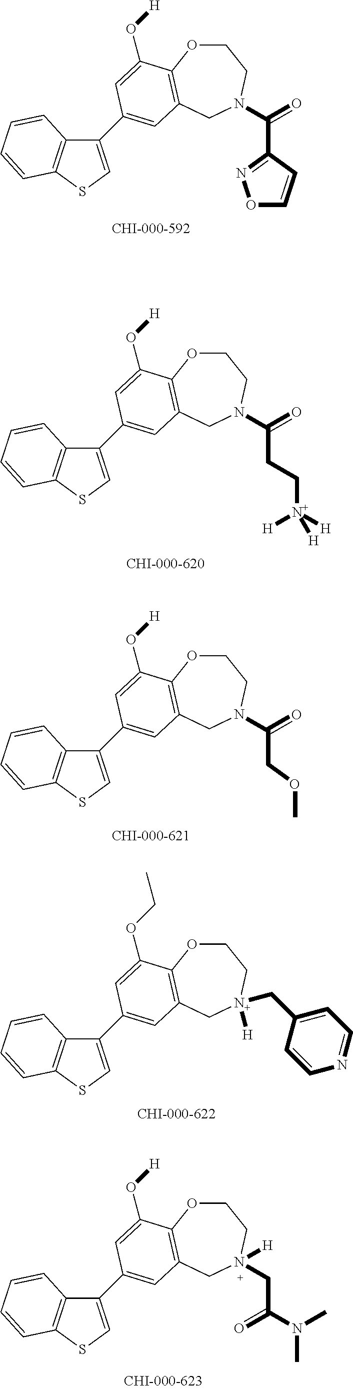

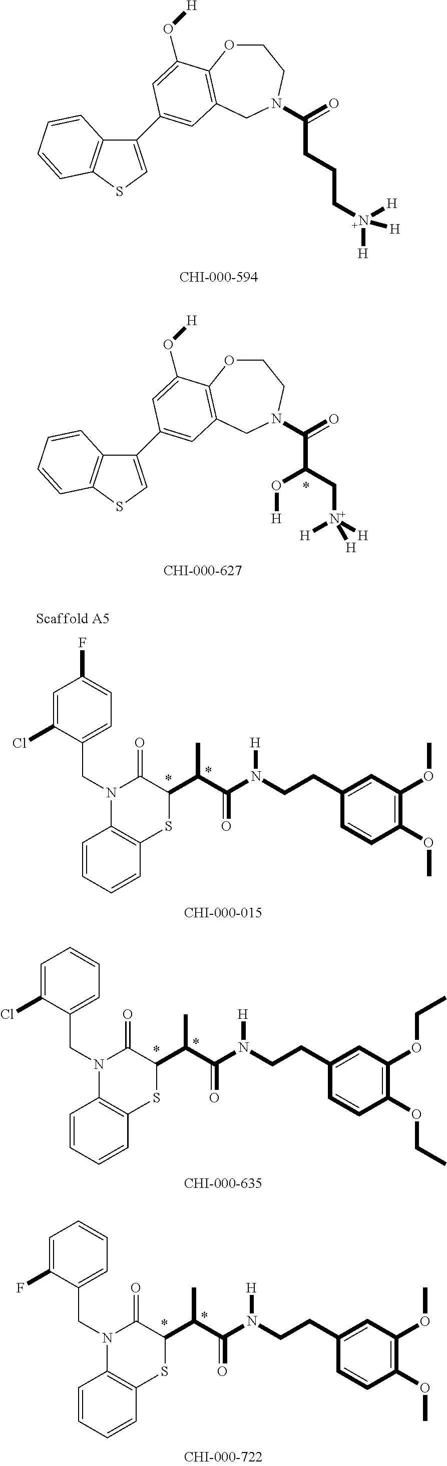

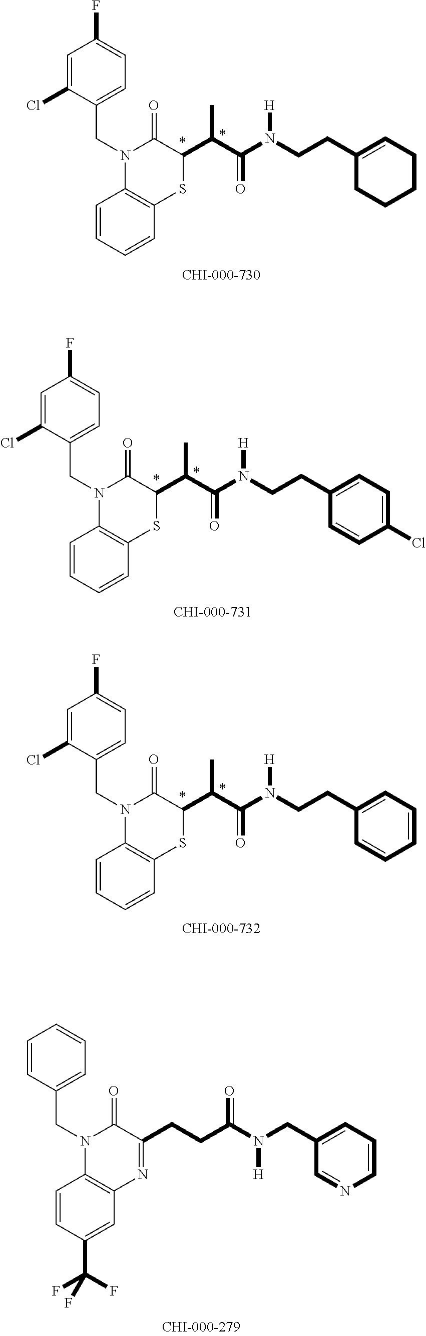

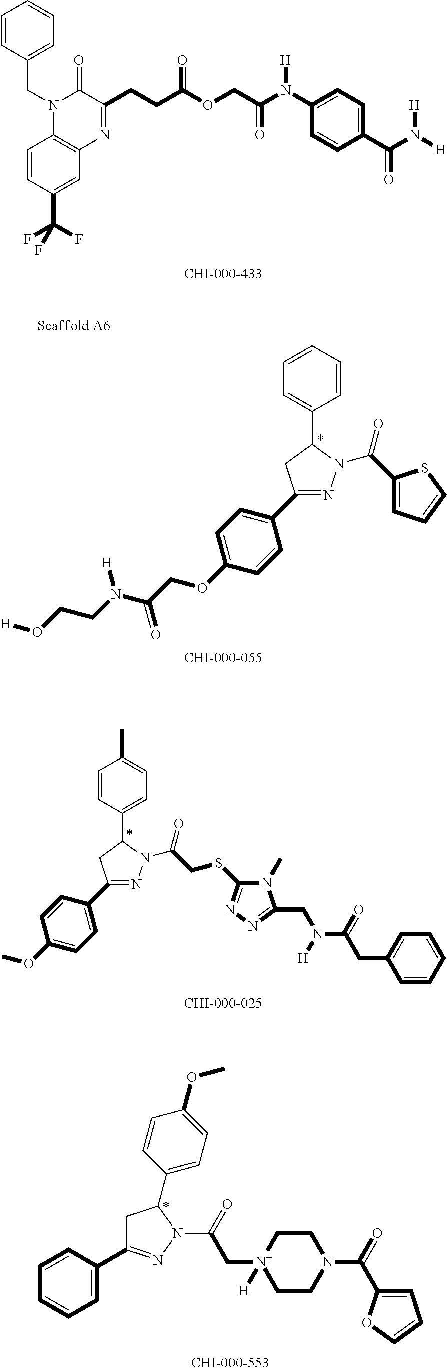

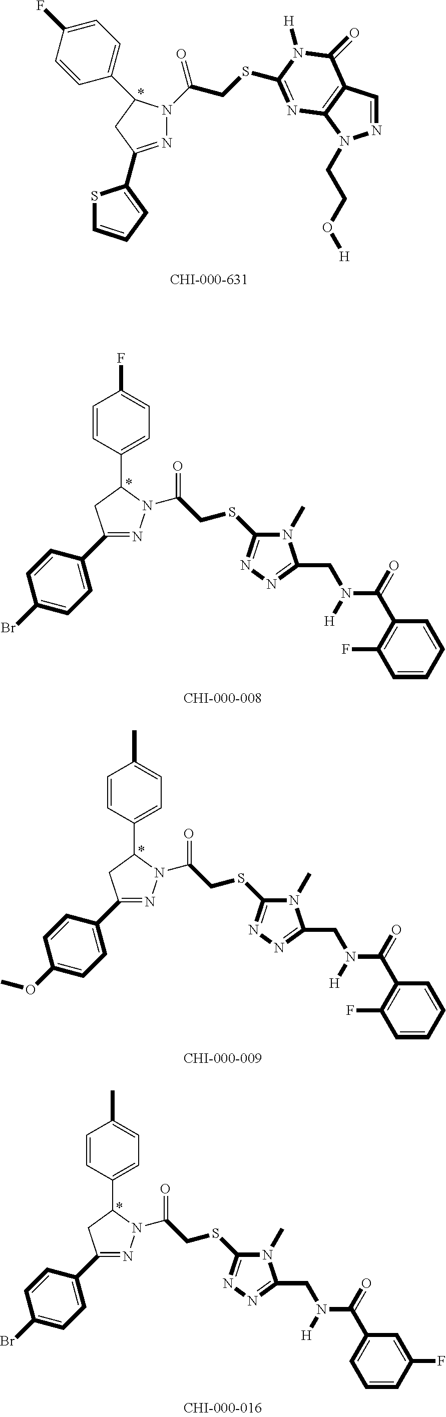

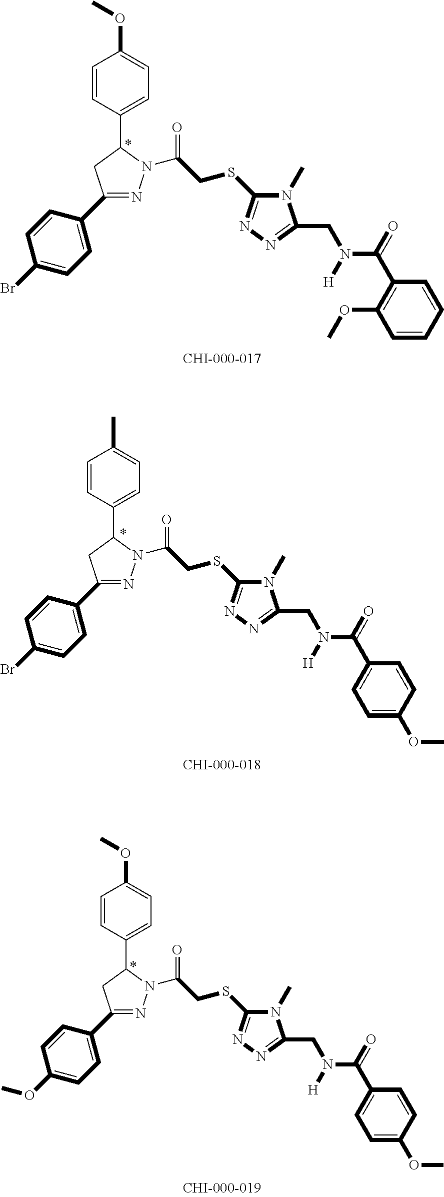

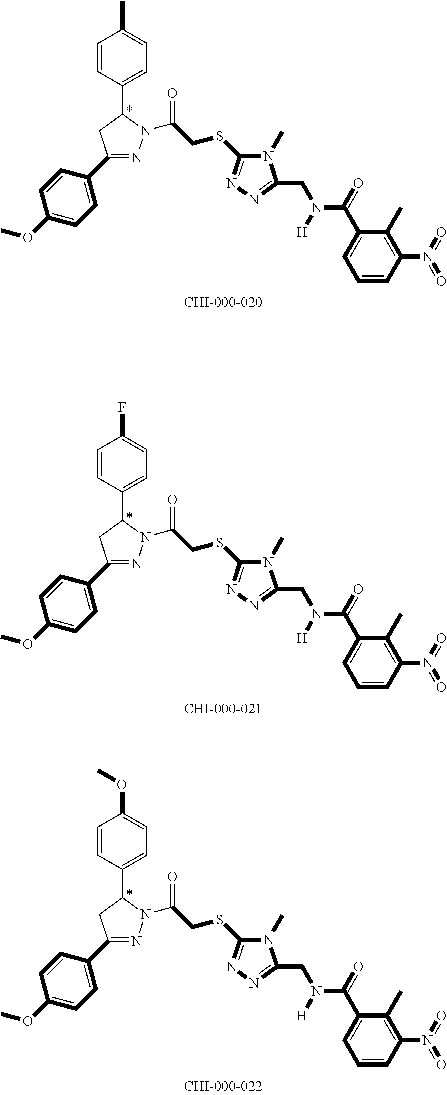

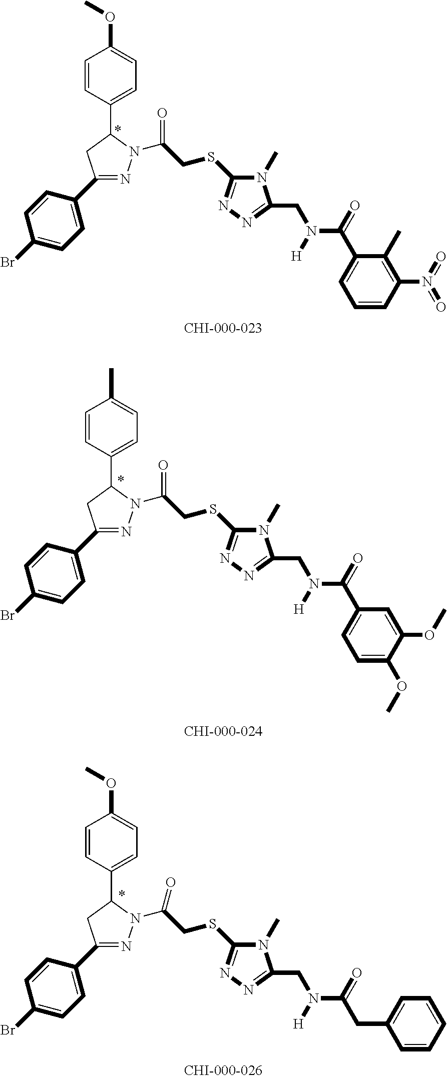

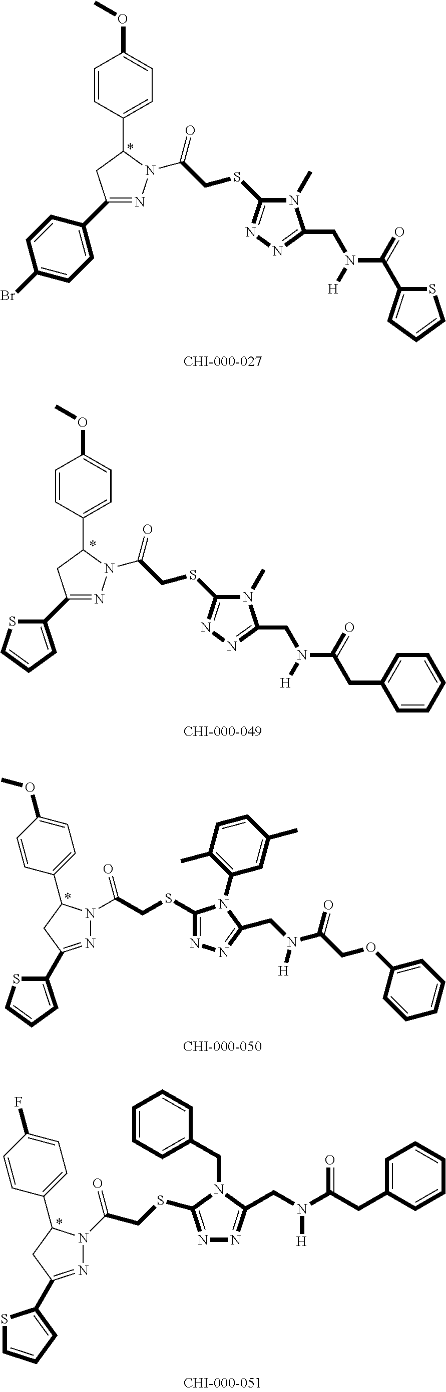

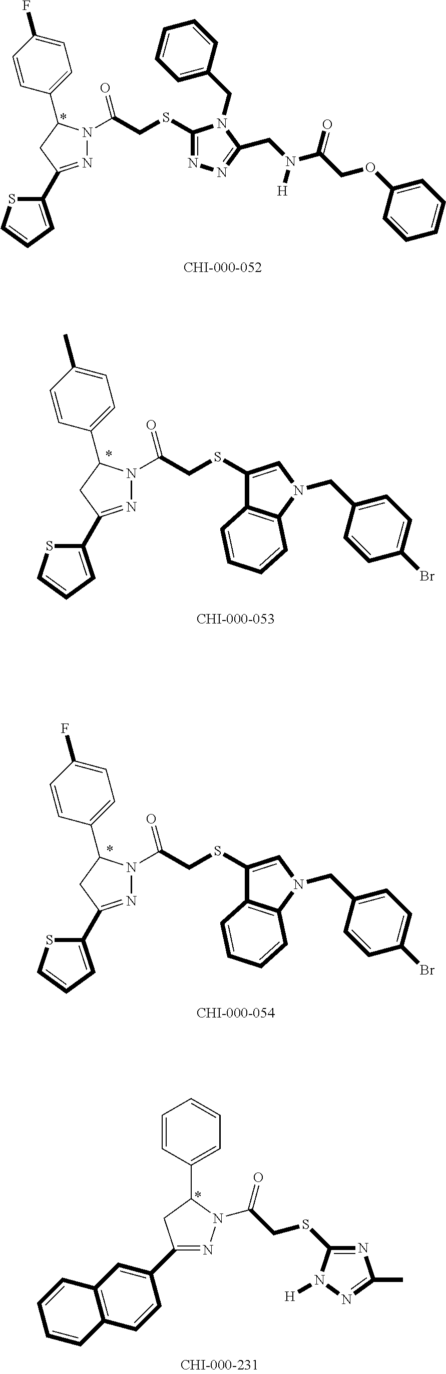

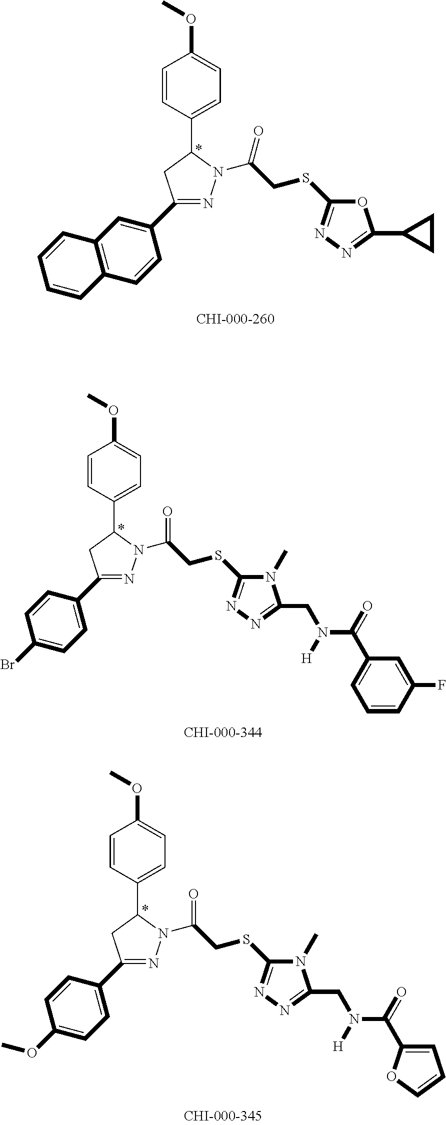

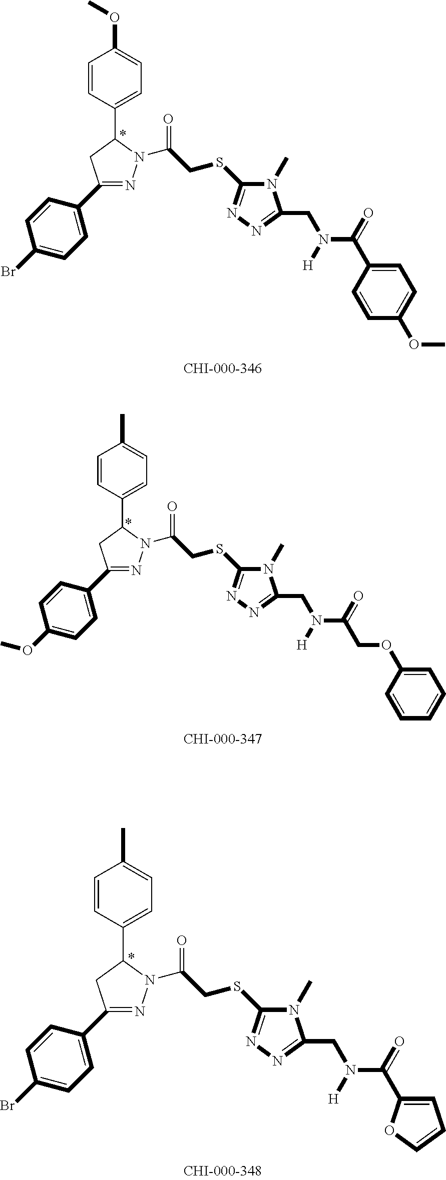

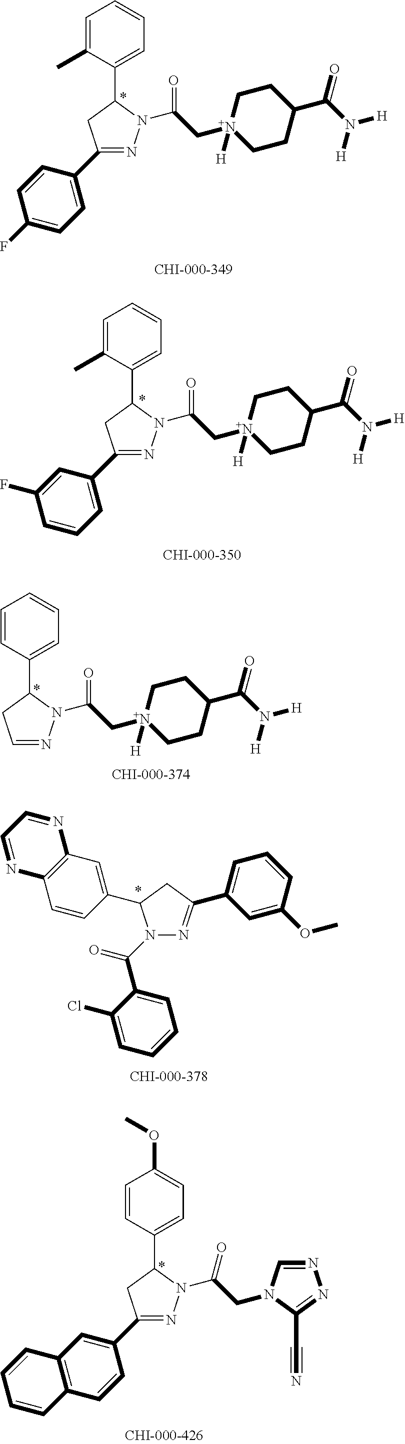

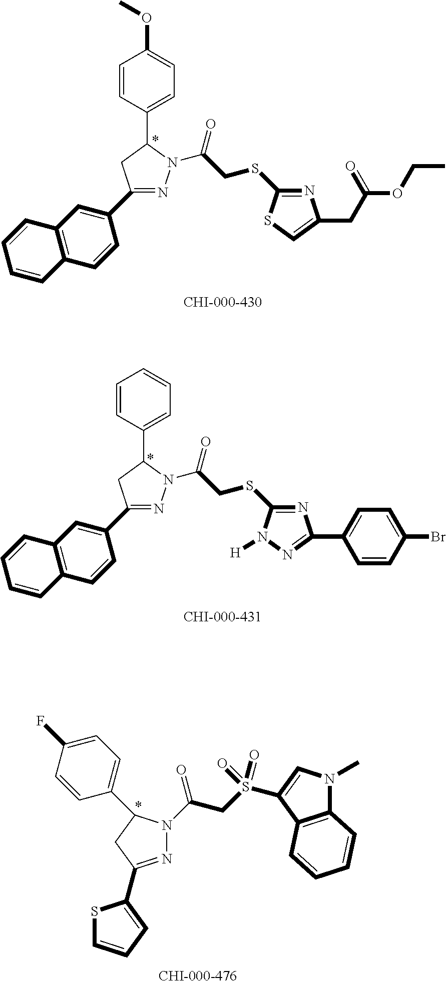

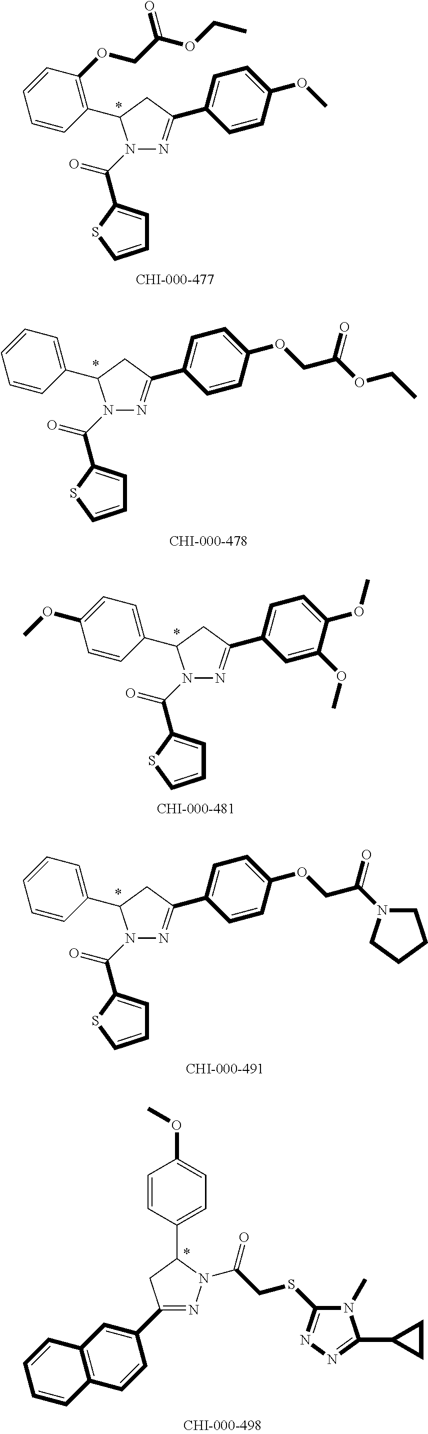

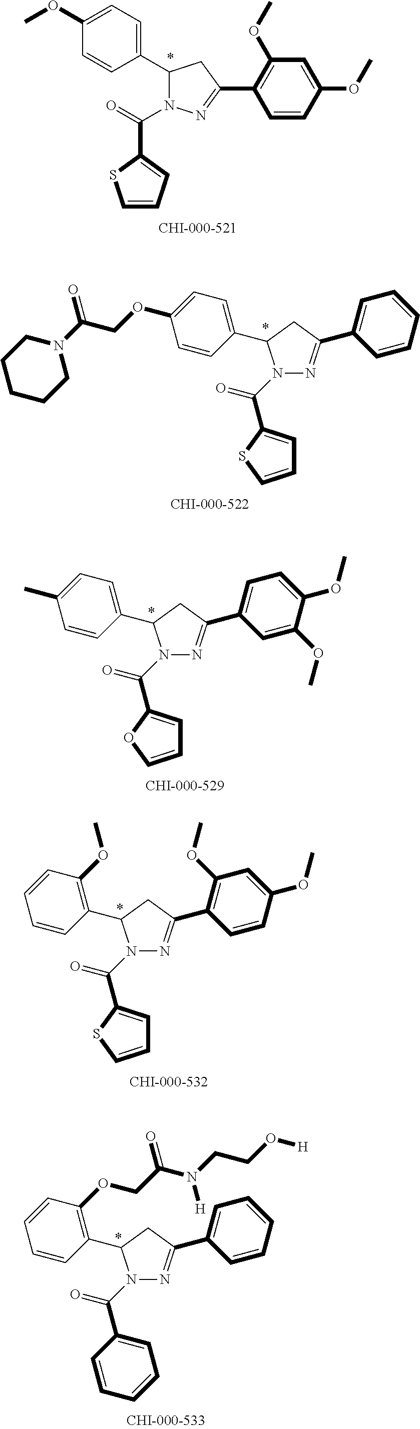

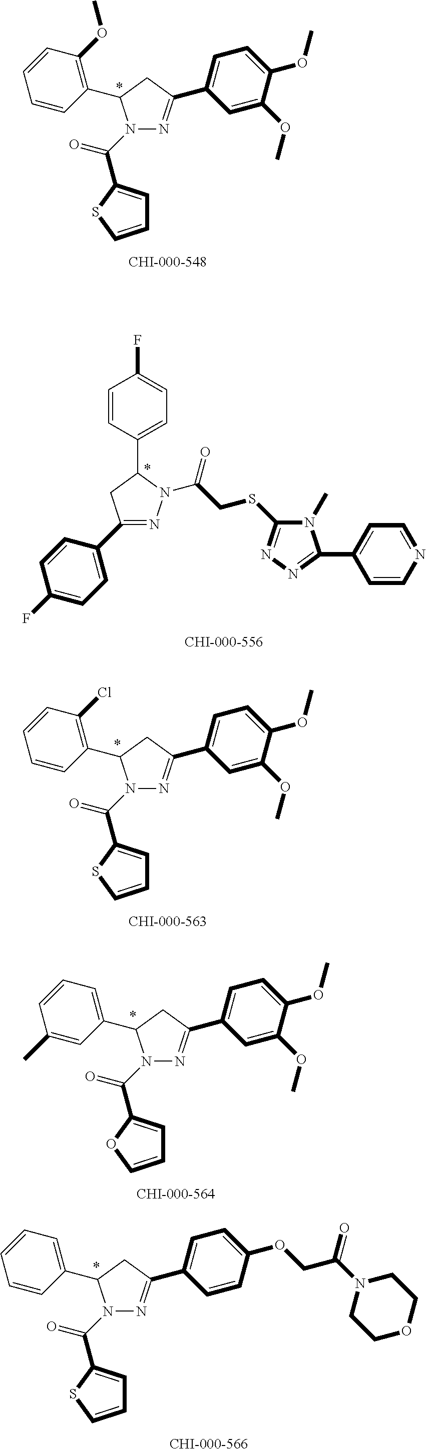

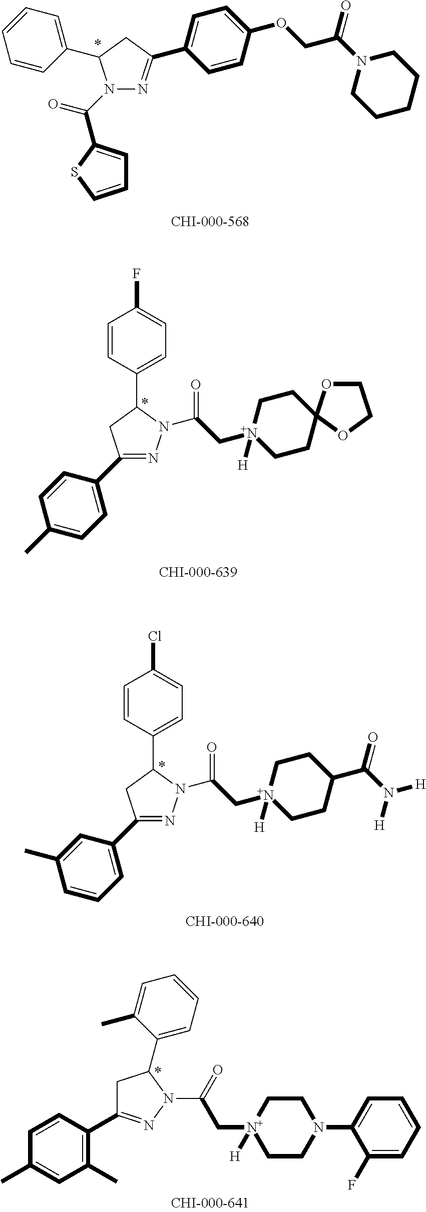

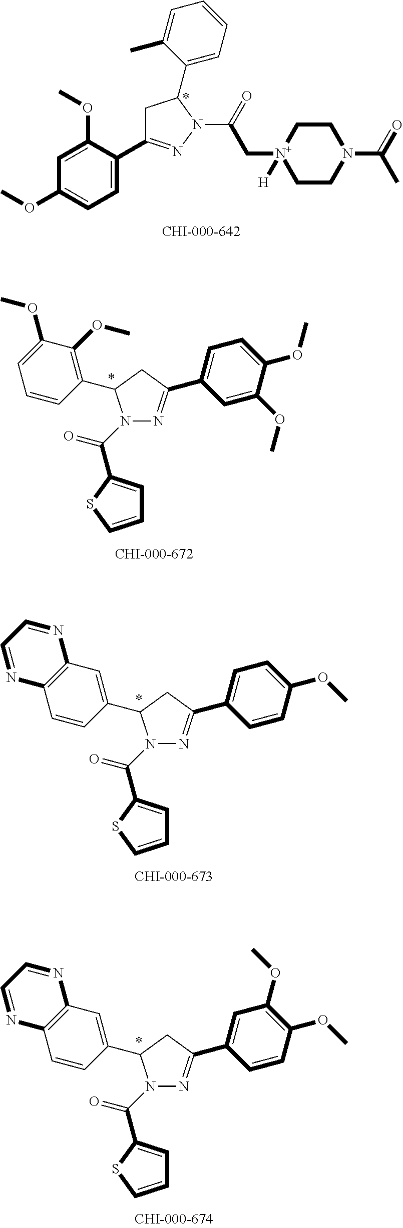

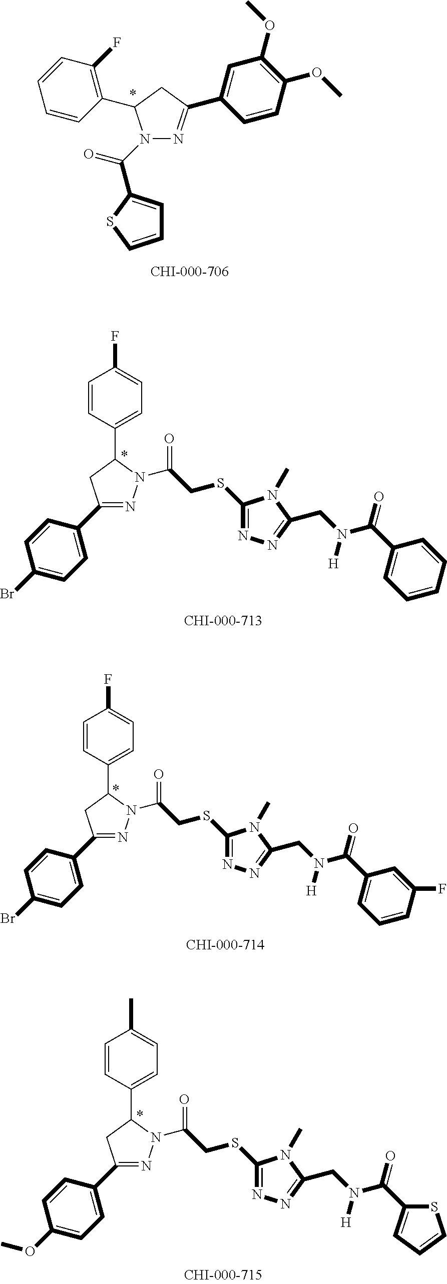

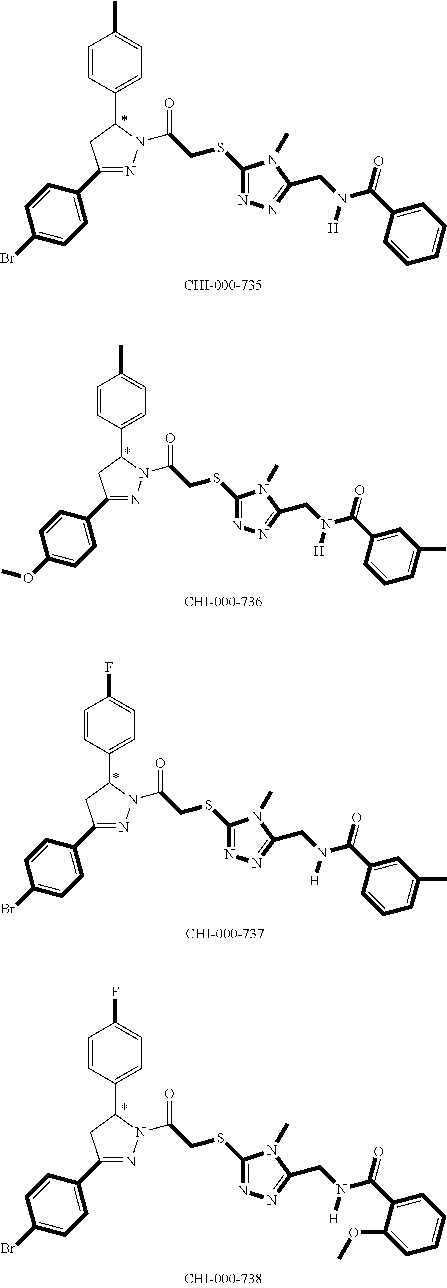

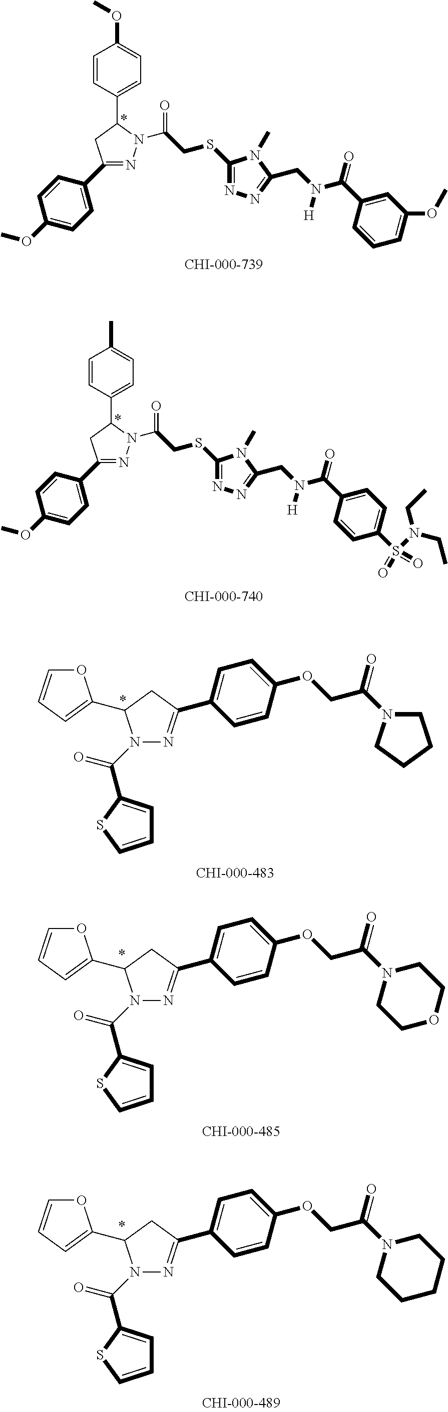

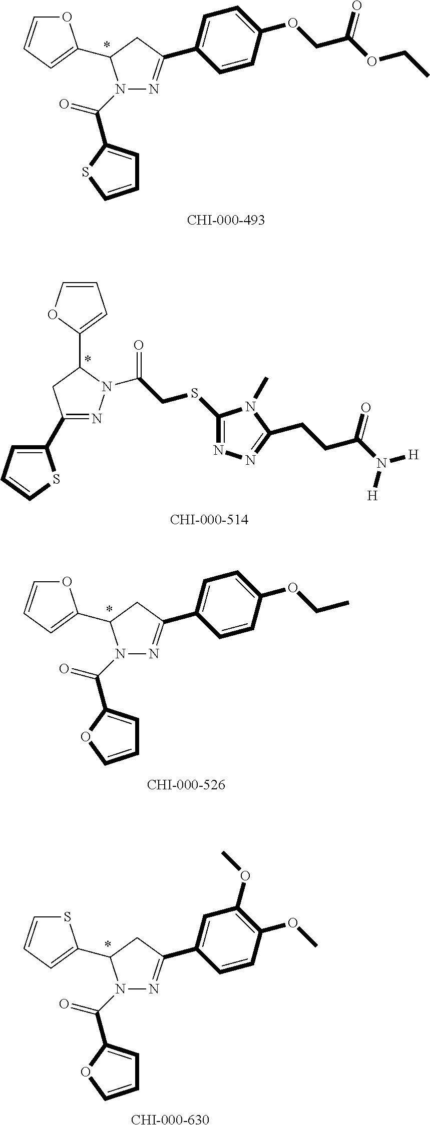

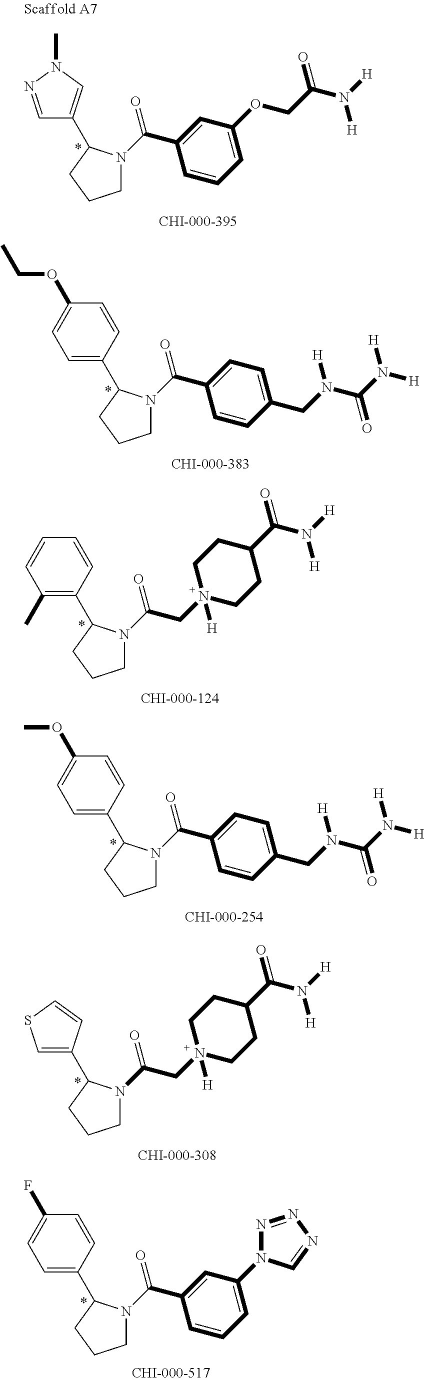

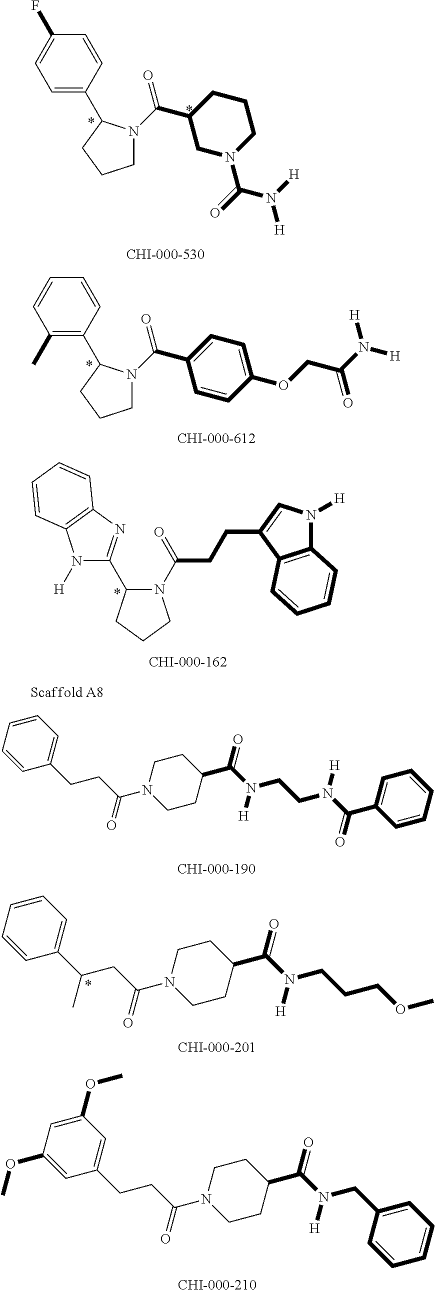

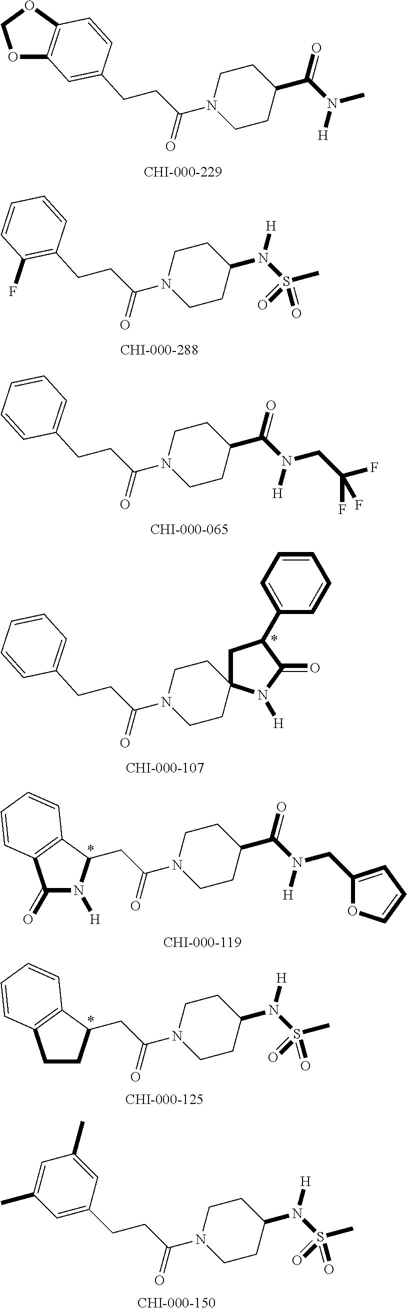

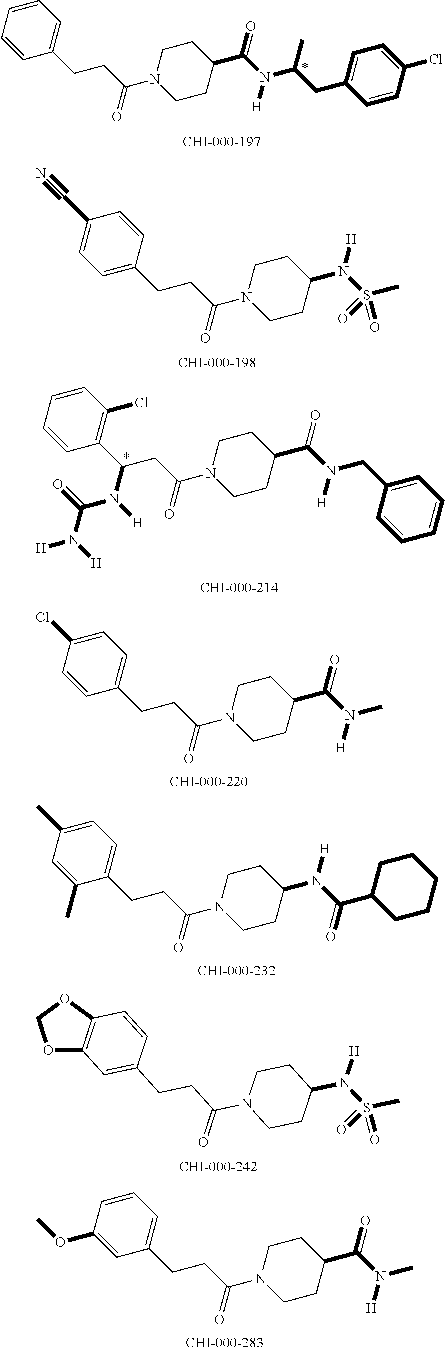

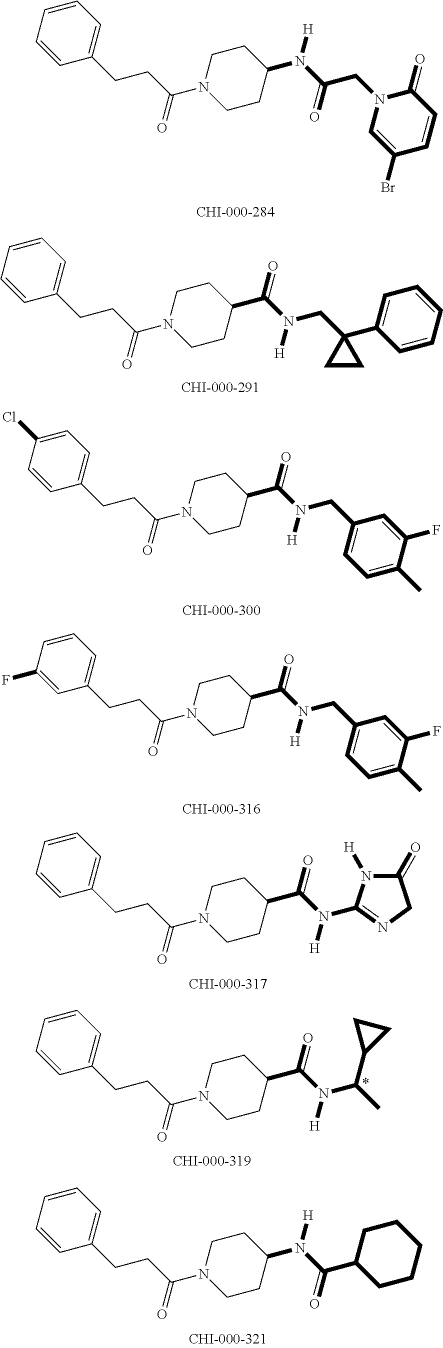

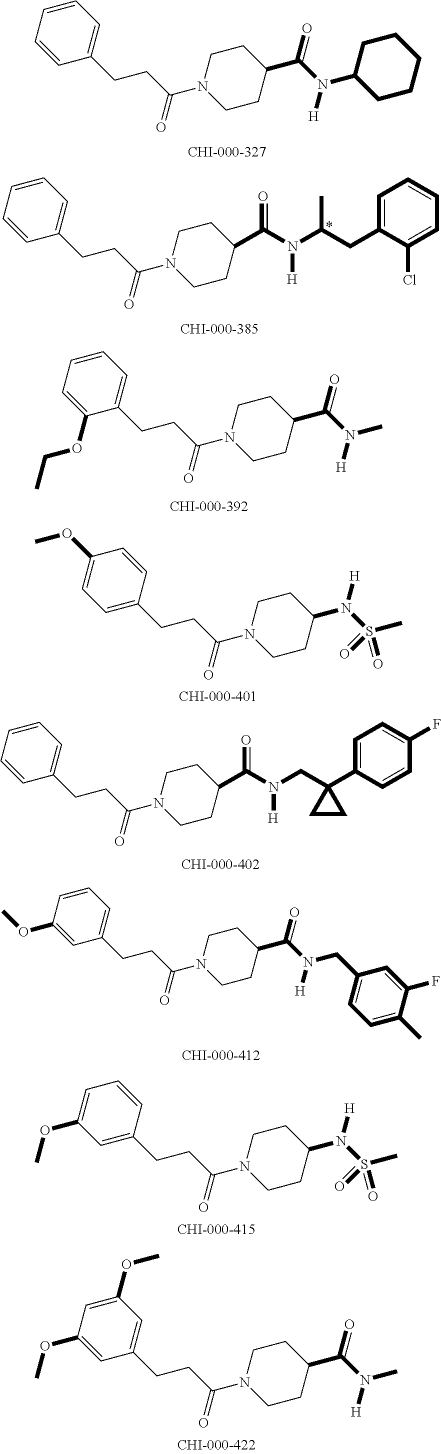

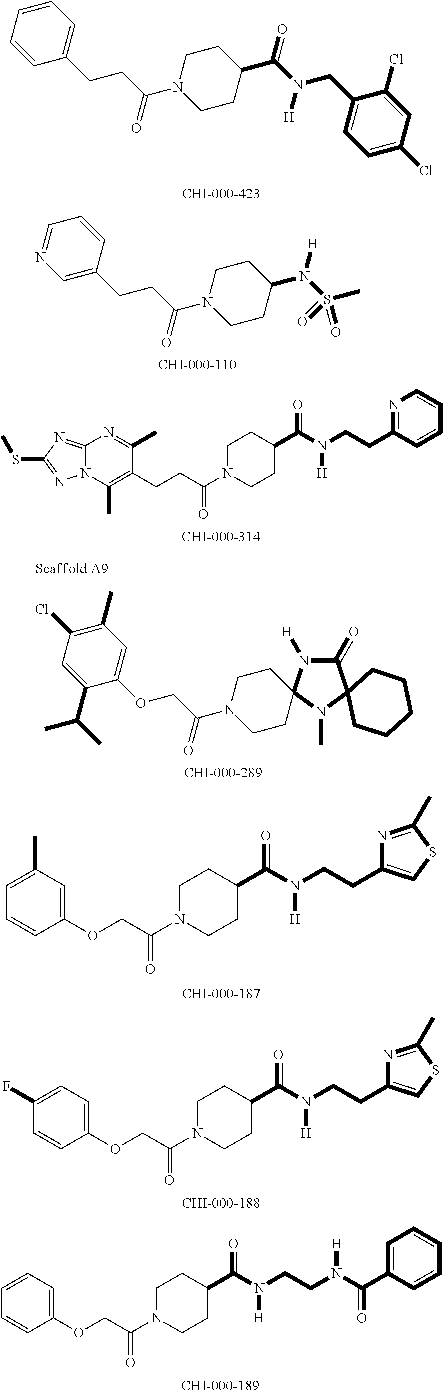

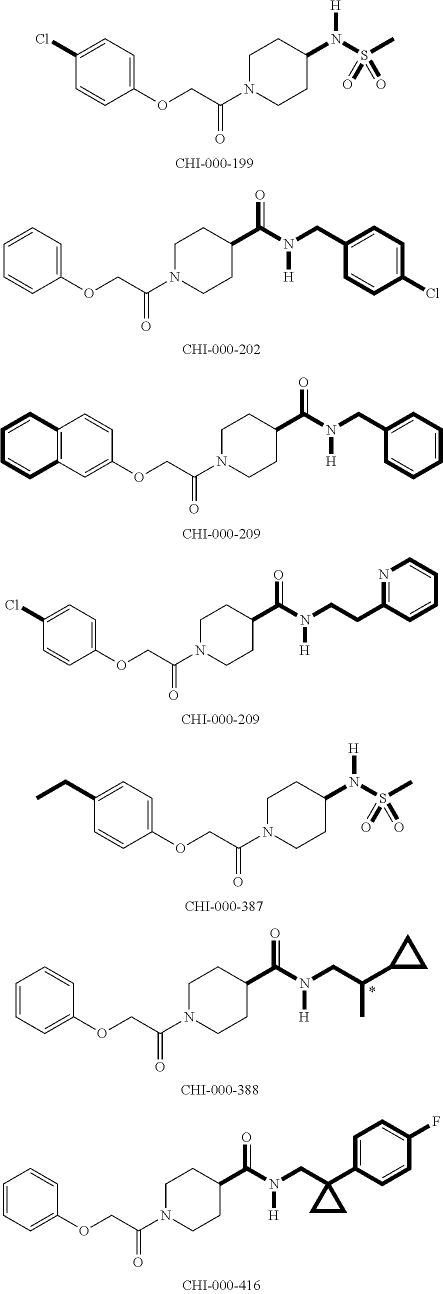

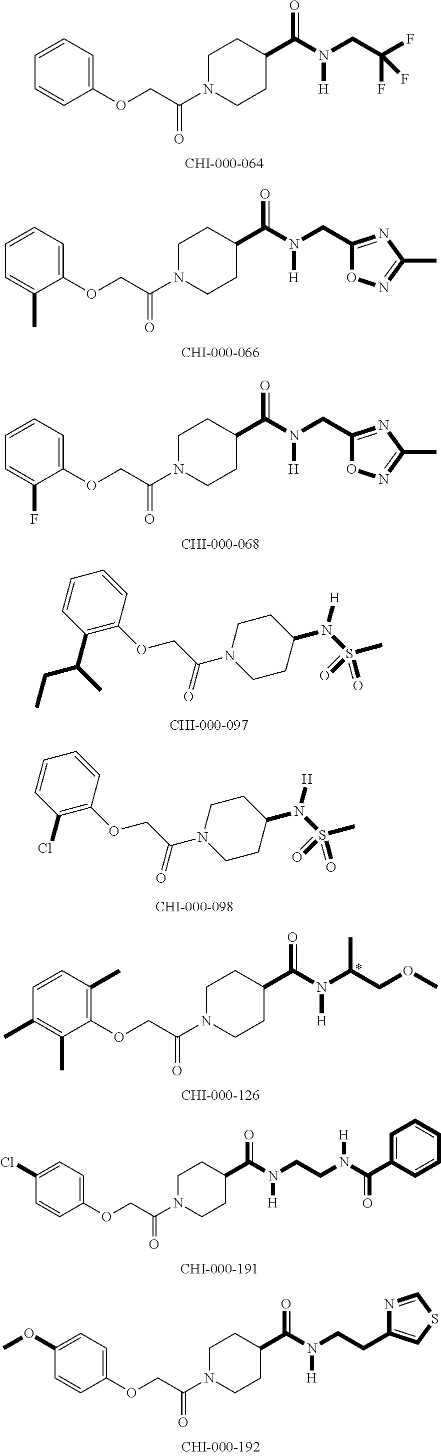

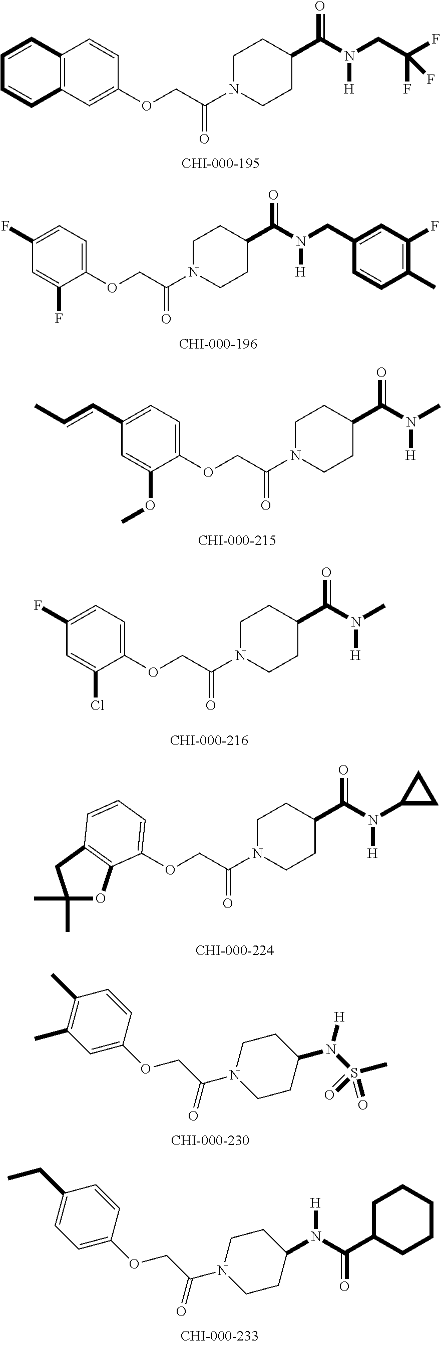

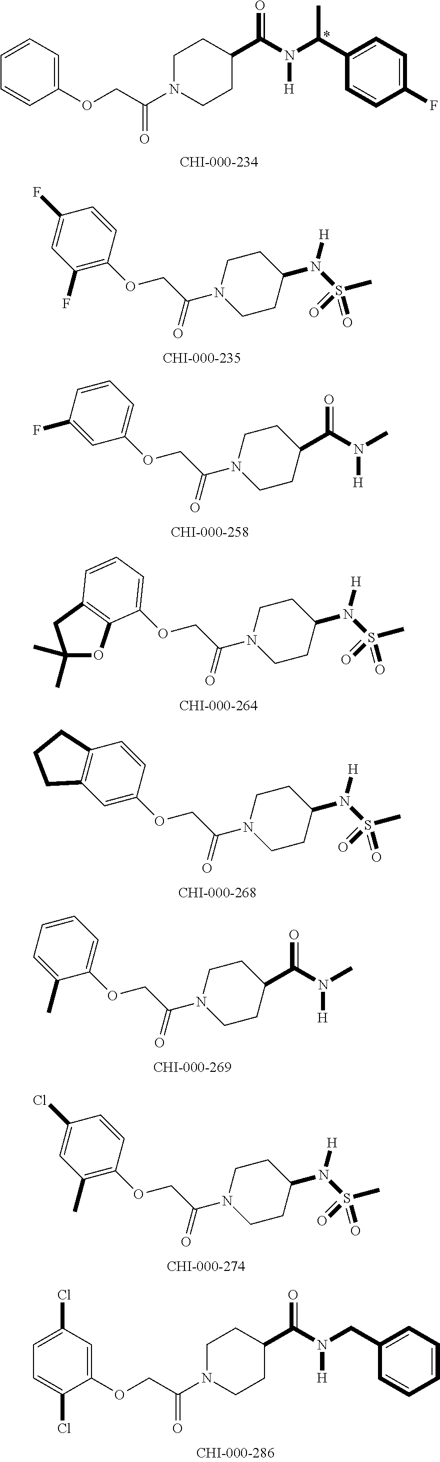

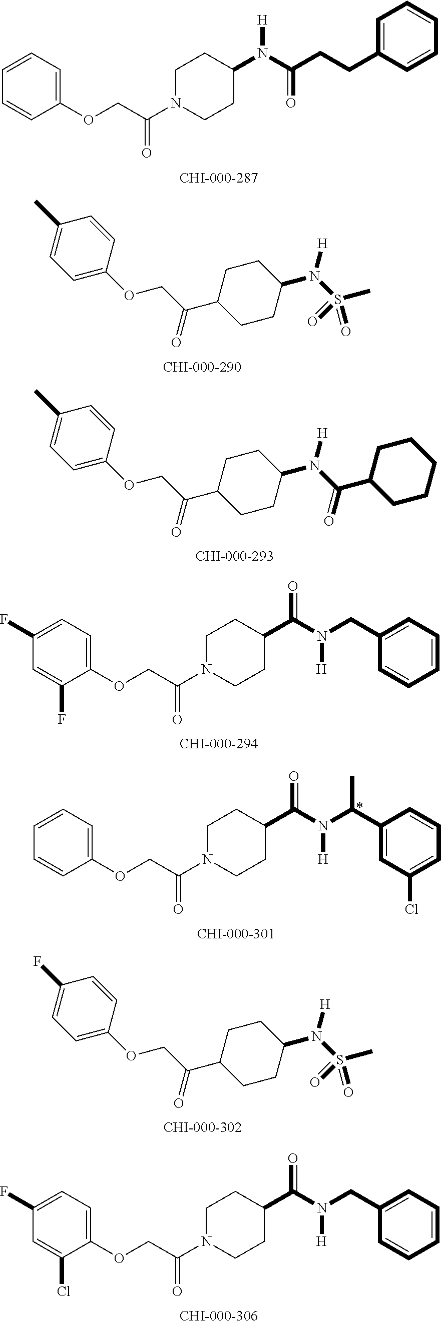

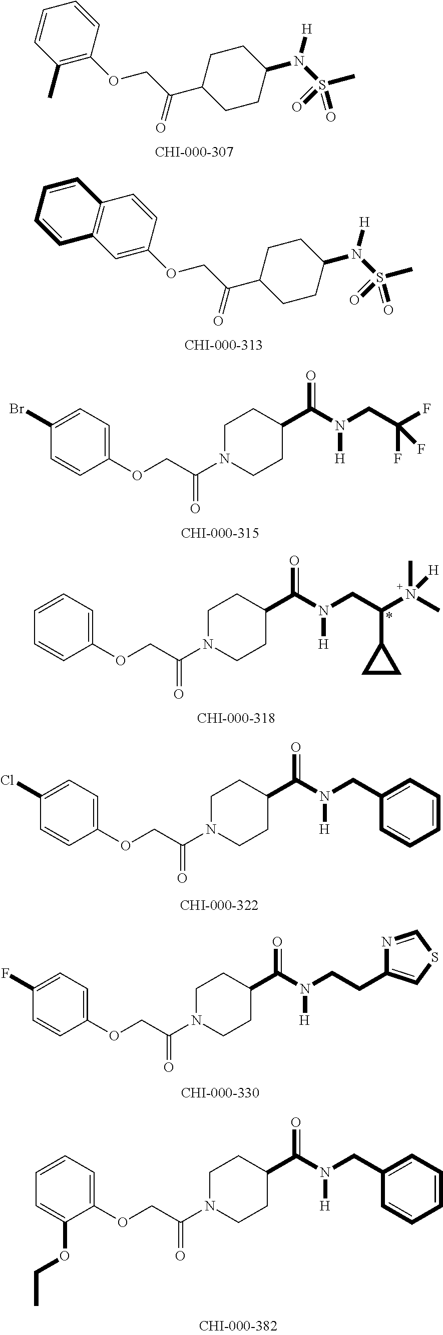

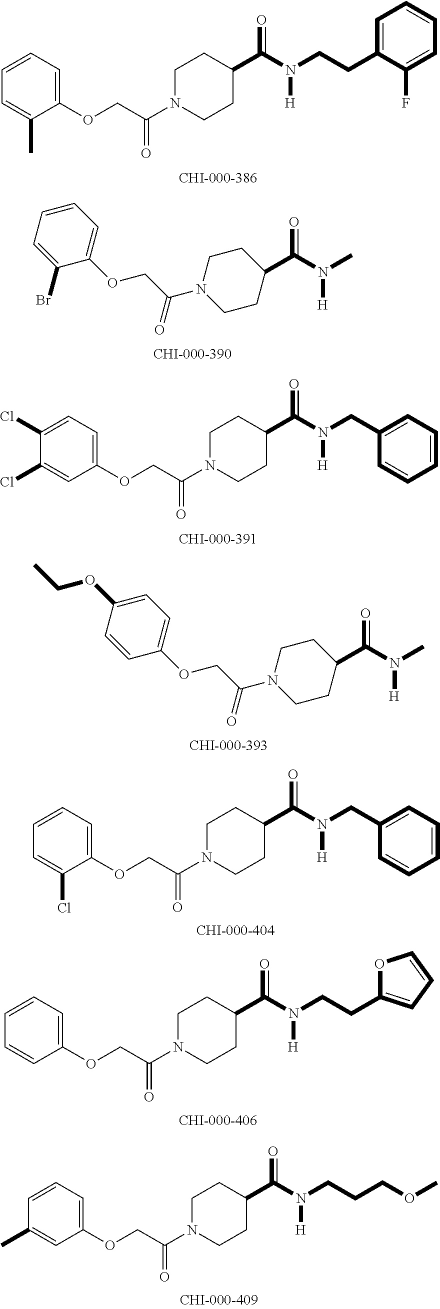

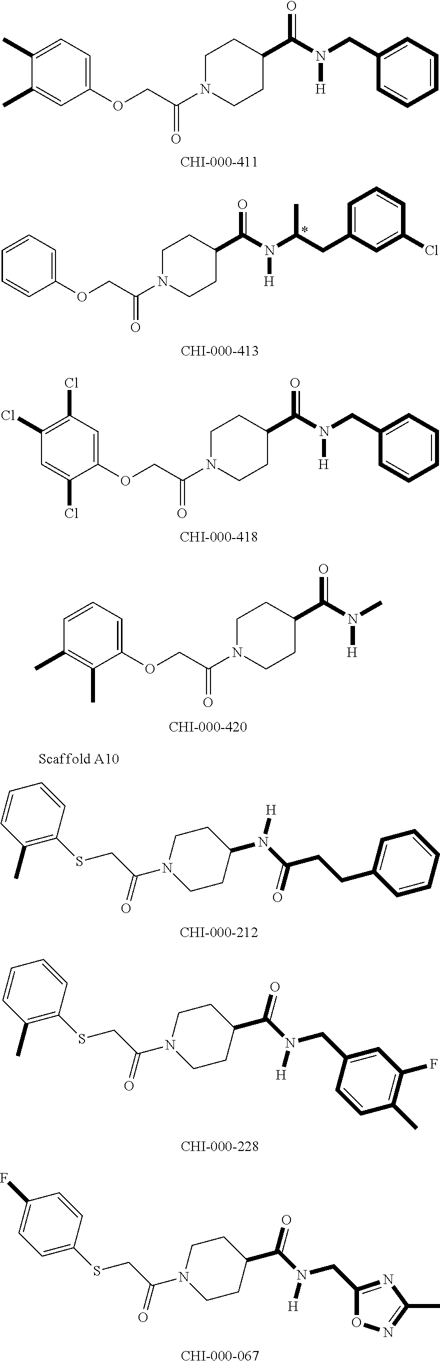

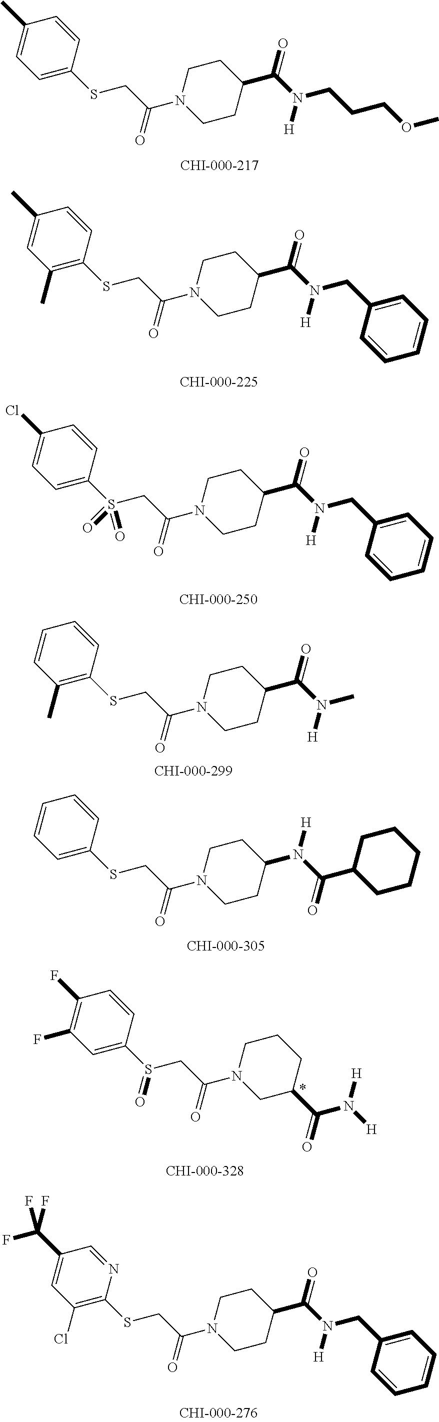

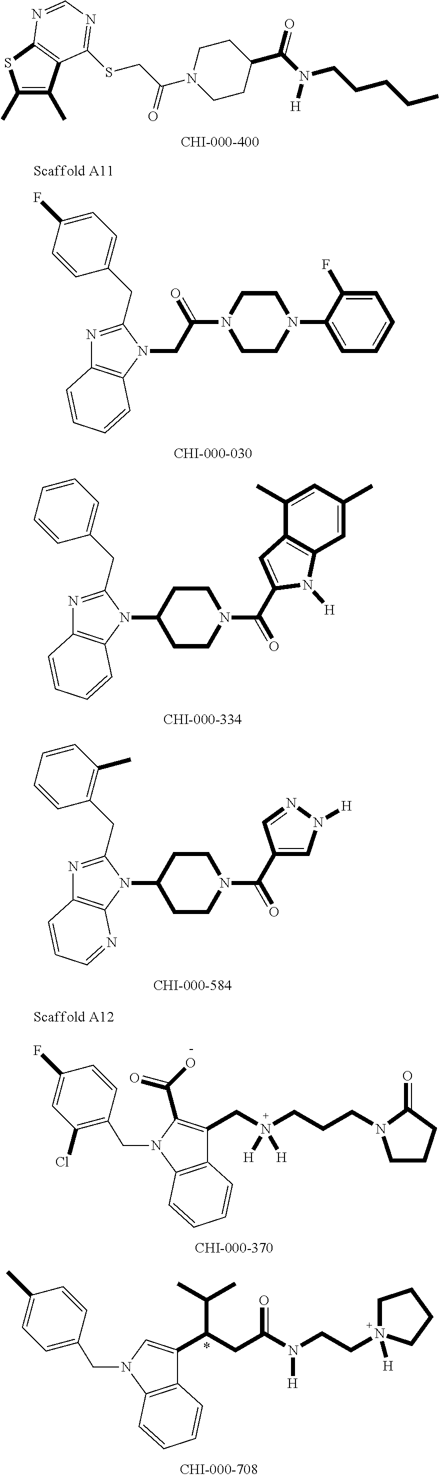

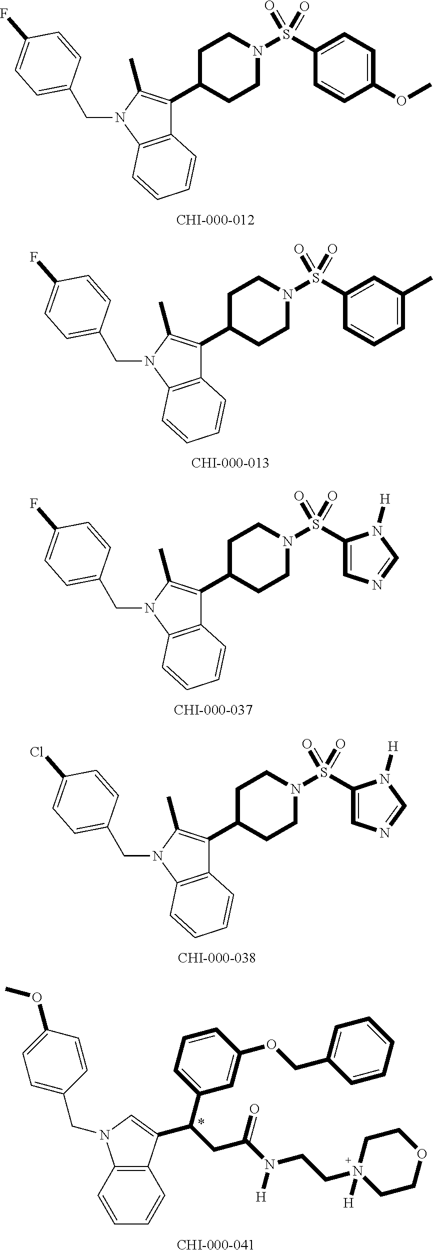

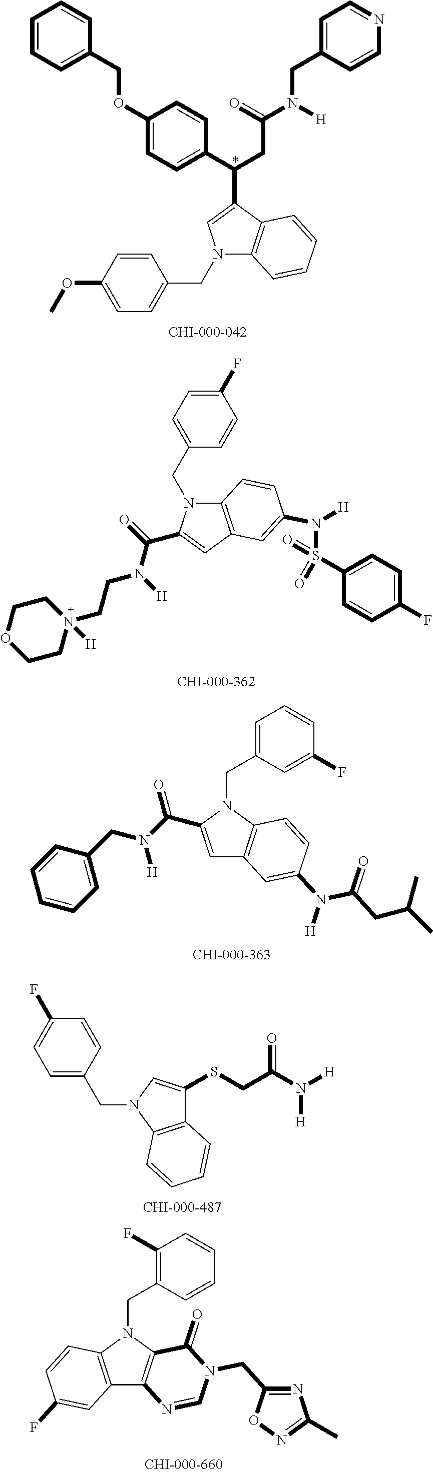

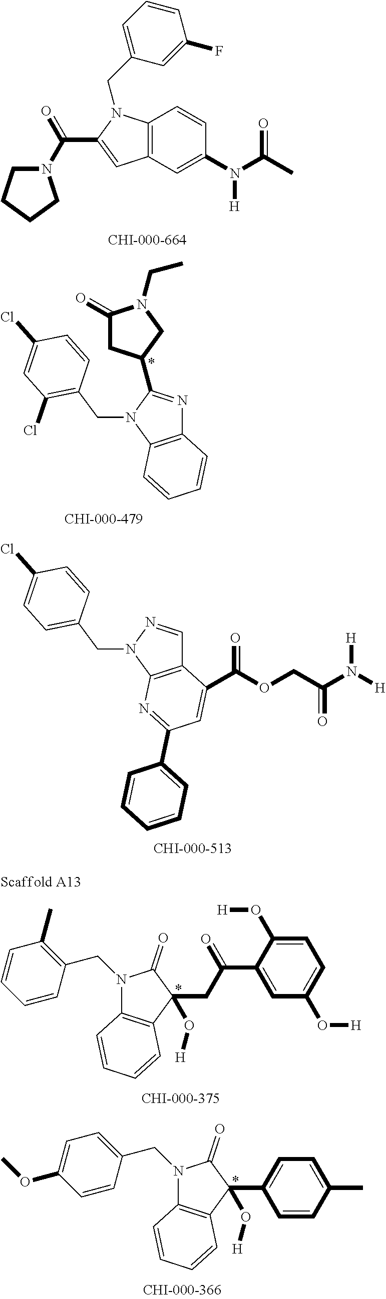

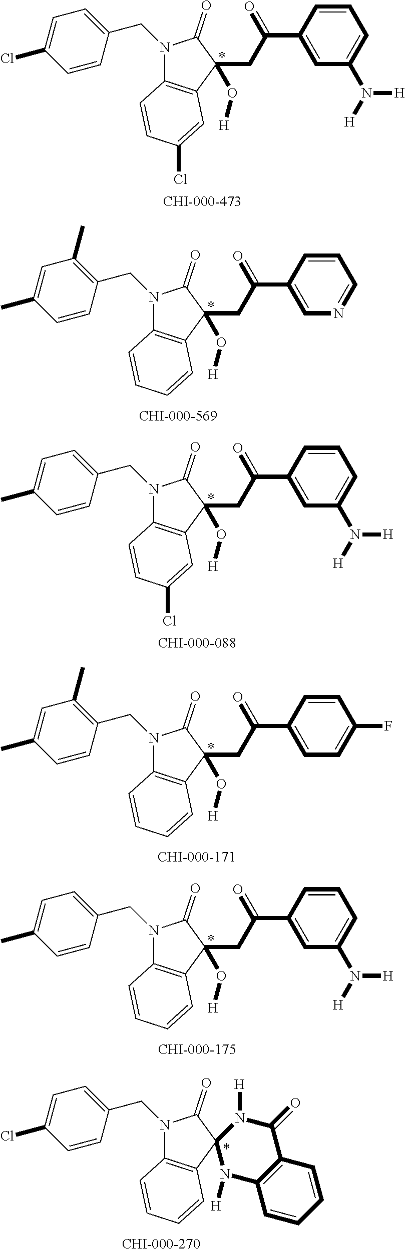

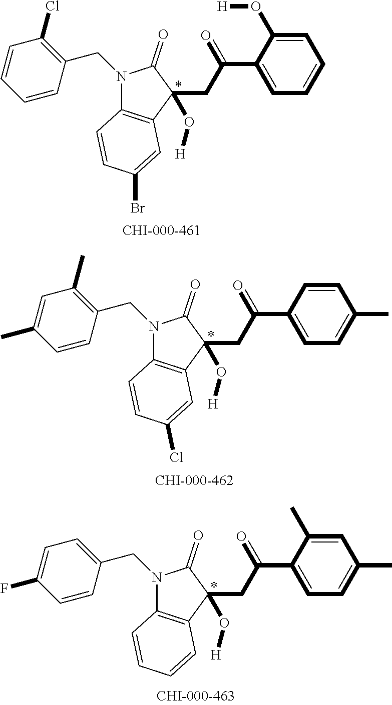

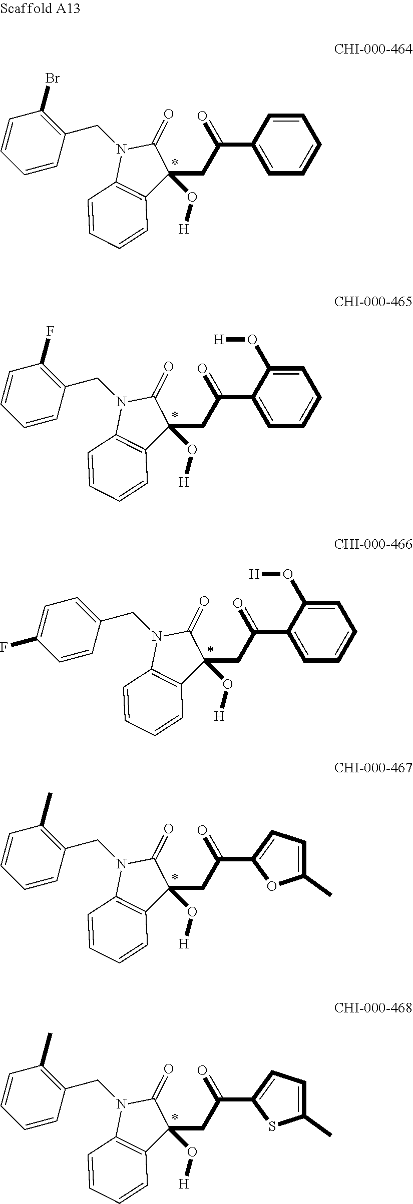

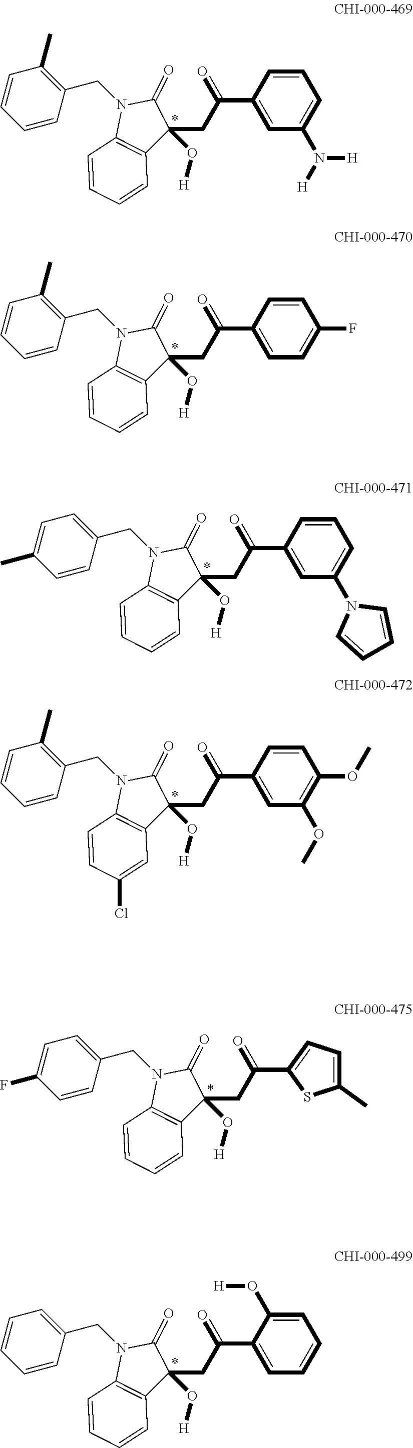

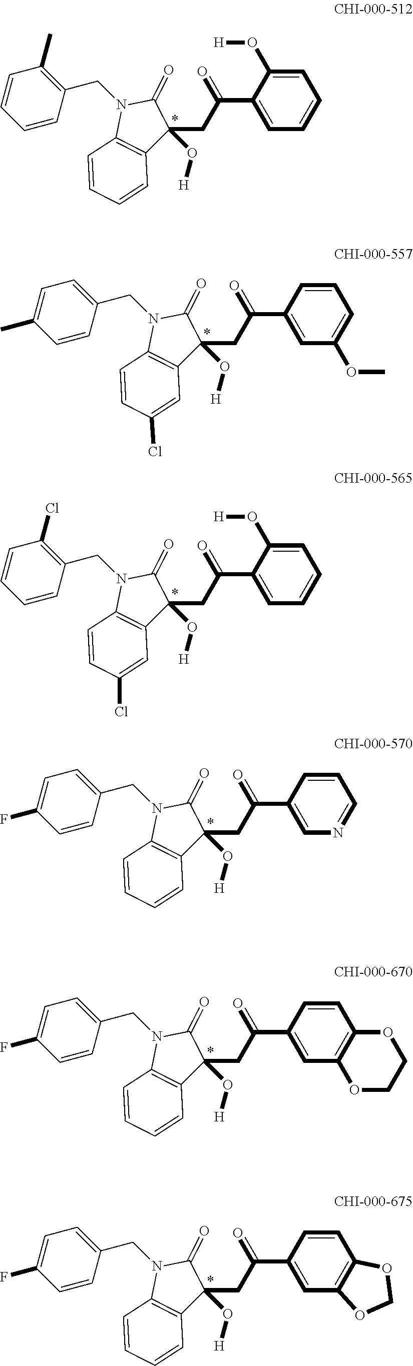

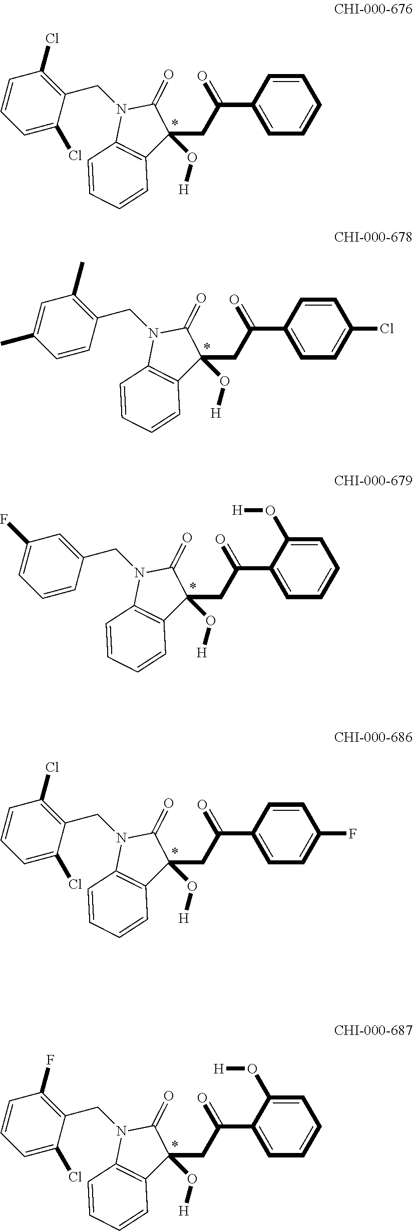

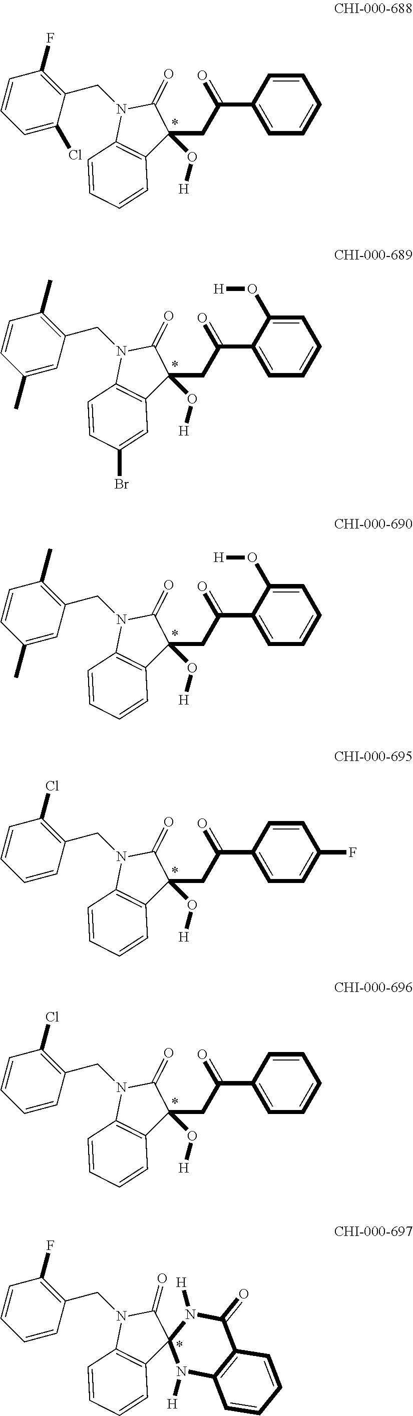

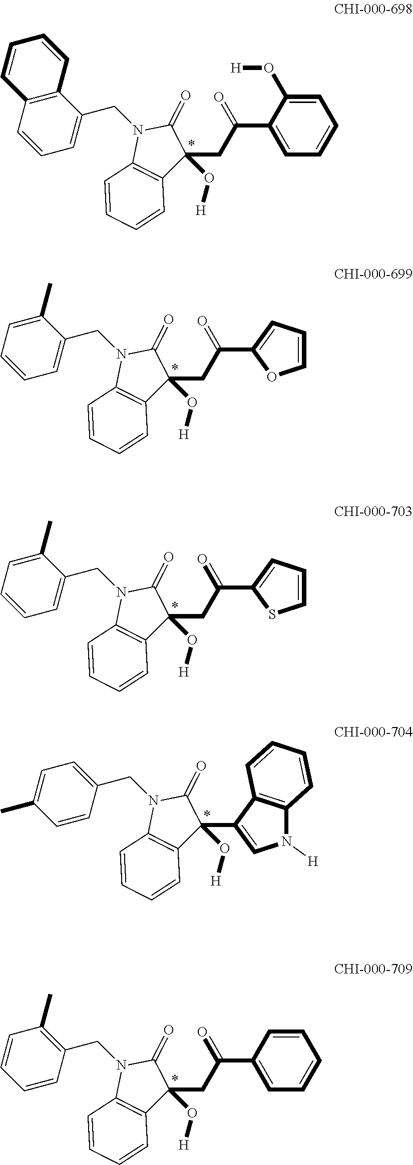

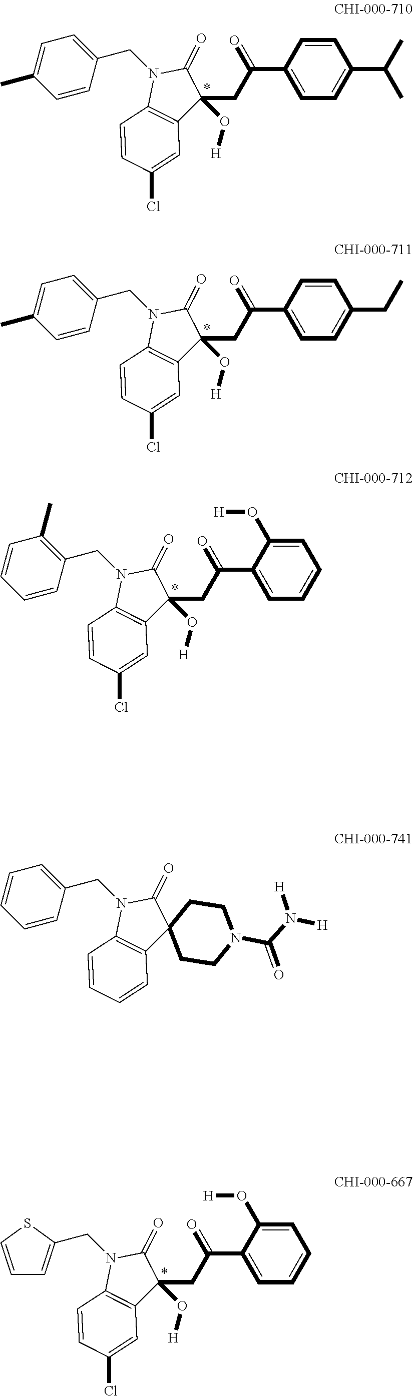

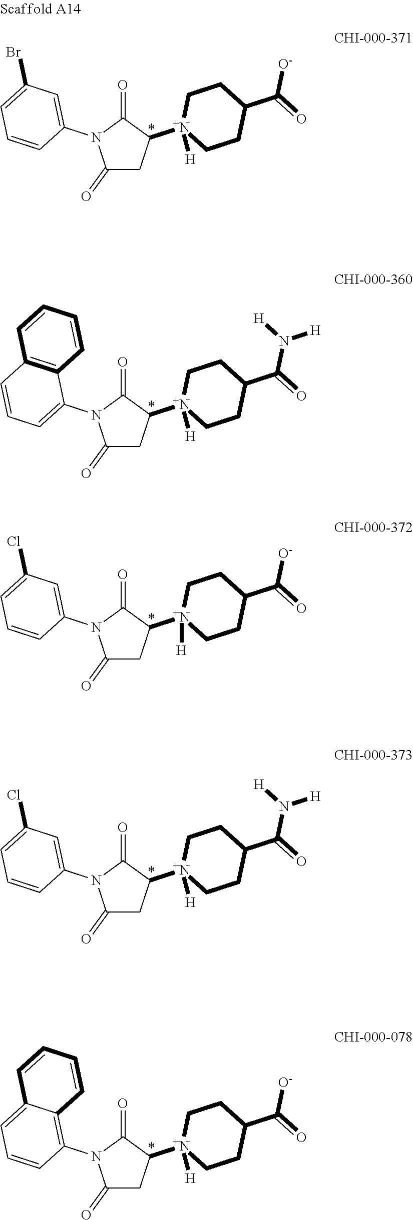

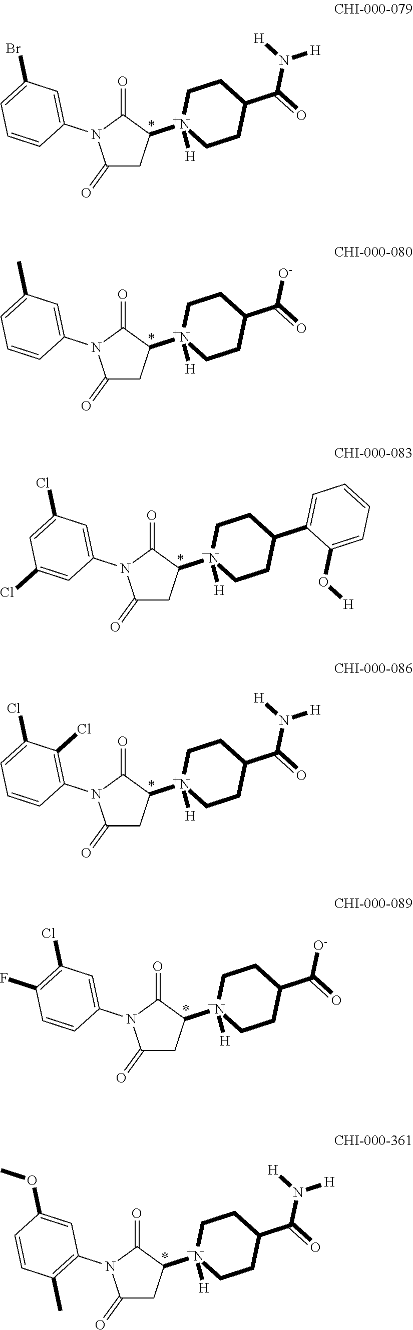

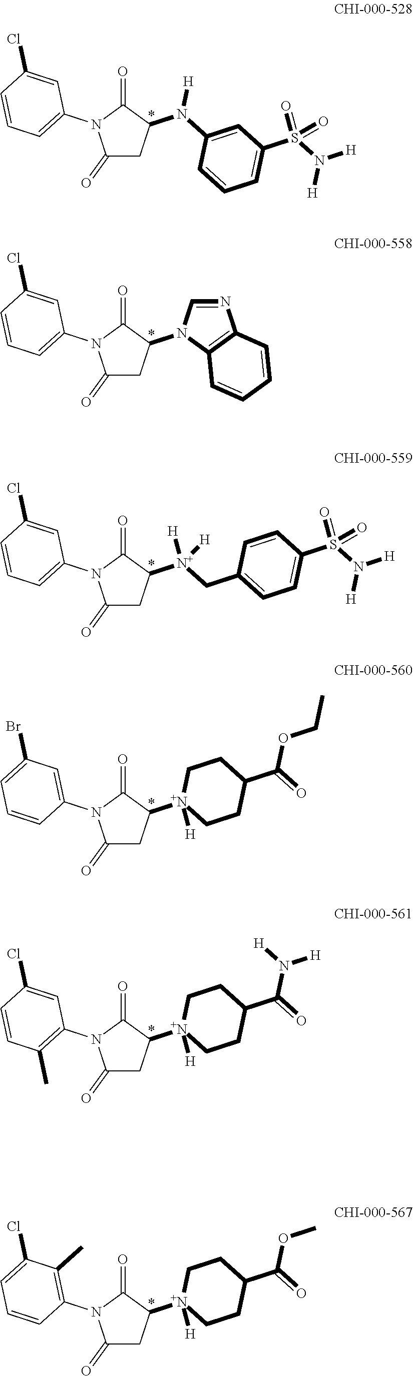

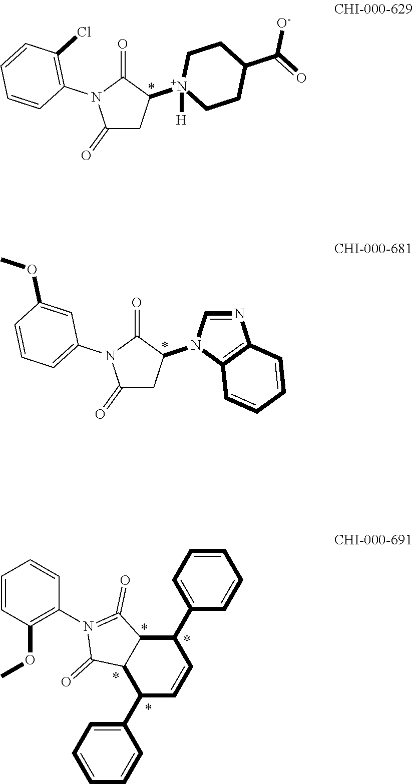

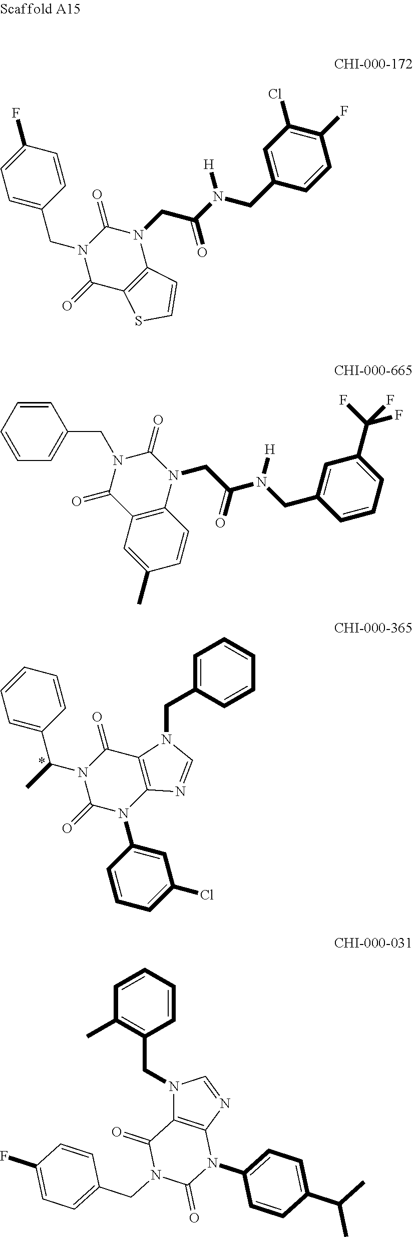

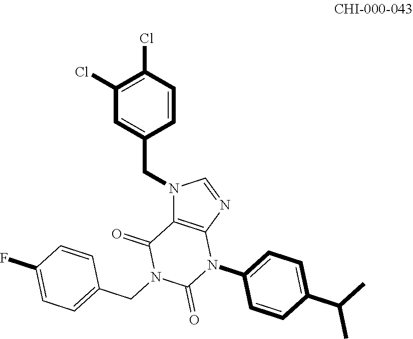

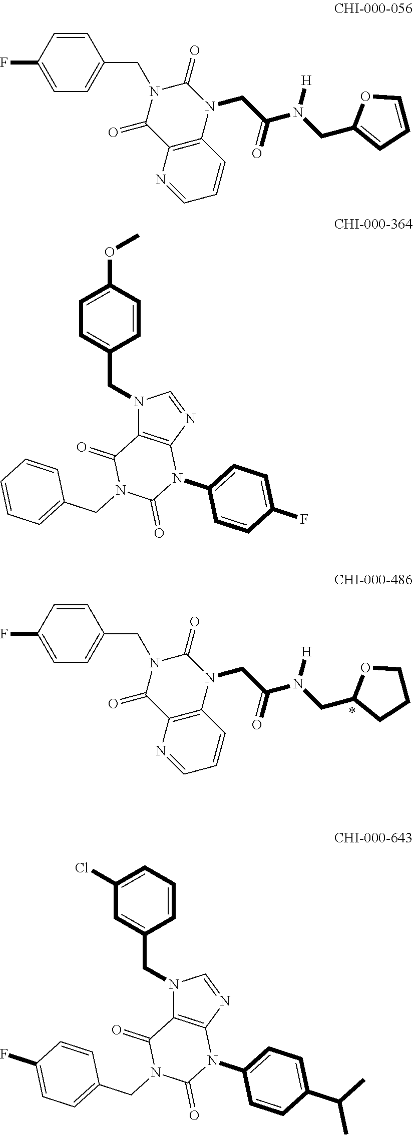

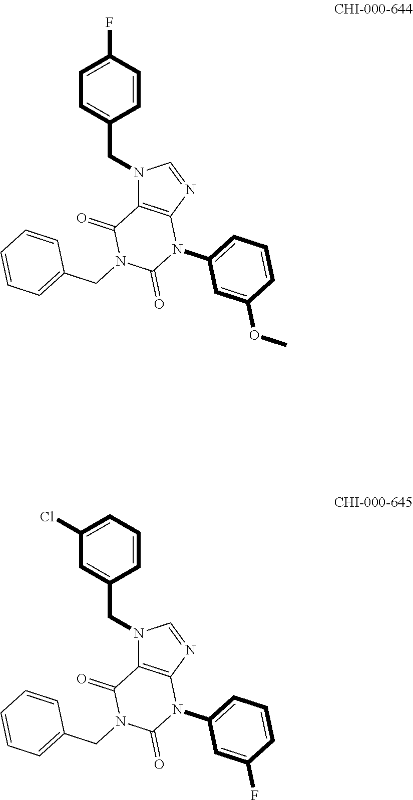

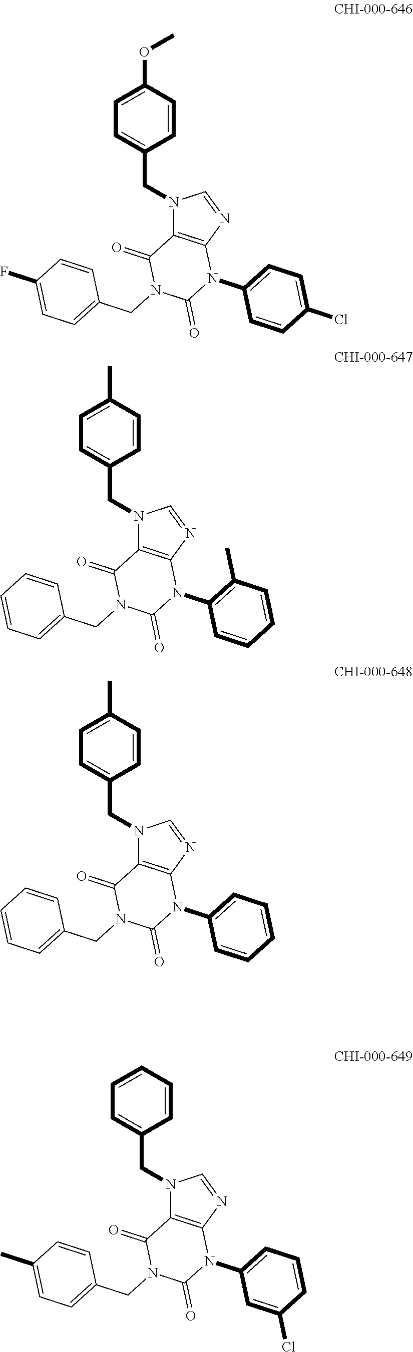

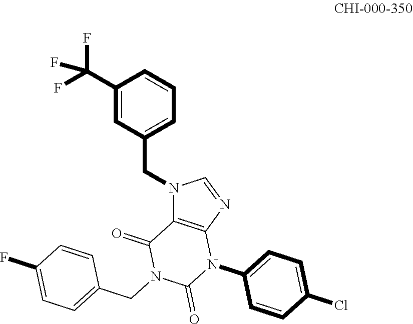

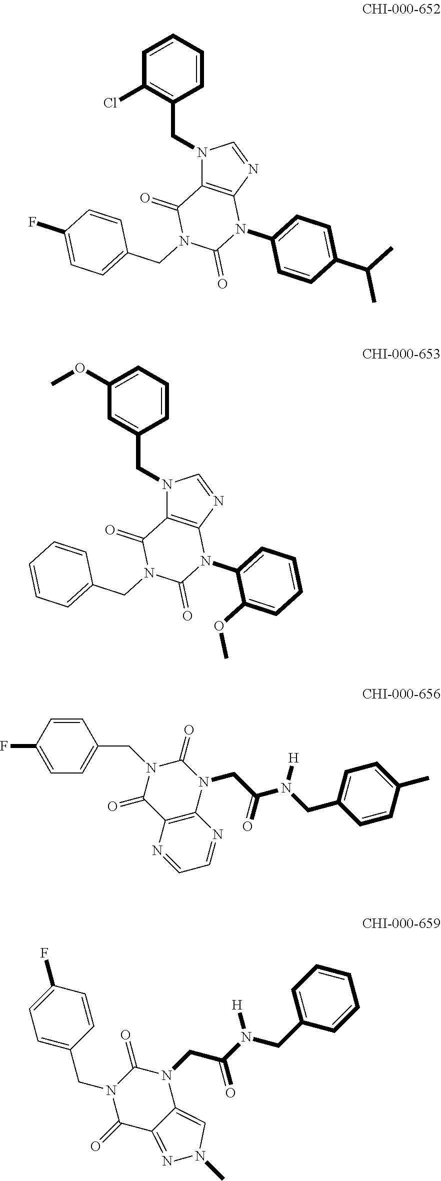

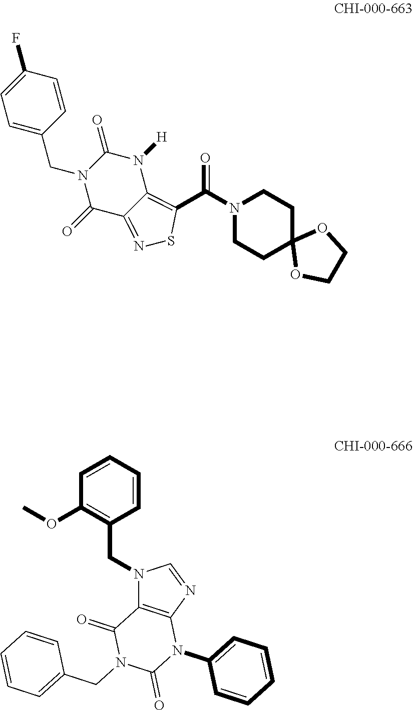

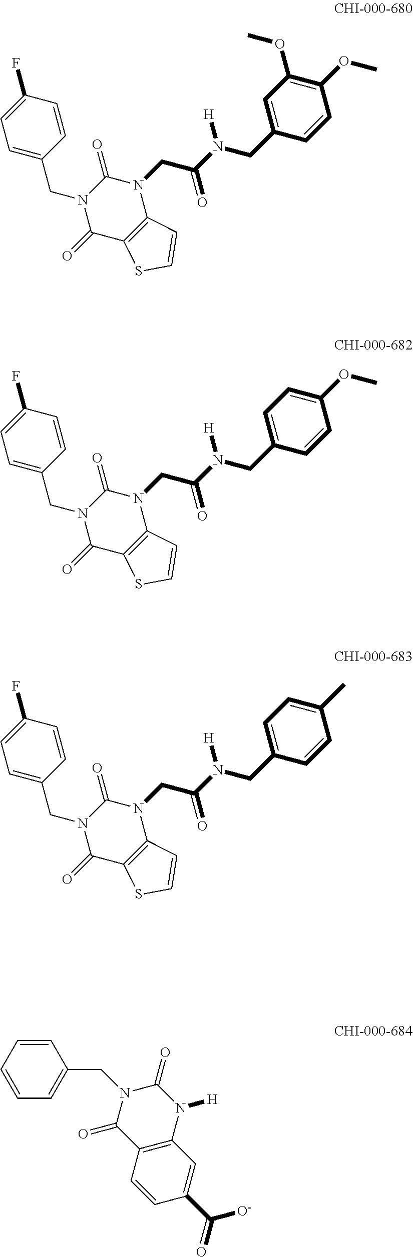

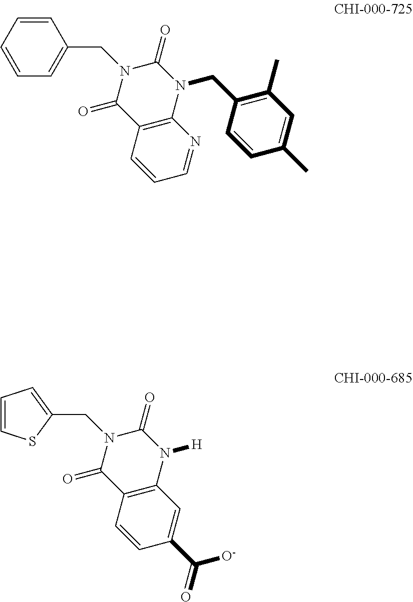

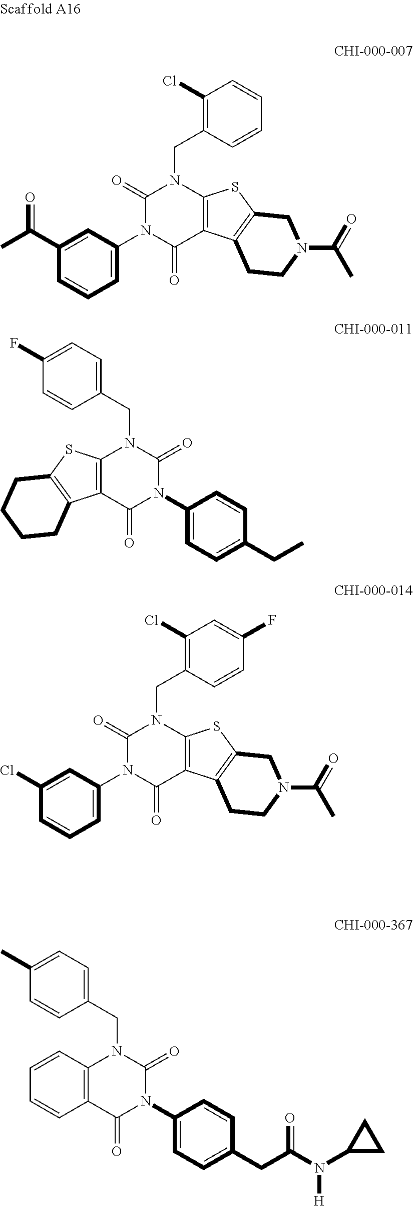

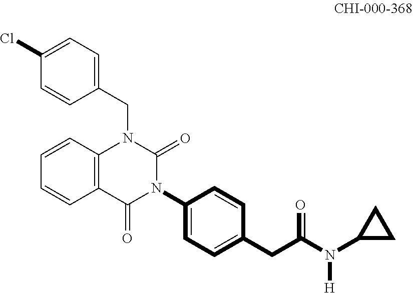

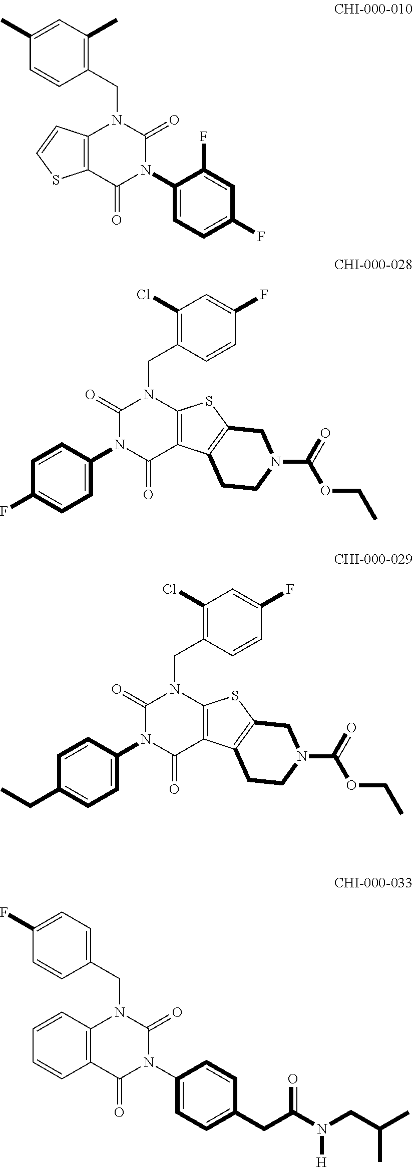

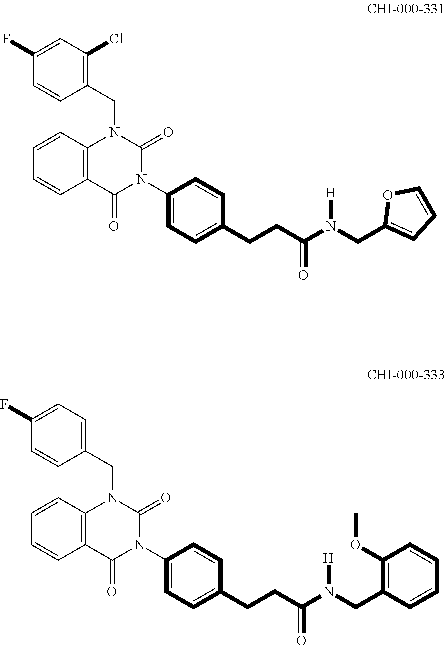

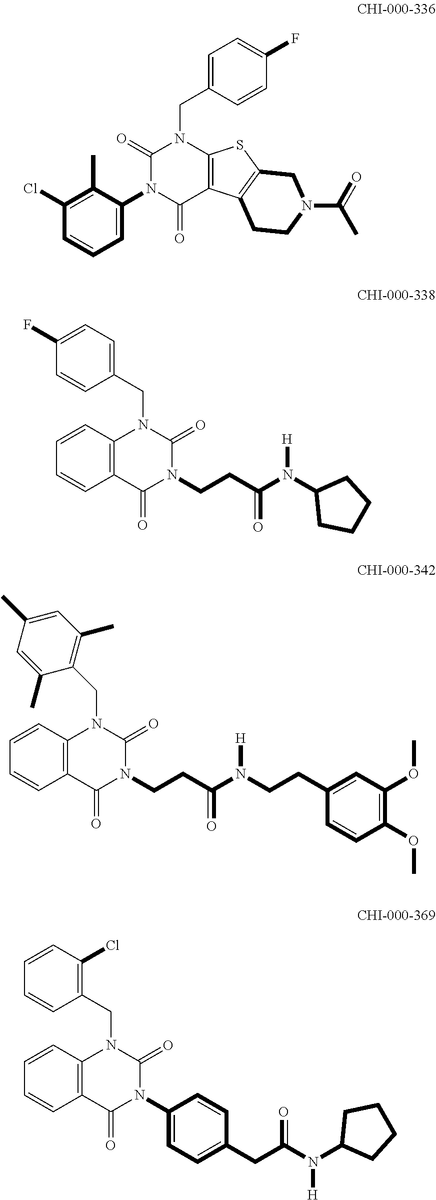

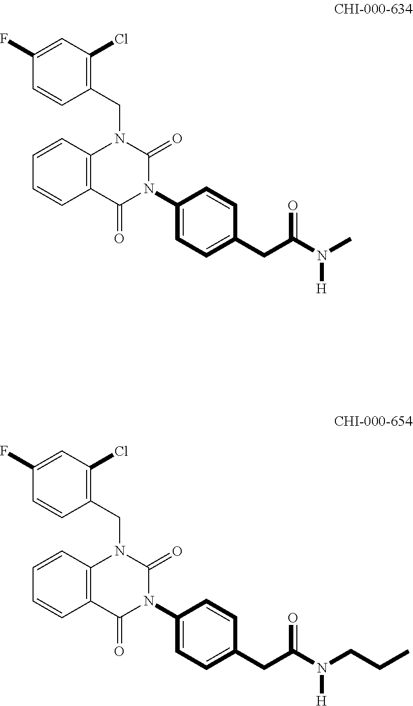

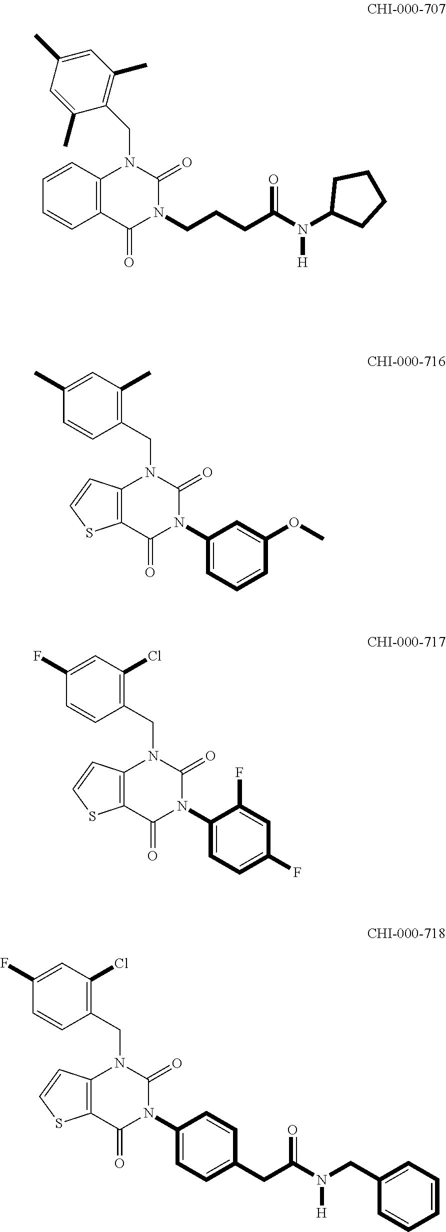

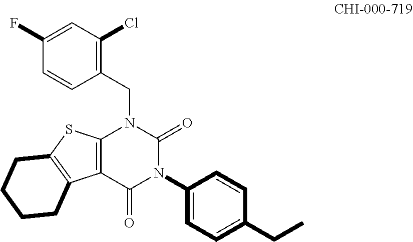

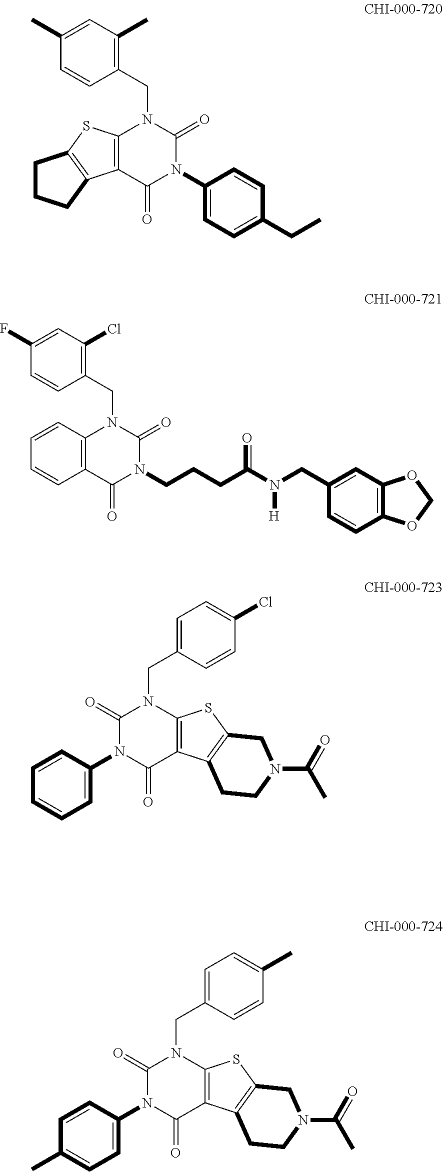

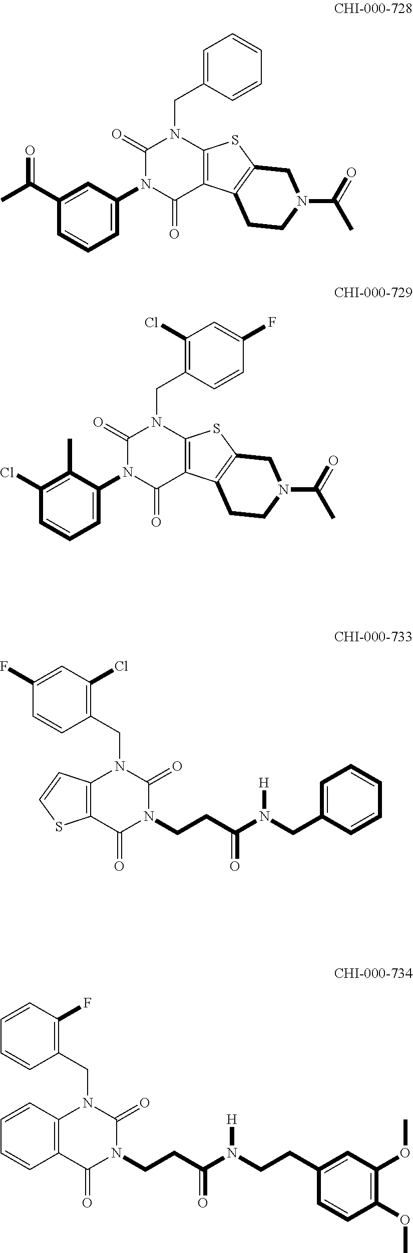

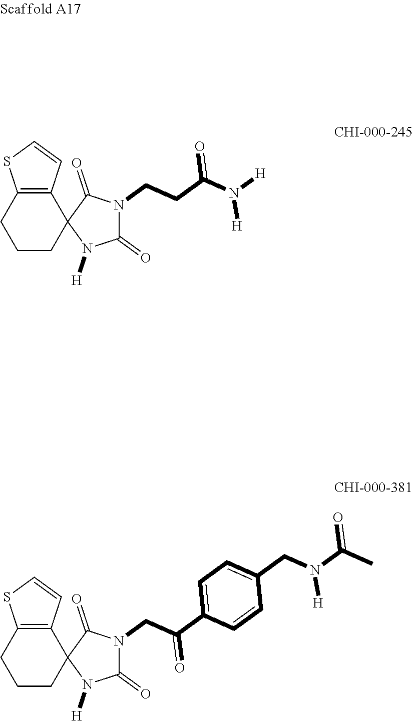

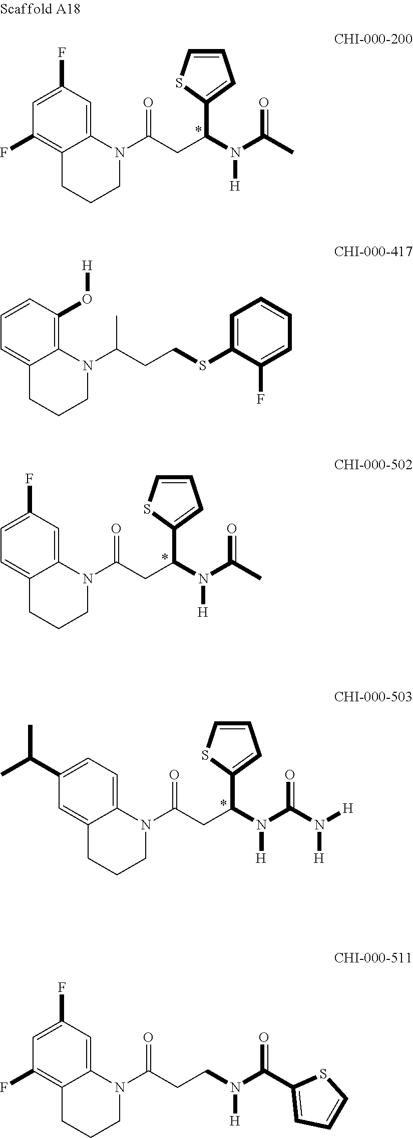

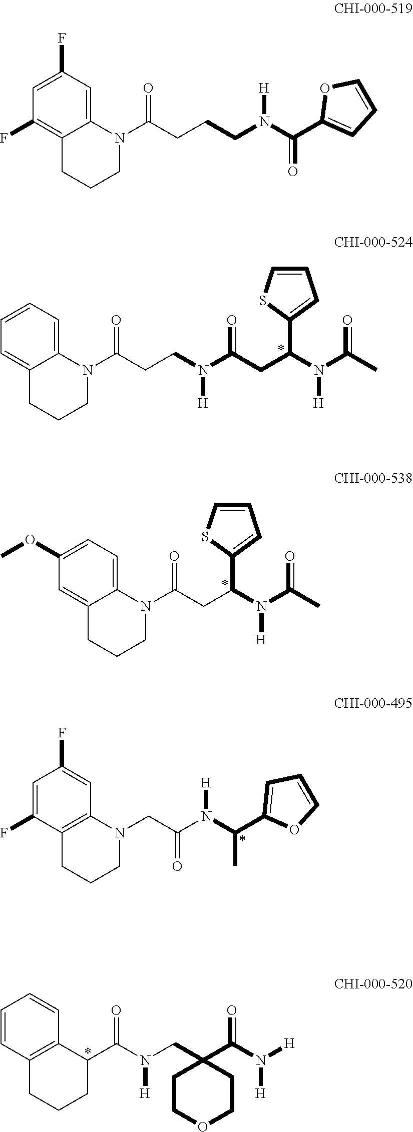

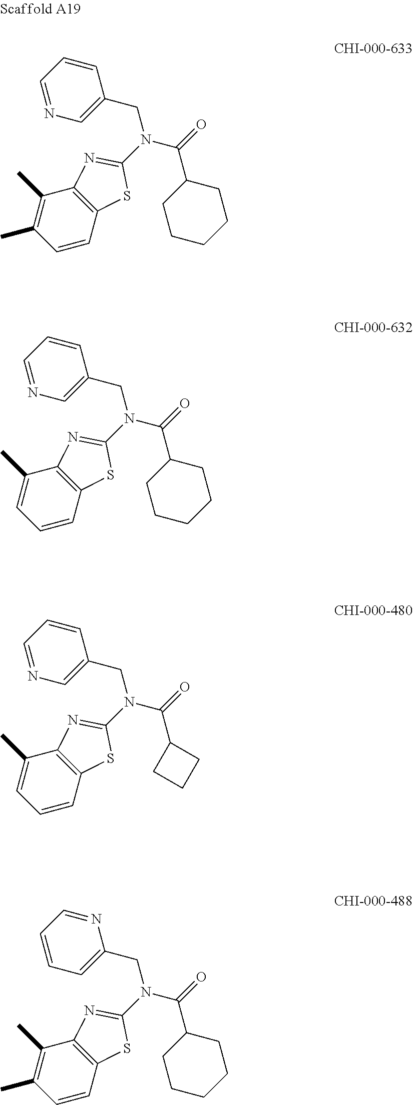

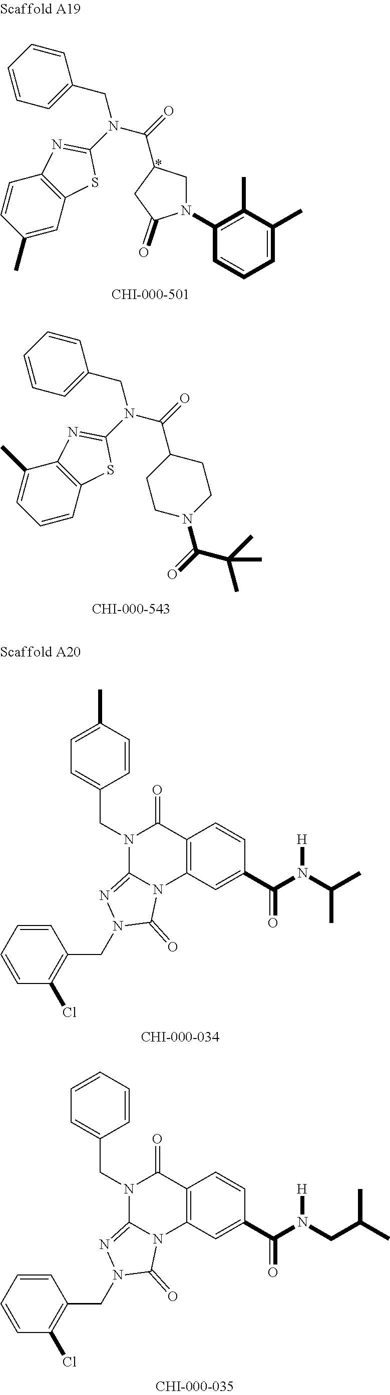

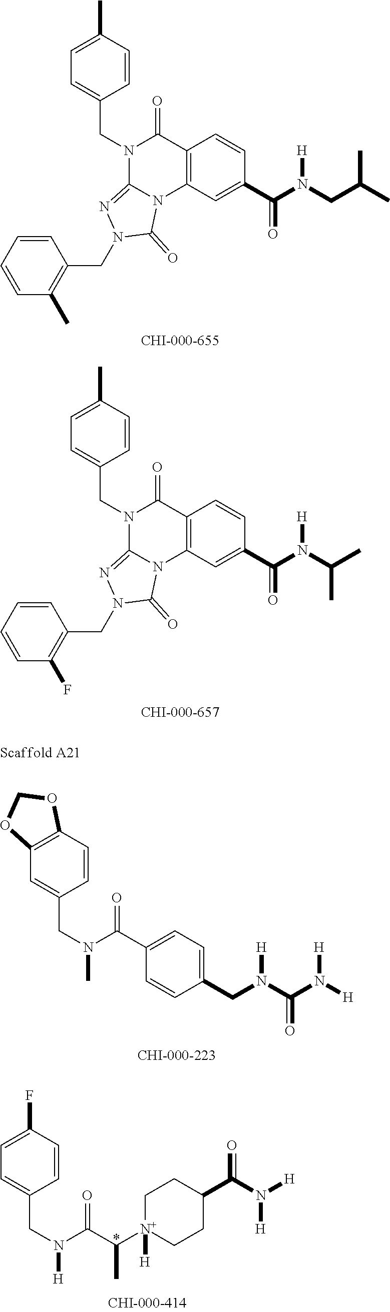

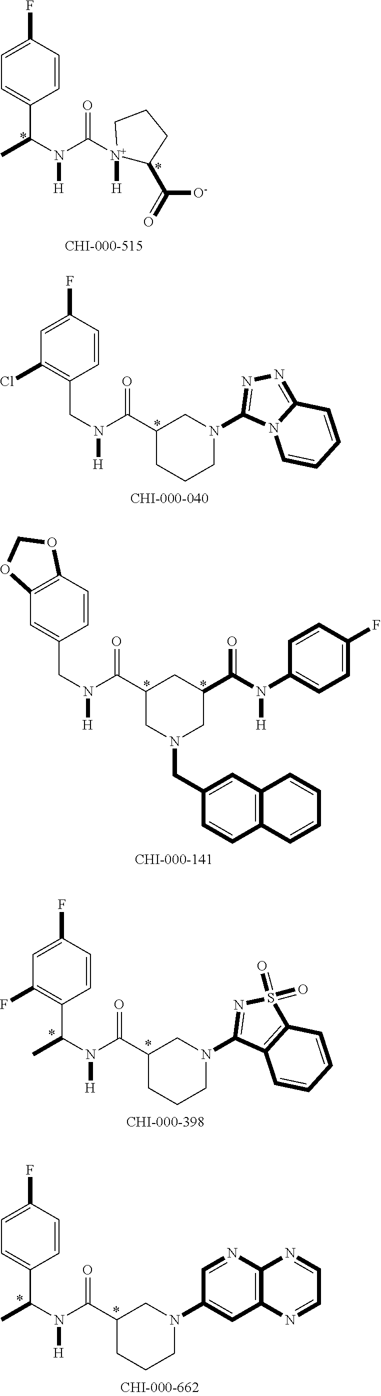

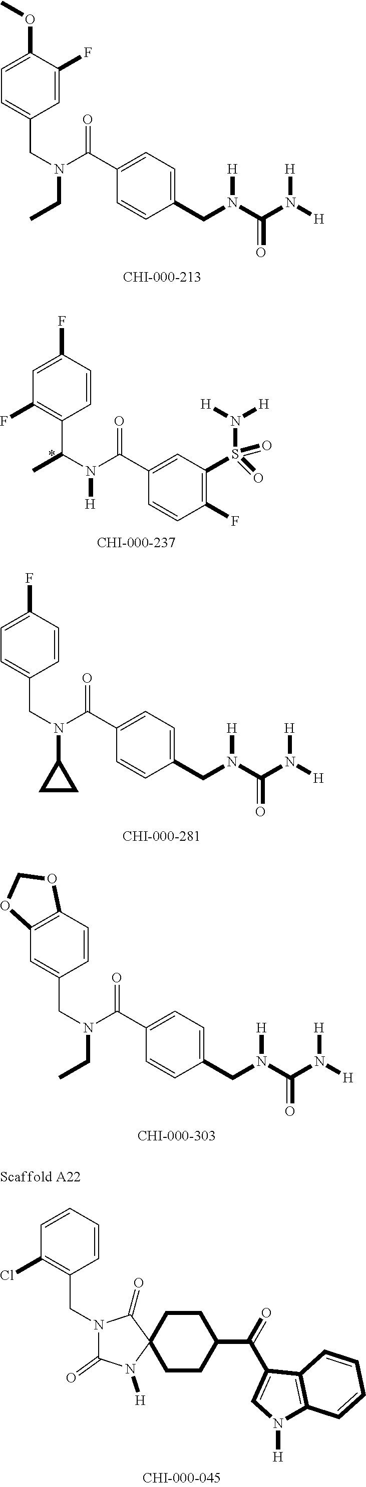

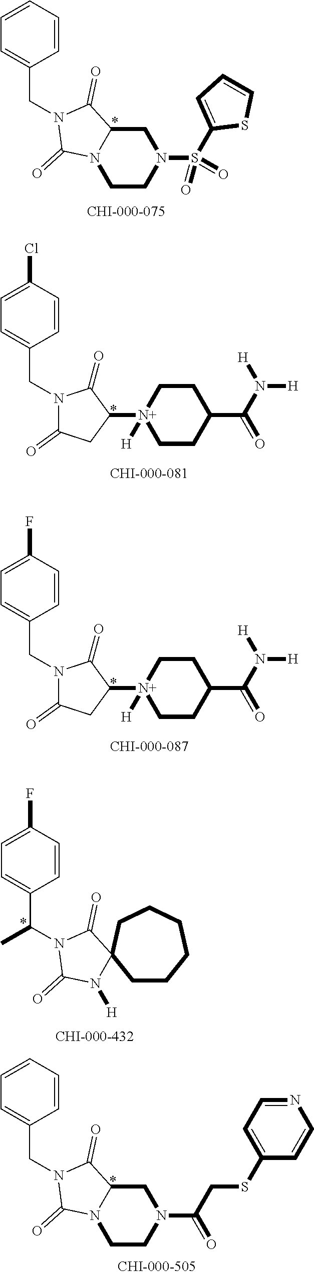

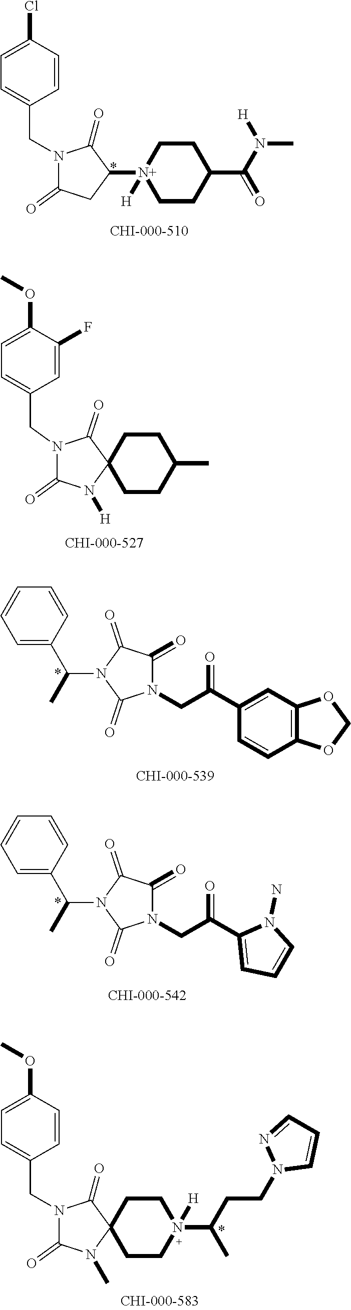

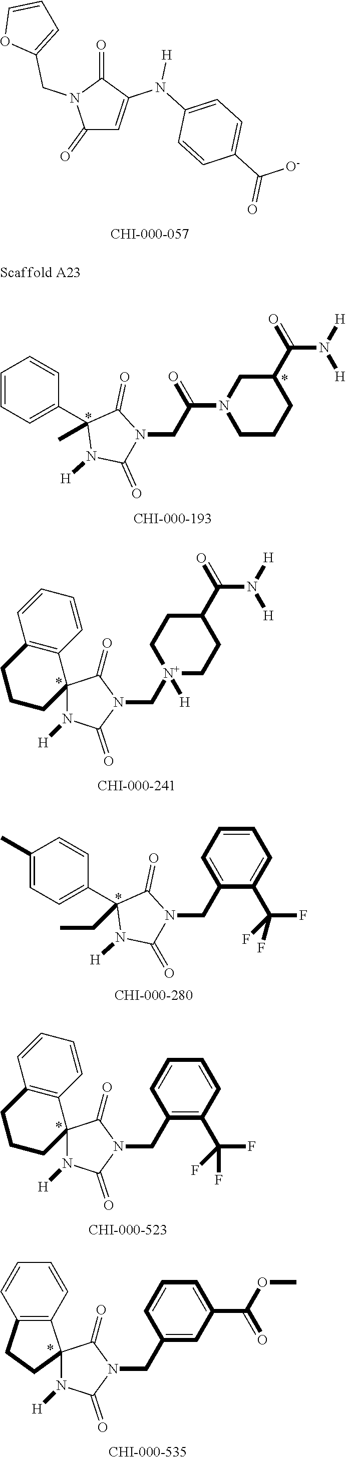

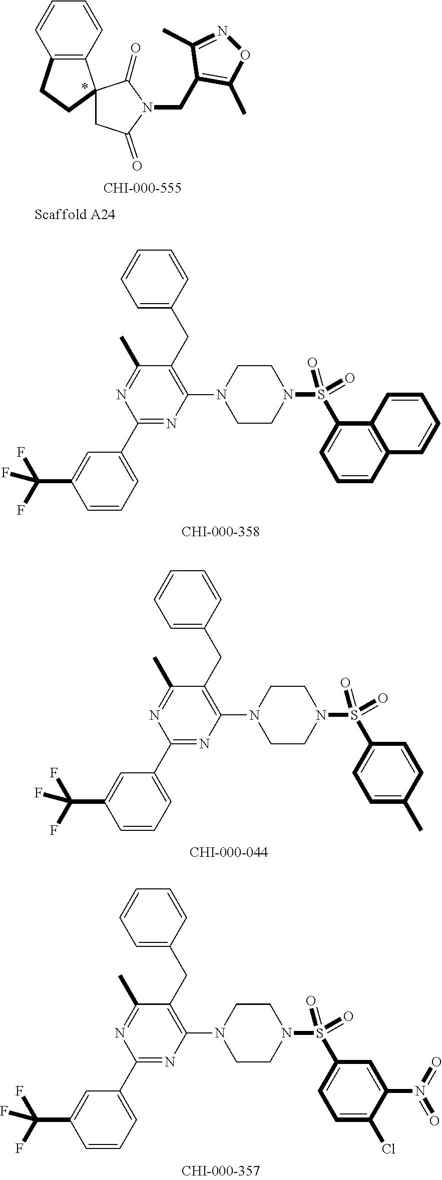

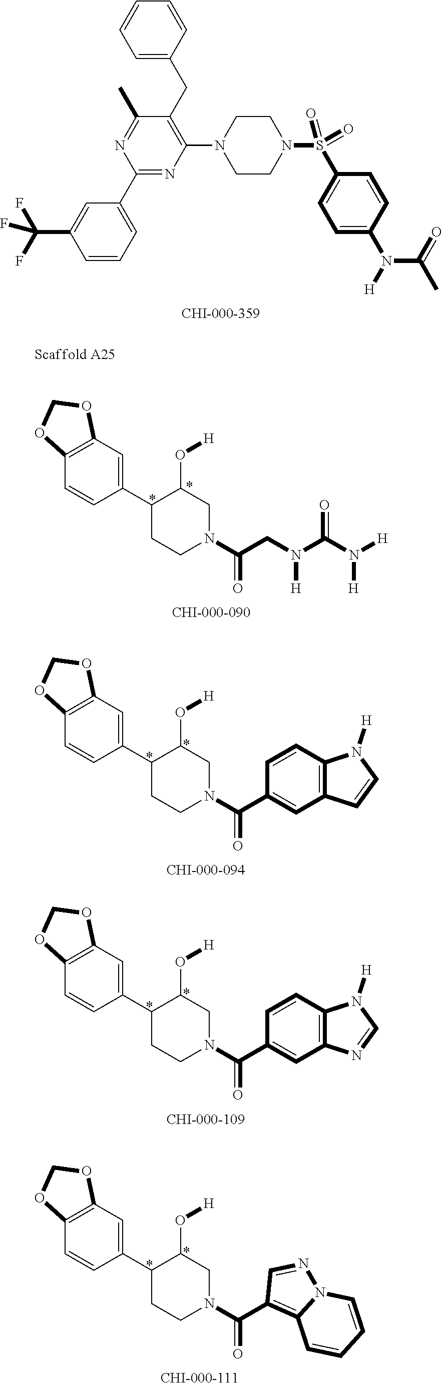

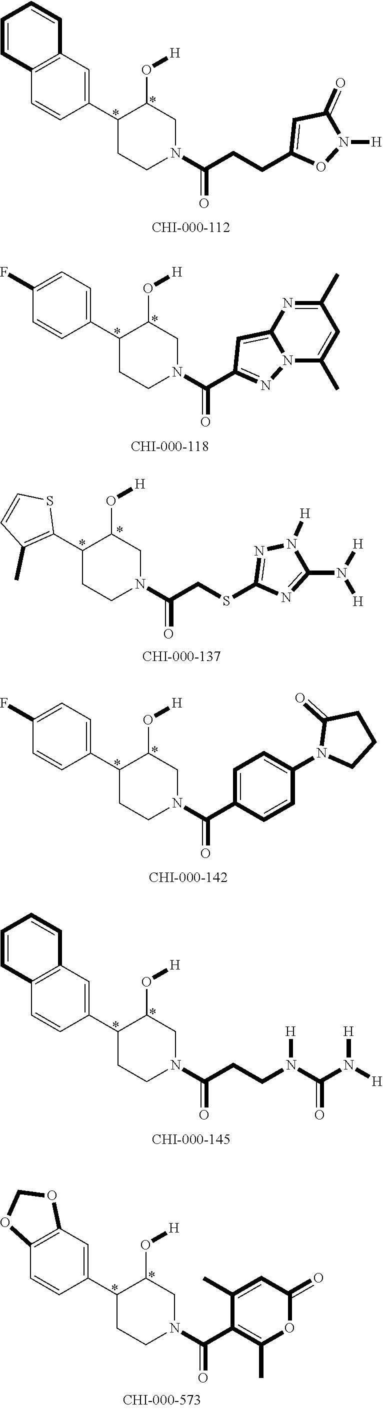

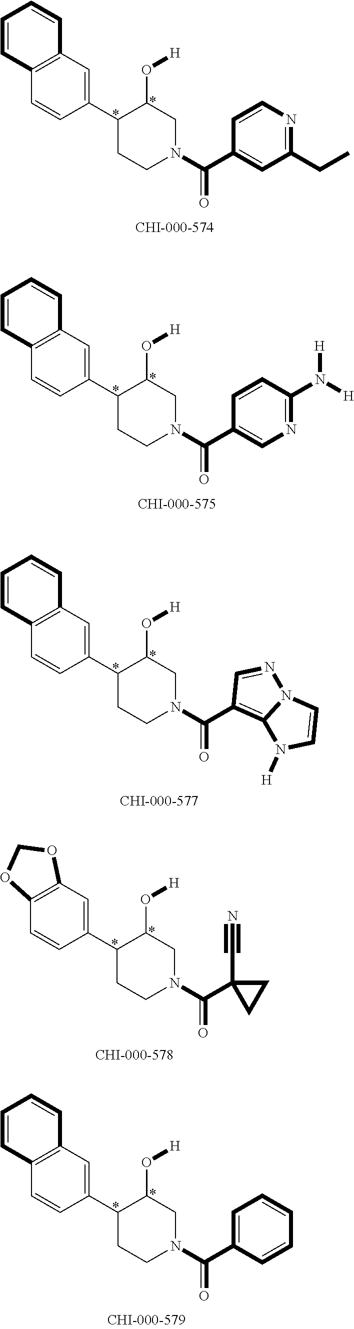

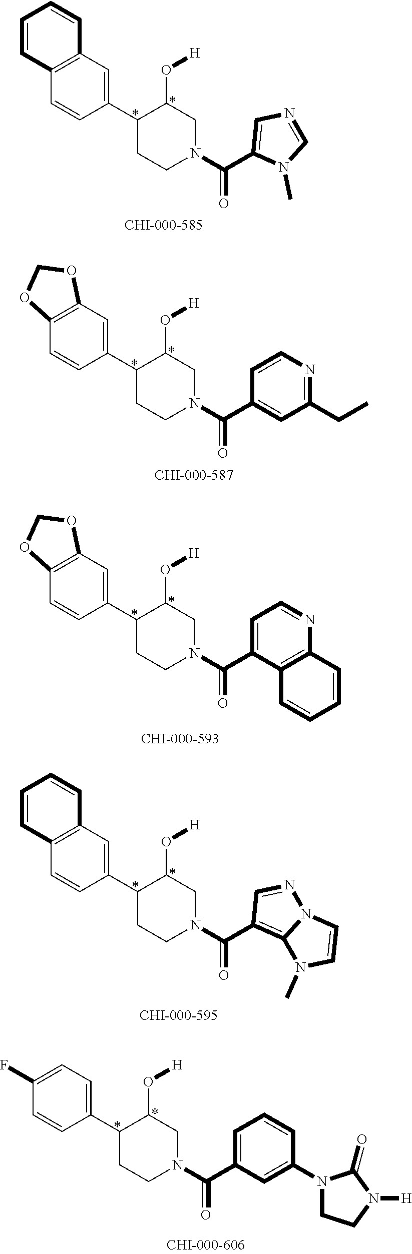

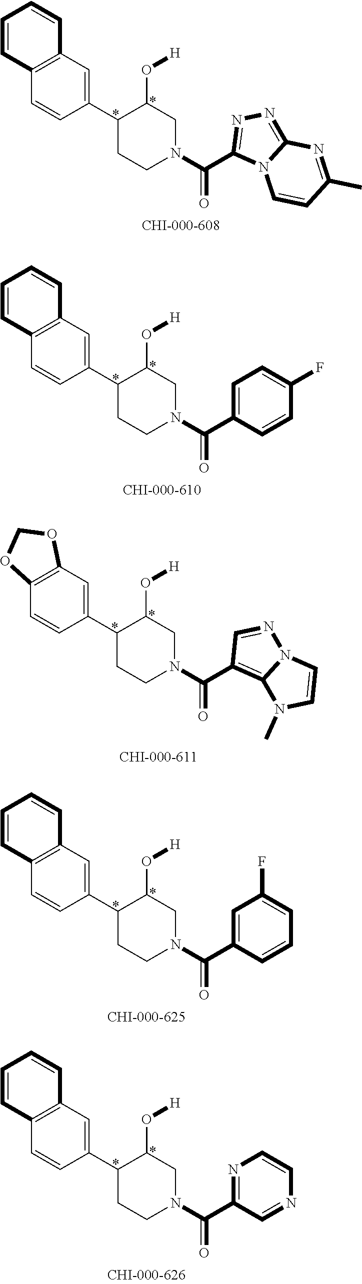

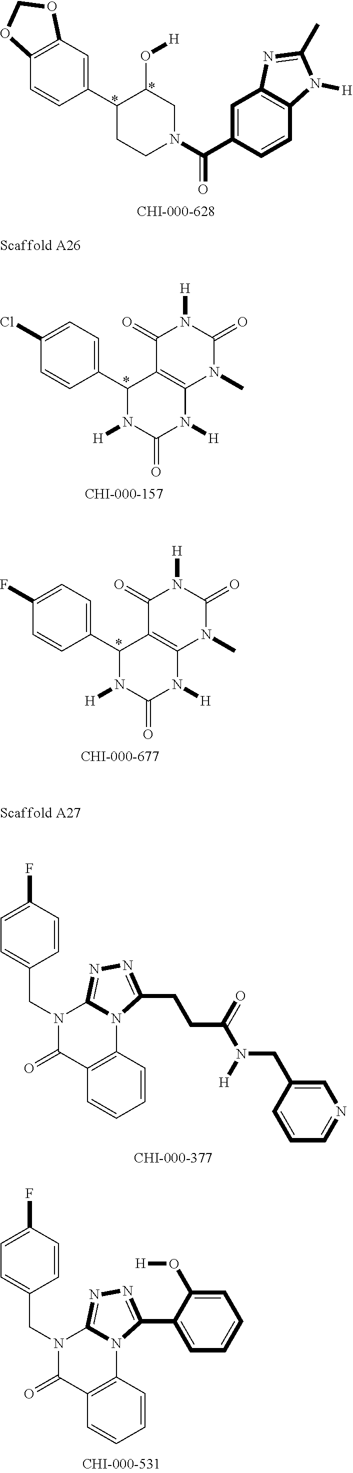

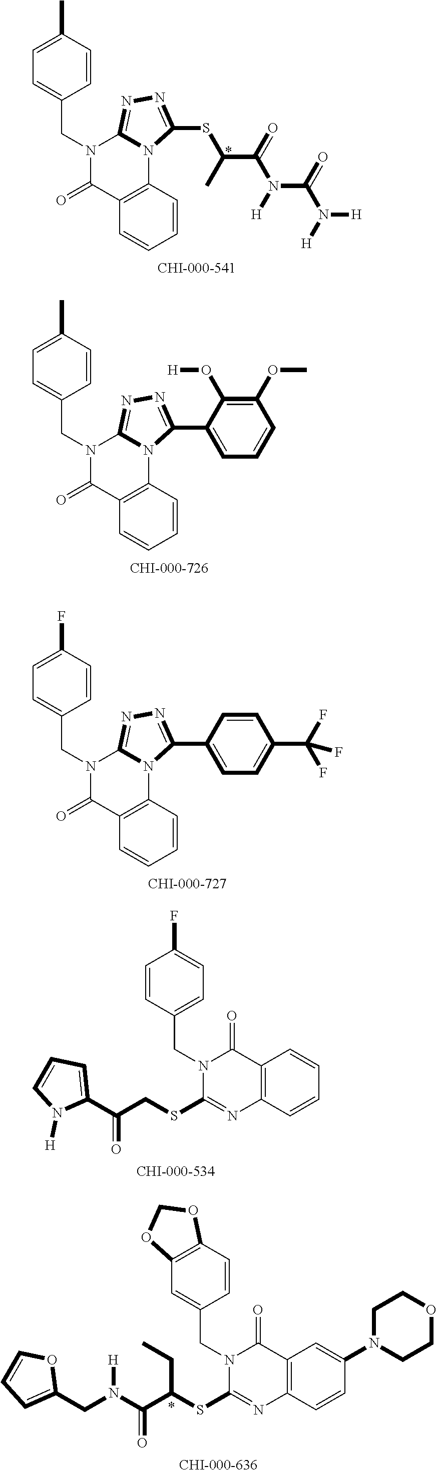

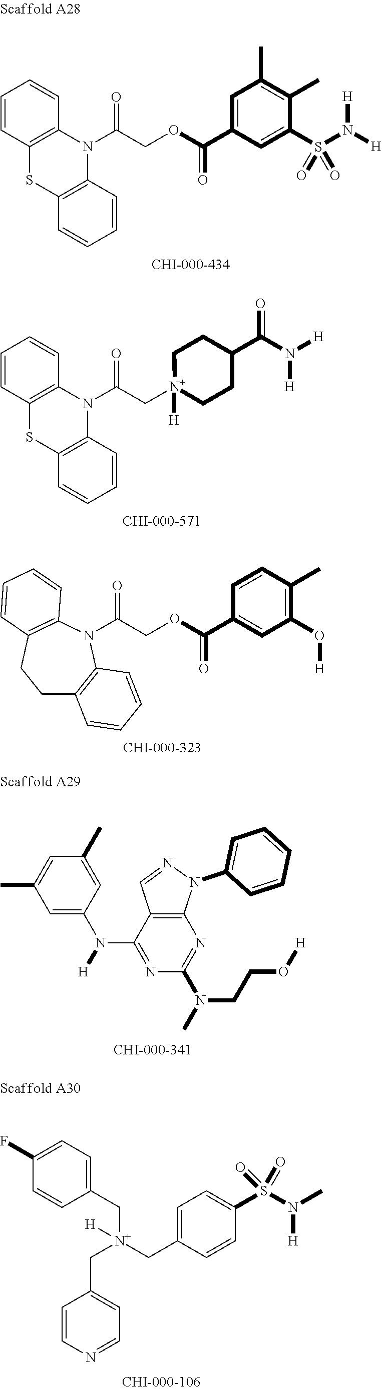

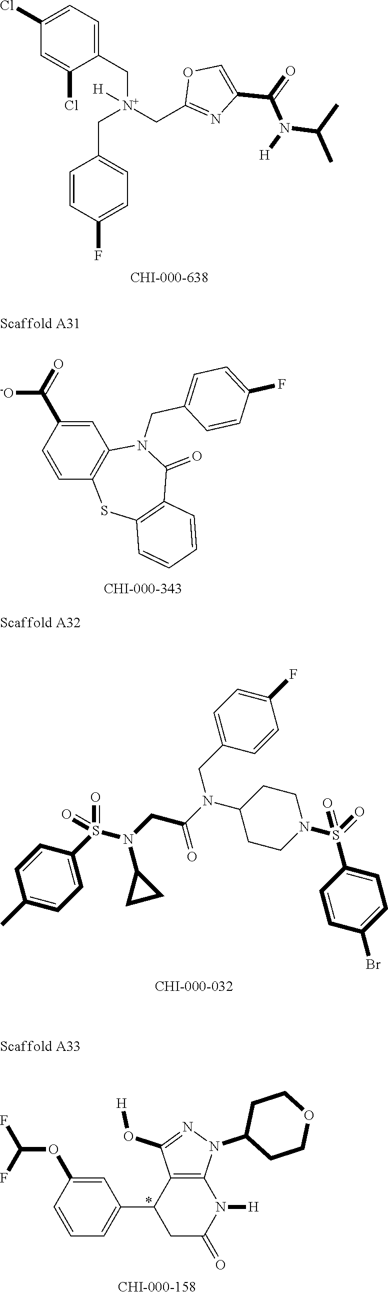

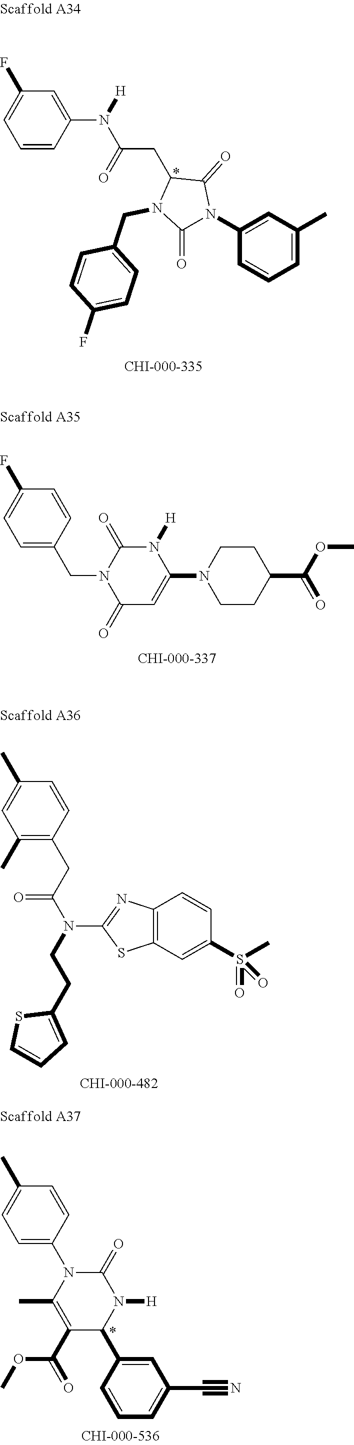

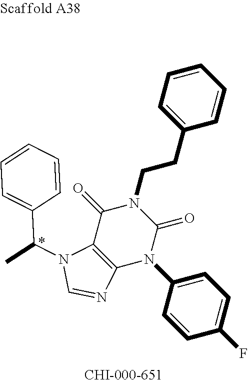

1. A pharmaceutical composition comprising a pharmaceutically acceptable carrier or excipient and a compound according to any of Embodiments EC-ELL, or a pharmaceutically acceptable salt thereof.

2. A method of treating cancer, comprising administering an effective amount of the compound or salt of any one of embodiments EC-ELL to a patient in need thereof.

3. The method of claim 2, wherein the cancer is associated with K-Ras wild-type or mutations, preferably, but not limited to K-Ras(G12C).

4. The compound of any one of embodiments EC-ELL, wherein the compound or salt non-covalently binds K-Ras wild-type or mutants, and preferably modulates the binding of GDP or GTP to K-Ras protein.

5. A pharmaceutical composition comprising a pharmaceutically acceptable excipient and a compound or salt of any one of embodiments EC-ELL.

6. A method of treating cancer, comprising administering an effective amount of a compound or salt of any one of embodiments EC-ELL to a patient in need thereof.

7. The method of claim 6, wherein the cancer is associated with a wild-type or mutant K-Ras.

8. The method of claim 6, wherein the cancer is associated with K-Ras(G12C).

9. A method of treating a disorder in a subject in need thereof, comprising:
a. determining the presence or absence of a K-Ras mutation in a malignant or neoplastic cell isolated from the subject; and
if the K-Ras mutation is determined to be present in the subject, administering to the subject a therapeutically effective amount of a compound of any one of embodiments EC-ELL, wherein the compound or salt non-covalently binds K-Ras wild-type or mutants.

10. A method of modulating an activity of a K-Ras protein, comprising contacting a K-Ras protein with an effective amount of a compound or salt of any one of embodiments EC-ELL.

11. The method of claim 10, wherein the K-Ras protein is K-Ras(G12C).

12. A method to increase the sensitivity towards inhibitors by introducing mutations into K-Ras proteins, comprising contacting a K-Ras protein with an effective amount of a compound or salt of any potential inhibitor.

13. A method to increase the sensitivity towards inhibitors when testing K-Ras proteins by introducing a small molecule primer, comprising contacting a K-Ras protein with an effective amount of a compound or salt of any potential inhibitor.
